SEQUENCE LISTING (1092 sequences)

SEQ ID NO: 1 - DNA, 713 bp, Abutilon theophrasti
tccataatccaacgcagacatttcagagattcatccccgaagaaatcaaaaatccctcaacaaaatgattcagatatgcttaaacaaaatatgaattgagcacaatatcatatttgacatttatgttgaagtagcaaaggttcgtatagtttatgtatgtgcaattaaaactaacagtaacggaattcattctgacactccctatatgctgctagcaactacctcgatctctcaagcctcgacaacggtggtggattctgtagattgtttggcttcaagagacttctcgtattcttcaatggatttgaagagctcggaaaagttgcctttcccgaaacctccgcatcctcctttctggtattgctttccttcttcatccttcaccatgcaccctaatctctgtattatttctatgaatatcgtaggcctatctccaatgggcttagtgaaaatctgaagcagagtgccttgatcatctctgtcaaccaaaatccccaactcttcacactccttaatctgctcatcgctcaaaatgtcccctgccctttgcttcaatttcttatagtaagtgggcggcggcgatggcatgaactcgaaaccacccaccaaacttctcttcctcatttctctcagcgttctgaatatatcttcactcaccagagccaaatgttgaaccccggcaccttcgttgtgttctaaatacgtttggattt

SEQ ID NO: 2 - DNA, 1234 bp, Amaranthus graecizans
cttcttccccgtctgagtttttattacttcactttctctctcatcatctaacatgggaactttgaaacccgaaactcaacccgactccgaattcaaactcgtgggttactccaacttcattcgggttaaccccaaatctgaccgttttactgttaagcgtttccatcatatagagttctggtgtggcgatgcaaccaatgttagcagacgcttttcttggggtcttggaatgcctaccgttgctaaatctgacctttctactggaaactctgttcacgcttcttttcttcttcgttccggtgacctttctttcctctttacttcaccttactctcctaccatgtccatcccttcttctgctgcaatcccctcgtttgatttcaatcattttaccaaatttgttacatcgcacggtcttggcgtgcgtgctgttgccgtcgaagtagaagacgcagaagctgcttttaatatcagcgtttcgcacggggctatcccctgtgtttctcctattcacttggaaaacggtgtcgttttatctgaggttcatttatatggggatgttgtgcttcgttatgtaagctacggaaatgaatgtggggatgtgttttttcttcctgggtttgagcaaatgcccgaggaatcctcgtttcgaggacttgatttcggccttcgaaggttggatcatgctgtagggaatgtccctgagttggctcctgcaattgcttatttgaagaagtttactgggtttcatgagtttgctgagtttacagctgaagatgttggaacgagtgaaagtgggttgaattcagccgtattggcaaataatgatgaaatggtgttgtttccaatgaatgaacctgtgtatgggacaaaaaggaagagtcaaattcaaacttatttggagcataatgaaggagctggtgtacaacatttggctttgatgagtgaagatatattttggactttaagggagatgaggaagagaagtggtcttggtgggtttgagtttatgccgtcgccgcctccgacttattaccggaatttgaggaacagagctgctgatgtattgagtgaggagcagatgaaggagtgtgaagagttggggattttggtggataaagatgatcagggtactttgcttcaaatcttcactaagcctattggtgacaggccaaccatatttatcgagattatacaaagaatcggttgcatgatgaaagatgaagacggcaag

SEQ ID NO: 3 - DNA, 547 bp, Amaranthus hybridus
tcttcctgggtttgaggaaatgccggaggaatcgtcgtttcgaggacttgatttcggccttcgaaggttggatcatgctgtagggaatgttcccgagttggctcctgcaattgcttatttgaagaagtttactgggtttcatgagtttgctgagtttacagctgaagatgttgggacgagtgaaagtgggttgaattcagccgtattggcaaataatgatgaaatggtgttgtttccaatgaatgaacctgtgtatgggacaaaaaggaagagccaaattcaaacttatttggagcataatgaaggagctggtgtacaacatttggctttgatgagtgaagatatattttggactttaagggagatgaggaagagaagtggtcttggtgggtttgagtttatgccgtcgccgcctccgacttattaccggaatttgaggaatagagctgctgatgtattgagtgaggagcagatgaaggagtgtgaagagttggggattttggtggataaagatgatcagggtactttgcttcaaatcttcactaag

SEQ ID NO: 4 - DNA, 1265 bp, Amaranthus palmeri
agatgtgtataagagacaggtcttggcgtgcgtgctgttgccgtcgaagtagaagacgccgaagctgcttttaatatcagcgtttcgcacggggctatcccctgtgtttctcctattcacttggaaaacggtgtcgttttatctgaggttcatttatatggggatgttgtgcttcggtatgtaagctacggaaatgaatgtggggatgtgtttttcttcctgggtttgaggaaatgccggaggaatcatcgtttcgaggacttgattttggcattcgaaggttggatcatgctgtagggaatgtccctgagttggctcctgcaattgcttatttgaagaagtttactgggtttcatgagtttgctgagtttacagctgaagatgttgggacgagtgaaagtggattgaattcagccgtattggcaaacaatgatgaaatggtgttgtttccaatgaatgaacctgtgtatgggacaaaaaggaagagccaaattcaaacttatttggagcataatgaaggagctggtgtacagcatttggctttgatgagtgaagacatattttggactttaagggagatgaggaagagaagtggtcttggtgggtttgagtttatgccgtcgccgcctccgacttattaccggaatttgaggaacagagctgctgatgtattgagtgaggagcagatgaaggagtgtgaagagttggggattttggtggataaagatgatcagggtactttgcttcaaatcttcaccaaacctattggagacaggccaaccatattcatcgagattatccaaagaattggttgcatgatgaaagatgaagacggcaagatgtaccaaaagggtggttgcggaggattcggaaagggaaacttttcagagctgttcaaatcaatagaggagtacgagaagactcttgaacgtaaacaggttccagatacagctgctgcatgatgagcagactaaaatattgttgtcttgctgatgaaatgatagaaaaggtttgtttcttggtacaatgctcaacttcaaaattttctttattaaataatgaagtgtaaacttatacaaactgtgtcatatatggtgattggtgatcatggtttatgtagaatgtataacataattgataatctgtgtatatgctgaatacttacatactgtgaaatcattctgatggaaacatacaattggtcagtagctgaggctggtagctcctcagattagtttttttcagtcaaaatcgtagatgtatat

SEQ ID NO: 5 - DNA, 3245 bp, Amaranthus palmeri
ttttctcttttatttttatccaaaaatatcatgggatgtctagattggaataattagggagtaaaaagtaccccttgattatgcagagcaaaaataaattgtctgaatttgaaataaatatctacaagagtaaatttttccatcttattcaaaggtaaatgtttgatccacctacctctatagatatatttcagggaactaaattgtcttcaccattaaatttggttacttggtctttaaaacccaaatcatagaaattaatgataaaatcaaaataaaaaagatatttaaattcaaattcaaactaactaattttaaattacaaaatgaatatctgtaatttacaaaagaaagtatcaaaaacatatgaaaatctcaacatctgaaaattacaaacaagtattctgttttttcattttttttttctttttcgctatttcctttcaaaaataaaagtaaataaaaatattcaaaagcaattccataaacaaaatcttagatatgtaaatcacaaaaacattagatctagaaaaaaaaaatttcttccattgcaaaccctttttcaaccttcataacttccactaccataatgaggccagtaaagagacaaaagtcattgagttgttgttgtgcagttgatgataaatgatgatagaagggtttattttttttttgaaatgaatggttagattttctgactttttatttaccctataatgaatatcaaacaattaactctataaattatttaatacattaaaatgtttcatgtaatatgtctcctatattatttaccctttaatttttaagtgggaaccaagtatgtcttaattatctttattttaatcaaatacgcggtatacatgaaataatcaacaaatgcaattactatgctcggacgagagtaaatataatgggaggaagttgtacatacaattacgaaatagtctaaataaataacgataatttgtaatataaacaaacaaatcacacttatataaacagattttatagggtggaatcattaggattctaatttatcttttttcttcttttgtttactttgctgatatttattttgtatttttcctatttctcaaaaggaagactaacactcaaataaaatgatattgaaatacaaagcatcaccggccaagccgagatgacgaaactattttgaataattatgatgatttacaactccaaatagaagtaattgatcaagactttaggacttggaagtgttgggcaaaatcttccagagtccaggataagtgataagtgacgtatttccgttactcttaagtgttaacagctttttgtcacgcaaggaaaagaagaccgtggacgtcaacgatgacgttgaatgttcatctttacagtcgcagtcaatcaatctctttttagatcgatcttccacctcaattctccgttacaaatcaaattccatctagaacttcttttttattattttgactcataaattcccccaaaaatacttctattttattataaataaattccaatttctatgttctccattcattaccacccattactccgttttccaaaccaccattttctctctcctcctttaccgctaacgctaccaccattttcgcttcttccccgtctgaattttattacttcgctttctctatcatcatctgacatgggtactttgaaacccgaaactcaacccgattccgaattcaaactcgtgggttactccaacttcgttcgggttaaccccaaatctgaccgttttgctgttaagcgtttccaccatatagagttttggtgtggcgatgcaaccaatgttagcagacgattttcttggggtcttggaatgcctaccgttgctaaatctgacctttctacaggaaactctgttcacgcttcttttcttcttcgttccggtgacctttcttttctctttacttcaccttactctcctaccatgtccatcccttcttctgctgcaatcccctcgtttgatttcaatcattttaccaaattccttacatcgcacggtcttggcgtgcgtgctgttgccgtcgaagtagaagacgcggaagctgcttttaacatcagcgtttcgcatggggctattccctgtgtttctcctattcaattggaaaacggtgtcgttttatctgaggttcatttatatggggatgttgtgcttcggtatgtaagctacggaaatgaatgtggggatgtgttttttcttcctgggtttgaggaaatgccggaggaatcatcgtttcgaggacttgattttggcattcgaaggttggatcatgctgtagggaatgtccctgagttggctcctgcaattgcttatttgaagaagtttactgggtttcatgagtttgctgagtttacagctgaagatgttgggacgagtgaaagtggattgaattcagccgtattggcaaacaatgatgaaatggtgttgtttccaatgaatgaacctgtgtatgggacaaaaaggaagagccaaattcaaacttatttggagcataatgaaggagctggtgtacagcatttggctttgatgagtgaagacatattttggactttaagggagatgaggaagagaagtggtcttggtgggtttgaatttatgccgtcgccgcctccgacttattaccggaatttgaggagcagagctgctgatgtattgagtgaggagcagatgaaggagtgtgaagagttggggattttggtggataaagatgatcagggcactttgcttcaaatcttcaccaaacctattggagacaggccaaccatattcatcgagattatccaaagaattggttgcatgatgaaagatgaagacggcaagatgtaccaaaagggtggttgcggaggatttggaaagggaaacttttcagagttgttcaaatcaattgaggagtacgagaagactcttgaacgtaaacaggttccagatacagctgctgcatgatgagcagactgaaatattgctgtcttgctggtggaagccatataatggtaatatgatagaaaaggtttgttgctcaaaattttctttattaaataatgaagtgtaaacttatacaaactgtgtcatatatggtgattgatgatcatgcatggttatgtagaatgtataacataattgataatctgtgtatatgctgaaaacttacatactgtgaaatcattctgatagaaacatacaattggtgagtagctgtctcttatacacatct

SEQ ID NO: 6 - DNA, 3416 bp, Amaranthus palmeri
ggaggaatttttttgtgcatgtaaaatgttttctctctattttttgatttatgctattttttctcttttatttttatccaaaaatatcatgggatgtctagattggaataattagggagtaaaaagtaccccttgattatgcagagcaaaaataaattgtctgaatttgaaataaatatctacaagagtaaatttttccatcttattcaaaggtaaatgtttgatccacctacctctatagatatatttcagggaactaaaattgtcttcaccattatatttagttacttggtctttaaaacccaaatcatagaaattaacgataaaatcaaaatataaagatatttaaattccaattcaaactaactaattttaaattacaaaatgaatatctgtaatttacaaaagaaagtatcaaaaacatatgaaaatctcaacatctgaaaattaaaaaacaagtattttgtttcttcatttttttctttttcgctatttcctttcaaaaataaaagtaaataaaaatattcaaaagcaattccataaacaaaatcttagatatgtaaatcacaaaaacattagatctagaaaaaaaaaatttcttccattgcaaaccctttttcaaccttcataacttccactaccataatgaggccagtaaagagacaaaagtcattgagttgttgttgtgcagttgatgataaatgatgatagaagggtttattttttttttgaaatgaatggttagattttctgactttttatttaccctatatagaatatcaaacaattaactctataaattatttaatacattaaaatgtttcatgtaatatgtctcctatattatttaccctttaatttttaagtgggaaccaagtatgtcttaattatctttattttaatcaaatacgcggtatacatgaaataatcaacaaatgcaattactatgctcggacgagagtaaatataatgggaggaagttgtacatacaattacgaaatagtctaaataaataacgataatttgtaatataaacaaacaaatcacacttatataaacagattttatagggtggaatcattaggattctaatttatcttttttcttcttttgtttactttgctgatatttattttgtatttttcctatttctcaaaaggaagactaacactcaaataaaatgatattgaaatacaaagcatcaccagccaagccgagatgacaaaactattggctaagtgataactgataagtgacgtatttccgttactctcaagtcttaacagctttttgtcacgcaaggaaaagaagaccgtggacgtcaacggtgacgttgaatgttcatctttacagtcgcagtcaatcaatctctttttagatcgatcttccacctcaattctccgttacaaatcaaattccatctagaacttctttttaattattttgactcataaattcccccaaaaatacttctattttattataaataaattccaatttctatgttctccattcatcaccacccattactccgttttccaaaccaccattttctctctcctccattacccctaacacaactaccattttcgcttcttccccgtctgagtcttattacatcgctttctctctcatcatctgacatgggaactttgaaacccgaaactcaacccgattccgaattcaaactcgtgggttactccaacttcgttcgggttaaccccaaatctgaccgttttgctgttaagcgtttccaccatatagagttttggtgtggcgatgcaaccaatgttagcagacgattttcttggggtcttggaatgcctatcgtcgctaaatctgacctgtctacaggaaactctgttcacgcttcttttcttcttcgttccggtgacctttcttttctctttacttcaccgtattctcctaccatgtccatcccttcttctgctgcaatcccctcgtttgatttcaatcattttaccaaattccttacatcgcacggtcttggcgtgcgtgctgttgccgtcgaagtagaagacgccgaagctgcttttaatatcagcgtttcgcacggggctatcccctgtgtttctcctattcacttggaaaacggtgtcgttttatctgaggttcatttatatggggatgttgtgcttcggtatgtaagctacggaaatgaatgtggggatgtgttttttcttcctgggtttgaggaaatgccggaggaatcatcgtttcgaggacttgattttggcattcgaaggttggatcatgctgtagggaatgtccctgagttggctcctgcaattgcttatttgaagaagtttactgggtttcatgagtttgctgagtttacagctgaagatgttgggacgagtgaaagtggattgaattcagccgtattggcaaacaatgatgaaatggtgttgtttccaatgaatgaacctgtgtatgggacaaaaaggaagagccaaattcaaacttatttggagcataatgaaggagctggtgtacagcatttggctttgatgagtgaagacatattttggactttaagggagatgaggaagagaagtggtcttggtgggtttgaatttatgccgtcgccgcctccgacttattaccggaatttgaggagcagagctgctgatgtattgagtgaggagcagatgaaggagtgtgaagagttggggattttggtggataaagatgatcagggcactttgcttcaaatcttcaccaaacctattggagacaggtagattttaatcttgctttcaattgcttttgcttgattgattgactagccaattttgattgcattttgttgcttatatgacttgatgataatagatggtttacctttctcagctgttcatttgtagccagtatagattcgttctaaaatatttgcaactgattatgacatgtagtagcagaaaatgtccctatattggatgtttggcataaattaagcttggttttgcacttatcctcatttatttataaattctaaaacttgttagttgtaattaagttaatgagaacaaagccttaatattccttcaaggtgattgtagttggggcactagttctaacaatggaaatttggaaatctattccaactggtcccaagttaatctttgtttgcaagcctgattggttcaaattaaggttattgtattcttgtatgaattcgactcaatgtaaatttgtttaatggagcatcaatttttaatagttttcgaccaagcagtattagatatattcgggattgaaataatgcatctatgagtgtataaaaccaaatggcacatttgattagaataaaagagagtataaggctaatttcgtttacctaatatttaaagcgacccctaaaattcaattggcctaaacccataaagttcaaa

SEQ ID NO: 7 - DNA, 3818 bp, Amaranthus palmeri
gagttcaagattaaaagttaaattacatttatgtgggttttatataggcaatgattgcattatattgtttttcttttggtgggaagattttcctttttaaaaaattttaatttccctacattttcaaatgatggaggaatttttttgtgcatgtaaaatgttttctctctattttttgatttatgctattttttctcttttatttttatccaaaaatatcatgggatgtctagattggaataattagggagtaaaaagtaccccttgattatgcagagcaaaaataaattgtctgaatttgaaataaatatctacaagagtaaatttttccatcttattcaaaggtaaatgtttgatccacctacctctatagatatatttcagggaactaaattgtcttcaccattaaatttggttacttggtctttaaaacccaaatcatagaaattaatgataaaatcaaaataaaaaagatatttaaattcaaattcaaactaactaattttaaattacaaaatgaatatctgtaatttacaaaagaaagtatcaaaaacatatgaaaatctcaacatctgaaaattaaaaaacaagtattttgtttcttcatttttttctttttcgctatttcctttcaaaaataaaagtaaataaaaatattcaaaagcaattccataaacaaaatcttagatatgtaaatcacaaaaacattagatctagaaaaaaaaaatttcttccattgcaaaccctttttcaaccttcataacttccactaccataatgaggccagtaaagagacaaaagtcattgagttgttgttgtgcagttgatgataaatgatgatagaagggtttattttttttttgaaatgaatggttagattttctgactttttatttaccctatatagaatatcaaacaattaactctataaattatttaatacattaaaatgtttcatgtaatatgtctcctatattatttaccctttaatttttaagtgggaaccaagtatgtcttaattatctttattttaatcaaatacgcggtatacatgaaataatcaacaaatgcaattactatgctcggacgagagtaaatataatgggaggaagttgtacatacaattacgaaatagtctaaataaataacgataatttgtaatataaacaaacaaatcacacttatataaacagattttatagggtggaatcattaggattctaatttatcttttttcttcttttgtttactttgctgatatttattttgtatttttcctatttctcaaaaggaagactaacactcaaataaaatgatattgaaatacaaagcatcaccagccaagccgagatgacaaaactattggctaagtgataactgataagtgacgtatttccgttactctcaagtcttaacagctttttgtcacgcaaggaaaagaagaccgtggacgtcaacggtgacgttgaatgttcatctttacagtcgcagtcaatcaatctctttttagatcgatcttccacctcaattctccgttacaaatcaaattccatctagaacttcttttttattattttgactcataaattcccccaaaaatacttctattttattataaataaattccaatttctatgttctccattcattaccacccattactccgttttccaaaccaccattttctctctcctcctttaccgctaacgctaccaccattttcgcttcttccccgtctgaattttattacttcgctttctctatcatcatctgacatgggaactttaaaacccgaaactcaacccgattccgaattcaaactcgtgggttactccaacttcgtccgggttaaccccaaatctgaccgttttgctgttaagcgtttccaccatatagagttttggtgtggcgatgcaaccaatgttagcagacgattttcttggggtcttggaatgcctaccgtcgctaaatctgacctgtctacaggaaactctgttcacgcttcttttcttcttagttccggtgacctttcttttctctttacttcaccttactctcctaccatgtccatcccttcttctgctgcaatcccctcgtttgatttcaatcattttaccaaattccttacatcgcacggtcttggcgtgcgtgctgttgccgtcgaagtagaagacgcagaagctgcttttaatatcagcgtttcgcacggggctatcccctgtgtttctcctattcacttggaaaacggtgtcgttttatctgaggttcatttatatggggatgttgtgcttcggtatgtaagctacggaaatgaatgtggggatgtgttttttcttcctgggtttgaggaaatgccggaggaatcatcgtttcgaggacttgattttggcattcgaaggttggatcatgctgtagggaatgtccctgagttggctcctgcaattgcttatttgaagaagtttactgggtttcatgagtttgctgagtttacagctgaagatgttgggacgagtgaaagtgggttgaattcagccgtattggcaaacaatgatgaaatggtgttgtttccgatgaatgaacctgtgtatgggacaaaaaggaagagccaaattcaaacttatttggagcataatgaaggagctggtgtacagcatttggctttgatgagtgaagacatattttggactttaagggagatgaggaagagaagtggtcttggtgggtttgagtttatgccgtcgccgcctccgacttattaccggaatttgaggaacagagctgctgatgtattgagtgaggagcagatgaaggagtgtgaagagttggggattttggtggataaagatgatcagggcactttgcttcaaatcttcaccaaacctattggagacaggtagattttaatcttgctttcaattgcttttgcttgatggattgactagccaatttgattgcattttgttgcttatatgacttgatgctagatagtttacctttctcagctgttaagttgtagcaagtatctaatacattcgttctgaaatatctgaaatatttgcaactgattatgacatgtagcgggagaaaatgtccgtttggcataaattaagcttggttttgcacttatcctcatttatttataaattctaaaacttgttagttgtaagctcctttcagttgtcctgaatttaattaagttaatgagaacaaagccttaatattccttcaaggtgattgtagttggggcactagttctaacaatggaaatttggaaatctattccaactggtcccaagttaatctttgtttgcaagcctgattggttcaaattaaggttattgtattcttgtatgaattcgactcaatgtaaatttgtttaatggagtatcaatttttaatagttttcaaccaagtagtattagatatattcgggattgaaataatgcatctatgagtgtacaaaaccaaatggcacatttgattagaataaaagagagtataaggctaatttcgtttacctaatatttaaagcgacccctaaaattcaattggcctaaacccataaagttcaaaagcagagaagaacatagaatagtgcaggtccattggtaatgcactaggagttggagcttttatgggtacaagtgtgtggctagttggggatgactgtctagcattgtctaggtgaaaagctgaagccttaagccatgaaggttttgagtagaggtgttcatttgggtcatcgggttgatttcgggtcagatgtttcgggtcggtttaaaatcgggttttgtgttcacattggtttttacgtaatt

SEQ ID NO: 8 - DNA, 775 bp, Amaranthus rudis
aaatgaatgtggggatgtgttttttcttcctgggtttgaggaaatgccggaggaatcatcgtttcgaggacttgatttcggccttcgaaggttggatcatgctgtagggaatgtccctgagttggctcctgcaattgcttatttgaagaagtttactgggtttcatgagtttgctgagtttacagctgaagatgttgggacgagtgaaagtgggttgaattcagccgtattggcaaataatgatgaaatggtgttgtttccaatgaatgaaccagtgtatgggacaaaaaggaagagtcaaattcaaacttatttggagcataatgaaggagctggtgtacaacatttggctttgatgagtgaagatatattctggactttaagggagatgaggaagagaagtggtcttggtgggtttgagtttatgccgtcgccgcctccgacttattaccggaatttgaggaacagagctgctgatgtattgagtgaggagcagatgaaggagtgtgaagagttggggattttggtggataaagatgatcagggtactttgcttcaaatcttcaccaaacctattggagacaggccaactatatttatcgagatcatccaaagaattggttgcatgatgaaagatgaagacggcaagatgtaccaaaagggtggttgcggaggatttggaaagggaaacttttcggagcttttcaaatcaattgaggagtacgagaagactcttgaacgtaaacaggttccagatacagctgctgcatgagtt

SEQ ID NO: 9 - DNA, 1204 bp, Amaranthus rudis
cttacatcgcacggtcttggtgtgcgtgctgttgctgtcgaagtagaggacgcagaagctgcttttaatatcagcgtttccaacggggctattccctgtgtttctcctattcaattggaaaacggtgtcgttttatctgaggttcatttatatggggatgttgtgcttcgctatgtaagctacggaaatgaacgtggggatgtgttttttcttcctgggtttgaggaaatgccggaggaatcgtcgtttcgaggacttgatttcggccttcgaaggttggatcatgctgtagggaatgttcccgagttggctcctgcaattgcttatttgaagaagtttactgggtttcatgagtttgctgagtttacagctgaagatgttgggacgagtgaaagtgggttgaattcagccgtattggcaaataatgatgaaatggtgttgtttccaatgaatgaacctgtgtatgggacaaaaaggaagagtcaaattcaaacttatttggagcataatgaaggagctggtgtacaacatttggctttgatgagtgaagatatattttggactttaagggagatgaggaagagaagtggtcttggtgggtttgagtttatgccgtcgccgcctccgacttattaccggaatttgaggaatagagctgctgatgtattgagtgaggagcagatgaaggagtgtgaagagttggggattttggtggataaagatgatcagggtactttgcttcaaatcttcactaagcctattggtgacaggccaactatatttatcgagatcatccaaagaattggttgcatgatgaaagatgaagacggcaagatgtaccaaaagggtggttgcggaggatttggaaagggaaacttttcggagcttttcaaatcaattgaggagtatgagaagactcttgaacgtaaacaggttccagatacagctgctgcatgagcagactaaaatattgctgaaacgcaggctgcagccatatgttagaacagtcattctgatggaaacactcaagcggtgagtagctgaggttggtgatgctgaagtcgagtcggtatttggatcatcttacaattacagtgcaaggatagtaatgaagcatgtaaacagctcctcagattagttttttccagtcataatcgtagatgtatatgagaaaatttaaattgctcttttaagttaatgaaaa

SEQ ID NO: 10 - DNA, 511 bp, Amaranthus rudis
tcatcaacaacaaaagagttgaaattctaataataatcccagtaagcagtgagtaagatcaaatggagagtcagttagtagctaatcatacaaaaccattaaagctacaaagttactctaatttcttaagatcaaacccaaaatctgatcgtttcaaagtgaaaaggttccaccacattgagttctggtgtggtgatgcaaccaacactagccttttattttcgttagggcttggcatgcctatggttgccaaatccgatctctccacaggcaaccttatacatgcctcctacgtcttacgtagtggcgaactttgtttcgtattcacggctccttactctccgtcatccatgctcacaactgcatcaattccttcgtttgactacagtgcgcacgcctcttttgtgtcctgtcatggccttggtgttcgcgctgtggcccttgaggtggagaacgctgaatccgcctttaggattagtgttgcagcaggagctcatccgtcagccccacc

SEQ ID NO: 11 - DNA, 770 bp, Amaranthus rudis
aatcattttaccaaattccttacatcgcacggtcttggcgtgcgtgctgttgctgtcgaagtagaagacgcagaagctgcttttaatatcagcgtttccaacggggctattccctgtgtttctcctattcacttggaaaacggtgtcgttttatctgaggttcatttatatggggatgttgtgcttcggtatgtaagctacggaaatgaatgtggggatgtgttttttcttcctgggtttgaggaaatgccggaggaatcatcgtttcgaggactagatttcggccttcgaaggttggatcatgctgtagggaatgtccctgagttggctcctgcaattgcttatttgaagaagtttactgggtttcatgagtttgctgagtttacagctgaagatgttgggacgagtgaaagtgggttgaattcagccgtattggcaaataatgatgaaatggtgttgtttccaatgaatgaaccagtgtatgggacaaaaaggaagagtcaaattcaaacttatttggagcataatgaaggagctggtgtacaacatttggctttgatgagtgaagatatattctggactttaagggagatgaggaagagaagtggtcttggtgggtttgagtttatgccgtcgccgcctccgacttattaccggaatttgaggaacagagctgctgatgtattgagtgaggagcagatgaaggagtgtgaagagttggggattttggtggataaagatgatcagggtactttgcttcaaatcttcac

SEQ ID NO: 12 - DNA, 1412 bp, Amaranthus rudis
accaccattttcgtttcttccccgtctgagttttattacttcactttctctctcatcatctgacatgggaactttgaaacccgaaaatcaacccgattccgaattcaaactcgtgggttactccaactttgttcgggttaaccccaaatctgaccgttttactgttaagcgtttccatcatatagagttttggtgtggcgacgcaaccaatgttagcagacgattttcttggggtcttggaatgcctaccgtcgctaaatctgacctttctacgggaaactctgttcacgcttcttttcttcttcgttccggtgacctttctttccttacttcaccttactcccctaccatgtccatcccttcttctgctgcaatcccctcgtttgatttcaatcattttaccaaattccttacatcgcacggtcttggcgtgcgtgctgttgctgtcgaagtagaagacgcagaagctgcttttaatatcagcgtttccaacggggctattccctgtgtttctcctattcacttggaaaacggtgtcgttttatctgaggttcatttatatggggatgttgtgcttcggtatgtaagctacggaaatgaatgtggggatgtgttttttcttcctgggtttgaggaaatgccggaggaatcttcgtttcgaggacttgatttcggccttcgaaggttggatcatgctgtagggaatgtccctgagttggctcctgcaattgcttatttgaagaagtttactgggtttcatgagtttgctgagtttacagctgaagatgttgggacgagtgaaagtgggttgaattcagccgtattggcaaataatgatgaaatggtgttgtttccaatgaatgaaccagtgtatgggacaaaaaggaagagtcaaattcaaacttatttggagcataatgaaggagctggtgtacaacatttggctttgatgagtgaagatatattctggactttaagggagatgaggaagagaagtggtcttggtgggtttgagtttatgccgtcgccgcctccgacttattaccggaatttgaggaacagagctgctgatgtattgagtgaggagcagatgaaggagtgtgaagagttggggattttggtggataaagatgatcagggtactttgcttcaaatcttcaccaaacctattggagacaggtagtttttaatcttgctttcaattgcttttgattaattgattgattagccaatttgatgattgcattttgttgcttgtatgacttgatgatatatggtttaccttttctcagctgttcagttgtagcaagtatttctaatccgttctgaaatactccattcgcaactgattgtgacatgttgtgcagaaaattatggaaaatgagaaaatgtccctatattggaagattggt

SEQ ID NO: 13 - DNA, 707 bp, Amaranthus thunbergii
cccgagttggctcctgcaattgcttatttgaagaagtttactgggtttcatgagtttgctgagtttacagctgaagatgttgggacgagtgaaagtgggttgaattcagccgtattggcaaataatgatgaaatggtgttgtttccaatgaatgaacctgtgtatgggacaaaaaggaagagccaaattcaaacttatttggagcataatgaaggagctggtgtacaacatttggctttgatgagtgaagatatattttggactttaagggagatgaggaagagaagtggtcttggtgggtttgagtttatgccgtcgccgcctccgacttattaccggaatttgaggaatagagctgctgatgtattgagtgaggagcagatgaaggagtgtgaagagttggggattttggtggataaagatgatcagggtactttgcttcaaatcttcactaagcctattggtgacaggccaaccatatttatcgagattatccaaagaattggatgcatgatgaaagatgaggacggcaagatgtaccaaaagggtggttgtggaggatttggaaagggaaacttttcggagctgttcaaatcaattgaggagtatgagaagactcttgaacgtaaacaggttccagatacagctgctgcatgagcagactaaaatattgctgaaacgcaggctgcagccatatgttagaacag

SEQ ID NO: 14 - DNA, 1267 bp, Amaranthus viridis
aaccaccattttctctctcctccattaccactaacactcccaccattttcgtttcttccccgtctgagtcttattacttcgctttctctcacatcatctgacatgggaactttgaaacccgaaactcagcccgattccgaattcaaactcgtgggatactccaacttcgttcgggttaaccccaaatctgaccgttttactgttaagcgttttcatcatatagagttttggtgtggcgatgcaaccaatgttagcagacgattttcttggggtcttggaatgcctaccgttgctaaatctgacctttctactggaaactctgttcacgcttcttttcttcttcgttccggtgacctttcttttctctttacttcaccttactctcctaccatgtccgtcccttcttctgctgcaatcccctcgtttgatttcaatcattttaccaaattccttacatcgcacggtcttggtgtgcgtgctgttgctgtcgaagtagaagacgcagaagctgcttttaatatcagcgtttccaacggggctattccctgtgtttctcctattcaattggaaaacggtgtcgttttatctgaggttcatttatatggggatgttgtgcttcggtatgtaagctacggaaatgaatgtggggatgtgtttttcttcctgggtttgaggaaatgccggaggaatcgtcgtttcgaggacttgatttcggccttcgaaggttggatcatgctgtagggaatgttcccgagttggctcctgcaattgcttatttgaaaaagtttactgggtttcatgagtttgctgagtttacagctgaagatgttgggacgagtgaaagtgggttgaattcagccgtattggcaaataatgatgaaatggtgttgtttccaatgaatgaacctgtgtatgggacaaaaaggaagagccaaattcaaacttatttggagcataatgaaggagctggtgtacaacatttggctttgatgagtgaagatatattttggactttaagggagatgaggaagagaagtggtcttggtgggtttgagtttatgccgtcgccgcctccgacttattaccggaatttgaggaatagagctgctgatgtattgagtgaggagcagatgaaggagtgtgaagagttggggattttggttgacagagatgatcaaggcactctgcttcaaattttcactaagcccattggagataggcctacgatattcatagaaataatacagagattagggtgcatggtgaa

SEQ ID NO: 15 - DNA, 637 bp, Ambrosia trifida
ccggtttacgggacgaagaggaagagtcagatacagacatatttggaacataatgaaggggcaggggtgcagcatttggcgttggcgagtgaggatatatttaggacattgagggagatgaggaaaaggagtggggtgggtgggtttgagtttatgccatctccgccgcctacttattatcggaatttgaagaacagggcgggcgatgtgttgtcggatgagcagattaaggagtgtgaggagttggggatattggtggataaggatgatcaggggactttgcttcagatttttaccaagcctgttggtgataggccgacgatattcatagagataattcagagagtaggatgtatgatgaaggatgaagaaggaaaggtgcagcagaaggcgggctgtggaggatttggtaaagggaacttttcggagctttttaaatcgattgaggaatatgaaaagacactggaagcaagaacctgaagccacatgaaaaccacacacaaataatctttcatgagattttaaaatctaatgattatgcatctgtggattctatacaacaaacagatcctgaaaatataggatatcaactttacaataagttatgtaatatgcatctcatgattatcaatgtatt

SEQ ID NO: 16 - DNA, 718 bp, Ambrosia trifida
tacctcacaaacccaatcctccatacttccgtgttctctactactgttccactttattaatcttacccaaccccttctcatctatttcccccctacctcacaaacataacccaaataaaaaataaaattaaaataaaaaataaaatggaactgaacccaccctcgttgaccccgtcgtcgccggagacaactccgaccactccaaccaccccatctccgccttcaagctagtcggcttcaaaaacttcatccgtaacaaccctaagtccgacaaattctccgtcaaatccttccaccacatcgagttctggtgctccgacgccaccaacgccgcccgtcgcttctcatggggtctcggcatgcctattacttacaaatccgacctttctaccggtaaccatacccacgcctcttatctccttaactccggccaccttaacttcctcttcaccgccccttattcccccaccatctccaccaccaccaccaccgcctccatcccgtcgttctcccactccgcctgccgtcacttcaccgcctcccacggccttgctgtccgtgccatcgccgttgaagttcaagacgctgaaatcgctttctccgttagtgtcgctaacggcgctaaaccctcatctccacctatcacccttggccacgacgacgtcgttttgtcagaagttcaactctacggtgacgtcgttttacgttatattagtta

SEQ ID NO: 17 - DNA, 54 bp, Ambrosia trifida
ctccgaggcgttttgggtccaaaatccgaagcgcggggcttaaagcgcgaggcg

SEQ ID NO: 18 - DNA, 719 bp, Ambrosia trifida
cttcatgtagttcagcgctttttggcaaaaaaagcgtttttcttggagccttgtgcctcaaacgcttaaagtgcgccccaggcgagcttttttaaaccaaggaacacgcacaagcactccaccaatagttcaatgccgtgctaaccagtgggattaactagtggaagctggattcaaacatgctagagtggaaatatattttttttaatgtttggatatgcatatttaaccattatagcataatattaatgacaagggttgtaaattggtatgcaaattgtttgagcaggccgacgatattcatagagataattcagagagtaggatgtatgatgaaggatgatgaaggaaaggtgcagcagaaggcgggttgtggaggatttggtaaagggaacttttcggagctttttaaatcgattgaggaatatgaaaagacactggaagcaagaacctgaagccacatgaaaaccacacaaataatctttaatgagatcttaaaatctaatgattatgcatctgtggattctatacaacaaacagatcctgaaaatataggatatcaactttacaataagttatgtaatatgcatctcatgattatcaatgtattaatttatttttatgttgctgttttcggtttagtttttgttcgttggtgttgggccggctcttcttctatttcgttcttgatccatttgaagttgaaggcgaaatttagg

SEQ ID NO: 19 - DNA, 845 bp, Ambrosia trifida
gcagttgagctctacggctcactgaaacagacctttaaaccatgaacaaggtttatgtgtaggtaaactaactttggaccaaaaagctgtttttttaaccttcacaatttgtttcatggaaggttttcctttttgttcagttgacatgtttctaaacgggacaaataaacatacatctatgcctattgcacatatagcatcaaccacctcactcccatgtggccctcacaacaaggttgtaaaaatcgggactagtcgccgactagtccccgataaatcggtgatcggaggtctactgattaatttttcattaatcactcaattagtcggaatcggcccaagccggtccaagccggccaaagtcggccgaagtcggagctagtcgccaaagaattccgtttgaagttggcttaaaaacatgctttcatcccacaaactgatccattaccccaaaaaaccaaagttaaaaacctgacctggatccctaaatgcttcataatccggcaaacctttctgttccggcgaacctttctgtttccggcgacccatttggagcaggtggagttggttttgttcagtcttttgcggcgtataatgaaagtggtagtgagtagggtttttaagtttggatctttttatatttaaccccttatagttttactagtttatattttgtcccttaacttatatatttatacataataagcataaagttaatgttatatatatacaaattaaccctctacatctttactagtctatatttggtcctctaacttatacatctatacaaaaaaagcataaaattaatgttatatatatataaatttataaaattatacata

SEQ ID NO: 20 - DNA, 1024 bp, Ambrosia trifida
tcgtcgtttcaggagctggattacggtatccggcggctagatcacgctgtggggaacgtaccggagttagcaccagcagtggaatatataaaatcgtttaccgggtttcacgagtttgctgagtttacagcagaggatgtgggaacgagtgagagtggactcaactcggtggtgttggcttgcaatagtgagatggtattaatacctctgaatgagccggtttacgggacgaagaggaagagtcagatacagacatatttggaacataatgaaggggcaggggtgcagcatttggcgttggcgagtgaggatatatttaggacattgagggagatgaggaaaaggagtggggtgggtgggtttgagtttatgccgtctccgccgcctacttattatcggaatttgaagaatagggcgggcgatgtgttgtcggatgagcagattaaggagtgtgaggagttggggatattggtggataaggatgatcaggggactttgcttcagatttttaccaagcctgttggtgataggtattgtttttcgttaatgtagcgttacttgaattcgtaattatgtgtttaaactttaaactagatagtattaaggcttatgcaatatgtattgttcccctagaggacgagggttctagactctagttccgtaatgtagtgataaaggtgtagattaagagtaggattagcgggtgtgttagttagcggttttaacaagaaaaacacataatatgttgaaagaacaataacaagaatggcaaacataaaaaaggaaaagccataaaggttgtcattctataatttgcttgcattcaagattcttactaggattgtattattatgtggttactaaactctattcaagtataaaaacatgaaaccccttcacataaggaaaaagaaatatgtatgttttaaaatggatcatcttttcccttgtgtcacgattgccaatgttttttttgttgaccatgagaaccgttggtgaacacgccttggtttaaaaaagcgcg

SEQ ID NO: 21 - DNA, 1610 bp, Conyza canadensis
tatggtattgcattgtttcattcaattgtacataatccacaaatacatatggtgataatcatgttctttgagtcaagaagatataaacattatacataggcagttttcatgcagtggcagttggttcagtggtgtttcgtgcttccagcgtcttctcgtattcttcaatagatttgaagagctctgagaaattgcccttgccaaatcctccacagcctggcttctgttgcaccttgccttcatcatctttcagcatacaccctactctctgaataatctctatgaatatagttggcctatcaccaacaggcttggtgaaaatctgaagcaaagttccctgatcatctctatcaaccaaaatccccaattcctcacattccttaatctgttcgtcactcaacacgtccccgaccctattcttcaaattcttataataagtaggcggtggagacggcataaactcaaaaccaccgacactactccgttttctcatctctctcaaagtcctaaaaatatcctcactcgccaacgccaaatgctgcacaccagctccctcattatgttccaaatacgtctgtatctggctcttccttttcgttccataaactggctcgttcatcggtatcaaaaccgtttcactattacacgctaatacaaccgaattaagcccgctctcactcgttcctacatcctctgccgtaaattcagcaaattcgtgaaatccagtgaaggacttcacgtagtctactgccggagctagctccggcacgtttcctacggcgtgatctaacttatgaattccgtaattaagttcatgaaacgacgacgttgcgtccacaggctcaaaacccggtaggaaattagtagtaacatcataattagtgtttttaaaactaacatatctcaaaacgacatcgccgtacagtttaacttcagccaaaacgacgtcgttatttcctagagtgaccggagctgacgacggtttagcaccgtgagcgatactaacagaataagcgatttcagcgtcttcaacttcaacggcgatagaccgtacagcgaggccgtgagtggcggtgaagttccggcaagcagtgtgagagaaagtaggaatagacgatgtggaaccggtggtggagatggtggtggaataaggtgcggtgaagaggaagttgagctggccggagttaataaggtaagaagcgtgagtgttgttaccggtggagagatcggatttgtaaagaagcggcatgccgagaccccaagagaagcggcgggcggtgttggtggcgtcggaacaccagaattcgacgtggtggaattttttgacggtgaatttgtcggatttagggttttgacggatgaagtttttgaagccgacgagtttgtaagtggcggaggtggtggtagtggtttgttgttgtccggtgagtgtgtttccgtttgctgcttcagttaccattttgtttttattaatttgaatgtatagatagaaatatgaagtagatatatagaatgatagatatagatacagtacaagtagttgaggggttggatgatggagatgaagaagaacacggaagtggtttattttctttgggtgctattaattg

SEQ ID NO: 22 - DNA, 4963 bp, Conyza canadensis
aacacgaatgggctagctagctgatgaatttaatgtatatatgtctttaaaaaccactttcatgggctacttgtgtttttgatgagtttttcaacatcattctcatcttaccttcttacaatggttcaccatattgcttcgtttgtcgtctcctgcatggttatcgaatttattttttcaaattatattactattatctttttgttttctgatttctcacatgtaatttacattatgagtttcaccatccgtatgtaaattgtaaaataaatcacaaatttggtttttaaccatcaactaaattcattgaaatccttaagttataacccatttgaggttatagattatgagagtagattcctattttctctttggcttatgtatccttaattatgtttgaattgattctaactttattatattttaggaattacttaacttcaaaaatctcaacacgtcatataatgctttttgtggatggatgcttttgacccatttcgttgaaaacttatgtcgacacacatatctgacaaacttgtactcgtatatcctttatcgcatatgctatagtataatattcgaaatttaatctcaagataattttcccaaaaggtcatattaattgaattgagattaattcctacctaacatatatagatatattgtgttggtgttatcctctttggagttttagtcgcgcatgtattctaagaaaatggtaattttgaacttatattgatggtctaaattttatcctatcactaaaatttcagtcatgtttcctagctaattaatttgggttcaatccaggtattctaaataagttttgtaaatacatgtctattatcataatgaaacatagaaaaacccgtaaagtaaaaatgtctatatttttagtggcaaattattaatgttgctataatgaaaagagaagattcaaccttttgtacatcataaaaaatgttattctacattatgctaattttacattagacttcatagttttgaaaactttcaaatgaagggacttaaattttaaatttaaagatgatgtcataaattagttagataaaagttaaataattaataagaaaaggtctaatgataccaaataaaattttcttaaaaaagtattaatgtcattacaattttattttatttatataagagaagataatagattttcttgtataaaaaacttacctgtaaacttctatataaactttttagaaaaataatttaaaacggtctcaaatatcgttatttccgaacatacaaaaacccaaaacctaatccatttaattatattgtaagacctcgattagcttaatttattttgaaacctatttacaaatgagtttggcatgatatgaacttttttgcatttacatatggctgtaaacgaactgaacagttcacgaacaattcatgaaccgttcggcgggaggttcgttcgtgttagttcgtttagttaaatgaatgaacatgaacaaagctctcgttcgttcatttacgttcatgaacgttcgatagtctgttcgtgaactttagttcgtctatgttcatgaactgtcgttcgtgaacaataatttgattatgttcatttacacacacatacataatcaactgtactataaaaatagtttaaatataaatatttttttatataatatatattattaatatctaattatttaagaaaaagtttaaataaaatacttaatttataaatatttcgttcgtttgtgttcgtgtacatttgttcatgaacacaaatgaacgaacatgaacaagtcattatgttcgtttatctgttcgttaagttaaatgaacgaacgaacacgaacaaggtctcgtttgtgttcgttcggttcgtttacggcccttaattacatgtgaagttaataaataaaaataaagttcatcaaaattaaacctatggttggagataaaaccagtcagccttacgtgtggaaactatcctactctacaattaatagcacccaaagaaaataaaccacttccgtgttcttcttcatctccatcatccaacccctcaactacttgtactgtatctatatctatcattctatatatctacttcatatttctatctatacattcaaattaataaaaacaaaatggtaactgaagcagcaaacggaaacacactcaccggacaacaacaaaccactaccaccacctccgccacttacaaactcgtcggcttcaaaaacttcatccgtcaaaaccctaaatccgacaaattcaccgtcaaaaaattccaccacgtcgaattctggtgttccgacgccaccaacaccgcccgccgcttctcttggggtctcggcatgccgcttctttacaaatccgatctctccaccggtaacaacactcacgcttcttaccttattaactccggccagctcaacttcctcttcaccgcaccttattccaccaccatctccaccaccggttccacatcgtctattcctactttctctcacactgcttgccggaacttcaccgccactcacggcctcgctgtacggtctatcgccgttgaagttgaagacgctgaaatcgcttattctgttagtatcgctcacggtgctaaaccgtcgtcagctccggtcactctaggaaataacgacgtcgttttggctgaagttaaactgtacggcgatgtcgttttgagatatgttagttttaaaaacactaattatgatgttactactaatttcctaccgggttttgagcctgtggacgcaacgtcgtcgtttcatgaacttaattacggaattcataagttagatcacgccgtaggaaacgtgccggagctagctccggcagtagactacgtgaagtccttcactggatttcacgaatttgctgaatttacggcagaggatgtaggaacgagtgagagcgggcttaattcggttgtattagcgtgtaatagtgaaacggttttgataccgatgaacgagccagtttatggaacgaaaaggaagagccagatacagacgtatttggaacataatgagggagctggtgtgcagcatttggcgttggcgagtgaggatatttttaggactttgagagagatgagaaaacggagtagtatcggtggttttgagtttatgccgtctccaccgcctacttattataagaatttgaagaatagggtcggggacgtgttgagtgacgaacagattaaggaatgtgaggaattggggattttggttgatagagatgatcagggaactttgcttcagattttcaccaagcctgttggtgataggtatgtctatgttaactttatcagtgattgtgcatttgtccattttgttgaattcgtaaaacatgagattaaatacctgcaatatgttttgcttacattgaatctaggccaggttttcagtgaacaagtaccataaatgtataatgtaggtctagtcttgttaatgatcagtgatcaatagtgcatacattgaatgacgaattgacaactagtttctaatagaatgtgaaatataaagtgaatgtcacaaaagctgttatcttatctattttagttgtaatggaccaatggttggactgtttgaaagtgtgcttacaatcaagattcgagattgttactaggatgctagcgctaggtatatatcatatggtgtctgagctctatggtagtataaattgtgaatacactaaacatctcccaaaaatatttgagtaaccgatcaatctactagttattgcaatgccaaacatctctttaaacaactacttattgtaaagccatactacatcaacacaccaaaaatctatctcccacaacccccgttttacttgggttatgaacttagtatagttattcaaaataatcactttcaaaatggatccaacacatctgaaactggtcacatttgagttcttatctttcaaataatttgtaattcatatcaggctcagcttcatggggtctggcctttgtctgtgtgtttgtgttgtgtcttttctggtgtgatctaattcggtatgcatattgtttgagcaggccaactatattcatagagattattcagagagtagggtgtatgctgaaagatgatgaaggcaaggtgcaacagaagccaggctgtggaggatttggcaagggcaatttctcagagctcttcaaatctattgaagaatacgagaagacgctggaagcacgaaacaccactgaaccaactgccactgcatgaaaactgcctatgtataatgtttatatcttcttgactcaaagaacatgattatcaccatatgtatttgtggattatgtacaattgaatgaaacaatgcaataccatatgtgatgtgatttatatagaataacaatagatgtcattcatatatgccgttcctgttttagtaattgtgtgttggtggtgtgttgcaagttccaatcagctgtatataatgcctaaatattcaatttgacctccattaaggatccattgtgcagattctttgtttctgtcttaaaagtgtgggagacttgaaacacatctgtaaaactgaaccagatctgaatcgagctgaatggaagcggcgcaggtctgctctgtggttccacttacaacaaacaattgttcatggtacgtcgcctcttcatagccagtgtttattgtaagaaagcattgtttaggcgaaaaaggaaaaggtgatttatgtaagaaaaggtagtgctattaaggttatgttccccatgaacaaacagggtaaattcatacaggatcaacttttaaatgatactattaaaggtcatctacttctgtttcccatgcttgtgccaagctcaaatttagacgaaaccaaaacaggatgagaaaactaattaacacatattttagtaagcaattacagctatatatcatactatcattaacaattacatcaacaatctgaaaagttgcat

SEQ ID NO: 23 - DNA, 5610 bp, Conyza canadensis
attaaaaacacgaatgggctagctagctgatgaatttaatgtatatatgtctttaaaaaccactttcatgggctacttgtgtttttgatgagtttttcaacatcattctcatcttaccttcttacaatggttcaccatattgcttcgtttgtcgtctcctgcatggttatcgaatttattttttcaaattatattactattatctttttgttttctgatttctcacatgtaatttacattatgagtttcaccatccgtatgtaaattgtaaaataaatcacaaatttggtttttaaccatcaactaaattcattgaaatccttaagttataacccatttgaggttatagattatgagagtagattcctattttctctttggcttatgtatccttaattatgtttgaattgattctaactttattatattttaggaattacttaacttcaaaaatctcaacacgtcatataatgctttttgtggatggatgcttttgacccatttcgttgaaaacttatgtcgacacacatatctgacaaacttgtactcgtatatcctttatcgcatatgctatagtataatattcgaaatttaatctcaagataattttcccaaaaggtcatattaattgaattgagattaattcctacctaacatatatagatatattgtgttggtgttatcctctttggagttttagtcgcgcatgtattctaagaaaatggtaattttgaacttatattgatggtctaaattttatcctatcactaaaatttcagtcatgtttcctagctaattaatttgggttcaatccaggtattctaaataagttttgtaaatacatgtctattatcataatgaaacatagaaaaacccgtaaagtaaaaatgtctatatttttagtggcaaattattaatgttgctataatgaaaagagaagattcaaccttttgtacatcataaaaaatgttattctacattatgctaattttacattagacttcatagttttgaaaactttcaaatgaagggacttaaattttaaatttaaagatgatgtcataaattagttagataaaagttaaataattaataagaaaaggtctaatgataccaaataaaattttcttaaaaaagtattaatgtcattacaattttattttatttatataagagaagataatagattttcttgtataaaaaacttacctgtaaacttctatataaactttttagaaaaataatttaaaacggtctcaaatatcgttatttccgaacatacaaaaacccaaaacctaatccatttaattatattgtaagacctcgattagcttaatttattttgaaacctatttacaaatgagtttggcatgatatgaacttttttgcatttacatatggctgtaaacgaactgaacagttcacgaacaattcatgaaccgttcggcgggaggttcgttcgtgttagttcgtttagttaaatgaatgaacatgaacaaagctctcgttcgttcatttacgttcatgaacgttcgatagtctgttcgtgaactttagttcgtctatgttcatgaactgtcgttcgtgaacaataatttgattatgttcatttacacacacatacataatcaactgtactataaaaatagtttaaatataaatatttttttatataatatatattattaatatctaattatttaagaaaaagtttaaataaaatacttaacttataaatatttcgttcgtttgtgttcgtgtacatttgttcatgaacacaaatgaacgaacatgaacaagtcattatgttcgtttatctgtttgttaagttaaatgaacgaacgaacacgaacaaggtctcgtttgtgttcgttcggttcgtttacggcccttaattacatgtgaagttaataaataaaaataaagttcatcaaaattaaacctatggttggagataaaaccagtcagccttacgtgtggaaactatcctactctacaattaatagcacccaaagaaaataaaccacttccgtgttcttcttcatctccatcatccaacccctcaactacttgtactgtatctatatctatcattctatatatctacttcatatttctatctatacattcaaattaataaaaacaaaatggtaactgaagcagcaaacggaaacacactcaccggacaacaacaaaccactaccaccacctccgccacttacaaactcgtcggcttcaaaaacttcatccgtcaaaaccctaaatccgacaaattcaccgtcaaaaaattccaccacgtcgaattctggtgttccgacgccaccaacaccgcccgccgcttctcttggggtctcggcatgccgcttctttacaaatccgatctctccaccggtaacaacactcacgcttcttaccttattaactccggccagctcaacttcctcttcaccgcaccttattccaccaccatctccaccaccggttccacatcgtctattcctactttctctcacactgcttgccggaacttcaccgccactcacggcctcgctgtacggtctatcgccgttgaagttgaagacgctgaaatcgcttattctgttagtatcgctcacggtgctaaaccgtcgtcagctccggtcactctaggaaataacgacgtcgttttggctgaagttaaactgtacggcgatgtcgttttgagatatgttagttttaaaaacactaattatgatgttactactaatttcctaccgggttttgagcctgtggacgcaacgtcgtcgtttcatgaacttaattacggaattcataagttagatcacgccgtaggaaacgtgccggagctagctccggcagtagactacgtgaagtccttcactggatttcacgaatttgctgaatttacggcagaggatgtaggaacgagtgagagcgggcttaattcggttgtattagcgtgtaatagtgaaacggttttgataccgatgaacgagccagtttatggaacgaaaaggaagagccagatacagacgtatttggaacataatgagggagctggtgtgcagcatttggcgttggcgagtgaggatatttttaggactttgagagagatgagaaaacggagtagtatcggtggttttgagtttatgccgtctccaccgcctacttattataagaatttgaagaatagggtcggggacgtgttgagtgacgaacagattaaggaatgtgaggaattggggattttggttgatagagatgatcagggaactttgcttcagattttcaccaagcctgttggtgataggaggccaactatattcatagagattattcagagagtagggtgtatgctgaaagatgatgaaggcaaggtgcaacagaagccaggctgtggaggatttggcaagggcaatttctcagagctcttcaaatctattgaagaatacgagaagacgctggaagcacgaaacaccactgaaccaactgccactgcatgaaaactgcctatgtataatgtttatatcttcttgactcaaagaacatgattatcaccatatgtatttgtggattatgtacaattgaatgaaacaatgcaataccatatgtgatgtgatttatatagaataacaatagatgtcattcatatatgccgttcctgttttagtaattgtgtgttggtggtgtgttgcaagttccaatcagctgtatataatgcctaaatattcaatttgacctccattaaggatccattgtgcagattctttgtttctgtcttaaaagtgtgggagacttgaaacacatctgtaaaactgaaccagatctgaatcgagctgaatggaagcggcgcaggtctgctctttggttccacttacaacaaacaattgttcatggtacgtcgcctcttcatggccagtgtttattgtaagaaagcattgtttaggcgaaaaaggaaaaggtgatttatgtaagaaaaggtagtgctattaaggttatgttccccatgaacaaacagggtaaattcatacaggatcaacttttaaatgatactattaaaggtcatctacttctgtttcccatgcttgtgccaagctcaaatttagacgaaaccaaaacaggatgaaaaaactaattaacacatattttagtaagcaattacagctatatatcatactatcatcaacaatctgaaaagttgcatacaatttatcgtttattcctgtcaagtgatgacatgaaaaataccatttagtttacacgtactcagatatgccaataatagagggactttttctatatgagaacatacatgatgttcataattcataatttacttgatacggcaaggtacttggtatggcgtccttgtgcaattgtttttccggtctttttgttccttaattcaacggtcacaacagcaatagccttcccaacacgcaatgtttttgcctctatctcaatctcgtcctgtaagaaaattgcatccttaacaaaattttactcaaagacgcatctatggtacgaagtacaaaccaaagattttcagtaagtcgtgcaaaacctttatgcatactgcaactactcaaacttcacatcttatgcctaaaatcaaagaccccaaagaagccttgagttcaaaatccaagaatttctgtcagctacaggggacaggttcaacccatttacatacaaatgggtcaaaatgggttatgttattctgtgtttctgccaaaccagacctggcacgtactaaaactcacccattacccaacccacctacccagcccccctttttaccaacatgcattttgcttttgcaaccctgttgcttcaagcattcaactattcaagtagcaagatcgaaagaacttatacaaaaagttgccatttatgattaagtgaagccttaatgacaccaaatcagttattatagcattacaatgggagagcttcaacagaaaagttatcaagtcttctccaacaagatcatgagctcattttcaattttatatcaaacggtagcaataacgaaagtaaatctggtggctcaaggtaaaacctcataacctttgatggcataataacttccagaccaactaaacaccaaggtggaagttattactttgtgctaatgatagcagttacaaacataaaaaatctggtcggcttcgttcagttttctgaaataccaatatcgtctgacaaggtgccatcttcatattcagagtcacttcatgatcaactcttgtggggtttcaaactcaagaacgttttaaatacattcatttaatatttttatctccagaatactactcaacactttcctgtaaggtcttcaatttcagggggaaaaaaacccaaaattcaaggaatcctgctattagtgtatcgtttcttgatagcaaagctttcatttacaatctctaacaacatttttacttcaaaattttatcataatgctctttaggaaaatgttgacggtatgaatatgagatcaggatatcaaaagaacaaaatatttattttgttcttaagttgcaaaccgcacaagagttgatcatgcagtgttcgcgaatttgaagatggcaccttgttaggcaacattgg

SEQ ID NO: 24 - DNA, 386 bp, Euphorbia heterophylla
ttgtttctgccgaaattcgagccggtagatgaggcgtcgtcgtttccgttggattacgggattcgacggctagatcacgcggttggaaatgtaccggatcttgctccggcggtttcgtatgtgaagaagttcaccggattccacgagttcgctgagttcacggcggaggatgtagggacgagtgagagcggattgaactcggtggtgttggctaacaacgaagaaacggtactgctgccgatgaatgaaccggtgtttgggacgaagaggaaaagtcagatacagacgtatttggagcacaatgaaggagctggagtacagcatttggcgcttgtgagtgctgatattttcaatactttgagagagatgaggaggaggagtgcgat

SEQ ID NO: 25 - DNA, 2639 bp, Euphorbia heterophylla
gacaattaataaaaaaaacgtaaagactacctttaattggaggagaggaaaacaccacgtttaaaaatcccgttgttatccgattgatgaaaaaaaagattaacacgttacgacttctccattcaataatccattttctttatcttataaataatttgaaatcccatcctcctcgttctccgttcaccagaaaaaacagaaatgggaaaagacacatcagctgccgccgaagcattcaagctcgtcggattttccaatttcgtcagaatcaatccccggtccgaccgtttcccggtcaagcgcttccaccacgtcgagttctggtgctccgacgcaaccaacaccgctcgccgcttctcatggggcctcgggatgccgttcgtcgccaaatcggatctctccaccggcaacgtcacccacgcctcctacctcctccgctccggcgacctcaatttcctcttcacagctccctactctccctccatagccgccatggagaatctctccgatactgctaccgcatcaatccctactttctcccgcgacgttttcctcgatttctccgccaaacacggcctcgccgtccgagctatagcaatcgaagtggaggacgctgcagttgccttcaatactagtgttgctcaaggcgcggttccggtggccggacctgtagtgctcgataatcgcgcttcggtagcggaggttcacttgtacggcgacgtcgttttgcggtacgtcagttacctaaactctgatgactgtttgtttctgccgaaatttgaggcggtagatgaggaggcgtcgtttccgttggattacgggatccggcggctagatcacgcggttggaaatgtaccggatcttgctccggcggtttcgtatgtgaagaagttcaccggattccacgagttcgctgagttcacggcggaggatgtagggacgagtgagagcggattgaactcggtggtgttggctaacaacgaagaaacggtactgctgccgatgaatgaaccggtgtttgggacgaagaggaaaagtcagatacagacgtatttggagcacaatgaaggagctggagtacagcatttggcgcttgtgagtgctgatattttcaatactttgagagagatgaggaggaggagtgcgattgggggatttgagtttatgccgtctcctccgccgacatattaccggaatttgaagaagagagttggagatattttgagtgatgaacagattaaggagtgtgaagaattagggattctggtggacagggatgatcaagggaccttgcttcagattttcactaaacctgtgggagataggtatttctatcttcttcttcattgttttcacttacaatacctttttccattgaaaattctttgtttcttgtgtttctcttatatgtgttggctgctatactatggttgatagagaattagaatttagcgtgttcaaaactcaattctttatctttatctttagccttaggtcattttgggattgctttcaaactgatttgattatgttactaattaattagtacgaaggactaacatgtgttgttctaagctaaaatatattctataaagctaaaatatcttaataagcaataccaaactagcccttaaatgcatagttagaccctaaatttgacaacttttataacttggcgcctctaatttacaaatgtcttcgccaacctcttgaactggcaaaagttttcactgcaacccctaggtgccacgtgtcaccttgtgattgatttgacttttcaaagatgtcaaaattttttattttcctccctctaatcaaaaggtgacaagtgatattgagtaggggtctgtagtgaatactttccccaagttgagacatccagaaatcggaggcgtcatgtgataaaagttgcttagttaagggagcttaatatgtattaagccttttcgctaccttgccatttaaatccttagtccaagcaaaatatacaaaaattagttagaactttatgattcagaacctacgtaactttaatttgaccagtagttctatcttatgcatgccagccatgttgactggtgaacttatattcaattttgctattcaagtaaatccttatccatcaatccgaatcaaaagattttaatgttaggttaaacttgtacgattcaaaacttaggttgtactttttgactagtagttctattatgtatgtgtatatataatggggttttttttctgtaggccaacgattttcgtagagataatacagagagtagggtgtatgctaaaggacgaagcagggagagaataccagaagggtggatgtggcggttttggtaagggaaacttttcggagttattcaaatccattgaagaatacgagaaaacactggaagccaaacgaactgcacaagcatagaggttagtagggaatttgatgatacccataaataatggttgttacttgttgccttgtaataagtttagttaaatgatgactgtgcctgcaaattggaatgttctctaattgttggttgatattgttgtaaaatgtaaactatgatgttactataaagtatagttgtggttatcgtgtctcttcttttggaatctgaagtttcgatatttttttgggg

SEQ ID NO: 26 - DNA, 6569 bp, Euphorbia heterophylla
tgactgaaattaaattttaattatttgcaaagcttacggtgaaattaaactctaaccattaaaattttaatgagtcaaaattgaccgaaatgttcatgtcataaatttagataatcctttcagatatgtacgtgaaaccatgtacctaaattgtagtcagattgctaaacccccttcgaaaatgaatgaaatgatatgatatgctatgctatgtattttaaggattattttatagtaactttgtttataatttactttatatgatcatcatccaattaactttcacctcactaattcaatgattgaaatggactaagtaattttacttaataaaaaaataaaatcactataacctacacatatttttaaaaataacaccataatttattaaaggacatttaattgaagtagaaaattatataattactcgctaataaattttcaattggaggagaggaaaacaccacgtttaaaaatcccgttgttatccgattgacgaaaaaaaagattaacacgtcacgacttctccattcaataatccattttctttatcttataaataatttgaaatcccatcctcctcgttctccgttcaccagaaaaaacagaaatgggaaaagacacgtcagccgctgccgaagcattcaagctcgtcggattttccaatttcgtcagaatcaatccccggtccgaccgtttcccggtcaagcgcttccaccacgtcgagttcaggtgctccgacgcaaccaacaccgctcgccgcttctcatggggcctcgggatgccgttcgtcgccaaatcggatctctccaccggcaacgtcacccacgcctcctacctcctccgctccggcgacctcaatttcctcttcacagctccctactctccctccatagccgccatggagaatctctccgatactgccaccgcatcaatccctactttctcccgcgacgttttcctcgatttctccgccaaacacggcctcgccgtccgagctatagccattgaagtggaagatgctgcgattgctttcactaccagcgttgctcaaggcgcgattccggtggccggacctattgtgctcgataatcgtgcttcagttgcggaggtccacttgtacggcgacgtcgttttgcggtacgtcagttatctaaactccgatagttgcttgtttctgccgaaattcgagccggtagatgaggcgtcgtctttcccgttggattacgggattcgacggctagatcacgcggttggaaatgtgccggaattatctccggcggtttcgtatgtgaaacagttcaccggattccatgagttcgccgagttcacggcggaggatgtggggacgagtgagagcggattgaactcggtggtgttggctaataacgaagaaacggttttactaccgttgaatgaaccggtgtatggcacaaagaggaaaagtcagatacaaacgtatttggagcacaacgaaggggctggagtacagcatttagcacttgtgagtgcggatatatttaacactttgagagagatgaggaagaggagtggcgttgggggatttgagtttatgccgtctcctccgcccacatattaccggaatttgaagaagagagtcggggatattttgagcgatgaacagattaaggaatgtgaagaattagggattttggtggatagggatgatcaagggaccttgcttcagattttcactaaacctgtgggagataggtatgtcaatcaatcttgtgaaatgattagttgttctataattacttatgtgatttacattcttagagtctgtaaattcttgtgatcataaatttgttggcttggataccatattaatattatccttaatttagaatgtcaaagttatgttctgtctttacaatgccgttgaaatcgttatttatcaagtccaagcaaacggtcctaaaatgtcctttaacttactggttcggaatctcatctgttcaatttgattagtagttccatcttttagtatatacgccaggcgccagacatgttgatttgtgagtttttgttatttggaatctcaaagttgagttttttctatctataaaaagtagctcaaatccttagttatcaagtcaaaacccaaagatcctattattaacttggatctgaaattatatcgctttctggtttatttctaaatttggtggtcacttgatttctcatcttgacaagtgaattcatctggacatagttggtcttttatgggatgaatggatgtgctgtttttgcaggccaaccattttcgttgaaataattcagcgagtcgggtgtatggtcaaggatgaagcagggagagaataccagaagggtgggtgtggcggtttcggtaagggaaacttctcggagttattcaaatcaatcgaggaatatgagaaaacattggaggccaaacgaactgcagaagcagctcgagcataaaataaaggttagtatggaagatgatatgcatacataaataaaggttggctgttgtttattacgttgtaatattctactccaatgatgctgttattgtctgaaaataccagtgcctgcgaattggaatgttctctaattgttaggaacaactggaatcattgtatatcgtaaactatggttcattaaaagttaaaaacatatatatgatatgattaatatagaattactccctttatttggaatctaaagattcatttctcagtttctttctgggcgattttgagcatttctgtacttaaattctgcgcgaatacacccaccaagccctagctagcttggtggttaccttttaagggtgttggaaacctaaattagaaaagatttcagtttaatttatgtcattttatatataaaagtttgaaacttttttattcaaaataagttgattgataaattgaaatcaattagctttgtagcagatgttcacccaaatctaatataaatatattataaatctaatatatttctgttaacaaacttataatatattaataaatacaattgcaaagacaaagagacttatattagaattataacataattttttataaatatattagatacgtatatcttataattgtaacatataaaagattttatagtaaatatgtgttataagtctaaaataagcctttttaagtttaatacaagtctctttgtgtaacaccccgggttcgaccgattgacccccacaaaccaacacaagtctttctagtaagctttgccttgagattgttccaagtcaaggacgctcaactttggagttctcccatataagctcctgaaaagaaggtgtaacttgttggtataggcagcagtaccaatcaaacctttttaagtccctttccactttaatgtccctttctattgatataattttattgactttatgtaatcaaatgtttattcatcccgtgtttttttattcattatgttttaattttttaattagtcaaaatataatatctaaaaaatctatcaaatttgggctcggctcggcttgactcattaacatccgactctctacctcttacttttattaaatacatttgtgtttttcagttgtacgttccttctcatatttacatttcccttttttcttttctttttaatttattttttatttatttcgcaaatcgttgattttctcggtatgctgctttataattttttgttcgtctcgtttttatcttcgaaagtatattcatgttgatatactttttcaagttcttattttcaaagatatgtaacaaatgacataaacattatccagcaaaaatcagtaaattactagcatttcaccaacaaaatactagcattttacccgtgacgaaaagtttgaaaaaaatatataaaaaaaatcaaacaaaaatcatttttataccaacattataccatgtatgaaaaaataaaactgatcataaacataaagaaatcaaacaaaattcaccttatcttaatattttaccaagagtgaaggaccaaacataaacatgttttacgaaaaaaatcaaacaaaaatcatttcacactagcattttattaacaaaatctcgatatattatcagtaaaataccaacattttatcaacaaaataccatcatttgagaggaggttcgagtgcgccttcgccgttctgcggcggcttctttgacggtggagaaagcatagctgcgtgatggaggcagaagaggggttgatgaaattgggttggattgaagtggaattgagtcaatgaggctaggtgatagggtgagatgatgcatgactcgaattggtggttgatcgcagaggtggaggtggcgattggccggcggagaagacggcaaatagatgtttttgggcggagtacaaagcgccttaaatcctagcaaaggctgatgaaaataaaaaagggtaatgctgcacttttatctactttgtgcaatatctatatacagtatttattatcatcttattccttttatttatttctttcatcttttatgggagttgaaattctttctagatattcttttctttttgctaaatttttctgctcattgtcatacacttgcttggtaaactgaatctcaaaccaaataatgaaatctgaagtatgtatccacagaatcgcaatgctcatcgacggaagtagaaggggaaaactgctgttggagttgaggtatataatctgcagttgaaatcgacgttgtgtatgtaattttgaattgaacaagagcgaaacctagttttctatgatctgtacctggcgtataagaattccatcaatatcggcagggcgatgcgttaatattgcattaacaccggtggtgagaagggagctaattgcttgaaatccgctggcatgccgcaatgttgatatgtcacataaatgcgactaagtgtgacttatcttgaagaggagtgaaattggggaaacgactaagagtgacttatcaagaagtgtgattaccatgcaactaagtgcgacacggtgaaacgactcgactcctgcagccaccaccaaatttcacaatttcatataggtaaaagggagacgatcagaaagaggggaaacaaaacacaaagagagggaaaagaaatgttggagggactgaaattatagaaggggtaaaattagaaacaaaaaaaattataaggtgttgtaagtagaaattaaattacgagcaaaagttggttgaaaataaaaatgaagatattgtgataaattatttctttttttttatatttgaccaacttacattgtaaagcctgtaatttttattaatttcgcattatagccataaaaagttattccaatctatgaagttgtgactttgcggaacatattttccgaaaaacttatttttcaagttttttaaatagtaagaaaaaggaaattttaaattataataacataataggtagtaaatataaatagtatatatttataattcaaataattatcaggaaatcttatttattttatgaaatttctaggtaaaattaatttagtcatgactaagtgttgtttctcacaaaattgtaatcataatcaaactaaattttgatatcgagaaacacaaaatgagcatttgttgaaagagacttataggtcgagtcttaacaaacttgataagattataaaattgtctagcataaactcctcatgcagcgaaacaataatgtatgattacaatcataagccaatgttcatgttggataaaccacacgataactaattctagaatctctcaagagtctaaataatttctatgcacacttttaaatttatttaagcttcatagtgaaatattagcttgtgctacactaatttacattgtacgttattttcaatggaatgacaactttattttgtgcccatagtgcataacagtaaaatttcagttcaatgatttcatatattattttttatattacgaagttttaggtttttggattttagattacaatcgagaccatagcaagagttgaaacatgcaaatgagatttaagcttaaaaaaatgttataaaattattaacatttagcacttatagtagctacaaaaatttgaaggattgaatatttctaaatattacaaagtataaacttaaatatcaacaaatatgattcatggagtttgagttaagtataatttaacaatatatcaatgatcgagttttttgcaaaaatcataaatattagctcaatttgaagaacttaacaaagcactcaaaactttaattgaaaacaaaaatttaaggttaagggcttatatatataaaaaaaaattcaaagtatgaaagcaatttcggacttcaatttttataatttttgattcataacgaactattaaattattgataaatatacttctgcatagagtccaaaaaattataaggccggcctaataaagcctatgatagaatcctatagaaacattgttgacccaagcacaggccgagtgaactcgtggctgagtttctatgctaagagttttgatcatgtgtaagaataaactaagtaagaataacattaaataaataattgaatctttacata

SEQ ID NO: 27 - DNA, 1567 bp, Xanthium strumarium
agaaataaaagacacatatgttcgtgaggttatgacacctttggttgatgttgtagccagtgattctagtgcaacactaatcgatttccacaccttgtgggtaagtcatcaatattcaagggtacctgtttttgaacaacgtgttgataatatagtcggtattgcatatgcaatggatctattagactatgttcaaaagggagaccttttagaaagtaccacggtgggggatatggctcataaacctgcttattttgtgcctgattcaatgtcggtatggaatcttcttagagaatttcgcattaggaaggtacacatggctgttgtcctgaacgaatacggtgggacagttggaattgtaacactagaagacgtggttgaagaaattgttggtgaaatttcttcaacccacgcctcttatctccttaaatccggccaacttaacttcctcttcaccgccccttattccccttccatctcatccaccacaaccaccgcctccatcccttccttttctcactccacctgtcgccacttcacctcctcccacggcctcgctgttcgtgccatcgccgttgaagtccatgacgccgaactcgctttctccgttagcgtcgctcacggcgctaaagcctccggataccataatccagctcttcaaacgacgacgtttctccacagcttcaaacccaggcaaaacatattgttattattattattgtttgtaactaatataacgcaaaacgacgtcgccgtagagttgaacttctgataagacgacgtcgttatgaccgagggtgataatccggagggttgaccacgcagtggggaacgtgccggagttagcaccggcagtggaatatataaaatcatttactggatttcacgagtttgctgagtttacggcggaggatgtgggaacgagtgagagtggactaaactcggtggttttggcttgcaatagtgagatggtattgataccgatgaatgaaccggtttacgggacgaagaggaagagtcagatacagacgtatttggaacataatgaaggggcgggggttcagcatttggcgttggctagtgaggatatatttaggacgttaagggagatgaggaaaaggagtggggttggtggctttgagtttatgccgtctccccctcctacttattataggaatttgaagaatagggtgggcgatgtgttgtctgatgaacagattaaggagtgtgaagaattggggatattggttgatagagatgatcaggggactttgcttcagatttttaccaagcctgttggtgacaggccgacgatattcatagagataattcagagagtagggtgtatggtgaaggataatgaaggaaaggagcagcagaaggcagggtgtggagggtttggcaaagggaacttctcagagctttttaaatccattgaggaatatgagaagacattggaagcaagagccaccactgaagccactgctgctgcatgaaaaccacccatgaataatcttcatgagattttataatctaatgattatgtatctgtggattctatacgaac

SEQ ID NO: 28 - DNA, 892 bp, Digitaria sanguinalis
ttgtgaagttggtgtcatatatttcaagatctgttcgtatagaatccacagatacataatcattagattataaaatctcatgaagattattcatgggtggttttcatgcagcagcagtggcttcagtggtggctcttgcttccaatgtcttctcatattcctcaatggatttaaaaagctctgagaagttccctttgccaaaccctccacaccctgccttctgctgctcctttccttcattatccttcaccatacaccctactctctgaattatctctatgaatatcgtcggcctgtcaccaacaggcttggtaaaaatctgaagcaaagtcccctgatcatctctatcaaccaatatccccaattcttcacactccttaatctgttcatcagacaacacatcgcccaccctattcttcaaattcctataataagtaggagggggagacggcataaactcaaagccaccaaccccactccttttcctcatctcccttaacgtcctaaatatatcctcactagccaacgccaaatgctgaacccccgccccttcattatgttccaaatacgtctgtatctgactcttcctcttcgtcccgtaaaccggttcattcatcggtatcaataccatctcactattgcaagccaaaaccaccgagtttagtccactctcactcgttcccacatcctccgccgtaaactcagcaaactcgtgaaatccagtaaatgattttatatattccactgccggtgctaactccggcacgttccccactgcgtggtcaagcctccggataccataatccagctcttcaaacgacgacgttttctccacagcttcaaacccaggcaaaaacatattgttattattattattgtttttgtaactaat

SEQ ID NO: 29 - DNA, 975 bp, Digitaria sanguinalis
gccgccaccgcctccctcccctccttctccgcccccgccgcgcgccgcttcgcctccgaccacggcctcgccgtgcgcgccgtagcgctccgcgtcgccgacgccgaggacgccttccgcgccagcgtcgccaacggggcgcgcccggcgttcgagcccgtcgagctcggcctcggcttccgcctcgccgaggtcgagctctacggcgacgtcgtgctccgctacgtcagctacccggacgccgcggatttgcccttcctgccgggcttcgaggacgtcgtcatcagcaacccaggggcggtggactacgggctgaggcgcttcgaccacatcgtcggcaacgtctcggagctggcgccggtggccgcgtacgtcgccggattcacggggttccacgagttcgccgagttcacggcggaggacgtgggcacggcggagagcgggctcaattccgtggtgctcgccaacaactccgagaacgtgctgctcccgctcaacgagccggtgcacggcaccaagcgccgcagccagatacagacatacctggaccaccacggcggccctggagtgcagcacatcgcgctggctagcgacgacgtgctcaggacactgcgggagatgcaggcgcgctccgccatgggtgggtttgagttcatggaggctccgccacccacttactatgagggtgtgaggcggcgcgccggggacgtgctctcggaggagcagataaaggagtgccagaaactgggggtgctggtggacagggatgaccagggagtgttgctccaaatcttcaccaagccagtgggggacccaacgtttttcttggagataatccaaaggattgggtgcatggagaaggatgagcagggaaaggactaccagaagggcggctgtggcgggtttggcaagggaaacttctcacagctgttcaagtctattgaggagtatgagaagtcccttgaagcgaagcaa

SEQ ID NO: 30 - DNA, 1266 bp, Kochia scoparia
accaccgcctcaaccgagttcaagctggtgggttactccaacttcgtccgagttaaccccatgtccgacctcttctccgtcaaaaaattccaccacatcgagttctggtgcggcgacgcgaccaacgtcagccgtcgcttctcatggggtctaggcatgccggcggtcgctaaatccgacctctccaccggaaactccgtacacgcttcgtacctccttcgctcaggcgacctctccttcctcttcacctccccgtactccccttctctctcctccccctcttccgctgcaatacccacgtttgatttctctctcttctcctcttttatcacctcccacggcatcggggttcgcgccgttgcactcgaggtcgacgatgccgaggttgctttcaatacgagcgtctcccacggcgcaatccctggttctcccccgattaagctcggaaacggcgtcgttttgtccgaggtcagcctctacggcgacgtcgttcttcgctacgtgagctacggaggtgagacagagaaagcggatacgaattcgaattcatggtttcttcctgggttcgaggaaatgccggaggaatcgtcgtttcgagggctcgatttcgggctccggagactagaccatgcggtggggaatgtcccggagctagggaaagcaatcgagtatgtgaagaagttcactgggtttcacgaatttgctgagtttacagctgacgatgttgggacgagtgaaagtgggctgaattcggctgtgctggcgaacaactcggagacggtgttgattccgatgaacgagccggtttacgggacgaagaggaagagtcaaattcagacatacttggagcacaatgaaggagctggggttcagcatttggcattgatgagtgaggatatattctggactcttagggagatgaggaagcggagcgggctcggtgggttcgagtttatgccagcaccgccgcctacgtattatcggaacctgaggaatcgtgtcggggatgtgttgagtgaagaacagatgaaggagtgtgaagagttggggatattggttgataaggatgatcagggcactctgctccagatttttactaagcatattggtgacccaactatgttcattgagattatccaaagaattggctgcatgatgaaagatgaagaaggcaaggtgtaccaaaagggaggctgtggaggatttgggaagggaaacttttcagagcttttcaaatctatcgaagagtatgagaagacacttgaa

SEQ ID NO: 31 - DNA, 783 bp, Lolium multiflorum
ctcggccacgggtttggcttcgcggaggtggagctagccggggacagcgttctccgcttcgtgagctacccggacggcaccgacgtgtccttcctgccggggttccaggacgtggcgagcgccggcggggcgccggacttcgggctcacccggtttgaccacgtcgacgttaacatcccggagctggcacccgtcgccgccaatgttgccggcttcaccgggttccacaaattctgggagttcaccgcggacgacgtgtgcccggaagagagcggggtgaacggcgtggtgatcgccaacaactcagagaacgtgctgctcagtatcttggagccggtgttcggcaccaagctgcggagccatgtcgagacgttcctggaccaccacggtggcccgggcatacagcacctggcaatgaccagccacgacatccttggcgcgctcaggaaaatccgagctcggtcctccatgggcgggtttgagctcctgccgccgccgccggccagctactatgacggtgtaaggcagcgcgccggggacgtgctgtcggaagaacagatcaaggagtgccaagagctgggcgtgcgggtggacagagggtatgaggacggagttgtgctccaagtcttcaccaaaccggcgggagacccaaccttactgttagagtttatccaaagaatcgggtgcatggtcaaggacgagaaccagcaggaataccagagaggtggatgtggcgggtttgccaaagggaacgtttctgaactcatcaaggacattgaggac

SEQ ID NO: 32 - DNA, 1041 bp, Lolium multiflorum
accgatcgcttccacgtgatggatttccaccacgtcgagttttggtgcgccgatgccgcctcggccgccgcacggttctccttcgggctcggcgtgccactcgccgcgcagtccgacctctccacggggaacactgcgcacgcctcacgcctactacgcgcacgctcgggctctctctcgttcctcttcaccgcgccgtacgcgccgcacgtcgccgactcggcgaccaccgcgtccctgccctccttctcggcggacgccgcgcggcgcttcacgggcacccacggcggcctggccgtgcgtgccgtggccgtccgcgtcgctgacgcggccgaggccttcgtcgcgagcgtggacgccggagcgcggccagcctgcgccccgactgatctcggccacgggtttggcttcgcggaggtggagctagccggggacagcgttctccgcttcgtgagctacccggacggcaccgacgtgtccttcctgccggggttccaggacgtggcgagcgccggcggggcgccggacttcgggctcacccggtttgaccacgtcgacgttaacatcccggagctggcacccgtcgccgccaatgttgccggcttcaccgggttccacaaattctgggagttcaccgcggacgacgtgtgcccggaagagagcggggtgaacggcgtggtgatcgccaacaactcagagaacgtgctgctcagtatcttggagccggtgttcggcaccaagctgcggagccatgtcgagacgttcctggaccaccacggtggcccgggcatacagcacctggcaatgaccagccacgacatccttggcgcgctcaggaaaatccgagctcggtcctccatgggcgggtttgagctcctgccgccgccgccggccagctactatgacggtgtaaggcagcgcgccggggacgtgctgtcggaagaacagatcaaggagtgccaagagctgggcgtgcgggtggacagagggtatgaggacggagttgtgctccaagtcttcaccaaaccggcgggagacagg

SEQ ID NO: 33 - DNA, 200 bp, Abutilon theophrasti
tccataatccaacgcagacatttcagagattcatccccgaagaaatcaaaaatccctcaacaaaatgattcagatatgcttaaacaaaatatgaattgagcacaatatcatatttgacatttatgttgaagtagcaaaggttcgtatagtttatgtatgtgcaattaaaactaacagtaacggaattcattctgacactc

SEQ ID NO: 34 - DNA, 200 bp, Abutilon theophrasti
gagtgtcagaatgaattccgttactgttagttttaattgcacatacataaactatacgaacctttgctacttcaacataaatgtcaaatatgatattgtgctcaattcatattttgtttaagcatatctgaatcattttgttgagggatttttgatttcttcggggatgaatctctgaaatgtctgcgttggattatgga

SEQ ID NO: 35 - DNA, 200 bp, Abutilon theophrasti
agtaacggaattcattctgacactccctatatgctgctagcaactacctcgatctctcaagcctcgacaacggtggtggattctgtagattgtttggcttcaagagacttctcgtattcttcaatggatttgaagagctcggaaaagttgcctttcccgaaacctccgcatcctcctttctggtattgctttccttcttc

SEQ ID NO: 36 - DNA, 200 bp, Abutilon theophrasti
gaagaaggaaagcaataccagaaaggaggatgcggaggtttcgggaaaggcaacttttccgagctcttcaaatccattgaagaatacgagaagtctcttgaagccaaacaatctacagaatccaccaccgttgtcgaggcttgagagatcgaggtagttgctagcagcatatagggagtgtcagaatgaattccgttact

SEQ ID NO: 37 - DNA, 200 bp, Abutilon theophrasti
ctttctggtattgctttccttcttcatccttcaccatgcaccctaatctctgtattatttctatgaatatcgtaggcctatctccaatgggcttagtgaaaatctgaagcagagtgccttgatcatctctgtcaaccaaaatccccaactcttcacactccttaatctgctcatcgctcaaaatgtcccctgccctttgc

SEQ ID NO: 38 - DNA, 200 bp, Abutilon theophrasti
gcaaagggcaggggacattttgagcgatgagcagattaaggagtgtgaagagttggggattttggttgacagagatgatcaaggcactctgcttcagattttcactaagcccattggagataggcctacgatattcatagaaataatacagagattagggtgcatggtgaaggatgaagaaggaaagcaataccagaaag

SEQ ID NO: 39 - DNA, 200 bp, Amaranthus graecizans
cttcttccccgtctgagtttttattacttcactttctctctcatcatctaacatgggaactttgaaacccgaaactcaacccgactccgaattcaaactcgtgggttactccaacttcattcgggttaaccccaaatctgaccgttttactgttaagcgtttccatcatatagagttctggtgtggcgatgcaaccaatg

SEQ ID NO: 40 - DNA, 200 bp, Amaranthus graecizans
cattggttgcatcgccacaccagaactctatatgatggaaacgcttaacagtaaaacggtcagatttggggttaacccgaatgaagttggagtaacccacgagtttgaattcggagtcgggttgagtttcgggtttcaaagttcccatgttagatgatgagagagaaagtgaagtaataaaaactcagacggggaagaag

SEQ ID NO: 41 - DNA, 200 bp, Amaranthus graecizans
ttctggtgtggcgatgcaaccaatgttagcagacgcttttcttggggtcttggaatgcctaccgttgctaaatctgacctttctactggaaactctgttcacgcttcttttcttcttcgttccggtgacctttctttcctctttacttcaccttactctcctaccatgtccatcccttcttctgctgcaatcccctcgtt

SEQ ID NO: 42 - DNA, 200 bp, Amaranthus graecizans
aacgaggggattgcagcagaagaagggatggacatggtaggagagtaaggtgaagtaaagaggaaagaaaggtcaccggaacgaagaagaaaagaagcgtgaacagagtttccagtagaaaggtcagatttagcaacggtaggcattccaagaccccaagaaaagcgtctgctaacattggttgcatcgccacaccagaa

SEQ ID NO: 43 - DNA, 200 bp, Amaranthus graecizans
cttcttctgctgcaatcccctcgtttgatttcaatcattttaccaaatttgttacatcgcacggtcttggcgtgcgtgctgttgccgtcgaagtagaagacgcagaagctgcttttaatatcagcgtttcgcacggggctatcccctgtgtttctcctattcacttggaaaacggtgtcgttttatctgaggttcattta

SEQ ID NO: 44 - DNA, 200 bp, Amaranthus graecizans
taaatgaacctcagataaaacgacaccgttttccaagtgaataggagaaacacaggggatagccccgtgcgaaacgctgatattaaaagcagcttctgcgtcttctacttcgacggcaacagcacgcacgccaagaccgtgcgatgtaacaaatttggtaaaatgattgaaatcaaacgaggggattgcagcagaagaag

SEQ ID NO: 45 - DNA, 200 bp, Amaranthus graecizans
tgtcgttttatctgaggttcatttatatggggatgttgtgcttcgttatgtaagctacggaaatgaatgtggggatgtgttttttcttcctgggtttgagcaaatgcccgaggaatcctcgtttcgaggacttgatttcggccttcgaaggttggatcatgctgtagggaatgtccctgagttggctcctgcaattgctt

SEQ ID NO: 46 - DNA, 200 bp, Amaranthus graecizans
aagcaattgcaggagccaactcagggacattccctacagcatgatccaaccttcgaaggccgaaatcaagtcctcgaaacgaggattcctcgggcatttgctcaaacccaggaagaaaaaacacatccccacattcatttccgtagcttacataacgaagcacaacatccccatataaatgaacctcagataaaacgaca

SEQ ID NO: 47 - DNA, 200 bp, Amaranthus graecizans
cctgagttggctcctgcaattgcttatttgaagaagtttactgggtttcatgagtttgctgagtttacagctgaagatgttggaacgagtgaaagtgggttgaattcagccgtattggcaaataatgatgaaatggtgttgtttccaatgaatgaacctgtgtatgggacaaaaaggaagagtcaaattcaaacttattt

SEQ ID NO: 48 - DNA, 200 bp, Amaranthus graecizans
aaataagtttgaatttgactcttcctttttgtcccatacacaggttcattcattggaaacaacaccatttcatcattatttgccaatacggctgaattcaacccactttcactcgttccaacatcttcagctgtaaactcagcaaactcatgaaacccagtaaacttcttcaaataagcaattgcaggagccaactcagg

SEQ ID NO: 49 - DNA, 200 bp, Amaranthus graecizans
ggaagagtcaaattcaaacttatttggagcataatgaaggagctggtgtacaacatttggctttgatgagtgaagatatattttggactttaagggagatgaggaagagaagtggtcttggtgggtttgagtttatgccgtcgccgcctccgacttattaccggaatttgaggaacagagctgctgatgtattgagtgag

SEQ ID NO: 50 - DNA, 200 bp, Amaranthus graecizans
ctcactcaatacatcagcagctctgttcctcaaattccggtaataagtcggaggcggcgacggcataaactcaaacccaccaagaccacttctcttcctcatctcccttaaagtccaaaatatatcttcactcatcaaagccaaatgttgtacaccagctccttcattatgctccaaataagtttgaatttgactcttcc

SEQ ID NO: 51 - DNA, 200 bp, Amaranthus hybridus
tcttcctgggtttgaggaaatgccggaggaatcgtcgtttcgaggacttgatttcggccttcgaaggttggatcatgctgtagggaatgttcccgagttggctcctgcaattgcttatttgaagaagtttactgggtttcatgagtttgctgagtttacagctgaagatgttgggacgagtgaaagtgggttgaattcag

SEQ ID NO: 52 - DNA, 200 bp, Amaranthus hybridus
ctgaattcaacccactttcactcgtcccaacatcttcagctgtaaactcagcaaactcatgaaacccagtaaacttcttcaaataagcaattgcaggagccaactcgggaacattccctacagcatgatccaaccttcgaaggccgaaatcaagtcctcgaaacgacgattcctccggcatttcctcaaacccaggaaga

SEQ ID NO: 53 - DNA, 200 bp, Amaranthus hybridus
acgagtgaaagtgggttgaattcagccgtattggcaaataatgatgaaatggtgttgtttccaatgaatgaacctgtgtatgggacaaaaaggaagagccaaattcaaacttatttggagcataatgaaggagctggtgtacaacatttggctttgatgagtgaagatatattttggactttaagggagatgaggaagag

SEQ ID NO: 54 - DNA, 200 bp, Amaranthus hybridus
ctcttcctcatctcccttaaagtccaaaatatatcttcactcatcaaagccaaatgttgtacaccagctccttcattatgctccaaataagtttgaatttggctcttcctttttgtcccatacacaggttcattcattggaaacaacaccatttcatcattatttgccaatacggctgaattcaacccactttcactcgt

SEQ ID NO: 55 - DNA, 200 bp, Amaranthus palmeri
agatgtgtataagagacaggtcttggcgtgcgtgctgttgccgtcgaagtagaagacgccgaagctgcttttaatatcagcgtttcgcacggggctatcccctgtgtttctcctattcacttggaaaacggtgtcgttttatctgaggttcatttatatggggatgttgtgcttcggtatgtaagctacggaaatgaatg

SEQ ID NO: 56 - DNA, 200 bp, Amaranthus palmeri
cattcatttccgtagcttacataccgaagcacaacatccccatataaatgaacctcagataaaacgacaccgttttccaagtgaataggagaaacacaggggatagccccgtgcgaaacgctgatattaaaagcagcttcggcgtcttctacttcgacggcaacagcacgcacgccaagacctgtctcttatacacatct

SEQ ID NO: 57 - DNA, 200 bp, Amaranthus palmeri
ggtatgtaagctacggaaatgaatgtggggatgtgtttttcttcctgggtttgaggaaatgccggaggaatcatcgtttcgaggacttgattttggcattcgaaggttggatcatgctgtagggaatgtccctgagttggctcctgcaattgcttatttgaagaagtttactgggtttcatgagtttgctgagtttacag

SEQ ID NO: 58 - DNA, 200 bp, Amaranthus palmeri
ctgtaaactcagcaaactcatgaaacccagtaaacttcttcaaataagcaattgcaggagccaactcagggacattccctacagcatgatccaaccttcgaatgccaaaatcaagtcctcgaaacgatgattcctccggcatttcctcaaacccaggaagaaaaacacatccccacattcatttccgtagcttacatacc

SEQ ID NO: 59 - DNA, 200 bp, Amaranthus palmeri
tttcatgagtttgctgagtttacagctgaagatgttgggacgagtgaaagtggattgaattcagccgtattggcaaacaatgatgaaatggtgttgtttccaatgaatgaacctgtgtatgggacaaaaaggaagagccaaattcaaacttatttggagcataatgaaggagctggtgtacagcatttggctttgatgag

SEQ ID NO: 60 - DNA, 200 bp, Amaranthus palmeri
ctcatcaaagccaaatgctgtacaccagctccttcattatgctccaaataagtttgaatttggctcttcctttttgtcccatacacaggttcattcattggaaacaacaccatttcatcattgtttgccaatacggctgaattcaatccactttcactcgtcccaacatcttcagctgtaaactcagcaaactcatgaaa

SEQ ID NO: 61 - DNA, 200 bp, Amaranthus palmeri
gtgtacagcatttggctttgatgagtgaagacatattttggactttaagggagatgaggaagagaagtggtcttggtgggtttgagtttatgccgtcgccgcctccgacttattaccggaatttgaggaacagagctgctgatgtattgagtgaggagcagatgaaggagtgtgaagagttggggattttggtggataaa

SEQ ID NO: 62 - DNA, 200 bp, Amaranthus palmeri
tttatccaccaaaatccccaactcttcacactccttcatctgctcctcactcaatacatcagcagctctgttcctcaaattccggtaataagtcggaggcggcgacggcataaactcaaacccaccaagaccacttctcttcctcatctcccttaaagtccaaaatatgtcttcactcatcaaagccaaatgctgtacac

SEQ ID NO: 63 - DNA, 200 bp, Amaranthus palmeri
agagttggggattttggtggataaagatgatcagggtactttgcttcaaatcttcaccaaacctattggagacaggccaaccatattcatcgagattatccaaagaattggttgcatgatgaaagatgaagacggcaagatgtaccaaaagggtggttgcggaggattcggaaagggaaacttttcagagctgttcaaat

SEQ ID NO: 64 - DNA, 200 bp, Amaranthus palmeri
atttgaacagctctgaaaagtttccctttccgaatcctccgcaaccacccttttggtacatcttgccgtcttcatctttcatcatgcaaccaattctttggataatctcgatgaatatggttggcctgtctccaataggtttggtgaagatttgaagcaaagtaccctgatcatctttatccaccaaaatccccaactct

SEQ ID NO: 65 - DNA, 200 bp, Amaranthus palmeri
ggaaacttttcagagctgttcaaatcaatagaggagtacgagaagactcttgaacgtaaacaggttccagatacagctgctgcatgatgagcagactaaaatattgttgtcttgctgatgaaatgatagaaaaggtttgtttcttggtacaatgctcaacttcaaaattttctttattaaataatgaagtgtaaacttat

SEQ ID NO: 66 - DNA, 200 bp, Amaranthus palmeri
ataagtttacacttcattatttaataaagaaaattttgaagttgagcattgtaccaagaaacaaaccttttctatcatttcatcagcaagacaacaatattttagtctgctcatcatgcagcagctgtatctggaacctgtttacgttcaagagtcttctcgtactcctctattgatttgaacagctctgaaaagtttcc

SEQ ID NO: 67 - DNA, 200 bp, Amaranthus palmeri
attaaataatgaagtgtaaacttatacaaactgtgtcatatatggtgattggtgatcatggtttatgtagaatgtataacataattgataatctgtgtatatgctgaatacttacatactgtgaaatcattctgatggaaacatacaattggtcagtagctgaggctggtagctcctcagattagtttttttcagtcaaa

SEQ ID NO: 68 - DNA, 200 bp, Amaranthus palmeri
tttgactgaaaaaaactaatctgaggagctaccagcctcagctactgaccaattgtatgtttccatcagaatgatttcacagtatgtaagtattcagcatatacacagattatcaattatgttatacattctacataaaccatgatcaccaatcaccatatatgacacagtttgtataagtttacacttcattatttaat

SEQ ID NO: 69 - DNA, 200 bp, Amaranthus palmeri
gagttcaagattaaaagttaaattacatttatgtgggttttatataggcaatgattgcattatattgtttttcttttggtgggaagattttcctttttaaaaaattttaatttccctacattttcaaatgatggaggaatttttttgtgcatgtaaaatgttttctctctattttttgatttatgctattttttctcttt

SEQ ID NO: 70 - DNA, 200 bp, Amaranthus palmeri
aaagagaaaaaatagcataaatcaaaaaatagagagaaaacattttacatgcacaaaaaaattcctccatcatttgaaaatgtagggaaattaaaattttttaaaaaggaaaatcttcccaccaaaagaaaaacaatataatgcaatcattgcctatataaaacccacataaatgtaatttaacttttaatcttgaactc

SEQ ID NO: 71 - DNA, 200 bp, Amaranthus palmeri
ttgatttatgctattttttctcttttatttttatccaaaaatatcatgggatgtctagattggaataattagggagtaaaaagtaccccttgattatgcagagcaaaaataaattgtctgaatttgaaataaatatctacaagagtaaatttttccatcttattcaaaggtaaatgtttgatccacctacctctatagat

SEQ ID NO: 72 - DNA, 200 bp, Amaranthus palmeri
atctatagaggtaggtggatcaaacatttacctttgaataagatggaaaaatttactcttgtagatatttatttcaaattcagacaatttatttttgctctgcataatcaaggggtactttttactccctaattattccaatctagacatcccatgatatttttggataaaaataaaagagaaaaaatagcataaatcaa

SEQ ID NO: 73 - DNA, 200 bp, Amaranthus palmeri
gtttgatccacctacctctatagatatatttcagggaactaaattgtcttcaccattaaatttggttacttggtctttaaaacccaaatcatagaaattaatgataaaatcaaaataaaaaagatatttaaattcaaattcaaactaactaattttaaattacaaaatgaatatctgtaatttacaaaagaaagtatcaa

SEQ ID NO: 74 - DNA, 200 bp, Amaranthus palmeri
ttgatactttcttttgtaaattacagatattcattttgtaatttaaaattagttagtttgaatttgaatttaaatatcttttttattttgattttatcattaatttctatgatttgggttttaaagaccaagtaaccaaatttaatggtgaagacaatttagttccctgaaatatatctatagaggtaggtggatcaaac

SEQ ID NO: 75 - DNA, 200 bp, Amaranthus palmeri
tgtaatttacaaaagaaagtatcaaaaacatatgaaaatctcaacatctgaaaattaaaaaacaagtattttgtttcttcatttttttctttttcgctatttcctttcaaaaataaaagtaaataaaaatattcaaaagcaattccataaacaaaatcttagatatgtaaatcacaaaaacattagatctagaaaaaaaa

SEQ ID NO: 76 - DNA, 200 bp, Amaranthus palmeri
ttttttttctagatctaatgtttttgtgatttacatatctaagattttgtttatggaattgcttttgaatatttttatttacttttatttttgaaaggaaatagcgaaaaagaaaaaaatgaagaaacaaaatacttgttttttaattttcagatgttgagattttcatatgtttttgatactttcttttgtaaattaca

SEQ ID NO: 77 - DNA, 200 bp, Amaranthus palmeri
aaaaacattagatctagaaaaaaaaaatttcttccattgcaaaccctttttcaaccttcataacttccactaccataatgaggccagtaaagagacaaaagtcattgagttgttgttgtgcagttgatgataaatgatgatagaagggtttattttttttttgaaatgaatggttagattttctgactttttatttaccc

SEQ ID NO: 78 - DNA, 200 bp, Amaranthus palmeri
gggtaaataaaaagtcagaaaatctaaccattcatttcaaaaaaaaaataaacccttctatcatcatttatcatcaactgcacaacaacaactcaatgacttttgtctctttactggcctcattatggtagtggaagttatgaaggttgaaaaagggtttgcaatggaagaaattttttttttctagatctaatgttttt

SEQ ID NO: 79 - DNA, 200 bp, Amaranthus palmeri
agattttctgactttttatttaccctatatagaatatcaaacaattaactctataaattatttaatacattaaaatgtttcatgtaatatgtctcctatattatttaccctttaatttttaagtgggaaccaagtatgtcttaattatctttattttaatcaaatacgcggtatacatgaaataatcaacaaatgcaatt

SEQ ID NO: 80 - DNA, 200 bp, Amaranthus palmeri
aattgcatttgttgattatttcatgtataccgcgtatttgattaaaataaagataattaagacatacttggttcccacttaaaaattaaagggtaaataatataggagacatattacatgaaacattttaatgtattaaataatttatagagttaattgtttgatattctatatagggtaaataaaaagtcagaaaatct

SEQ ID NO: 81 - DNA, 200 bp, Amaranthus palmeri
catgaaataatcaacaaatgcaattactatgctcggacgagagtaaatataatgggaggaagttgtacatacaattacgaaatagtctaaataaataacgataatttgtaatataaacaaacaaatcacacttatataaacagattttatagggtggaatcattaggattctaatttatcttttttcttcttttgtttac

SEQ ID NO: 82 - DNA, 200 bp, Amaranthus palmeri
gtaaacaaaagaagaaaaaagataaattagaatcctaatgattccaccctataaaatctgtttatataagtgtgatttgtttgtttatattacaaattatcgttatttatttagactatttcgtaattgtatgtacaacttcctcccattatatttactctcgtccgagcatagtaattgcatttgttgattatttcatg

SEQ ID NO: 83 - DNA, 200 bp, Amaranthus palmeri
ttatcttttttcttcttttgtttactttgctgatatttattttgtatttttcctatttctcaaaaggaagactaacactcaaataaaatgatattgaaatacaaagcatcaccagccaagccgagatgacaaaactattggctaagtgataactgataagtgacgtatttccgttactctcaagtcttaacagctttttg

SEQ ID NO: 84 - DNA, 200 bp, Amaranthus palmeri
caaaaagctgttaagacttgagagtaacggaaatacgtcacttatcagttatcacttagccaatagttttgtcatctcggcttggctggtgatgctttgtatttcaatatcattttatttgagtgttagtcttccttttgagaaataggaaaaatacaaaataaatatcagcaaagtaaacaaaagaagaaaaaagataa

SEQ ID NO: 85 - DNA, 200 bp, Amaranthus palmeri
actctcaagtcttaacagctttttgtcacgcaaggaaaagaagaccgtggacgtcaacggtgacgttgaatgttcatctttacagtcgcagtcaatcaatctctttttagatcgatcttccacctcaattctccgttacaaatcaaattccatctagaacttcttttttattattttgactcataaattcccccaaaaat

SEQ ID NO: 86 - DNA, 200 bp, Amaranthus palmeri
atttttgggggaatttatgagtcaaaataataaaaaagaagttctagatggaatttgatttgtaacggagaattgaggtggaagatcgatctaaaaagagattgattgactgcgactgtaaagatgaacattcaacgtcaccgttgacgtccacggtcttcttttccttgcgtgacaaaaagctgttaagacttgagagt

SEQ ID NO: 87 - DNA, 200 bp, Amaranthus palmeri
ttgactcataaattcccccaaaaatacttctattttattataaataaattccaatttctatgttctccattcattaccacccattactccgttttccaaaccaccattttctctctcctcctttaccgctaacgctaccaccattttcgcttcttccccgtctgaattttattacttcgctttctctatcatcatctgac

SEQ ID NO: 88 - DNA, 200 bp, Amaranthus palmeri
gtcagatgatgatagagaaagcgaagtaataaaattcagacggggaagaagcgaaaatggtggtagcgttagcggtaaaggaggagagagaaaatggtggtttggaaaacggagtaatgggtggtaatgaatggagaacatagaaattggaatttatttataataaaatagaagtatttttgggggaatttatgagtcaa

SEQ ID NO: 89 - DNA, 200 bp, Amaranthus palmeri
ttcgctttctctatcatcatctgacatgggaactttaaaacccgaaactcaacccgattccgaattcaaactcgtgggttactccaacttcgtccgggttaaccccaaatctgaccgttttgctgttaagcgtttccaccatatagagttttggtgtggcgatgcaaccaatgttagcagacgattttcttggggtcttg

SEQ ID NO: 90 - DNA, 200 bp, Amaranthus palmeri
caagaccccaagaaaatcgtctgctaacattggttgcatcgccacaccaaaactctatatggtggaaacgcttaacagcaaaacggtcagatttggggttaacccggacgaagttggagtaacccacgagtttgaattcggaatcgggttgagtttcgggttttaaagttcccatgtcagatgatgatagagaaagcgaa

SEQ ID NO: 91 - DNA, 200 bp, Amaranthus palmeri
agcagacgattttcttggggtcttggaatgcctaccgtcgctaaatctgacctgtctacaggaaactctgttcacgcttcttttcttcttagttccggtgacctttcttttctctttacttcaccttactctcctaccatgtccatcccttcttctgctgcaatcccctcgtttgatttcaatcattttaccaaattcct

SEQ ID NO: 92 - DNA, 200 bp, Amaranthus palmeri
aggaatttggtaaaatgattgaaatcaaacgaggggattgcagcagaagaagggatggacatggtaggagagtaaggtgaagtaaagagaaaagaaaggtcaccggaactaagaagaaaagaagcgtgaacagagtttcctgtagacaggtcagatttagcgacggtaggcattccaagaccccaagaaaatcgtctgct

SEQ ID NO: 93 - DNA, 200 bp, Amaranthus palmeri
atttcaatcattttaccaaattccttacatcgcacggtcttggcgtgcgtgctgttgccgtcgaagtagaagacgcagaagctgcttttaatatcagcgtttcgcacggggctatcccctgtgtttctcctattcacttggaaaacggtgtcgttttatctgaggttcatttatatggggatgttgtgcttcggtatgta

SEQ ID NO: 94 - DNA, 200 bp, Amaranthus palmeri
tacataccgaagcacaacatccccatataaatgaacctcagataaaacgacaccgttttccaagtgaataggagaaacacaggggatagccccgtgcgaaacgctgatattaaaagcagcttctgcgtcttctacttcgacggcaacagcacgcacgccaagaccgtgcgatgtaaggaatttggtaaaatgattgaaat

SEQ ID NO: 95 - DNA, 200 bp, Amaranthus palmeri
tggggatgttgtgcttcggtatgtaagctacggaaatgaatgtggggatgtgttttttcttcctgggtttgaggaaatgccggaggaatcatcgtttcgaggacttgattttggcattcgaaggttggatcatgctgtagggaatgtccctgagttggctcctgcaattgcttatttgaagaagtttactgggtttcatg

SEQ ID NO: 96 - DNA, 200 bp, Amaranthus palmeri
catgaaacccagtaaacttcttcaaataagcaattgcaggagccaactcagggacattccctacagcatgatccaaccttcgaatgccaaaatcaagtcctcgaaacgatgattcctccggcatttcctcaaacccaggaagaaaaaacacatccccacattcatttccgtagcttacataccgaagcacaacatcccca

SEQ ID NO: 97 - DNA, 200 bp, Amaranthus palmeri
ttgaagaagtttactgggtttcatgagtttgctgagtttacagctgaagatgttgggacgagtgaaagtgggttgaattcagccgtattggcaaacaatgatgaaatggtgttgtttccgatgaatgaacctgtgtatgggacaaaaaggaagagccaaattcaaacttatttggagcataatgaaggagctggtgtaca

SEQ ID NO: 98 - DNA, 200 bp, Amaranthus palmeri
tgtacaccagctccttcattatgctccaaataagtttgaatttggctcttcctttttgtcccatacacaggttcattcatcggaaacaacaccatttcatcattgtttgccaatacggctgaattcaacccactttcactcgtcccaacatcttcagctgtaaactcagcaaactcatgaaacccagtaaacttcttcaa

SEQ ID NO: 99 - DNA, 200 bp, Amaranthus palmeri
agcataatgaaggagctggtgtacagcatttggctttgatgagtgaagacatattttggactttaagggagatgaggaagagaagtggtcttggtgggtttgagtttatgccgtcgccgcctccgacttattaccggaatttgaggaacagagctgctgatgtattgagtgaggagcagatgaaggagtgtgaagagttg

SEQ ID NO: 100 - DNA, 200 bp, Amaranthus palmeri
caactcttcacactccttcatctgctcctcactcaatacatcagcagctctgttcctcaaattccggtaataagtcggaggcggcgacggcataaactcaaacccaccaagaccacttctcttcctcatctcccttaaagtccaaaatatgtcttcactcatcaaagccaaatgctgtacaccagctccttcattatgct

SEQ ID NO: 101 - DNA, 200 bp, Amaranthus palmeri
gcagatgaaggagtgtgaagagttggggattttggtggataaagatgatcagggcactttgcttcaaatcttcaccaaacctattggagacaggtagattttaatcttgctttcaattgcttttgcttgatggattgactagccaatttgattgcattttgttgcttatatgacttgatgctagatagtttacctttctc

SEQ ID NO: 102 - DNA, 200 bp, Amaranthus palmeri
gagaaaggtaaactatctagcatcaagtcatataagcaacaaaatgcaatcaaattggctagtcaatccatcaagcaaaagcaattgaaagcaagattaaaatctacctgtctccaataggtttggtgaagatttgaagcaaagtgccctgatcatctttatccaccaaaatccccaactcttcacactccttcatctgc

SEQ ID NO: 103 - DNA, 200 bp, Amaranthus palmeri
tgatgctagatagtttacctttctcagctgttaagttgtagcaagtatctaatacattcgttctgaaatatctgaaatatttgcaactgattatgacatgtagcgggagaaaatgtccgtttggcataaattaagcttggttttgcacttatcctcatttatttataaattctaaaacttgttagttgtaagctcctttc

SEQ ID NO: 104 - DNA, 200 bp, Amaranthus palmeri
gaaaggagcttacaactaacaagttttagaatttataaataaatgaggataagtgcaaaaccaagcttaatttatgccaaacggacattttctcccgctacatgtcataatcagttgcaaatatttcagatatttcagaacgaatgtattagatacttgctacaacttaacagctgagaaaggtaaactatctagcatca

SEQ ID NO: 105 - DNA, 200 bp, Amaranthus palmeri
aacttgttagttgtaagctcctttcagttgtcctgaatttaattaagttaatgagaacaaagccttaatattccttcaaggtgattgtagttggggcactagttctaacaatggaaatttggaaatctattccaactggtcccaagttaatctttgtttgcaagcctgattggttcaaattaaggttattgtattcttgt

SEQ ID NO: 106 - DNA, 200 bp, Amaranthus palmeri
acaagaatacaataaccttaatttgaaccaatcaggcttgcaaacaaagattaacttgggaccagttggaatagatttccaaatttccattgttagaactagtgccccaactacaatcaccttgaaggaatattaaggctttgttctcattaacttaattaaattcaggacaactgaaaggagcttacaactaacaagtt

SEQ ID NO: 107 - DNA, 200 bp, Amaranthus palmeri
caaattaaggttattgtattcttgtatgaattcgactcaatgtaaatttgtttaatggagtatcaatttttaatagttttcaaccaagtagtattagatatattcgggattgaaataatgcatctatgagtgtacaaaaccaaatggcacatttgattagaataaaagagagtataaggctaatttcgtttacctaatat

SEQ ID NO: 108 - DNA, 200 bp, Amaranthus palmeri
atattaggtaaacgaaattagccttatactctcttttattctaatcaaatgtgccatttggttttgtacactcatagatgcattatttcaatcccgaatatatctaatactacttggttgaaaactattaaaaattgatactccattaaacaaatttacattgagtcgaattcatacaagaatacaataaccttaatttg

SEQ ID NO: 109 - DNA, 200 bp, Amaranthus palmeri
aaggctaatttcgtttacctaatatttaaagcgacccctaaaattcaattggcctaaacccataaagttcaaaagcagagaagaacatagaatagtgcaggtccattggtaatgcactaggagttggagcttttatgggtacaagtgtgtggctagttggggatgactgtctagcattgtctaggtgaaaagctgaagcc

SEQ ID NO: 110 - DNA, 200 bp, Amaranthus palmeri
ggcttcagcttttcacctagacaatgctagacagtcatccccaactagccacacacttgtacccataaaagctccaactcctagtgcattaccaatggacctgcactattctatgttcttctctgcttttgaactttatgggtttaggccaattgaattttaggggtcgctttaaatattaggtaaacgaaattagcctt

SEQ ID NO: 111 - DNA, 200 bp, Amaranthus palmeri
ggaggaatttttttgtgcatgtaaaatgttttctctctattttttgatttatgctattttttctcttttatttttatccaaaaatatcatgggatgtctagattggaataattagggagtaaaaagtaccccttgattatgcagagcaaaaataaattgtctgaatttgaaataaatatctacaagagtaaatttttcca

SEQ ID NO: 112 - DNA, 200 bp, Amaranthus palmeri
tggaaaaatttactcttgtagatatttatttcaaattcagacaatttatttttgctctgcataatcaaggggtactttttactccctaattattccaatctagacatcccatgatatttttggataaaaataaaagagaaaaaatagcataaatcaaaaaatagagagaaaacattttacatgcacaaaaaaattcctcc

SEQ ID NO: 113 - DNA, 200 bp, Amaranthus palmeri
atatctacaagagtaaatttttccatcttattcaaaggtaaatgtttgatccacctacctctatagatatatttcagggaactaaaattgtcttcaccattatatttagttacttggtctttaaaacccaaatcatagaaattaacgataaaatcaaaatataaagatatttaaattccaattcaaactaactaatttta

SEQ ID NO: 114 - DNA, 200 bp, Amaranthus palmeri
taaaattagttagtttgaattggaatttaaatatctttatattttgattttatcgttaatttctatgatttgggttttaaagaccaagtaactaaatataatggtgaagacaattttagttccctgaaatatatctatagaggtaggtggatcaaacatttacctttgaataagatggaaaaatttactcttgtagatat

SEQ ID NO: 115 - DNA, 200 bp, Amaranthus palmeri
ttccaattcaaactaactaattttaaattacaaaatgaatatctgtaatttacaaaagaaagtatcaaaaacatatgaaaatctcaacatctgaaaattaaaaaacaagtattttgtttcttcatttttttctttttcgctatttcctttcaaaaataaaagtaaataaaaatattcaaaagcaattccataaacaaaat

SEQ ID NO: 116 - DNA, 200 bp, Amaranthus palmeri
attttgtttatggaattgcttttgaatatttttatttacttttatttttgaaaggaaatagcgaaaaagaaaaaaatgaagaaacaaaatacttgttttttaattttcagatgttgagattttcatatgtttttgatactttcttttgtaaattacagatattcattttgtaatttaaaattagttagtttgaattggaa

SEQ ID NO: 117 - DNA, 200 bp, Amaranthus palmeri
tcaaaagcaattccataaacaaaatcttagatatgtaaatcacaaaaacattagatctagaaaaaaaaaatttcttccattgcaaaccctttttcaaccttcataacttccactaccataatgaggccagtaaagagacaaaagtcattgagttgttgttgtgcagttgatgataaatgatgatagaagggtttattttt

SEQ ID NO: 118 - DNA, 200 bp, Amaranthus palmeri
aaaaataaacccttctatcatcatttatcatcaactgcacaacaacaactcaatgacttttgtctctttactggcctcattatggtagtggaagttatgaaggttgaaaaagggtttgcaatggaagaaattttttttttctagatctaatgtttttgtgatttacatatctaagattttgtttatggaattgcttttga

SEQ ID NO: 119 - DNA, 200 bp, Amaranthus palmeri
aatgatgatagaagggtttattttttttttgaaatgaatggttagattttctgactttttatttaccctatatagaatatcaaacaattaactctataaattatttaatacattaaaatgtttcatgtaatatgtctcctatattatttaccctttaatttttaagtgggaaccaagtatgtcttaattatctttatttt

SEQ ID NO: 120 - DNA, 200 bp, Amaranthus palmeri
aaaataaagataattaagacatacttggttcccacttaaaaattaaagggtaaataatataggagacatattacatgaaacattttaatgtattaaataatttatagagttaattgtttgatattctatatagggtaaataaaaagtcagaaaatctaaccattcatttcaaaaaaaaaataaacccttctatcatcatt

SEQ ID NO: 121 - DNA, 200 bp, Amaranthus palmeri
agtatgtcttaattatctttattttaatcaaatacgcggtatacatgaaataatcaacaaatgcaattactatgctcggacgagagtaaatataatgggaggaagttgtacatacaattacgaaatagtctaaataaataacgataatttgtaatataaacaaacaaatcacacttatataaacagattttatagggtgg

SEQ ID NO: 122 - DNA, 200 bp, Amaranthus palmeri
ccaccctataaaatctgtttatataagtgtgatttgtttgtttatattacaaattatcgttatttatttagactatttcgtaattgtatgtacaacttcctcccattatatttactctcgtccgagcatagtaattgcatttgttgattatttcatgtataccgcgtatttgattaaaataaagataattaagacatact

SEQ ID NO: 123 - DNA, 200 bp, Amaranthus palmeri
tatataaacagattttatagggtggaatcattaggattctaatttatcttttttcttcttttgtttactttgctgatatttattttgtatttttcctatttctcaaaaggaagactaacactcaaataaaatgatattgaaatacaaagcatcaccagccaagccgagatgacaaaactattggctaagtgataactgat

SEQ ID NO: 124 - DNA, 200 bp, Amaranthus palmeri
atcagttatcacttagccaatagttttgtcatctcggcttggctggtgatgctttgtatttcaatatcattttatttgagtgttagtcttccttttgagaaataggaaaaatacaaaataaatatcagcaaagtaaacaaaagaagaaaaaagataaattagaatcctaatgattccaccctataaaatctgtttatata

SEQ ID NO: 125 - DNA, 200 bp, Amaranthus palmeri
aactattggctaagtgataactgataagtgacgtatttccgttactctcaagtcttaacagctttttgtcacgcaaggaaaagaagaccgtggacgtcaacggtgacgttgaatgttcatctttacagtcgcagtcaatcaatctctttttagatcgatcttccacctcaattctccgttacaaatcaaattccatctag

SEQ ID NO: 126 - DNA, 200 bp, Amaranthus palmeri
ctagatggaatttgatttgtaacggagaattgaggtggaagatcgatctaaaaagagattgattgactgcgactgtaaagatgaacattcaacgtcaccgttgacgtccacggtcttcttttccttgcgtgacaaaaagctgttaagacttgagagtaacggaaatacgtcacttatcagttatcacttagccaatagtt

SEQ ID NO: 127 - DNA, 200 bp, Amaranthus palmeri
ccgttacaaatcaaattccatctagaacttctttttaattattttgactcataaattcccccaaaaatacttctattttattataaataaattccaatttctatgttctccattcatcaccacccattactccgttttccaaaccaccattttctctctcctccattacccctaacacaactaccattttcgcttcttcc

SEQ ID NO: 128 - DNA, 200 bp, Amaranthus palmeri
ggaagaagcgaaaatggtagttgtgttaggggtaatggaggagagagaaaatggtggtttggaaaacggagtaatgggtggtgatgaatggagaacatagaaattggaatttatttataataaaatagaagtatttttgggggaatttatgagtcaaaataattaaaaagaagttctagatggaatttgatttgtaacgg

SEQ ID NO: 129 - DNA, 200 bp, Amaranthus palmeri
cacaactaccattttcgcttcttccccgtctgagtcttattacatcgctttctctctcatcatctgacatgggaactttgaaacccgaaactcaacccgattccgaattcaaactcgtgggttactccaacttcgttcgggttaaccccaaatctgaccgttttgctgttaagcgtttccaccatatagagttttggtgt

SEQ ID NO: 130 - DNA, 200 bp, Amaranthus palmeri
acaccaaaactctatatggtggaaacgcttaacagcaaaacggtcagatttggggttaacccgaacgaagttggagtaacccacgagtttgaattcggaatcgggttgagtttcgggtttcaaagttcccatgtcagatgatgagagagaaagcgatgtaataagactcagacggggaagaagcgaaaatggtagttgtg

SEQ ID NO: 131 - DNA, 200 bp, Amaranthus palmeri
tttccaccatatagagttttggtgtggcgatgcaaccaatgttagcagacgattttcttggggtcttggaatgcctatcgtcgctaaatctgacctgtctacaggaaactctgttcacgcttcttttcttcttcgttccggtgacctttcttttctctttacttcaccgtattctcctaccatgtccatcccttcttctg

SEQ ID NO: 132 - DNA, 200 bp, Amaranthus palmeri
cagaagaagggatggacatggtaggagaatacggtgaagtaaagagaaaagaaaggtcaccggaacgaagaagaaaagaagcgtgaacagagtttcctgtagacaggtcagatttagcgacgataggcattccaagaccccaagaaaatcgtctgctaacattggttgcatcgccacaccaaaactctatatggtggaaa

SEQ ID NO: 133 - DNA, 200 bp, Amaranthus palmeri
cctaccatgtccatcccttcttctgctgcaatcccctcgtttgatttcaatcattttaccaaattccttacatcgcacggtcttggcgtgcgtgctgttgccgtcgaagtagaagacgccgaagctgcttttaatatcagcgtttcgcacggggctatcccctgtgtttctcctattcacttggaaaacggtgtcgtttt

SEQ ID NO: 134 - DNA, 200 bp, Amaranthus palmeri
aaaacgacaccgttttccaagtgaataggagaaacacaggggatagccccgtgcgaaacgctgatattaaaagcagcttcggcgtcttctacttcgacggcaacagcacgcacgccaagaccgtgcgatgtaaggaatttggtaaaatgattgaaatcaaacgaggggattgcagcagaagaagggatggacatggtagg

SEQ ID NO: 135 - DNA, 200 bp, Amaranthus palmeri
ttcacttggaaaacggtgtcgttttatctgaggttcatttatatggggatgttgtgcttcggtatgtaagctacggaaatgaatgtggggatgtgttttttcttcctgggtttgaggaaatgccggaggaatcatcgtttcgaggacttgattttggcattcgaaggttggatcatgctgtagggaatgtccctgagttg

SEQ ID NO: 136 - DNA, 200 bp, Amaranthus palmeri
caactcagggacattccctacagcatgatccaaccttcgaatgccaaaatcaagtcctcgaaacgatgattcctccggcatttcctcaaacccaggaagaaaaaacacatccccacattcatttccgtagcttacataccgaagcacaacatccccatataaatgaacctcagataaaacgacaccgttttccaagtgaa

SEQ ID NO: 137 - DNA, 200 bp, Amaranthus palmeri
tgctgtagggaatgtccctgagttggctcctgcaattgcttatttgaagaagtttactgggtttcatgagtttgctgagtttacagctgaagatgttgggacgagtgaaagtggattgaattcagccgtattggcaaacaatgatgaaatggtgttgtttccaatgaatgaacctgtgtatgggacaaaaaggaagagcc

SEQ ID NO: 138 - DNA, 200 bp, Amaranthus palmeri
ggctcttcctttttgtcccatacacaggttcattcattggaaacaacaccatttcatcattgtttgccaatacggctgaattcaatccactttcactcgtcccaacatcttcagctgtaaactcagcaaactcatgaaacccagtaaacttcttcaaataagcaattgcaggagccaactcagggacattccctacagca

SEQ ID NO: 139 - DNA, 200 bp, Amaranthus palmeri
gtgtatgggacaaaaaggaagagccaaattcaaacttatttggagcataatgaaggagctggtgtacagcatttggctttgatgagtgaagacatattttggactttaagggagatgaggaagagaagtggtcttggtgggtttgaatttatgccgtcgccgcctccgacttattaccggaatttgaggagcagagctgc

SEQ ID NO: 140 - DNA, 200 bp, Amaranthus palmeri
gcagctctgctcctcaaattccggtaataagtcggaggcggcgacggcataaattcaaacccaccaagaccacttctcttcctcatctcccttaaagtccaaaatatgtcttcactcatcaaagccaaatgctgtacaccagctccttcattatgctccaaataagtttgaatttggctcttcctttttgtcccatacac

SEQ ID NO: 141 - DNA, 200 bp, Amaranthus palmeri
accggaatttgaggagcagagctgctgatgtattgagtgaggagcagatgaaggagtgtgaagagttggggattttggtggataaagatgatcagggcactttgcttcaaatcttcaccaaacctattggagacaggtagattttaatcttgctttcaattgcttttgcttgattgattgactagccaattttgattgca

SEQ ID NO: 142 - DNA, 200 bp, Amaranthus palmeri
tgcaatcaaaattggctagtcaatcaatcaagcaaaagcaattgaaagcaagattaaaatctacctgtctccaataggtttggtgaagatttgaagcaaagtgccctgatcatctttatccaccaaaatccccaactcttcacactccttcatctgctcctcactcaatacatcagcagctctgctcctcaaattccggt

SEQ ID NO: 143 - DNA, 200 bp, Amaranthus palmeri
gattgactagccaattttgattgcattttgttgcttatatgacttgatgataatagatggtttacctttctcagctgttcatttgtagccagtatagattcgttctaaaatatttgcaactgattatgacatgtagtagcagaaaatgtccctatattggatgtttggcataaattaagcttggttttgcacttatcctc

SEQ ID NO: 144 - DNA, 200 bp, Amaranthus palmeri
gaggataagtgcaaaaccaagcttaatttatgccaaacatccaatatagggacattttctgctactacatgtcataatcagttgcaaatattttagaacgaatctatactggctacaaatgaacagctgagaaaggtaaaccatctattatcatcaagtcatataagcaacaaaatgcaatcaaaattggctagtcaatc

SEQ ID NO: 145 - DNA, 200 bp, Amaranthus palmeri
taagcttggttttgcacttatcctcatttatttataaattctaaaacttgttagttgtaattaagttaatgagaacaaagccttaatattccttcaaggtgattgtagttggggcactagttctaacaatggaaatttggaaatctattccaactggtcccaagttaatctttgtttgcaagcctgattggttcaaatta

SEQ ID NO: 146 - DNA, 200 bp, Amaranthus palmeri
taatttgaaccaatcaggcttgcaaacaaagattaacttgggaccagttggaatagatttccaaatttccattgttagaactagtgccccaactacaatcaccttgaaggaatattaaggctttgttctcattaacttaattacaactaacaagttttagaatttataaataaatgaggataagtgcaaaaccaagctta

SEQ ID NO: 147 - DNA, 200 bp, Amaranthus palmeri
ttgcaagcctgattggttcaaattaaggttattgtattcttgtatgaattcgactcaatgtaaatttgtttaatggagcatcaatttttaatagttttcgaccaagcagtattagatatattcgggattgaaataatgcatctatgagtgtataaaaccaaatggcacatttgattagaataaaagagagtataaggcta

SEQ ID NO: 148 - DNA, 200 bp, Amaranthus palmeri
tagccttatactctcttttattctaatcaaatgtgccatttggttttatacactcatagatgcattatttcaatcccgaatatatctaatactgcttggtcgaaaactattaaaaattgatgctccattaaacaaatttacattgagtcgaattcatacaagaatacaataaccttaatttgaaccaatcaggcttgcaa

SEQ ID NO: 149 - DNA, 200 bp, Amaranthus palmeri
ttttctcttttatttttatccaaaaatatcatgggatgtctagattggaataattagggagtaaaaagtaccccttgattatgcagagcaaaaataaattgtctgaatttgaaataaatatctacaagagtaaatttttccatcttattcaaaggtaaatgtttgatccacctacctctatagatatatttcagggaact

SEQ ID NO: 150 - DNA, 200 bp, Amaranthus palmeri
agttccctgaaatatatctatagaggtaggtggatcaaacatttacctttgaataagatggaaaaatttactcttgtagatatttatttcaaattcagacaatttatttttgctctgcataatcaaggggtactttttactccctaattattccaatctagacatcccatgatatttttggataaaaataaaagagaaaa

SEQ ID NO: 151 - DNA, 200 bp, Amaranthus palmeri
ctctatagatatatttcagggaactaaattgtcttcaccattaaatttggttacttggtctttaaaacccaaatcatagaaattaatgataaaatcaaaataaaaaagatatttaaattcaaattcaaactaactaattttaaattacaaaatgaatatctgtaatttacaaaagaaagtatcaaaaacatatgaaaatc

SEQ ID NO: 152 - DNA, 200 bp, Amaranthus palmeri
gattttcatatgtttttgatactttcttttgtaaattacagatattcattttgtaatttaaaattagttagtttgaatttgaatttaaatatcttttttattttgattttatcattaatttctatgatttgggttttaaagaccaagtaaccaaatttaatggtgaagacaatttagttccctgaaatatatctatagag

SEQ ID NO: 153 - DNA, 200 bp, Amaranthus palmeri
aaagtatcaaaaacatatgaaaatctcaacatctgaaaattacaaacaagtattctgttttttcattttttttttctttttcgctatttcctttcaaaaataaaagtaaataaaaatattcaaaagcaattccataaacaaaatcttagatatgtaaatcacaaaaacattagatctagaaaaaaaaaatttcttccatt

SEQ ID NO: 154 - DNA, 200 bp, Amaranthus palmeri
aatggaagaaattttttttttctagatctaatgtttttgtgatttacatatctaagattttgtttatggaattgcttttgaatatttttatttacttttatttttgaaaggaaatagcgaaaaagaaaaaaaaaatgaaaaaacagaatacttgtttgtaattttcagatgttgagattttcatatgtttttgatacttt

SEQ ID NO: 155 - DNA, 200 bp, Amaranthus palmeri
ctagaaaaaaaaaatttcttccattgcaaaccctttttcaaccttcataacttccactaccataatgaggccagtaaagagacaaaagtcattgagttgttgttgtgcagttgatgataaatgatgatagaagggtttattttttttttgaaatgaatggttagattttctgactttttatttaccctataatgaatatc

SEQ ID NO: 156 - DNA, 200 bp, Amaranthus palmeri
gatattcattatagggtaaataaaaagtcagaaaatctaaccattcatttcaaaaaaaaaataaacccttctatcatcatttatcatcaactgcacaacaacaactcaatgacttttgtctctttactggcctcattatggtagtggaagttatgaaggttgaaaaagggtttgcaatggaagaaattttttttttctag

SEQ ID NO: 157 - DNA, 200 bp, Amaranthus palmeri
ttttatttaccctataatgaatatcaaacaattaactctataaattatttaatacattaaaatgtttcatgtaatatgtctcctatattatttaccctttaatttttaagtgggaaccaagtatgtcttaattatctttattttaatcaaatacgcggtatacatgaaataatcaacaaatgcaattactatgctcggac

SEQ ID NO: 158 - DNA, 200 bp, Amaranthus palmeri
gtccgagcatagtaattgcatttgttgattatttcatgtataccgcgtatttgattaaaataaagataattaagacatacttggttcccacttaaaaattaaagggtaaataatataggagacatattacatgaaacattttaatgtattaaataatttatagagttaattgtttgatattcattatagggtaaataaaa

SEQ ID NO: 159 - DNA, 200 bp, Amaranthus palmeri
acaaatgcaattactatgctcggacgagagtaaatataatgggaggaagttgtacatacaattacgaaatagtctaaataaataacgataatttgtaatataaacaaacaaatcacacttatataaacagattttatagggtggaatcattaggattctaatttatcttttttcttcttttgtttactttgctgatattt

SEQ ID NO: 160 - DNA, 200 bp, Amaranthus palmeri
aaatatcagcaaagtaaacaaaagaagaaaaaagataaattagaatcctaatgattccaccctataaaatctgtttatataagtgtgatttgtttgtttatattacaaattatcgttatttatttagactatttcgtaattgtatgtacaacttcctcccattatatttactctcgtccgagcatagtaattgcatttgt

SEQ ID NO: 161 - DNA, 200 bp, Amaranthus palmeri
tcttttgtttactttgctgatatttattttgtatttttcctatttctcaaaaggaagactaacactcaaataaaatgatattgaaatacaaagcatcaccggccaagccgagatgacgaaactattttgaataattatgatgatttacaactccaaatagaagtaattgatcaagactttaggacttggaagtgttgggc

SEQ ID NO: 162 - DNA, 200 bp, Amaranthus palmeri
gcccaacacttccaagtcctaaagtcttgatcaattacttctatttggagttgtaaatcatcataattattcaaaatagtttcgtcatctcggcttggccggtgatgctttgtatttcaatatcattttatttgagtgttagtcttccttttgagaaataggaaaaatacaaaataaatatcagcaaagtaaacaaaaga

SEQ ID NO: 163 - DNA, 200 bp, Amaranthus palmeri
actttaggacttggaagtgttgggcaaaatcttccagagtccaggataagtgataagtgacgtatttccgttactcttaagtgttaacagctttttgtcacgcaaggaaaagaagaccgtggacgtcaacgatgacgttgaatgttcatctttacagtcgcagtcaatcaatctctttttagatcgatcttccacctcaa

SEQ ID NO: 164 - DNA, 200 bp, Amaranthus palmeri
ttgaggtggaagatcgatctaaaaagagattgattgactgcgactgtaaagatgaacattcaacgtcatcgttgacgtccacggtcttcttttccttgcgtgacaaaaagctgttaacacttaagagtaacggaaatacgtcacttatcacttatcctggactctggaagattttgcccaacacttccaagtcctaaagt

SEQ ID NO: 165 - DNA, 200 bp, Amaranthus palmeri
tttttagatcgatcttccacctcaattctccgttacaaatcaaattccatctagaacttcttttttattattttgactcataaattcccccaaaaatacttctattttattataaataaattccaatttctatgttctccattcattaccacccattactccgttttccaaaccaccattttctctctcctcctttaccg

SEQ ID NO: 166 - DNA, 200 bp, Amaranthus palmeri
cggtaaaggaggagagagaaaatggtggtttggaaaacggagtaatgggtggtaatgaatggagaacatagaaattggaatttatttataataaaatagaagtatttttgggggaatttatgagtcaaaataataaaaaagaagttctagatggaatttgatttgtaacggagaattgaggtggaagatcgatctaaaaa

SEQ ID NO: 167 - DNA, 200 bp, Amaranthus palmeri
ccattttctctctcctcctttaccgctaacgctaccaccattttcgcttcttccccgtctgaattttattacttcgctttctctatcatcatctgacatgggtactttgaaacccgaaactcaacccgattccgaattcaaactcgtgggttactccaacttcgttcgggttaaccccaaatctgaccgttttgctgtta

SEQ ID NO: 168 - DNA, 200 bp, Amaranthus palmeri
taacagcaaaacggtcagatttggggttaacccgaacgaagttggagtaacccacgagtttgaattcggaatcgggttgagtttcgggtttcaaagtacccatgtcagatgatgatagagaaagcgaagtaataaaattcagacggggaagaagcgaaaatggtggtagcgttagcggtaaaggaggagagagaaaatgg

SEQ ID NO: 169 - DNA, 200 bp, Amaranthus palmeri
cccaaatctgaccgttttgctgttaagcgtttccaccatatagagttttggtgtggcgatgcaaccaatgttagcagacgattttcttggggtcttggaatgcctaccgttgctaaatctgacctttctacaggaaactctgttcacgcttcttttcttcttcgttccggtgacctttcttttctctttacttcacctta

SEQ ID NO: 170 - DNA, 200 bp, Amaranthus palmeri
taaggtgaagtaaagagaaaagaaaggtcaccggaacgaagaagaaaagaagcgtgaacagagtttcctgtagaaaggtcagatttagcaacggtaggcattccaagaccccaagaaaatcgtctgctaacattggttgcatcgccacaccaaaactctatatggtggaaacgcttaacagcaaaacggtcagatttggg

SEQ ID NO: 171 - DNA, 200 bp, Amaranthus palmeri
tttcttttctctttacttcaccttactctcctaccatgtccatcccttcttctgctgcaatcccctcgtttgatttcaatcattttaccaaattccttacatcgcacggtcttggcgtgcgtgctgttgccgtcgaagtagaagacgcggaagctgcttttaacatcagcgtttcgcatggggctattccctgtgtttct

SEQ ID NO: 172 - DNA, 200 bp, Amaranthus palmeri
agaaacacagggaatagccccatgcgaaacgctgatgttaaaagcagcttccgcgtcttctacttcgacggcaacagcacgcacgccaagaccgtgcgatgtaaggaatttggtaaaatgattgaaatcaaacgaggggattgcagcagaagaagggatggacatggtaggagagtaaggtgaagtaaagagaaaagaaa

SEQ ID NO: 173 - DNA, 200 bp, Amaranthus palmeri
gcatggggctattccctgtgtttctcctattcaattggaaaacggtgtcgttttatctgaggttcatttatatggggatgttgtgcttcggtatgtaagctacggaaatgaatgtggggatgtgttttttcttcctgggtttgaggaaatgccggaggaatcatcgtttcgaggacttgattttggcattcgaaggttgg

SEQ ID NO: 174 - DNA, 200 bp, Amaranthus palmeri
ccaaccttcgaatgccaaaatcaagtcctcgaaacgatgattcctccggcatttcctcaaacccaggaagaaaaaacacatccccacattcatttccgtagcttacataccgaagcacaacatccccatataaatgaacctcagataaaacgacaccgttttccaattgaataggagaaacacagggaatagccccatgc

SEQ ID NO: 175 - DNA, 200 bp, Amaranthus palmeri
cttgattttggcattcgaaggttggatcatgctgtagggaatgtccctgagttggctcctgcaattgcttatttgaagaagtttactgggtttcatgagtttgctgagtttacagctgaagatgttgggacgagtgaaagtggattgaattcagccgtattggcaaacaatgatgaaatggtgttgtttccaatgaatga

SEQ ID NO: 176 - DNA, 200 bp, Amaranthus palmeri
tcattcattggaaacaacaccatttcatcattgtttgccaatacggctgaattcaatccactttcactcgtcccaacatcttcagctgtaaactcagcaaactcatgaaacccagtaaacttcttcaaataagcaattgcaggagccaactcagggacattccctacagcatgatccaaccttcgaatgccaaaatcaag

SEQ ID NO: 177 - DNA, 200 bp, Amaranthus palmeri
aaatggtgttgtttccaatgaatgaacctgtgtatgggacaaaaaggaagagccaaattcaaacttatttggagcataatgaaggagctggtgtacagcatttggctttgatgagtgaagacatattttggactttaagggagatgaggaagagaagtggtcttggtgggtttgaatttatgccgtcgccgcctccgact

SEQ ID NO: 178 - DNA, 200 bp, Amaranthus palmeri
agtcggaggcggcgacggcataaattcaaacccaccaagaccacttctcttcctcatctcccttaaagtccaaaatatgtcttcactcatcaaagccaaatgctgtacaccagctccttcattatgctccaaataagtttgaatttggctcttcctttttgtcccatacacaggttcattcattggaaacaacaccattt

SEQ ID NO: 179 - DNA, 200 bp, Amaranthus palmeri
atttatgccgtcgccgcctccgacttattaccggaatttgaggagcagagctgctgatgtattgagtgaggagcagatgaaggagtgtgaagagttggggattttggtggataaagatgatcagggcactttgcttcaaatcttcaccaaacctattggagacaggccaaccatattcatcgagattatccaaagaattg

SEQ ID NO: 180 - DNA, 200 bp, Amaranthus palmeri
caattctttggataatctcgatgaatatggttggcctgtctccaataggtttggtgaagatttgaagcaaagtgccctgatcatctttatccaccaaaatccccaactcttcacactccttcatctgctcctcactcaatacatcagcagctctgctcctcaaattccggtaataagtcggaggcggcgacggcataaat

SEQ ID NO: 181 - DNA, 200 bp, Amaranthus palmeri
ttcatcgagattatccaaagaattggttgcatgatgaaagatgaagacggcaagatgtaccaaaagggtggttgcggaggatttggaaagggaaacttttcagagttgttcaaatcaattgaggagtacgagaagactcttgaacgtaaacaggttccagatacagctgctgcatgatgagcagactgaaatattgctgt

SEQ ID NO: 182 - DNA, 200 bp, Amaranthus palmeri
acagcaatatttcagtctgctcatcatgcagcagctgtatctggaacctgtttacgttcaagagtcttctcgtactcctcaattgatttgaacaactctgaaaagtttccctttccaaatcctccgcaaccacccttttggtacatcttgccgtcttcatctttcatcatgcaaccaattctttggataatctcgatgaa

SEQ ID NO: 183 - DNA, 200 bp, Amaranthus palmeri
gatgagcagactgaaatattgctgtcttgctggtggaagccatataatggtaatatgatagaaaaggtttgttgctcaaaattttctttattaaataatgaagtgtaaacttatacaaactgtgtcatatatggtgattgatgatcatgcatggttatgtagaatgtataacataattgataatctgtgtatatgctgaa

SEQ ID NO: 184 - DNA, 200 bp, Amaranthus palmeri
ttcagcatatacacagattatcaattatgttatacattctacataaccatgcatgatcatcaatcaccatatatgacacagtttgtataagtttacacttcattatttaataaagaaaattttgagcaacaaaccttttctatcatattaccattatatggcttccaccagcaagacagcaatatttcagtctgctcatc

SEQ ID NO: 185 - DNA, 200 bp, Amaranthus rudis
aaatgaatgtggggatgtgttttttcttcctgggtttgaggaaatgccggaggaatcatcgtttcgaggacttgatttcggccttcgaaggttggatcatgctgtagggaatgtccctgagttggctcctgcaattgcttatttgaagaagtttactgggtttcatgagtttgctgagtttacagctgaagatgttggga

SEQ ID NO: 186 - DNA, 200 bp, Amaranthus rudis
tcccaacatcttcagctgtaaactcagcaaactcatgaaacccagtaaacttcttcaaataagcaattgcaggagccaactcagggacattccctacagcatgatccaaccttcgaaggccgaaatcaagtcctcgaaacgatgattcctccggcatttcctcaaacccaggaagaaaaaacacatccccacattcattt

SEQ ID NO: 187 - DNA, 200 bp, Amaranthus rudis
gagtttacagctgaagatgttgggacgagtgaaagtgggttgaattcagccgtattggcaaataatgatgaaatggtgttgtttccaatgaatgaaccagtgtatgggacaaaaaggaagagtcaaattcaaacttatttggagcataatgaaggagctggtgtacaacatttggctttgatgagtgaagatatattctg

SEQ ID NO: 188 - DNA, 200 bp, Amaranthus rudis
cagaatatatcttcactcatcaaagccaaatgttgtacaccagctccttcattatgctccaaataagtttgaatttgactcttcctttttgtcccatacactggttcattcattggaaacaacaccatttcatcattatttgccaatacggctgaattcaacccactttcactcgtcccaacatcttcagctgtaaactc

SEQ ID NO: 189 - DNA, 200 bp, Amaranthus rudis
ctttgatgagtgaagatatattctggactttaagggagatgaggaagagaagtggtcttggtgggtttgagtttatgccgtcgccgcctccgacttattaccggaatttgaggaacagagctgctgatgtattgagtgaggagcagatgaaggagtgtgaagagttggggattttggtggataaagatgatcagggtact

SEQ ID NO: 190 - DNA, 200 bp, Amaranthus rudis
agtaccctgatcatctttatccaccaaaatccccaactcttcacactccttcatctgctcctcactcaatacatcagcagctctgttcctcaaattccggtaataagtcggaggcggcgacggcataaactcaaacccaccaagaccacttctcttcctcatctcccttaaagtccagaatatatcttcactcatcaaag

SEQ ID NO: 191 - DNA, 200 bp, Amaranthus rudis
ggtggataaagatgatcagggtactttgcttcaaatcttcaccaaacctattggagacaggccaactatatttatcgagatcatccaaagaattggttgcatgatgaaagatgaagacggcaagatgtaccaaaagggtggttgcggaggatttggaaagggaaacttttcggagcttttcaaatcaattgaggagtacg

SEQ ID NO: 192 - DNA, 200 bp, Amaranthus rudis
cgtactcctcaattgatttgaaaagctccgaaaagtttccctttccaaatcctccgcaaccacccttttggtacatcttgccgtcttcatctttcatcatgcaaccaattctttggatgatctcgataaatatagttggcctgtctccaataggtttggtgaagatttgaagcaaagtaccctgatcatctttatccacc

SEQ ID NO: 193 - DNA, 200 bp, Amaranthus rudis
aatcattttaccaaattccttacatcgcacggtcttggcgtgcgtgctgttgctgtcgaagtagaagacgcagaagctgcttttaatatcagcgtttccaacggggctattccctgtgtttctcctattcacttggaaaacggtgtcgttttatctgaggttcatttatatggggatgttgtgcttcggtatgtaagcta

SEQ ID NO: 194 - DNA, 200 bp, Amaranthus rudis
tagcttacataccgaagcacaacatccccatataaatgaacctcagataaaacgacaccgttttccaagtgaataggagaaacacagggaatagccccgttggaaacgctgatattaaaagcagcttctgcgtcttctacttcgacagcaacagcacgcacgccaagaccgtgcgatgtaaggaatttggtaaaatgatt

SEQ ID NO: 195 - DNA, 200 bp, Amaranthus rudis
atgttgtgcttcggtatgtaagctacggaaatgaatgtggggatgtgttttttcttcctgggtttgaggaaatgccggaggaatcatcgtttcgaggactagatttcggccttcgaaggttggatcatgctgtagggaatgtccctgagttggctcctgcaattgcttatttgaagaagtttactgggtttcatgagttt

SEQ ID NO: 196 - DNA, 200 bp, Amaranthus rudis
aaactcatgaaacccagtaaacttcttcaaataagcaattgcaggagccaactcagggacattccctacagcatgatccaaccttcgaaggccgaaatctagtcctcgaaacgatgattcctccggcatttcctcaaacccaggaagaaaaaacacatccccacattcatttccgtagcttacataccgaagcacaacat

SEQ ID NO: 197 - DNA, 200 bp, Amaranthus rudis
gaagtttactgggtttcatgagtttgctgagtttacagctgaagatgttgggacgagtgaaagtgggttgaattcagccgtattggcaaataatgatgaaatggtgttgtttccaatgaatgaaccagtgtatgggacaaaaaggaagagtcaaattcaaacttatttggagcataatgaaggagctggtgtacaacatt

SEQ ID NO: 198 - DNA, 200 bp, Amaranthus rudis
aatgttgtacaccagctccttcattatgctccaaataagtttgaatttgactcttcctttttgtcccatacactggttcattcattggaaacaacaccatttcatcattatttgccaatacggctgaattcaacccactttcactcgtcccaacatcttcagctgtaaactcagcaaactcatgaaacccagtaaacttc

SEQ ID NO: 199 - DNA, 200 bp, Amaranthus rudis
aatgaaggagctggtgtacaacatttggctttgatgagtgaagatatattctggactttaagggagatgaggaagagaagtggtcttggtgggtttgagtttatgccgtcgccgcctccgacttattaccggaatttgaggaacagagctgctgatgtattgagtgaggagcagatgaaggagtgtgaagagttggggat

SEQ ID NO: 200 - DNA, 200 bp, Amaranthus rudis
atccccaactcttcacactccttcatctgctcctcactcaatacatcagcagctctgttcctcaaattccggtaataagtcggaggcggcgacggcataaactcaaacccaccaagaccacttctcttcctcatctcccttaaagtccagaatatatcttcactcatcaaagccaaatgttgtacaccagctccttcatt

SEQ ID NO: 201 - DNA, 200 bp, Amaranthus rudis
accaccattttcgtttcttccccgtctgagttttattacttcactttctctctcatcatctgacatgggaactttgaaacccgaaaatcaacccgattccgaattcaaactcgtgggttactccaactttgttcgggttaaccccaaatctgaccgttttactgttaagcgtttccatcatatagagttttggtgtggcg

SEQ ID NO: 202 - DNA, 200 bp, Amaranthus rudis
cgccacaccaaaactctatatgatggaaacgcttaacagtaaaacggtcagatttggggttaacccgaacaaagttggagtaacccacgagtttgaattcggaatcgggttgattttcgggtttcaaagttcccatgtcagatgatgagagagaaagtgaagtaataaaactcagacggggaagaaacgaaaatggtggt

SEQ ID NO: 203 - DNA, 200 bp, Amaranthus rudis
catcatatagagttttggtgtggcgacgcaaccaatgttagcagacgattttcttggggtcttggaatgcctaccgtcgctaaatctgacctttctacgggaaactctgttcacgcttcttttcttcttcgttccggtgacctttctttccttacttcaccttactcccctaccatgtccatcccttcttctgctgcaat

SEQ ID NO: 204 - DNA, 200 bp, Amaranthus rudis
attgcagcagaagaagggatggacatggtaggggagtaaggtgaagtaaggaaagaaaggtcaccggaacgaagaagaaaagaagcgtgaacagagtttcccgtagaaaggtcagatttagcgacggtaggcattccaagaccccaagaaaatcgtctgctaacattggttgcgtcgccacaccaaaactctatatgatg

SEQ ID NO: 205 - DNA, 200 bp, Amaranthus rudis
tgtccatcccttcttctgctgcaatcccctcgtttgatttcaatcattttaccaaattccttacatcgcacggtcttggcgtgcgtgctgttgctgtcgaagtagaagacgcagaagctgcttttaatatcagcgtttccaacggggctattccctgtgtttctcctattcacttggaaaacggtgtcgttttatctgag

SEQ ID NO: 206 - DNA, 200 bp, Amaranthus rudis
ctcagataaaacgacaccgttttccaagtgaataggagaaacacagggaatagccccgttggaaacgctgatattaaaagcagcttctgcgtcttctacttcgacagcaacagcacgcacgccaagaccgtgcgatgtaaggaatttggtaaaatgattgaaatcaaacgaggggattgcagcagaagaagggatggaca

SEQ ID NO: 207 - DNA, 200 bp, Amaranthus rudis
ggaaaacggtgtcgttttatctgaggttcatttatatggggatgttgtgcttcggtatgtaagctacggaaatgaatgtggggatgtgttttttcttcctgggtttgaggaaatgccggaggaatcttcgtttcgaggacttgatttcggccttcgaaggttggatcatgctgtagggaatgtccctgagttggctcctg

SEQ ID NO: 208 - DNA, 200 bp, Amaranthus rudis
caggagccaactcagggacattccctacagcatgatccaaccttcgaaggccgaaatcaagtcctcgaaacgaagattcctccggcatttcctcaaacccaggaagaaaaaacacatccccacattcatttccgtagcttacataccgaagcacaacatccccatataaatgaacctcagataaaacgacaccgttttcc

SEQ ID NO: 209 - DNA, 200 bp, Amaranthus rudis
gggaatgtccctgagttggctcctgcaattgcttatttgaagaagtttactgggtttcatgagtttgctgagtttacagctgaagatgttgggacgagtgaaagtgggttgaattcagccgtattggcaaataatgatgaaatggtgttgtttccaatgaatgaaccagtgtatgggacaaaaaggaagagtcaaattca

SEQ ID NO: 210 - DNA, 200 bp, Amaranthus rudis
tgaatttgactcttcctttttgtcccatacactggttcattcattggaaacaacaccatttcatcattatttgccaatacggctgaattcaacccactttcactcgtcccaacatcttcagctgtaaactcagcaaactcatgaaacccagtaaacttcttcaaataagcaattgcaggagccaactcagggacattccc

SEQ ID NO: 211 - DNA, 200 bp, Amaranthus rudis
ggacaaaaaggaagagtcaaattcaaacttatttggagcataatgaaggagctggtgtacaacatttggctttgatgagtgaagatatattctggactttaagggagatgaggaagagaagtggtcttggtgggtttgagtttatgccgtcgccgcctccgacttattaccggaatttgaggaacagagctgctgatgta

SEQ ID NO: 212 - DNA, 200 bp, Amaranthus rudis
tacatcagcagctctgttcctcaaattccggtaataagtcggaggcggcgacggcataaactcaaacccaccaagaccacttctcttcctcatctcccttaaagtccagaatatatcttcactcatcaaagccaaatgttgtacaccagctccttcattatgctccaaataagtttgaatttgactcttcctttttgtcc

SEQ ID NO: 213 - DNA, 200 bp, Amaranthus rudis
tttgaggaacagagctgctgatgtattgagtgaggagcagatgaaggagtgtgaagagttggggattttggtggataaagatgatcagggtactttgcttcaaatcttcaccaaacctattggagacaggtagtttttaatcttgctttcaattgcttttgattaattgattgattagccaatttgatgattgcattttg

SEQ ID NO: 214 - DNA, 200 bp, Amaranthus rudis
caaaatgcaatcatcaaattggctaatcaatcaattaatcaaaagcaattgaaagcaagattaaaaactacctgtctccaataggtttggtgaagatttgaagcaaagtaccctgatcatctttatccaccaaaatccccaactcttcacactccttcatctgctcctcactcaatacatcagcagctctgttcctcaaa

SEQ ID NO: 215 - DNA, 200 bp, Amaranthus rudis
cttacatcgcacggtcttggtgtgcgtgctgttgctgtcgaagtagaggacgcagaagctgcttttaatatcagcgtttccaacggggctattccctgtgtttctcctattcaattggaaaacggtgtcgttttatctgaggttcatttatatggggatgttgtgcttcgctatgtaagctacggaaatgaacgtgggga

SEQ ID NO: 216 - DNA, 200 bp, Amaranthus rudis
tccccacgttcatttccgtagcttacatagcgaagcacaacatccccatataaatgaacctcagataaaacgacaccgttttccaattgaataggagaaacacagggaatagccccgttggaaacgctgatattaaaagcagcttctgcgtcctctacttcgacagcaacagcacgcacaccaagaccgtgcgatgtaag

SEQ ID NO: 217 - DNA, 200 bp, Amaranthus rudis
taagctacggaaatgaacgtggggatgtgttttttcttcctgggtttgaggaaatgccggaggaatcgtcgtttcgaggacttgatttcggccttcgaaggttggatcatgctgtagggaatgttcccgagttggctcctgcaattgcttatttgaagaagtttactgggtttcatgagtttgctgagtttacagctgaa

SEQ ID NO: 218 - DNA, 200 bp, Amaranthus rudis
ttcagctgtaaactcagcaaactcatgaaacccagtaaacttcttcaaataagcaattgcaggagccaactcgggaacattccctacagcatgatccaaccttcgaaggccgaaatcaagtcctcgaaacgacgattcctccggcatttcctcaaacccaggaagaaaaaacacatccccacgttcatttccgtagctta

SEQ ID NO: 219 - DNA, 200 bp, Amaranthus rudis
tgagtttgctgagtttacagctgaagatgttgggacgagtgaaagtgggttgaattcagccgtattggcaaataatgatgaaatggtgttgtttccaatgaatgaacctgtgtatgggacaaaaaggaagagtcaaattcaaacttatttggagcataatgaaggagctggtgtacaacatttggctttgatgagtgaag

SEQ ID NO: 220 - DNA, 200 bp, Amaranthus rudis
cttcactcatcaaagccaaatgttgtacaccagctccttcattatgctccaaataagtttgaatttgactcttcctttttgtcccatacacaggttcattcattggaaacaacaccatttcatcattatttgccaatacggctgaattcaacccactttcactcgtcccaacatcttcagctgtaaactcagcaaactca

SEQ ID NO: 221 - DNA, 200 bp, Amaranthus rudis
caacatttggctttgatgagtgaagatatattttggactttaagggagatgaggaagagaagtggtcttggtgggtttgagtttatgccgtcgccgcctccgacttattaccggaatttgaggaatagagctgctgatgtattgagtgaggagcagatgaaggagtgtgaagagttggggattttggtggataaagatga

SEQ ID NO: 222 - DNA, 200 bp, Amaranthus rudis
tcatctttatccaccaaaatccccaactcttcacactccttcatctgctcctcactcaatacatcagcagctctattcctcaaattccggtaataagtcggaggcggcgacggcataaactcaaacccaccaagaccacttctcttcctcatctcccttaaagtccaaaatatatcttcactcatcaaagccaaatgttg

SEQ ID NO: 223 - DNA, 200 bp, Amaranthus rudis
tggggattttggtggataaagatgatcagggtactttgcttcaaatcttcactaagcctattggtgacaggccaactatatttatcgagatcatccaaagaattggttgcatgatgaaagatgaagacggcaagatgtaccaaaagggtggttgcggaggatttggaaagggaaacttttcggagcttttcaaatcaatt

SEQ ID NO: 224 - DNA, 200 bp, Amaranthus rudis
aattgatttgaaaagctccgaaaagtttccctttccaaatcctccgcaaccacccttttggtacatcttgccgtcttcatctttcatcatgcaaccaattctttggatgatctcgataaatatagttggcctgtcaccaataggcttagtgaagatttgaagcaaagtaccctgatcatctttatccaccaaaatcccca

SEQ ID NO: 225 - DNA, 200 bp, Amaranthus rudis
cttttcggagcttttcaaatcaattgaggagtatgagaagactcttgaacgtaaacaggttccagatacagctgctgcatgagcagactaaaatattgctgaaacgcaggctgcagccatatgttagaacagtcattctgatggaaacactcaagcggtgagtagctgaggttggtgatgctgaagtcgagtcggtattt

SEQ ID NO: 226 - DNA, 200 bp, Amaranthus rudis
aaataccgactcgacttcagcatcaccaacctcagctactcaccgcttgagtgtttccatcagaatgactgttctaacatatggctgcagcctgcgtttcagcaatattttagtctgctcatgcagcagctgtatctggaacctgtttacgttcaagagtcttctcatactcctcaattgatttgaaaagctccgaaaag

SEQ ID NO: 227 - DNA, 200 bp, Amaranthus rudis
tcatcaacaacaaaagagttgaaattctaataataatcccagtaagcagtgagtaagatcaaatggagagtcagttagtagctaatcatacaaaaccattaaagctacaaagttactctaatttcttaagatcaaacccaaaatctgatcgtttcaaagtgaaaaggttccaccacattgagttctggtgtggtgatgca

SEQ ID NO: 228 - DNA, 200 bp, Amaranthus rudis
tgcatcaccacaccagaactcaatgtggtggaaccttttcactttgaaacgatcagattttgggtttgatcttaagaaattagagtaactttgtagctttaatggttttgtatgattagctactaactgactctccatttgatcttactcactgcttactgggattattattagaatttcaactcttttgttgttgatga

SEQ ID NO: 229 - DNA, 200 bp, Amaranthus rudis
cattgagttctggtgtggtgatgcaaccaacactagccttttattttcgttagggcttggcatgcctatggttgccaaatccgatctctccacaggcaaccttatacatgcctcctacgtcttacgtagtggcgaactttgtttcgtattcacggctccttactctccgtcatccatgctcacaactgcatcaattcctt

SEQ ID NO: 230 - DNA, 200 bp, Amaranthus rudis
aaggaattgatgcagttgtgagcatggatgacggagagtaaggagccgtgaatacgaaacaaagttcgccactacgtaagacgtaggaggcatgtataaggttgcctgtggagagatcggatttggcaaccataggcatgccaagccctaacgaaaataaaaggctagtgttggttgcatcaccacaccagaactcaatg

SEQ ID NO: 231 - DNA, 200 bp, Amaranthus thunbergii
cccgagttggctcctgcaattgcttatttgaagaagtttactgggtttcatgagtttgctgagtttacagctgaagatgttgggacgagtgaaagtgggttgaattcagccgtattggcaaataatgatgaaatggtgttgtttccaatgaatgaacctgtgtatgggacaaaaaggaagagccaaattcaaacttattt

SEQ ID NO: 232 - DNA, 200 bp, Amaranthus thunbergii
aaataagtttgaatttggctcttcctttttgtcccatacacaggttcattcattggaaacaacaccatttcatcattatttgccaatacggctgaattcaacccactttcactcgtcccaacatcttcagctgtaaactcagcaaactcatgaaacccagtaaacttcttcaaataagcaattgcaggagccaactcggg

SEQ ID NO: 233 - DNA, 200 bp, Amaranthus thunbergii
ggaagagccaaattcaaacttatttggagcataatgaaggagctggtgtacaacatttggctttgatgagtgaagatatattttggactttaagggagatgaggaagagaagtggtcttggtgggtttgagtttatgccgtcgccgcctccgacttattaccggaatttgaggaatagagctgctgatgtattgagtgag

SEQ ID NO: 234 - DNA, 200 bp, Amaranthus thunbergii
ctcactcaatacatcagcagctctattcctcaaattccggtaataagtcggaggcggcgacggcataaactcaaacccaccaagaccacttctcttcctcatctcccttaaagtccaaaatatatcttcactcatcaaagccaaatgttgtacaccagctccttcattatgctccaaataagtttgaatttggctcttcc

SEQ ID NO: 235 - DNA, 200 bp, Amaranthus thunbergii
tagagctgctgatgtattgagtgaggagcagatgaaggagtgtgaagagttggggattttggtggataaagatgatcagggtactttgcttcaaatcttcactaagcctattggtgacaggccaaccatatttatcgagattatccaaagaattggatgcatgatgaaagatgaggacggcaagatgtaccaaaagggtg

SEQ ID NO: 236 - DNA, 200 bp, Amaranthus thunbergii
cacccttttggtacatcttgccgtcctcatctttcatcatgcatccaattctttggataatctcgataaatatggttggcctgtcaccaataggcttagtgaagatttgaagcaaagtaccctgatcatctttatccaccaaaatccccaactcttcacactccttcatctgctcctcactcaatacatcagcagctcta

SEQ ID NO: 237 - DNA, 200 bp, Amaranthus viridis
aaccaccattttctctctcctccattaccactaacactcccaccattttcgtttcttccccgtctgagtcttattacttcgctttctctcacatcatctgacatgggaactttgaaacccgaaactcagcccgattccgaattcaaactcgtgggatactccaacttcgttcgggttaaccccaaatctgaccgttttac

SEQ ID NO: 238 - DNA, 200 bp, Amaranthus viridis
gtaaaacggtcagatttggggttaacccgaacgaagttggagtatcccacgagtttgaattcggaatcgggctgagtttcgggtttcaaagttcccatgtcagatgatgtgagagaaagcgaagtaataagactcagacggggaagaaacgaaaatggtgggagtgttagtggtaatggaggagagagaaaatggtggtt

SEQ ID NO: 239 - DNA, 200 bp, Amaranthus viridis
ttaaccccaaatctgaccgttttactgttaagcgttttcatcatatagagttttggtgtggcgatgcaaccaatgttagcagacgattttcttggggtcttggaatgcctaccgttgctaaatctgacctttctactggaaactctgttcacgcttcttttcttcttcgttccggtgacctttcttttctctttacttca

SEQ ID NO: 240 - DNA, 200 bp, Amaranthus viridis
tgaagtaaagagaaaagaaaggtcaccggaacgaagaagaaaagaagcgtgaacagagtttccagtagaaaggtcagatttagcaacggtaggcattccaagaccccaagaaaatcgtctgctaacattggttgcatcgccacaccaaaactctatatgatgaaaacgcttaacagtaaaacggtcagatttggggttaa

SEQ ID NO: 241 - DNA, 200 bp, Amaranthus viridis
tgacctttcttttctctttacttcaccttactctcctaccatgtccgtcccttcttctgctgcaatcccctcgtttgatttcaatcattttaccaaattccttacatcgcacggtcttggtgtgcgtgctgttgctgtcgaagtagaagacgcagaagctgcttttaatatcagcgtttccaacggggctattccctgtg

SEQ ID NO: 242 - DNA, 200 bp, Amaranthus viridis
cacagggaatagccccgttggaaacgctgatattaaaagcagcttctgcgtcttctacttcgacagcaacagcacgcacaccaagaccgtgcgatgtaaggaatttggtaaaatgattgaaatcaaacgaggggattgcagcagaagaagggacggacatggtaggagagtaaggtgaagtaaagagaaaagaaaggtca

SEQ ID NO: 243 - DNA, 200 bp, Amaranthus viridis
gtttccaacggggctattccctgtgtttctcctattcaattggaaaacggtgtcgttttatctgaggttcatttatatggggatgttgtgcttcggtatgtaagctacggaaatgaatgtggggatgtgtttttcttcctgggtttgaggaaatgccggaggaatcgtcgtttcgaggacttgatttcggccttcgaagg

SEQ ID NO: 244 - DNA, 200 bp, Amaranthus viridis
ccttcgaaggccgaaatcaagtcctcgaaacgacgattcctccggcatttcctcaaacccaggaagaaaaacacatccccacattcatttccgtagcttacataccgaagcacaacatccccatataaatgaacctcagataaaacgacaccgttttccaattgaataggagaaacacagggaatagccccgttggaaac

SEQ ID NO: 245 - DNA, 200 bp, Amaranthus viridis
aggacttgatttcggccttcgaaggttggatcatgctgtagggaatgttcccgagttggctcctgcaattgcttatttgaaaaagtttactgggtttcatgagtttgctgagtttacagctgaagatgttgggacgagtgaaagtgggttgaattcagccgtattggcaaataatgatgaaatggtgttgtttccaatga

SEQ ID NO: 246 - DNA, 200 bp, Amaranthus viridis
tcattggaaacaacaccatttcatcattatttgccaatacggctgaattcaacccactttcactcgtcccaacatcttcagctgtaaactcagcaaactcatgaaacccagtaaactttttcaaataagcaattgcaggagccaactcgggaacattccctacagcatgatccaaccttcgaaggccgaaatcaagtcct

SEQ ID NO: 247 - DNA, 200 bp, Amaranthus viridis
gatgaaatggtgttgtttccaatgaatgaacctgtgtatgggacaaaaaggaagagccaaattcaaacttatttggagcataatgaaggagctggtgtacaacatttggctttgatgagtgaagatatattttggactttaagggagatgaggaagagaagtggtcttggtgggtttgagtttatgccgtcgccgcctcc

SEQ ID NO: 248 - DNA, 200 bp, Amaranthus viridis
ggaggcggcgacggcataaactcaaacccaccaagaccacttctcttcctcatctcccttaaagtccaaaatatatcttcactcatcaaagccaaatgttgtacaccagctccttcattatgctccaaataagtttgaatttggctcttcctttttgtcccatacacaggttcattcattggaaacaacaccatttcatc

SEQ ID NO: 249 - DNA, 200 bp, Amaranthus viridis
ttgagtttatgccgtcgccgcctccgacttattaccggaatttgaggaatagagctgctgatgtattgagtgaggagcagatgaaggagtgtgaagagttggggattttggttgacagagatgatcaaggcactctgcttcaaattttcactaagcccattggagataggcctacgatattcatagaaataatacagaga

SEQ ID NO: 250 - DNA, 200 bp, Amaranthus viridis
tctctgtattatttctatgaatatcgtaggcctatctccaatgggcttagtgaaaatttgaagcagagtgccttgatcatctctgtcaaccaaaatccccaactcttcacactccttcatctgctcctcactcaatacatcagcagctctattcctcaaattccggtaataagtcggaggcggcgacggcataaactcaa

SEQ ID NO: 251 - DNA, 200 bp, Ambrosia trifida
ccggtttacgggacgaagaggaagagtcagatacagacatatttggaacataatgaaggggcaggggtgcagcatttggcgttggcgagtgaggatatatttaggacattgagggagatgaggaaaaggagtggggtgggtgggtttgagtttatgccatctccgccgcctacttattatcggaatttgaagaacagggc

SEQ ID NO: 252 - DNA, 200 bp, Ambrosia trifida
gccctgttcttcaaattccgataataagtaggcggcggagatggcataaactcaaacccacccaccccactccttttcctcatctccctcaatgtcctaaatatatcctcactcgccaacgccaaatgctgcacccctgccccttcattatgttccaaatatgtctgtatctgactcttcctcttcgtcccgtaaaccgg

SEQ ID NO: 253 - DNA, 200 bp, Ambrosia trifida
attatcggaatttgaagaacagggcgggcgatgtgttgtcggatgagcagattaaggagtgtgaggagttggggatattggtggataaggatgatcaggggactttgcttcagatttttaccaagcctgttggtgataggccgacgatattcatagagataattcagagagtaggatgtatgatgaaggatgaagaagga

SEQ ID NO: 254 - DNA, 200 bp, Ambrosia trifida
tccttcttcatccttcatcatacatcctactctctgaattatctctatgaatatcgtcggcctatcaccaacaggcttggtaaaaatctgaagcaaagtcccctgatcatccttatccaccaatatccccaactcctcacactccttaatctgctcatccgacaacacatcgcccgccctgttcttcaaattccgataat

SEQ ID NO: 255 - DNA, 200 bp, Ambrosia trifida
atgtatgatgaaggatgaagaaggaaaggtgcagcagaaggcgggctgtggaggatttggtaaagggaacttttcggagctttttaaatcgattgaggaatatgaaaagacactggaagcaagaacctgaagccacatgaaaaccacacacaaataatctttcatgagattttaaaatctaatgattatgcatctgtgga

SEQ ID NO: 256 - DNA, 200 bp, Ambrosia trifida
tccacagatgcataatcattagattttaaaatctcatgaaagattatttgtgtgtggttttcatgtggcttcaggttcttgcttccagtgtcttttcatattcctcaatcgatttaaaaagctccgaaaagttccctttaccaaatcctccacagcccgccttctgctgcacctttccttcttcatccttcatcatacat

SEQ ID NO: 257 - DNA, 54 bp, Ambrosia trifida; identical to SEQ ID NO: 17

SEQ ID NO: 258 - DNA, 54 bp, Ambrosia trifida
cgcctcgcgctttaagccccgcgcttcggattttggacccaaaacgcctcggag

SEQ ID NO: 259 - DNA, 200 bp, Ambrosia trifida
cttcatgtagttcagcgctttttggcaaaaaaagcgtttttcttggagccttgtgcctcaaacgcttaaagtgcgccccaggcgagcttttttaaaccaaggaacacgcacaagcactccaccaatagttcaatgccgtgctaaccagtgggattaactagtggaagctggattcaaacatgctagagtggaaatatatt

SEQ ID NO: 260 - DNA, 200 bp, Ambrosia trifida
aatatatttccactctagcatgtttgaatccagcttccactagttaatcccactggttagcacggcattgaactattggtggagtgcttgtgcgtgttccttggtttaaaaaagctcgcctggggcgcactttaagcgtttgaggcacaaggctccaagaaaaacgctttttttgccaaaaagcgctgaactacatgaag

SEQ ID NO: 261 - DNA, 200 bp, Ambrosia trifida
aaacatgctagagtggaaatatattttttttaatgtttggatatgcatatttaaccattatagcataatattaatgacaagggttgtaaattggtatgcaaattgtttgagcaggccgacgatattcatagagataattcagagagtaggatgtatgatgaaggatgatgaaggaaaggtgcagcagaaggcgggttgtg

SEQ ID NO: 262 - DNA, 200 bp, Ambrosia trifida
cacaacccgccttctgctgcacctttccttcatcatccttcatcatacatcctactctctgaattatctctatgaatatcgtcggcctgctcaaacaatttgcataccaatttacaacccttgtcattaatattatgctataatggttaaatatgcatatccaaacattaaaaaaaatatatttccactctagcatgttt

SEQ ID NO: 263 - DNA, 200 bp, Ambrosia trifida
aaggtgcagcagaaggcgggttgtggaggatttggtaaagggaacttttcggagctttttaaatcgattgaggaatatgaaaagacactggaagcaagaacctgaagccacatgaaaaccacacaaataatctttaatgagatcttaaaatctaatgattatgcatctgtggattctatacaacaaacagatcctgaaaa

SEQ ID NO: 264 - DNA, 200 bp, Ambrosia trifida
ttttcaggatctgtttgttgtatagaatccacagatgcataatcattagattttaagatctcattaaagattatttgtgtggttttcatgtggcttcaggttcttgcttccagtgtcttttcatattcctcaatcgatttaaaaagctccgaaaagttccctttaccaaatcctccacaacccgccttctgctgcacctt

SEQ ID NO: 265 - DNA, 200 bp, Ambrosia trifida
gcagttgagctctacggctcactgaaacagacctttaaaccatgaacaaggtttatgtgtaggtaaactaactttggaccaaaaagctgtttttttaaccttcacaatttgtttcatggaaggttttcctttttgttcagttgacatgtttctaaacgggacaaataaacatacatctatgcctattgcacatatagcat

SEQ ID NO: 266 - DNA, 200 bp, Ambrosia trifida
atgctatatgtgcaataggcatagatgtatgtttatttgtcccgtttagaaacatgtcaactgaacaaaaaggaaaaccttccatgaaacaaattgtgaaggttaaaaaaacagctttttggtccaaagttagtttacctacacataaaccttgttcatggtttaaaggtctgtttcagtgagccgtagagctcaactgc

SEQ ID NO: 267 - DNA, 200 bp, Ambrosia trifida
tctatgcctattgcacatatagcatcaaccacctcactcccatgtggccctcacaacaaggttgtaaaaatcgggactagtcgccgactagtccccgataaatcggtgatcggaggtctactgattaatttttcattaatcactcaattagtcggaatcggcccaagccggtccaagccggccaaagtcggccgaagtcg

SEQ ID NO: 268 - DNA, 200 bp, Ambrosia trifida
cgacttcggccgactttggccggcttggaccggcttgggccgattccgactaattgagtgattaatgaaaaattaatcagtagacctccgatcaccgatttatcggggactagtcggcgactagtcccgatttttacaaccttgttgtgagggccacatgggagtgaggtggttgatgctatatgtgcaataggcataga

SEQ ID NO: 269 - DNA, 200 bp, Ambrosia trifida
agccggccaaagtcggccgaagtcggagctagtcgccaaagaattccgtttgaagttggcttaaaaacatgctttcatcccacaaactgatccattaccccaaaaaaccaaagttaaaaacctgacctggatccctaaatgcttcataatccggcaaacctttctgttccggcgaacctttctgtttccggcgacccatt

SEQ ID NO: 270 - DNA, 200 bp, Ambrosia trifida
aatgggtcgccggaaacagaaaggttcgccggaacagaaaggtttgccggattatgaagcatttagggatccaggtcaggtttttaactttggttttttggggtaatggatcagtttgtgggatgaaagcatgtttttaagccaacttcaaacggaattctttggcgactagctccgacttcggccgactttggccggct

SEQ ID NO: 271 - DNA, 200 bp, Ambrosia trifida
acctttctgtttccggcgacccatttggagcaggtggagttggttttgttcagtcttttgcggcgtataatgaaagtggtagtgagtagggtttttaagtttggatctttttatatttaaccccttatagttttactagtttatattttgtcccttaacttatatatttatacataataagcataaagttaatgttatat

SEQ ID NO: 272 - DNA, 200 bp, Ambrosia trifida
atataacattaactttatgcttattatgtataaatatataagttaagggacaaaatataaactagtaaaactataaggggttaaatataaaaagatccaaacttaaaaaccctactcactaccactttcattatacgccgcaaaagactgaacaaaaccaactccacctgctccaaatgggtcgccggaaacagaaaggt

SEQ ID NO: 273 - DNA, 200 bp, Ambrosia trifida
tacctcacaaacccaatcctccatacttccgtgttctctactactgttccactttattaatcttacccaaccccttctcatctatttcccccctacctcacaaacataacccaaataaaaaataaaattaaaataaaaaataaaatggaactgaacccaccctcgttgaccccgtcgtcgccggagacaactccgaccac

SEQ ID NO: 274 - DNA, 200 bp, Ambrosia trifida
gtggtcggagttgtctccggcgacgacggggtcaacgagggtgggttcagttccattttattttttattttaattttattttttatttgggttatgtttgtgaggtaggggggaaatagatgagaaggggttgggtaagattaataaagtggaacagtagtagagaacacggaagtatggaggattgggtttgtgaggta

SEQ ID NO: 275 - DNA, 200 bp, Ambrosia trifida
cgtcgccggagacaactccgaccactccaaccaccccatctccgccttcaagctagtcggcttcaaaaacttcatccgtaacaaccctaagtccgacaaattctccgtcaaatccttccaccacatcgagttctggtgctccgacgccaccaacgccgcccgtcgcttctcatggggtctcggcatgcctattacttaca

SEQ ID NO: 276 - DNA, 200 bp, Ambrosia trifida
tgtaagtaataggcatgccgagaccccatgagaagcgacgggcggcgttggtggcgtcggagcaccagaactcgatgtggtggaaggatttgacggagaatttgtcggacttagggttgttacggatgaagtttttgaagccgactagcttgaaggcggagatggggtggttggagtggtcggagttgtctccggcgacg

SEQ ID NO: 277 - DNA, 200 bp, Ambrosia trifida
ggtctcggcatgcctattacttacaaatccgacctttctaccggtaaccatacccacgcctcttatctccttaactccggccaccttaacttcctcttcaccgccccttattcccccaccatctccaccaccaccaccaccgcctccatcccgtcgttctcccactccgcctgccgtcacttcaccgcctcccacggcct

SEQ ID NO: 278 - DNA, 200 bp, Ambrosia trifida
aggccgtgggaggcggtgaagtgacggcaggcggagtgggagaacgacgggatggaggcggtggtggtggtggtggagatggtgggggaataaggggcggtgaagaggaagttaaggtggccggagttaaggagataagaggcgtgggtatggttaccggtagaaaggtcggatttgtaagtaataggcatgccgagacc

SEQ ID NO: 279 - DNA, 200 bp, Ambrosia trifida
tcgtcgtttcaggagctggattacggtatccggcggctagatcacgctgtggggaacgtaccggagttagcaccagcagtggaatatataaaatcgtttaccgggtttcacgagtttgctgagtttacagcagaggatgtgggaacgagtgagagtggactcaactcggtggtgttggcttgcaatagtgagatggtatt

SEQ ID NO: 280 - DNA, 200 bp, Ambrosia trifida
aataccatctcactattgcaagccaacaccaccgagttgagtccactctcactcgttcccacatcctctgctgtaaactcagcaaactcgtgaaacccggtaaacgattttatatattccactgctggtgctaactccggtacgttccccacagcgtgatctagccgccggataccgtaatccagctcctgaaacgacga

SEQ ID NO: 281 - DNA, 200 bp, Ambrosia trifida
tggcttgcaatagtgagatggtattaatacctctgaatgagccggtttacgggacgaagaggaagagtcagatacagacatatttggaacataatgaaggggcaggggtgcagcatttggcgttggcgagtgaggatatatttaggacattgagggagatgaggaaaaggagtggggtgggtgggtttgagtttatgccg

SEQ ID NO: 282 - DNA, 200 bp, Ambrosia trifida
cggcataaactcaaacccacccaccccactccttttcctcatctccctcaatgtcctaaatatatcctcactcgccaacgccaaatgctgcacccctgccccttcattatgttccaaatatgtctgtatctgactcttcctcttcgtcccgtaaaccggctcattcagaggtattaataccatctcactattgcaagcca

SEQ ID NO: 283 - DNA, 200 bp, Ambrosia trifida
ggtgggtgggtttgagtttatgccgtctccgccgcctacttattatcggaatttgaagaatagggcgggcgatgtgttgtcggatgagcagattaaggagtgtgaggagttggggatattggtggataaggatgatcaggggactttgcttcagatttttaccaagcctgttggtgataggtattgtttttcgttaatgt

SEQ ID NO: 284 - DNA, 200 bp, Ambrosia trifida
acattaacgaaaaacaatacctatcaccaacaggcttggtaaaaatctgaagcaaagtcccctgatcatccttatccaccaatatccccaactcctcacactccttaatctgctcatccgacaacacatcgcccgccctattcttcaaattccgataataagtaggcggcggagacggcataaactcaaacccacccacc

SEQ ID NO: 285 - DNA, 200 bp, Ambrosia trifida
gataggtattgtttttcgttaatgtagcgttacttgaattcgtaattatgtgtttaaactttaaactagatagtattaaggcttatgcaatatgtattgttcccctagaggacgagggttctagactctagttccgtaatgtagtgataaaggtgtagattaagagtaggattagcgggtgtgttagttagcggttttaa

SEQ ID NO: 286 - DNA, 200 bp, Ambrosia trifida
ttaaaaccgctaactaacacacccgctaatcctactcttaatctacacctttatcactacattacggaactagagtctagaaccctcgtcctctaggggaacaatacatattgcataagccttaatactatctagtttaaagtttaaacacataattacgaattcaagtaacgctacattaacgaaaaacaatacctatc

SEQ ID NO: 287 - DNA, 200 bp, Ambrosia trifida
cgggtgtgttagttagcggttttaacaagaaaaacacataatatgttgaaagaacaataacaagaatggcaaacataaaaaaggaaaagccataaaggttgtcattctataatttgcttgcattcaagattcttactaggattgtattattatgtggttactaaactctattcaagtataaaaacatgaaaccccttcac

SEQ ID NO: 288 - DNA, 200 bp, Ambrosia trifida
gtgaaggggtttcatgtttttatacttgaatagagtttagtaaccacataataatacaatcctagtaagaatcttgaatgcaagcaaattatagaatgacaacctttatggcttttccttttttatgtttgccattcttgttattgttctttcaacatattatgtgtttttcttgttaaaaccgctaactaacacacccg

SEQ ID NO: 289 - DNA, 200 bp, Conyza canadensis
aacacgaatgggctagctagctgatgaatttaatgtatatatgtctttaaaaaccactttcatgggctacttgtgtttttgatgagtttttcaacatcattctcatcttaccttcttacaatggttcaccatattgcttcgtttgtcgtctcctgcatggttatcgaatttattttttcaaattatattactattatctt

SEQ ID NO: 290 - DNA, 200 bp, Conyza canadensis
aagataatagtaatataatttgaaaaaataaattcgataaccatgcaggagacgacaaacgaagcaatatggtgaaccattgtaagaaggtaagatgagaatgatgttgaaaaactcatcaaaaacacaagtagcccatgaaagtggtttttaaagacatatatacattaaattcatcagctagctagcccattcgtgtt

SEQ ID NO: 291 - DNA, 200 bp, Conyza canadensis
tttcaaattatattactattatctttttgttttctgatttctcacatgtaatttacattatgagtttcaccatccgtatgtaaattgtaaaataaatcacaaatttggtttttaaccatcaactaaattcattgaaatccttaagttataacccatttgaggttatagattatgagagtagattcctattttctctttgg

SEQ ID NO: 292 - DNA, 200 bp, Conyza canadensis
ccaaagagaaaataggaatctactctcataatctataacctcaaatgggttataacttaaggatttcaatgaatttagttgatggttaaaaaccaaatttgtgatttattttacaatttacatacggatggtgaaactcataatgtaaattacatgtgagaaatcagaaaacaaaaagataatagtaatataatttgaaa

SEQ ID NO: 293 - DNA, 200 bp, Conyza canadensis
gagtagattcctattttctctttggcttatgtatccttaattatgtttgaattgattctaactttattatattttaggaattacttaacttcaaaaatctcaacacgtcatataatgctttttgtggatggatgcttttgacccatttcgttgaaaacttatgtcgacacacatatctgacaaacttgtactcgtatatc

SEQ ID NO: 294 - DNA, 200 bp, Conyza canadensis
gatatacgagtacaagtttgtcagatatgtgtgtcgacataagttttcaacgaaatgggtcaaaagcatccatccacaaaaagcattatatgacgtgttgagatttttgaagttaagtaattcctaaaatataataaagttagaatcaattcaaacataattaaggatacataagccaaagagaaaataggaatctactc

SEQ ID NO: 295 - DNA, 200 bp, Conyza canadensis
tctgacaaacttgtactcgtatatcctttatcgcatatgctatagtataatattcgaaatttaatctcaagataattttcccaaaaggtcatattaattgaattgagattaattcctacctaacatatatagatatattgtgttggtgttatcctctttggagttttagtcgcgcatgtattctaagaaaatggtaattt

SEQ ID NO: 296 - DNA, 200 bp, Conyza canadensis
aaattaccattttcttagaatacatgcgcgactaaaactccaaagaggataacaccaacacaatatatctatatatgttaggtaggaattaatctcaattcaattaatatgaccttttgggaaaattatcttgagattaaatttcgaatattatactatagcatatgcgataaaggatatacgagtacaagtttgtcaga

SEQ ID NO: 297 - DNA, 200 bp, Conyza canadensis
atgtattctaagaaaatggtaattttgaacttatattgatggtctaaattttatcctatcactaaaatttcagtcatgtttcctagctaattaatttgggttcaatccaggtattctaaataagttttgtaaatacatgtctattatcataatgaaacatagaaaaacccgtaaagtaaaaatgtctatatttttagtgg

SEQ ID NO: 298 - DNA, 200 bp, Conyza canadensis
ccactaaaaatatagacatttttactttacgggtttttctatgtttcattatgataatagacatgtatttacaaaacttatttagaatacctggattgaacccaaattaattagctaggaaacatgactgaaattttagtgataggataaaatttagaccatcaatataagttcaaaattaccattttcttagaatacat

SEQ ID NO: 299 - DNA, 200 bp, Conyza canadensis
gtaaaaatgtctatatttttagtggcaaattattaatgttgctataatgaaaagagaagattcaaccttttgtacatcataaaaaatgttattctacattatgctaattttacattagacttcatagttttgaaaactttcaaatgaagggacttaaattttaaatttaaagatgatgtcataaattagttagataaaag

SEQ ID NO: 300 - DNA, 200 bp, Conyza canadensis
cttttatctaactaatttatgacatcatctttaaatttaaaatttaagtcccttcatttgaaagttttcaaaactatgaagtctaatgtaaaattagcataatgtagaataacattttttatgatgtacaaaaggttgaatcttctcttttcattatagcaacattaataatttgccactaaaaatatagacatttttac

SEQ ID NO: 301 - DNA, 200 bp, Conyza canadensis
atgtcataaattagttagataaaagttaaataattaataagaaaaggtctaatgataccaaataaaattttcttaaaaaagtattaatgtcattacaattttattttatttatataagagaagataatagattttcttgtataaaaaacttacctgtaaacttctatataaactttttagaaaaataatttaaaacggtc

SEQ ID NO: 302 - DNA, 200 bp, Conyza canadensis
gaccgttttaaattatttttctaaaaagtttatatagaagtttacaggtaagttttttatacaagaaaatctattatcttctcttatataaataaaataaaattgtaatgacattaatacttttttaagaaaattttatttggtatcattagaccttttcttattaattatttaacttttatctaactaatttatgacat

SEQ ID NO: 303 - DNA, 200 bp, Conyza canadensis
tttagaaaaataatttaaaacggtctcaaatatcgttatttccgaacatacaaaaacccaaaacctaatccatttaattatattgtaagacctcgattagcttaatttattttgaaacctatttacaaatgagtttggcatgatatgaacttttttgcatttacatatggctgtaaacgaactgaacagttcacgaacaa

SEQ ID NO: 304 - DNA, 200 bp, Conyza canadensis
ttgttcgtgaactgttcagttcgtttacagccatatgtaaatgcaaaaaagttcatatcatgccaaactcatttgtaaataggtttcaaaataaattaagctaatcgaggtcttacaatataattaaatggattaggttttgggtttttgtatgttcggaaataacgatatttgagaccgttttaaattatttttctaaa

SEQ ID NO: 305 - DNA, 200 bp, Conyza canadensis
aacgaactgaacagttcacgaacaattcatgaaccgttcggcgggaggttcgttcgtgttagttcgtttagttaaatgaatgaacatgaacaaagctctcgttcgttcatttacgttcatgaacgttcgatagtctgttcgtgaactttagttcgtctatgttcatgaactgtcgttcgtgaacaataatttgattatgt

SEQ ID NO: 306 - DNA, 200 bp, Conyza canadensis
acataatcaaattattgttcacgaacgacagttcatgaacatagacgaactaaagttcacgaacagactatcgaacgttcatgaacgtaaatgaacgaacgagagctttgttcatgttcattcatttaactaaacgaactaacacgaacgaacctcccgccgaacggttcatgaattgttcgtgaactgttcagttcgtt

SEQ ID NO: 307 - DNA, 200 bp, Conyza canadensis
ttcgtgaacaataatttgattatgttcatttacacacacatacataatcaactgtactataaaaatagtttaaatataaatatttttttatataatatatattattaatatctaattatttaagaaaaagtttaaataaaatacttaatttataaatatttcgttcgtttgtgttcgtgtacatttgttcatgaacacaa

SEQ ID NO: 308 - DNA, 200 bp, Conyza canadensis
ttgtgttcatgaacaaatgtacacgaacacaaacgaacgaaatatttataaattaagtattttatttaaactttttcttaaataattagatattaataatatatattatataaaaaaatatttatatttaaactatttttatagtacagttgattatgtatgtgtgtgtaaatgaacataatcaaattattgttcacgaa

SEQ ID NO: 309 - DNA, 200 bp, Conyza canadensis
cgtgtacatttgttcatgaacacaaatgaacgaacatgaacaagtcattatgttcgtttatctgttcgttaagttaaatgaacgaacgaacacgaacaaggtctcgtttgtgttcgttcggttcgtttacggcccttaattacatgtgaagttaataaataaaaataaagttcatcaaaattaaacctatggttggagat

SEQ ID NO: 310 - DNA, 200 bp, Conyza canadensis
atctccaaccataggtttaattttgatgaactttatttttatttattaacttcacatgtaattaagggccgtaaacgaaccgaacgaacacaaacgagaccttgttcgtgttcgttcgttcatttaacttaacgaacagataaacgaacataatgacttgttcatgttcgttcatttgtgttcatgaacaaatgtacacg

SEQ ID NO: 311 - DNA, 200 bp, Conyza canadensis
caaaattaaacctatggttggagataaaaccagtcagccttacgtgtggaaactatcctactctacaattaatagcacccaaagaaaataaaccacttccgtgttcttcttcatctccatcatccaacccctcaactacttgtactgtatctatatctatcattctatatatctacttcatatttctatctatacattca

SEQ ID NO: 312 - DNA, 200 bp, Conyza canadensis
tgaatgtatagatagaaatatgaagtagatatatagaatgatagatatagatacagtacaagtagttgaggggttggatgatggagatgaagaagaacacggaagtggtttattttctttgggtgctattaattgtagagtaggatagtttccacacgtaaggctgactggttttatctccaaccataggtttaattttg

SEQ ID NO: 313 - DNA, 200 bp, Conyza canadensis
cttcatatttctatctatacattcaaattaataaaaacaaaatggtaactgaagcagcaaacggaaacacactcaccggacaacaacaaaccactaccaccacctccgccacttacaaactcgtcggcttcaaaaacttcatccgtcaaaaccctaaatccgacaaattcaccgtcaaaaaattccaccacgtcgaattc

SEQ ID NO: 314 - DNA, 200 bp, Conyza canadensis
gaattcgacgtggtggaattttttgacggtgaatttgtcggatttagggttttgacggatgaagtttttgaagccgacgagtttgtaagtggcggaggtggtggtagtggtttgttgttgtccggtgagtgtgtttccgtttgctgcttcagttaccattttgtttttattaatttgaatgtatagatagaaatatgaag

SEQ ID NO: 315 - DNA, 200 bp, Conyza canadensis
caaaaaattccaccacgtcgaattctggtgttccgacgccaccaacaccgcccgccgcttctcttggggtctcggcatgccgcttctttacaaatccgatctctccaccggtaacaacactcacgcttcttaccttattaactccggccagctcaacttcctcttcaccgcaccttattccaccaccatctccaccaccg

SEQ ID NO: 316 - DNA, 200 bp, Conyza canadensis
cggtggtggagatggtggtggaataaggtgcggtgaagaggaagttgagctggccggagttaataaggtaagaagcgtgagtgttgttaccggtggagagatcggatttgtaaagaagcggcatgccgagaccccaagagaagcggcgggcggtgttggtggcgtcggaacaccagaattcgacgtggtggaattttttg

SEQ ID NO: 317 - DNA, 200 bp, Conyza canadensis
tattccaccaccatctccaccaccggttccacatcgtctattcctactttctctcacactgcttgccggaacttcaccgccactcacggcctcgctgtacggtctatcgccgttgaagttgaagacgctgaaatcgcttattctgttagtatcgctcacggtgctaaaccgtcgtcagctccggtcactctaggaaataa

SEQ ID NO: 318 - DNA, 200 bp, Conyza canadensis
ttatttcctagagtgaccggagctgacgacggtttagcaccgtgagcgatactaacagaataagcgatttcagcgtcttcaacttcaacggcgatagaccgtacagcgaggccgtgagtggcggtgaagttccggcaagcagtgtgagagaaagtaggaatagacgatgtggaaccggtggtggagatggtggtggaata

SEQ ID NO: 319 - DNA, 200 bp, Conyza canadensis
cagctccggtcactctaggaaataacgacgtcgttttggctgaagttaaactgtacggcgatgtcgttttgagatatgttagttttaaaaacactaattatgatgttactactaatttcctaccgggttttgagcctgtggacgcaacgtcgtcgtttcatgaacttaattacggaattcataagttagatcacgccgta

SEQ ID NO: 320 - DNA, 200 bp, Conyza canadensis
tacggcgtgatctaacttatgaattccgtaattaagttcatgaaacgacgacgttgcgtccacaggctcaaaacccggtaggaaattagtagtaacatcataattagtgtttttaaaactaacatatctcaaaacgacatcgccgtacagtttaacttcagccaaaacgacgtcgttatttcctagagtgaccggagctg

SEQ ID NO: 321 - DNA, 200 bp, Conyza canadensis
aattcataagttagatcacgccgtaggaaacgtgccggagctagctccggcagtagactacgtgaagtccttcactggatttcacgaatttgctgaatttacggcagaggatgtaggaacgagtgagagcgggcttaattcggttgtattagcgtgtaatagtgaaacggttttgataccgatgaacgagccagtttatg

SEQ ID NO: 322 - DNA, 200 bp, Conyza canadensis
cataaactggctcgttcatcggtatcaaaaccgtttcactattacacgctaatacaaccgaattaagcccgctctcactcgttcctacatcctctgccgtaaattcagcaaattcgtgaaatccagtgaaggacttcacgtagtctactgccggagctagctccggcacgtttcctacggcgtgatctaacttatgaatt

SEQ ID NO: 323 - DNA, 200 bp, Conyza canadensis
ataccgatgaacgagccagtttatggaacgaaaaggaagagccagatacagacgtatttggaacataatgagggagctggtgtgcagcatttggcgttggcgagtgaggatatttttaggactttgagagagatgagaaaacggagtagtatcggtggttttgagtttatgccgtctccaccgcctacttattataagaa

SEQ ID NO: 324 - DNA, 200 bp, Conyza canadensis
ttcttataataagtaggcggtggagacggcataaactcaaaaccaccgatactactccgttttctcatctctctcaaagtcctaaaaatatcctcactcgccaacgccaaatgctgcacaccagctccctcattatgttccaaatacgtctgtatctggctcttccttttcgttccataaactggctcgttcatcggtat

SEQ ID NO: 325 - DNA, 200 bp, Conyza canadensis
ctccaccgcctacttattataagaatttgaagaatagggtcggggacgtgttgagtgacgaacagattaaggaatgtgaggaattggggattttggttgatagagatgatcagggaactttgcttcagattttcaccaagcctgttggtgataggtatgtctatgttaactttatcagtgattgtgcatttgtccatttt

SEQ ID NO: 326 - DNA, 200 bp, Conyza canadensis
aaaatggacaaatgcacaatcactgataaagttaacatagacatacctatcaccaacaggcttggtgaaaatctgaagcaaagttccctgatcatctctatcaaccaaaatccccaattcctcacattccttaatctgttcgtcactcaacacgtccccgaccctattcttcaaattcttataataagtaggcggtggag

SEQ ID NO: 327 - DNA, 200 bp, Conyza canadensis
cagtgattgtgcatttgtccattttgttgaattcgtaaaacatgagattaaatacctgcaatatgttttgcttacattgaatctaggccaggttttcagtgaacaagtaccataaatgtataatgtaggtctagtcttgttaatgatcagtgatcaatagtgcatacattgaatgacgaattgacaactagtttctaata

SEQ ID NO: 328 - DNA, 200 bp, Conyza canadensis
tattagaaactagttgtcaattcgtcattcaatgtatgcactattgatcactgatcattaacaagactagacctacattatacatttatggtacttgttcactgaaaacctggcctagattcaatgtaagcaaaacatattgcaggtatttaatctcatgttttacgaattcaacaaaatggacaaatgcacaatcactg

SEQ ID NO: 329 - DNA, 200 bp, Conyza canadensis
acgaattgacaactagtttctaatagaatgtgaaatataaagtgaatgtcacaaaagctgttatcttatctattttagttgtaatggaccaatggttggactgtttgaaagtgtgcttacaatcaagattcgagattgttactaggatgctagcgctaggtatatatcatatggtgtctgagctctatggtagtataaat

SEQ ID NO: 330 - DNA, 200 bp, Conyza canadensis
atttatactaccatagagctcagacaccatatgatatatacctagcgctagcatcctagtaacaatctcgaatcttgattgtaagcacactttcaaacagtccaaccattggtccattacaactaaaatagataagataacagcttttgtgacattcactttatatttcacattctattagaaactagttgtcaattcgt

SEQ ID NO: 331 - DNA, 200 bp, Conyza canadensis
gtctgagctctatggtagtataaattgtgaatacactaaacatctcccaaaaatatttgagtaaccgatcaatctactagttattgcaatgccaaacatctctttaaacaactacttattgtaaagccatactacatcaacacaccaaaaatctatctcccacaacccccgttttacttgggttatgaacttagtatagt

SEQ ID NO: 332 - DNA, 200 bp, Conyza canadensis
actatactaagttcataacccaagtaaaacgggggttgtgggagatagatttttggtgtgttgatgtagtatggctttacaataagtagttgtttaaagagatgtttggcattgcaataactagtagattgatcggttactcaaatatttttgggagatgtttagtgtattcacaatttatactaccatagagctcagac

SEQ ID NO: 333 - DNA, 200 bp, Conyza canadensis
acttgggttatgaacttagtatagttattcaaaataatcactttcaaaatggatccaacacatctgaaactggtcacatttgagttcttatctttcaaataatttgtaattcatatcaggctcagcttcatggggtctggcctttgtctgtgtgtttgtgttgtgtcttttctggtgtgatctaattcggtatgcatatt

SEQ ID NO: 334 - DNA, 200 bp, Conyza canadensis
aatatgcataccgaattagatcacaccagaaaagacacaacacaaacacacagacaaaggccagaccccatgaagctgagcctgatatgaattacaaattatttgaaagataagaactcaaatgtgaccagtttcagatgtgttggatccattttgaaagtgattattttgaataactatactaagttcataacccaagt

SEQ ID NO: 335 - DNA, 200 bp, Conyza canadensis
tgtgatctaattcggtatgcatattgtttgagcaggccaactatattcatagagattattcagagagtagggtgtatgctgaaagatgatgaaggcaaggtgcaacagaagccaggctgtggaggatttggcaagggcaatttctcagagctcttcaaatctattgaagaatacgagaagacgctggaagcacgaaacac

SEQ ID NO: 336 - DNA, 200 bp, Conyza canadensis
gtgtttcgtgcttccagcgtcttctcgtattcttcaatagatttgaagagctctgagaaattgcccttgccaaatcctccacagcctggcttctgttgcaccttgccttcatcatctttcagcatacaccctactctctgaataatctctatgaatatagttggcctgctcaaacaatatgcataccgaattagatcaca

SEQ ID NO: 337 - DNA, 200 bp, Conyza canadensis
agaagacgctggaagcacgaaacaccactgaaccaactgccactgcatgaaaactgcctatgtataatgtttatatcttcttgactcaaagaacatgattatcaccatatgtatttgtggattatgtacaattgaatgaaacaatgcaataccatatgtgatgtgatttatatagaataacaatagatgtcattcatata

SEQ ID NO: 338 - DNA, 200 bp, Conyza canadensis
tatatgaatgacatctattgttattctatataaatcacatcacatatggtattgcattgtttcattcaattgtacataatccacaaatacatatggtgataatcatgttctttgagtcaagaagatataaacattatacataggcagttttcatgcagtggcagttggttcagtggtgtttcgtgcttccagcgtcttct

SEQ ID NO: 339 - DNA, 200 bp, Conyza canadensis
aataacaatagatgtcattcatatatgccgttcctgttttagtaattgtgtgttggtggtgtgttgcaagttccaatcagctgtatataatgcctaaatattcaatttgacctccattaaggatccattgtgcagattctttgtttctgtcttaaaagtgtgggagacttgaaacacatctgtaaaactgaaccagatct

SEQ ID NO: 340 - DNA, 200 bp, Conyza canadensis
agatctggttcagttttacagatgtgtttcaagtctcccacacttttaagacagaaacaaagaatctgcacaatggatccttaatggaggtcaaattgaatatttaggcattatatacagctgattggaacttgcaacacaccaccaacacacaattactaaaacaggaacggcatatatgaatgacatctattgttatt

SEQ ID NO: 341 - DNA, 200 bp, Conyza canadensis
acatctgtaaaactgaaccagatctgaatcgagctgaatggaagcggcgcaggtctgctctgtggttccacttacaacaaacaattgttcatggtacgtcgcctcttcatagccagtgtttattgtaagaaagcattgtttaggcgaaaaaggaaaaggtgatttatgtaagaaaaggtagtgctattaaggttatgttc

SEQ ID NO: 342 - DNA, 200 bp, Conyza canadensis
gaacataaccttaatagcactaccttttcttacataaatcaccttttcctttttcgcctaaacaatgctttcttacaataaacactggctatgaagaggcgacgtaccatgaacaattgtttgttgtaagtggaaccacagagcagacctgcgccgcttccattcagctcgattcagatctggttcagttttacagatgt

SEQ ID NO: 343 - DNA, 200 bp, Conyza canadensis
aggtagtgctattaaggttatgttccccatgaacaaacagggtaaattcatacaggatcaacttttaaatgatactattaaaggtcatctacttctgtttcccatgcttgtgccaagctcaaatttagacgaaaccaaaacaggatgagaaaactaattaacacatattttagtaagcaattacagctatatatcatact

SEQ ID NO: 344 - DNA, 200 bp, Conyza canadensis
agtatgatatatagctgtaattgcttactaaaatatgtgttaattagttttctcatcctgttttggtttcgtctaaatttgagcttggcacaagcatgggaaacagaagtagatgacctttaatagtatcatttaaaagttgatcctgtatgaatttaccctgtttgttcatggggaacataaccttaatagcactacct

SEQ ID NO: 345 - DNA, 200 bp, Conyza canadensis
attaaaaacacgaatgggctagctagctgatgaatttaatgtatatatgtctttaaaaaccactttcatgggctacttgtgtttttgatgagtttttcaacatcattctcatcttaccttcttacaatggttcaccatattgcttcgtttgtcgtctcctgcatggttatcgaatttattttttcaaattatattactat

SEQ ID NO: 346 - DNA, 200 bp, Conyza canadensis
atagtaatataatttgaaaaaataaattcgataaccatgcaggagacgacaaacgaagcaatatggtgaaccattgtaagaaggtaagatgagaatgatgttgaaaaactcatcaaaaacacaagtagcccatgaaagtggtttttaaagacatatatacattaaattcatcagctagctagcccattcgtgtttttaat

SEQ ID NO: 347 - DNA, 200 bp, Conyza canadensis
ttattttttcaaattatattactattatctttttgttttctgatttctcacatgtaatttacattatgagtttcaccatccgtatgtaaattgtaaaataaatcacaaatttggtttttaaccatcaactaaattcattgaaatccttaagttataacccatttgaggttatagattatgagagtagattcctattttct

SEQ ID NO: 348 - DNA, 200 bp, Conyza canadensis
agaaaataggaatctactctcataatctataacctcaaatgggttataacttaaggatttcaatgaatttagttgatggttaaaaaccaaatttgtgatttattttacaatttacatacggatggtgaaactcataatgtaaattacatgtgagaaatcagaaaacaaaaagataatagtaatataatttgaaaaaataa

SEQ ID NO: 349 - DNA, 200 bp, Conyza canadensis
ttatgagagtagattcctattttctctttggcttatgtatccttaattatgtttgaattgattctaactttattatattttaggaattacttaacttcaaaaatctcaacacgtcatataatgctttttgtggatggatgcttttgacccatttcgttgaaaacttatgtcgacacacatatctgacaaacttgtactcg

SEQ ID NO: 350 - DNA, 200 bp, Conyza canadensis
cgagtacaagtttgtcagatatgtgtgtcgacataagttttcaacgaaatgggtcaaaagcatccatccacaaaaagcattatatgacgtgttgagatttttgaagttaagtaattcctaaaatataataaagttagaatcaattcaaacataattaaggatacataagccaaagagaaaataggaatctactctcataa

SEQ ID NO: 351 - DNA, 200 bp, Conyza canadensis
cacatatctgacaaacttgtactcgtatatcctttatcgcatatgctatagtataatattcgaaatttaatctcaagataattttcccaaaaggtcatattaattgaattgagattaattcctacctaacatatatagatatattgtgttggtgttatcctctttggagttttagtcgcgcatgtattctaagaaaatgg

SEQ ID NO: 352 - DNA, 200 bp, Conyza canadensis
ccattttcttagaatacatgcgcgactaaaactccaaagaggataacaccaacacaatatatctatatatgttaggtaggaattaatctcaattcaattaatatgaccttttgggaaaattatcttgagattaaatttcgaatattatactatagcatatgcgataaaggatatacgagtacaagtttgtcagatatgtg

SEQ ID NO: 353 - DNA, 200 bp, Conyza canadensis
tcgcgcatgtattctaagaaaatggtaattttgaacttatattgatggtctaaattttatcctatcactaaaatttcagtcatgtttcctagctaattaatttgggttcaatccaggtattctaaataagttttgtaaatacatgtctattatcataatgaaacatagaaaaacccgtaaagtaaaaatgtctatatttt

SEQ ID NO: 354 - DNA, 200 bp, Conyza canadensis
aaaatatagacatttttactttacgggtttttctatgtttcattatgataatagacatgtatttacaaaacttatttagaatacctggattgaacccaaattaattagctaggaaacatgactgaaattttagtgataggataaaatttagaccatcaatataagttcaaaattaccattttcttagaatacatgcgcga

SEQ ID NO: 355 - DNA, 200 bp, Conyza canadensis
cgtaaagtaaaaatgtctatatttttagtggcaaattattaatgttgctataatgaaaagagaagattcaaccttttgtacatcataaaaaatgttattctacattatgctaattttacattagacttcatagttttgaaaactttcaaatgaagggacttaaattttaaatttaaagatgatgtcataaattagttaga

SEQ ID NO: 356 - DNA, 200 bp, Conyza canadensis
tctaactaatttatgacatcatctttaaatttaaaatttaagtcccttcatttgaaagttttcaaaactatgaagtctaatgtaaaattagcataatgtagaataacattttttatgatgtacaaaaggttgaatcttctcttttcattatagcaacattaataatttgccactaaaaatatagacatttttactttacg

SEQ ID NO: 357 - DNA, 200 bp, Conyza canadensis
aagatgatgtcataaattagttagataaaagttaaataattaataagaaaaggtctaatgataccaaataaaattttcttaaaaaagtattaatgtcattacaattttattttatttatataagagaagataatagattttcttgtataaaaaacttacctgtaaacttctatataaactttttagaaaaataatttaaa

SEQ ID NO: 358 - DNA, 200 bp, Conyza canadensis
tttaaattatttttctaaaaagtttatatagaagtttacaggtaagttttttatacaagaaaatctattatcttctcttatataaataaaataaaattgtaatgacattaatacttttttaagaaaattttatttggtatcattagaccttttcttattaattatttaacttttatctaactaatttatgacatcatctt

SEQ ID NO: 359 - DNA, 200 bp, Conyza canadensis
aaactttttagaaaaataatttaaaacggtctcaaatatcgttatttccgaacatacaaaaacccaaaacctaatccatttaattatattgtaagacctcgattagcttaatttattttgaaacctatttacaaatgagtttggcatgatatgaacttttttgcatttacatatggctgtaaacgaactgaacagttcac

SEQ ID NO: 360 - DNA, 200 bp, Conyza canadensis
gtgaactgttcagttcgtttacagccatatgtaaatgcaaaaaagttcatatcatgccaaactcatttgtaaataggtttcaaaataaattaagctaatcgaggtcttacaatataattaaatggattaggttttgggtttttgtatgttcggaaataacgatatttgagaccgttttaaattatttttctaaaaagttt

SEQ ID NO: 361 - DNA, 200 bp, Conyza canadensis
gctgtaaacgaactgaacagttcacgaacaattcatgaaccgttcggcgggaggttcgttcgtgttagttcgtttagttaaatgaatgaacatgaacaaagctctcgttcgttcatttacgttcatgaacgttcgatagtctgttcgtgaactttagttcgtctatgttcatgaactgtcgttcgtgaacaataatttga

SEQ ID NO: 362 - DNA, 200 bp, Conyza canadensis
tcaaattattgttcacgaacgacagttcatgaacatagacgaactaaagttcacgaacagactatcgaacgttcatgaacgtaaatgaacgaacgagagctttgttcatgttcattcatttaactaaacgaactaacacgaacgaacctcccgccgaacggttcatgaattgttcgtgaactgttcagttcgtttacagc

SEQ ID NO: 363 - DNA, 200 bp, Conyza canadensis
ctgtcgttcgtgaacaataatttgattatgttcatttacacacacatacataatcaactgtactataaaaatagtttaaatataaatatttttttatataatatatattattaatatctaattatttaagaaaaagtttaaataaaatacttaacttataaatatttcgttcgtttgtgttcgtgtacatttgttcatga

SEQ ID NO: 364 - DNA, 200 bp, Conyza canadensis
tcatgaacaaatgtacacgaacacaaacgaacgaaatatttataagttaagtattttatttaaactttttcttaaataattagatattaataatatatattatataaaaaaatatttatatttaaactatttttatagtacagttgattatgtatgtgtgtgtaaatgaacataatcaaattattgttcacgaacgacag

SEQ ID NO: 365 - DNA, 200 bp, Conyza canadensis
tgtgttcgtgtacatttgttcatgaacacaaatgaacgaacatgaacaagtcattatgttcgtttatctgtttgttaagttaaatgaacgaacgaacacgaacaaggtctcgtttgtgttcgttcggttcgtttacggcccttaattacatgtgaagttaataaataaaaataaagttcatcaaaattaaacctatggtt

SEQ ID NO: 366 - DNA, 200 bp, Conyza canadensis
aaccataggtttaattttgatgaactttatttttatttattaacttcacatgtaattaagggccgtaaacgaaccgaacgaacacaaacgagaccttgttcgtgttcgttcgttcatttaacttaacaaacagataaacgaacataatgacttgttcatgttcgttcatttgtgttcatgaacaaatgtacacgaacaca

SEQ ID NO: 367 - DNA, 200 bp, Conyza canadensis
gttcatcaaaattaaacctatggttggagataaaaccagtcagccttacgtgtggaaactatcctactctacaattaatagcacccaaagaaaataaaccacttccgtgttcttcttcatctccatcatccaacccctcaactacttgtactgtatctatatctatcattctatatatctacttcatatttctatctata

SEQ ID NO: 368 - DNA, 200 bp, Conyza canadensis
tatagatagaaatatgaagtagatatatagaatgatagatatagatacagtacaagtagttgaggggttggatgatggagatgaagaagaacacggaagtggtttattttctttgggtgctattaattgtagagtaggatagtttccacacgtaaggctgactggttttatctccaaccataggtttaattttgatgaac

SEQ ID NO: 369 - DNA, 200 bp, Conyza canadensis
tatctacttcatatttctatctatacattcaaattaataaaaacaaaatggtaactgaagcagcaaacggaaacacactcaccggacaacaacaaaccactaccaccacctccgccacttacaaactcgtcggcttcaaaaacttcatccgtcaaaaccctaaatccgacaaattcaccgtcaaaaaattccaccacgtc

SEQ ID NO: 370 - DNA, 200 bp, Conyza canadensis
gacgtggtggaattttttgacggtgaatttgtcggatttagggttttgacggatgaagtttttgaagccgacgagtttgtaagtggcggaggtggtggtagtggtttgttgttgtccggtgagtgtgtttccgtttgctgcttcagttaccattttgtttttattaatttgaatgtatagatagaaatatgaagtagata

SEQ ID NO: 371 - DNA, 200 bp, Conyza canadensis
caccgtcaaaaaattccaccacgtcgaattctggtgttccgacgccaccaacaccgcccgccgcttctcttggggtctcggcatgccgcttctttacaaatccgatctctccaccggtaacaacactcacgcttcttaccttattaactccggccagctcaacttcctcttcaccgcaccttattccaccaccatctcca

SEQ ID NO: 372 - DNA, 200 bp, Conyza canadensis
tggagatggtggtggaataaggtgcggtgaagaggaagttgagctggccggagttaataaggtaagaagcgtgagtgttgttaccggtggagagatcggatttgtaaagaagcggcatgccgagaccccaagagaagcggcgggcggtgttggtggcgtcggaacaccagaattcgacgtggtggaattttttgacggtg

SEQ ID NO: 373 - DNA, 200 bp, Conyza canadensis
gcaccttattccaccaccatctccaccaccggttccacatcgtctattcctactttctctcacactgcttgccggaacttcaccgccactcacggcctcgctgtacggtctatcgccgttgaagttgaagacgctgaaatcgcttattctgttagtatcgctcacggtgctaaaccgtcgtcagctccggtcactctagg

SEQ ID NO: 374 - DNA, 200 bp, Conyza canadensis
cctagagtgaccggagctgacgacggtttagcaccgtgagcgatactaacagaataagcgatttcagcgtcttcaacttcaacggcgatagaccgtacagcgaggccgtgagtggcggtgaagttccggcaagcagtgtgagagaaagtaggaatagacgatgtggaaccggtggtggagatggtggtggaataaggtgc

SEQ ID NO: 375 - DNA, 200 bp, Conyza canadensis
cgtcgtcagctccggtcactctaggaaataacgacgtcgttttggctgaagttaaactgtacggcgatgtcgttttgagatatgttagttttaaaaacactaattatgatgttactactaatttcctaccgggttttgagcctgtggacgcaacgtcgtcgtttcatgaacttaattacggaattcataagttagatcac

SEQ ID NO: 376 - DNA, 200 bp, Conyza canadensis
gtgatctaacttatgaattccgtaattaagttcatgaaacgacgacgttgcgtccacaggctcaaaacccggtaggaaattagtagtaacatcataattagtgtttttaaaactaacatatctcaaaacgacatcgccgtacagtttaacttcagccaaaacgacgtcgttatttcctagagtgaccggagctgacgacg

SEQ ID NO: 377 - DNA, 200 bp, Conyza canadensis
ttacggaattcataagttagatcacgccgtaggaaacgtgccggagctagctccggcagtagactacgtgaagtccttcactggatttcacgaatttgctgaatttacggcagaggatgtaggaacgagtgagagcgggcttaattcggttgtattagcgtgtaatagtgaaacggttttgataccgatgaacgagccag

SEQ ID NO: 378 - DNA, 200 bp, Conyza canadensis
ctggctcgttcatcggtatcaaaaccgtttcactattacacgctaatacaaccgaattaagcccgctctcactcgttcctacatcctctgccgtaaattcagcaaattcgtgaaatccagtgaaggacttcacgtagtctactgccggagctagctccggcacgtttcctacggcgtgatctaacttatgaattccgtaa

SEQ ID NO: 379 - DNA, 200 bp, Conyza canadensis
gttttgataccgatgaacgagccagtttatggaacgaaaaggaagagccagatacagacgtatttggaacataatgagggagctggtgtgcagcatttggcgttggcgagtgaggatatttttaggactttgagagagatgagaaaacggagtagtatcggtggttttgagtttatgccgtctccaccgcctacttatta

SEQ ID NO: 380 - DNA, 200 bp, Conyza canadensis
taataagtaggcggtggagacggcataaactcaaaaccaccgatactactccgttttctcatctctctcaaagtcctaaaaatatcctcactcgccaacgccaaatgctgcacaccagctccctcattatgttccaaatacgtctgtatctggctcttccttttcgttccataaactggctcgttcatcggtatcaaaac

SEQ ID NO: 381 - DNA, 200 bp, Conyza canadensis
tgccgtctccaccgcctacttattataagaatttgaagaatagggtcggggacgtgttgagtgacgaacagattaaggaatgtgaggaattggggattttggttgatagagatgatcagggaactttgcttcagattttcaccaagcctgttggtgataggaggccaactatattcatagagattattcagagagtaggg

SEQ ID NO: 382 - DNA, 200 bp, Conyza canadensis
ccctactctctgaataatctctatgaatatagttggcctcctatcaccaacaggcttggtgaaaatctgaagcaaagttccctgatcatctctatcaaccaaaatccccaattcctcacattccttaatctgttcgtcactcaacacgtccccgaccctattcttcaaattcttataataagtaggcggtggagacggca

SEQ ID NO: 383 - DNA, 200 bp, Conyza canadensis
catagagattattcagagagtagggtgtatgctgaaagatgatgaaggcaaggtgcaacagaagccaggctgtggaggatttggcaagggcaatttctcagagctcttcaaatctattgaagaatacgagaagacgctggaagcacgaaacaccactgaaccaactgccactgcatgaaaactgcctatgtataatgttt

SEQ ID NO: 384 - DNA, 200 bp, Conyza canadensis
aaacattatacataggcagttttcatgcagtggcagttggttcagtggtgtttcgtgcttccagcgtcttctcgtattcttcaatagatttgaagagctctgagaaattgcccttgccaaatcctccacagcctggcttctgttgcaccttgccttcatcatctttcagcatacaccctactctctgaataatctctatg

SEQ ID NO: 385 - DNA, 200 bp, Conyza canadensis
tgaaaactgcctatgtataatgtttatatcttcttgactcaaagaacatgattatcaccatatgtatttgtggattatgtacaattgaatgaaacaatgcaataccatatgtgatgtgatttatatagaataacaatagatgtcattcatatatgccgttcctgttttagtaattgtgtgttggtggtgtgttgcaagtt

SEQ ID NO: 386 - DNA, 200 bp, Conyza canadensis
aacttgcaacacaccaccaacacacaattactaaaacaggaacggcatatatgaatgacatctattgttattctatataaatcacatcacatatggtattgcattgtttcattcaattgtacataatccacaaatacatatggtgataatcatgttctttgagtcaagaagatataaacattatacataggcagttttca

SEQ ID NO: 387 - DNA, 200 bp, Conyza canadensis
gtgtgttggtggtgtgttgcaagttccaatcagctgtatataatgcctaaatattcaatttgacctccattaaggatccattgtgcagattctttgtttctgtcttaaaagtgtgggagacttgaaacacatctgtaaaactgaaccagatctgaatcgagctgaatggaagcggcgcaggtctgctctttggttccact

SEQ ID NO: 388 - DNA, 200 bp, Conyza canadensis
agtggaaccaaagagcagacctgcgccgcttccattcagctcgattcagatctggttcagttttacagatgtgtttcaagtctcccacacttttaagacagaaacaaagaatctgcacaatggatccttaatggaggtcaaattgaatatttaggcattatatacagctgattggaacttgcaacacaccaccaacacac

SEQ ID NO: 389 - DNA, 200 bp, Conyza canadensis
cgcaggtctgctctttggttccacttacaacaaacaattgttcatggtacgtcgcctcttcatggccagtgtttattgtaagaaagcattgtttaggcgaaaaaggaaaaggtgatttatgtaagaaaaggtagtgctattaaggttatgttccccatgaacaaacagggtaaattcatacaggatcaacttttaaatga

SEQ ID NO: 390 - DNA, 200 bp, Conyza canadensis
tcatttaaaagttgatcctgtatgaatttaccctgtttgttcatggggaacataaccttaatagcactaccttttcttacataaatcaccttttcctttttcgcctaaacaatgctttcttacaataaacactggccatgaagaggcgacgtaccatgaacaattgtttgttgtaagtggaaccaaagagcagacctgcg

SEQ ID NO: 391 - DNA, 200 bp, Conyza canadensis
tcatacaggatcaacttttaaatgatactattaaaggtcatctacttctgtttcccatgcttgtgccaagctcaaatttagacgaaaccaaaacaggatgaaaaaactaattaacacatattttagtaagcaattacagctatatatcatactatcatcaacaatctgaaaagttgcatacaatttatcgtttattcctg

SEQ ID NO: 392 - DNA, 200 bp, Conyza canadensis
caggaataaacgataaattgtatgcaacttttcagattgttgatgatagtatgatatatagctgtaattgcttactaaaatatgtgttaattagttttttcatcctgttttggtttcgtctaaatttgagcttggcacaagcatgggaaacagaagtagatgacctttaatagtatcatttaaaagttgatcctgtatga

SEQ ID NO: 393 - DNA, 200 bp, Conyza canadensis
gcatacaatttatcgtttattcctgtcaagtgatgacatgaaaaataccatttagtttacacgtactcagatatgccaataatagagggactttttctatatgagaacatacatgatgttcataattcataatttacttgatacggcaaggtacttggtatggcgtccttgtgcaattgtttttccggtctttttgttcc

SEQ ID NO: 394 - DNA, 200 bp, Conyza canadensis
ggaacaaaaagaccggaaaaacaattgcacaaggacgccataccaagtaccttgccgtatcaagtaaattatgaattatgaacatcatgtatgttctcatatagaaaaagtccctctattattggcatatctgagtacgtgtaaactaaatggtatttttcatgtcatcacttgacaggaataaacgataaattgtatgc

SEQ ID NO: 395 - DNA, 200 bp, Conyza canadensis
attgtttttccggtctttttgttccttaattcaacggtcacaacagcaatagccttcccaacacgcaatgtttttgcctctatctcaatctcgtcctgtaagaaaattgcatccttaacaaaattttactcaaagacgcatctatggtacgaagtacaaaccaaagattttcagtaagtcgtgcaaaacctttatgcata

SEQ ID NO: 396 - DNA, 200 bp, Conyza canadensis
tatgcataaaggttttgcacgacttactgaaaatctttggtttgtacttcgtaccatagatgcgtctttgagtaaaattttgttaaggatgcaattttcttacaggacgagattgagatagaggcaaaaacattgcgtgttgggaaggctattgctgttgtgaccgttgaattaaggaacaaaaagaccggaaaaacaat

SEQ ID NO: 397 - DNA, 200 bp, Conyza canadensis
aagtcgtgcaaaacctttatgcatactgcaactactcaaacttcacatcttatgcctaaaatcaaagaccccaaagaagccttgagttcaaaatccaagaatttctgtcagctacaggggacaggttcaacccatttacatacaaatgggtcaaaatgggttatgttattctgtgtttctgccaaaccagacctggcacg

SEQ ID NO: 398 - DNA, 200 bp, Conyza canadensis
cgtgccaggtctggtttggcagaaacacagaataacataacccattttgacccatttgtatgtaaatgggttgaacctgtcccctgtagctgacagaaattcttggattttgaactcaaggcttctttggggtctttgattttaggcataagatgtgaagtttgagtagttgcagtatgcataaaggttttgcacgactt

SEQ ID NO: 399 - DNA, 200 bp, Conyza canadensis
tttctgccaaaccagacctggcacgtactaaaactcacccattacccaacccacctacccagcccccctttttaccaacatgcattttgcttttgcaaccctgttgcttcaagcattcaactattcaagtagcaagatcgaaagaacttatacaaaaagttgccatttatgattaagtgaagccttaatgacaccaaatc

SEQ ID NO: 400 - DNA, 200 bp, Conyza canadensis
gatttggtgtcattaaggcttcacttaatcataaatggcaactttttgtataagttctttcgatcttgctacttgaatagttgaatgcttgaagcaacagggttgcaaaagcaaaatgcatgttggtaaaaaggggggctgggtaggtgggttgggtaatgggtgagttttagtacgtgccaggtctggtttggcagaaa

SEQ ID NO: 401 - DNA, 200 bp, Conyza canadensis
agtgaagccttaatgacaccaaatcagttattatagcattacaatgggagagcttcaacagaaaagttatcaagtcttctccaacaagatcatgagctcattttcaattttatatcaaacggtagcaataacgaaagtaaatctggtggctcaaggtaaaacctcataacctttgatggcataataacttccagaccaac

SEQ ID NO: 402 - DNA, 200 bp, Conyza canadensis
gttggtctggaagttattatgccatcaaaggttatgaggttttaccttgagccaccagatttactttcgttattgctaccgtttgatataaaattgaaaatgagctcatgatcttgttggagaagacttgataacttttctgttgaagctctcccattgtaatgctataataactgatttggtgtcattaaggcttcact

SEQ ID NO: 403 - DNA, 200 bp, Conyza canadensis
atggcataataacttccagaccaactaaacaccaaggtggaagttattactttgtgctaatgatagcagttacaaacataaaaaatctggtcggcttcgttcagttttctgaaataccaatatcgtctgacaaggtgccatcttcatattcagagtcacttcatgatcaactcttgtggggtttcaaactcaagaacgtt

SEQ ID NO: 404 - DNA, 200 bp, Conyza canadensis
aacgttcttgagtttgaaaccccacaagagttgatcatgaagtgactctgaatatgaagatggcaccttgtcagacgatattggtatttcagaaaactgaacgaagccgaccagattttttatgtttgtaactgctatcattagcacaaagtaataacttccaccttggtgtttagttggtctggaagttattatgccat

SEQ ID NO: 405 - DNA, 200 bp, Conyza canadensis
gtggggtttcaaactcaagaacgttttaaatacattcatttaatatttttatctccagaatactactcaacactttcctgtaaggtcttcaatttcagggggaaaaaaacccaaaattcaaggaatcctgctattagtgtatcgtttcttgatagcaaagctttcatttacaatctctaacaacatttttacttcaaaat

SEQ ID NO: 406 - DNA, 200 bp, Conyza canadensis
attttgaagtaaaaatgttgttagagattgtaaatgaaagctttgctatcaagaaacgatacactaatagcaggattccttgaattttgggtttttttccccctgaaattgaagaccttacaggaaagtgttgagtagtattctggagataaaaatattaaatgaatgtatttaaaacgttcttgagtttgaaaccccac

SEQ ID NO: 407 - DNA, 200 bp, Conyza canadensis
tatggtattgcattgtttcattcaattgtacataatccacaaatacatatggtgataatcatgttctttgagtcaagaagatataaacattatacataggcagttttcatgcagtggcagttggttcagtggtgtttcgtgcttccagcgtcttctcgtattcttcaatagatttgaagagctctgagaaattgcccttg

SEQ ID NO: 408 - DNA, 200 bp, Conyza canadensis
caagggcaatttctcagagctcttcaaatctattgaagaatacgagaagacgctggaagcacgaaacaccactgaaccaactgccactgcatgaaaactgcctatgtataatgtttatatcttcttgactcaaagaacatgattatcaccatatgtatttgtggattatgtacaattgaatgaaacaatgcaataccata

SEQ ID NO: 409 - DNA, 200 bp, Conyza canadensis
gaagagctctgagaaattgcccttgccaaatcctccacagcctggcttctgttgcaccttgccttcatcatctttcagcatacaccctactctctgaataatctctatgaatatagttggcctatcaccaacaggcttggtgaaaatctgaagcaaagttccctgatcatctctatcaaccaaaatccccaattcctcac

SEQ ID NO: 410 - DNA, 200 bp, Conyza canadensis
gtgaggaattggggattttggttgatagagatgatcagggaactttgcttcagattttcaccaagcctgttggtgataggccaactatattcatagagattattcagagagtagggtgtatgctgaaagatgatgaaggcaaggtgcaacagaagccaggctgtggaggatttggcaagggcaatttctcagagctcttc

SEQ ID NO: 411 - DNA, 200 bp, Conyza canadensis
tcaaccaaaatccccaattcctcacattccttaatctgttcgtcactcaacacgtccccgaccctattcttcaaattcttataataagtaggcggtggagacggcataaactcaaaaccaccgacactactccgttttctcatctctctcaaagtcctaaaaatatcctcactcgccaacgccaaatgctgcacaccagc

SEQ ID NO: 412 - DNA, 200 bp, Conyza canadensis
gctggtgtgcagcatttggcgttggcgagtgaggatatttttaggactttgagagagatgagaaaacggagtagtgtcggtggttttgagtttatgccgtctccaccgcctacttattataagaatttgaagaatagggtcggggacgtgttgagtgacgaacagattaaggaatgtgaggaattggggattttggttga

SEQ ID NO: 413 - DNA, 200 bp, Conyza canadensis
ccaacgccaaatgctgcacaccagctccctcattatgttccaaatacgtctgtatctggctcttccttttcgttccataaactggctcgttcatcggtatcaaaaccgtttcactattacacgctaatacaaccgaattaagcccgctctcactcgttcctacatcctctgccgtaaattcagcaaattcgtgaaatcca

SEQ ID NO: 414 - DNA, 200 bp, Conyza canadensis
tggatttcacgaatttgctgaatttacggcagaggatgtaggaacgagtgagagcgggcttaattcggttgtattagcgtgtaatagtgaaacggttttgataccgatgaacgagccagtttatggaacgaaaaggaagagccagatacagacgtatttggaacataatgagggagctggtgtgcagcatttggcgttgg

SEQ ID NO: 415 - DNA, 200 bp, Conyza canadensis
aaattcagcaaattcgtgaaatccagtgaaggacttcacgtagtctactgccggagctagctccggcacgtttcctacggcgtgatctaacttatgaattccgtaattaagttcatgaaacgacgacgttgcgtccacaggctcaaaacccggtaggaaattagtagtaacatcataattagtgtttttaaaactaacat

SEQ ID NO: 416 - DNA, 200 bp, Conyza canadensis
atgttagttttaaaaacactaattatgatgttactactaatttcctaccgggttttgagcctgtggacgcaacgtcgtcgtttcatgaacttaattacggaattcataagttagatcacgccgtaggaaacgtgccggagctagctccggcagtagactacgtgaagtccttcactggatttcacgaatttgctgaattt

SEQ ID NO: 417 - DNA, 200 bp, Conyza canadensis
taattagtgtttttaaaactaacatatctcaaaacgacatcgccgtacagtttaacttcagccaaaacgacgtcgttatttcctagagtgaccggagctgacgacggtttagcaccgtgagcgatactaacagaataagcgatttcagcgtcttcaacttcaacggcgatagaccgtacagcgaggccgtgagtggcggt

SEQ ID NO: 418 - DNA, 200 bp, Conyza canadensis
accgccactcacggcctcgctgtacggtctatcgccgttgaagttgaagacgctgaaatcgcttattctgttagtatcgctcacggtgctaaaccgtcgtcagctccggtcactctaggaaataacgacgtcgttttggctgaagttaaactgtacggcgatgtcgttttgagatatgttagttttaaaaacactaatta

SEQ ID NO: 419 - DNA, 200 bp, Conyza canadensis
gtacagcgaggccgtgagtggcggtgaagttccggcaagcagtgtgagagaaagtaggaatagacgatgtggaaccggtggtggagatggtggtggaataaggtgcggtgaagaggaagttgagctggccggagttaataaggtaagaagcgtgagtgttgttaccggtggagagatcggatttgtaaagaagcggcatg

SEQ ID NO: 420 - DNA, 200 bp, Conyza canadensis
catgccgcttctttacaaatccgatctctccaccggtaacaacactcacgcttcttaccttattaactccggccagctcaacttcctcttcaccgcaccttattccaccaccatctccaccaccggttccacatcgtctattcctactttctctcacactgcttgccggaacttcaccgccactcacggcctcgctgtac

SEQ ID NO: 421 - DNA, 200 bp, Conyza canadensis
atcggatttgtaaagaagcggcatgccgagaccccaagagaagcggcgggcggtgttggtggcgtcggaacaccagaattcgacgtggtggaattttttgacggtgaatttgtcggatttagggttttgacggatgaagtttttgaagccgacgagtttgtaagtggcggaggtggtggtagtggtttgttgttgtccgg

SEQ ID NO: 422 - DNA, 200 bp, Conyza canadensis
ccggacaacaacaaaccactaccaccacctccgccacttacaaactcgtcggcttcaaaaacttcatccgtcaaaaccctaaatccgacaaattcaccgtcaaaaaattccaccacgtcgaattctggtgttccgacgccaccaacaccgcccgccgcttctcttggggtctcggcatgccgcttctttacaaatccgat

SEQ ID NO: 423 - DNA, 200 bp, Conyza canadensis
gtggtagtggtttgttgttgtccggtgagtgtgtttccgtttgctgcttcagttaccattttgtttttattaatttgaatgtatagatagaaatatgaagtagatatatagaatgatagatatagatacagtacaagtagttgaggggttggatgatggagatgaagaagaacacggaagtggtttattttctttgggtg

SEQ ID NO: 424 - DNA, 200 bp, Conyza canadensis
cacccaaagaaaataaaccacttccgtgttcttcttcatctccatcatccaacccctcaactacttgtactgtatctatatctatcattctatatatctacttcatatttctatctatacattcaaattaataaaaacaaaatggtaactgaagcagcaaacggaaacacactcaccggacaacaacaaaccactaccac

SEQ ID NO: 425 - DNA, 200 bp, Digitaria sanguinalis
gccgccaccgcctccctcccctccttctccgcccccgccgcgcgccgcttcgcctccgaccacggcctcgccgtgcgcgccgtagcgctccgcgtcgccgacgccgaggacgccttccgcgccagcgtcgccaacggggcgcgcccggcgttcgagcccgtcgagctcggcctcggcttccgcctcgccgaggtcgagct

SEQ ID NO: 426 - DNA, 200 bp, Digitaria sanguinalis
agctcgacctcggcgaggcggaagccgaggccgagctcgacgggctcgaacgccgggcgcgccccgttggcgacgctggcgcggaaggcgtcctcggcgtcggcgacgcggagcgctacggcgcgcacggcgaggccgtggtcggaggcgaagcggcgcgcggcgggggcggagaaggaggggagggaggcggtggcggc

SEQ ID NO: 427 - DNA, 200 bp, Digitaria sanguinalis
gcttccgcctcgccgaggtcgagctctacggcgacgtcgtgctccgctacgtcagctacccggacgccgcggatttgcccttcctgccgggcttcgaggacgtcgtcatcagcaacccaggggcggtggactacgggctgaggcgcttcgaccacatcgtcggcaacgtctcggagctggcgccggtggccgcgtacgtc

SEQ ID NO: 428 - DNA, 200 bp, Digitaria sanguinalis
gacgtacgcggccaccggcgccagctccgagacgttgccgacgatgtggtcgaagcgcctcagcccgtagtccaccgcccctgggttgctgatgacgacgtcctcgaagcccggcaggaagggcaaatccgcggcgtccgggtagctgacgtagcggagcacgacgtcgccgtagagctcgacctcggcgaggcggaagc

SEQ ID NO: 429 - DNA, 200 bp, Digitaria sanguinalis
gctggcgccggtggccgcgtacgtcgccggattcacggggttccacgagttcgccgagttcacggcggaggacgtgggcacggcggagagcgggctcaattccgtggtgctcgccaacaactccgagaacgtgctgctcccgctcaacgagccggtgcacggcaccaagcgccgcagccagatacagacatacctggacc

SEQ ID NO: 430 - DNA, 200 bp, Digitaria sanguinalis
ggtccaggtatgtctgtatctggctgcggcgcttggtgccgtgcaccggctcgttgagcgggagcagcacgttctcggagttgttggcgagcaccacggaattgagcccgctctccgccgtgcccacgtcctccgccgtgaactcggcgaactcgtggaaccccgtgaatccggcgacgtacgcggccaccggcgccagc

SEQ ID NO: 431 - DNA, 200 bp, Digitaria sanguinalis
agccagatacagacatacctggaccaccacggcggccctggagtgcagcacatcgcgctggctagcgacgacgtgctcaggacactgcgggagatgcaggcgcgctccgccatgggtgggtttgagttcatggaggctccgccacccacttactatgagggtgtgaggcggcgcgccggggacgtgctctcggaggagca

SEQ ID NO: 432 - DNA, 200 bp, Digitaria sanguinalis
tgctcctccgagagcacgtccccggcgcgccgcctcacaccctcatagtaagtgggtggcggagcctccatgaactcaaacccacccatggcggagcgcgcctgcatctcccgcagtgtcctgagcacgtcgtcgctagccagcgcgatgtgctgcactccagggccgccgtggtggtccaggtatgtctgtatctggct

SEQ ID NO: 433 - DNA, 200 bp, Digitaria sanguinalis
ccggggacgtgctctcggaggagcagataaaggagtgccagaaactgggggtgctggtggacagggatgaccagggagtgttgctccaaatcttcaccaagccagtgggggacccaacgtttttcttggagataatccaaaggattgggtgcatggagaaggatgagcagggaaaggactaccagaagggcggctgtggc

SEQ ID NO: 434 - DNA, 200 bp, Digitaria sanguinalis
gccacagccgcccttctggtagtcctttccctgctcatccttctccatgcacccaatcctttggattatctccaagaaaaacgttgggtcccccactggcttggtgaagatttggagcaacactccctggtcatccctgtccaccagcacccccagtttctggcactcctttatctgctcctccgagagcacgtccccgg

SEQ ID NO: 435 - DNA, 200 bp, Digitaria sanguinalis
ttgtgaagttggtgtcatatatttcaagatctgttcgtatagaatccacagatacataatcattagattataaaatctcatgaagattattcatgggtggttttcatgcagcagcagtggcttcagtggtggctcttgcttccaatgtcttctcatattcctcaatggatttaaaaagctctgagaagttccctttgcca

SEQ ID NO: 436 - DNA, 200 bp, Digitaria sanguinalis
tggcaaagggaacttctcagagctttttaaatccattgaggaatatgagaagacattggaagcaagagccaccactgaagccactgctgctgcatgaaaaccacccatgaataatcttcatgagattttataatctaatgattatgtatctgtggattctatacgaacagatcttgaaatatatgacaccaacttcacaa

SEQ ID NO: 437 - DNA, 200 bp, Digitaria sanguinalis
aagctctgagaagttccctttgccaaaccctccacaccctgccttctgctgctcctttccttcattatccttcaccatacaccctactctctgaattatctctatgaatatcgtcggcctgtcaccaacaggcttggtaaaaatctgaagcaaagtcccctgatcatctctatcaaccaatatccccaattcttcacact

SEQ ID NO: 438 - DNA, 200 bp, Digitaria sanguinalis
agtgtgaagaattggggatattggttgatagagatgatcaggggactttgcttcagatttttaccaagcctgttggtgacaggccgacgatattcatagagataattcagagagtagggtgtatggtgaaggataatgaaggaaaggagcagcagaaggcagggtgtggagggtttggcaaagggaacttctcagagctt

SEQ ID NO: 439 - DNA, 200 bp, Digitaria sanguinalis
accaatatccccaattcttcacactccttaatctgttcatcagacaacacatcgcccaccctattcttcaaattcctataataagtaggagggggagacggcataaactcaaagccaccaaccccactccttttcctcatctcccttaacgtcctaaatatatcctcactagccaacgccaaatgctgaacccccgcccc

SEQ ID NO: 440 - DNA, 200 bp, Digitaria sanguinalis
ggggcgggggttcagcatttggcgttggctagtgaggatatatttaggacgttaagggagatgaggaaaaggagtggggttggtggctttgagtttatgccgtctccccctcctacttattataggaatttgaagaatagggtgggcgatgtgttgtctgatgaacagattaaggagtgtgaagaattggggatattggt

SEQ ID NO: 441 - DNA, 200 bp, Digitaria sanguinalis
acgccaaatgctgaacccccgccccttcattatgttccaaatacgtctgtatctgactcttcctcttcgtcccgtaaaccggttcattcatcggtatcaataccatctcactattgcaagccaaaaccaccgagtttagtccactctcactcgttcccacatcctccgccgtaaactcagcaaactcgtgaaatccagta

SEQ ID NO: 442 - DNA, 200 bp, Digitaria sanguinalis
tactggatttcacgagtttgctgagtttacggcggaggatgtgggaacgagtgagagtggactaaactcggtggttttggcttgcaatagtgagatggtattgataccgatgaatgaaccggtttacgggacgaagaggaagagtcagatacagacgtatttggaacataatgaaggggcgggggttcagcatttggcgt

SEQ ID NO: 443 - DNA, 200 bp, Euphorbia heterophylla
gacaattaataaaaaaaacgtaaagactacctttaattggaggagaggaaaacaccacgtttaaaaatcccgttgttatccgattgatgaaaaaaaagattaacacgttacgacttctccattcaataatccattttctttatcttataaataatttgaaatcccatcctcctcgttctccgttcaccagaaaaaacaga

SEQ ID NO: 444 - DNA, 200 bp, Euphorbia heterophylla
tctgttttttctggtgaacggagaacgaggaggatgggatttcaaattatttataagataaagaaaatggattattgaatggagaagtcgtaacgtgttaatcttttttttcatcaatcggataacaacgggatttttaaacgtggtgttttcctctcctccaattaaaggtagtctttacgttttttttattaattgtc

SEQ ID NO: 445 - DNA, 200 bp, Euphorbia heterophylla
ttctccgttcaccagaaaaaacagaaatgggaaaagacacatcagctgccgccgaagcattcaagctcgtcggattttccaatttcgtcagaatcaatccccggtccgaccgtttcccggtcaagcgcttccaccacgtcgagttctggtgctccgacgcaaccaacaccgctcgccgcttctcatggggcctcgggatg

SEQ ID NO: 446 - DNA, 200 bp, Euphorbia heterophylla
catcccgaggccccatgagaagcggcgagcggtgttggttgcgtcggagcaccagaactcgacgtggtggaagcgcttgaccgggaaacggtcggaccggggattgattctgacgaaattggaaaatccgacgagcttgaatgcttcggcggcagctgatgtgtcttttcccatttctgttttttctggtgaacggagaa

SEQ ID NO: 447 - DNA, 200 bp, Euphorbia heterophylla
ccgcttctcatggggcctcgggatgccgttcgtcgccaaatcggatctctccaccggcaacgtcacccacgcctcctacctcctccgctccggcgacctcaatttcctcttcacagctccctactctccctccatagccgccatggagaatctctccgatactgctaccgcatcaatccctactttctcccgcgacgttt

SEQ ID NO: 448 - DNA, 200 bp, Euphorbia heterophylla
aaacgtcgcgggagaaagtagggattgatgcggtagcagtatcggagagattctccatggcggctatggagggagagtagggagctgtgaagaggaaattgaggtcgccggagcggaggaggtaggaggcgtgggtgacgttgccggtggagagatccgatttggcgacgaacggcatcccgaggccccatgagaagcgg

SEQ ID NO: 449 - DNA, 200 bp, Euphorbia heterophylla
atccctactttctcccgcgacgttttcctcgatttctccgccaaacacggcctcgccgtccgagctatagcaatcgaagtggaggacgctgcagttgccttcaatactagtgttgctcaaggcgcggttccggtggccggacctgtagtgctcgataatcgcgcttcggtagcggaggttcacttgtacggcgacgtcgt

SEQ ID NO: 450 - DNA, 200 bp, Euphorbia heterophylla
acgacgtcgccgtacaagtgaacctccgctaccgaagcgcgattatcgagcactacaggtccggccaccggaaccgcgccttgagcaacactagtattgaaggcaactgcagcgtcctccacttcgattgctatagctcggacggcgaggccgtgtttggcggagaaatcgaggaaaacgtcgcgggagaaagtagggat

SEQ ID NO: 451 - DNA, 200 bp, Euphorbia heterophylla
aggttcacttgtacggcgacgtcgttttgcggtacgtcagttacctaaactctgatgactgtttgtttctgccgaaatttgaggcggtagatgaggaggcgtcgtttccgttggattacgggatccggcggctagatcacgcggttggaaatgtaccggatcttgctccggcggtttcgtatgtgaagaagttcaccgga

SEQ ID NO: 452 - DNA, 200 bp, Euphorbia heterophylla
tccggtgaacttcttcacatacgaaaccgccggagcaagatccggtacatttccaaccgcgtgatctagccgccggatcccgtaatccaacggaaacgacgcctcctcatctaccgcctcaaatttcggcagaaacaaacagtcatcagagtttaggtaactgacgtaccgcaaaacgacgtcgccgtacaagtgaacct

SEQ ID NO: 453 - DNA, 200 bp, Euphorbia heterophylla
ttcgtatgtgaagaagttcaccggattccacgagttcgctgagttcacggcggaggatgtagggacgagtgagagcggattgaactcggtggtgttggctaacaacgaagaaacggtactgctgccgatgaatgaaccggtgtttgggacgaagaggaaaagtcagatacagacgtatttggagcacaatgaaggagctg

SEQ ID NO: 454 - DNA, 200 bp, Euphorbia heterophylla
cagctccttcattgtgctccaaatacgtctgtatctgacttttcctcttcgtcccaaacaccggttcattcatcggcagcagtaccgtttcttcgttgttagccaacaccaccgagttcaatccgctctcactcgtccctacatcctccgccgtgaactcagcgaactcgtggaatccggtgaacttcttcacatacgaa

SEQ ID NO: 455 - DNA, 200 bp, Euphorbia heterophylla
tatttggagcacaatgaaggagctggagtacagcatttggcgcttgtgagtgctgatattttcaatactttgagagagatgaggaggaggagtgcgattgggggatttgagtttatgccgtctcctccgccgacatattaccggaatttgaagaagagagttggagatattttgagtgatgaacagattaaggagtgtga

SEQ ID NO: 456 - DNA, 200 bp, Euphorbia heterophylla
tcacactccttaatctgttcatcactcaaaatatctccaactctcttcttcaaattccggtaatatgtcggcggaggagacggcataaactcaaatcccccaatcgcactcctcctcctcatctctctcaaagtattgaaaatatcagcactcacaagcgccaaatgctgtactccagctccttcattgtgctccaaata

SEQ ID NO: 457 - DNA, 200 bp, Euphorbia heterophylla
gtgatgaacagattaaggagtgtgaagaattagggattctggtggacagggatgatcaagggaccttgcttcagattttcactaaacctgtgggagataggtatttctatcttcttcttcattgttttcacttacaatacctttttccattgaaaattctttgtttcttgtgtttctcttatatgtgttggctgctatac

SEQ ID NO: 458 - DNA, 200 bp, Euphorbia heterophylla
gtatagcagccaacacatataagagaaacacaagaaacaaagaattttcaatggaaaaaggtattgtaagtgaaaacaatgaagaagaagatagaaatacctatctcccacaggtttagtgaaaatctgaagcaaggtcccttgatcatccctgtccaccagaatccctaattcttcacactccttaatctgttcatcac

SEQ ID NO: 459 - DNA, 200 bp, Euphorbia heterophylla
ctcttatatgtgttggctgctatactatggttgatagagaattagaatttagcgtgttcaaaactcaattctttatctttatctttagccttaggtcattttgggattgctttcaaactgatttgattatgttactaattaattagtacgaaggactaacatgtgttgttctaagctaaaatatattctataaagctaaa

SEQ ID NO: 460 - DNA, 200 bp, Euphorbia heterophylla
tttagctttatagaatatattttagcttagaacaacacatgttagtccttcgtactaattaattagtaacataatcaaatcagtttgaaagcaatcccaaaatgacctaaggctaaagataaagataaagaattgagttttgaacacgctaaattctaattctctatcaaccatagtatagcagccaacacatataagag

SEQ ID NO: 461 - DNA, 200 bp, Euphorbia heterophylla
ctaaaatatattctataaagctaaaatatcttaataagcaataccaaactagcccttaaatgcatagttagaccctaaatttgacaacttttataacttggcgcctctaatttacaaatgtcttcgccaacctcttgaactggcaaaagttttcactgcaacccctaggtgccacgtgtcaccttgtgattgatttgact

SEQ ID NO: 462 - DNA, 200 bp, Euphorbia heterophylla
agtcaaatcaatcacaaggtgacacgtggcacctaggggttgcagtgaaaacttttgccagttcaagaggttggcgaagacatttgtaaattagaggcgccaagttataaaagttgtcaaatttagggtctaactatgcatttaagggctagtttggtattgcttattaagatattttagctttatagaatatattttag

SEQ ID NO: 463 - DNA, 200 bp, Euphorbia heterophylla
gtgtcaccttgtgattgatttgacttttcaaagatgtcaaaattttttattttcctccctctaatcaaaaggtgacaagtgatattgagtaggggtctgtagtgaatactttccccaagttgagacatccagaaatcggaggcgtcatgtgataaaagttgcttagttaagggagcttaatatgtattaagccttttcgc

SEQ ID NO: 464 - DNA, 200 bp, Euphorbia heterophylla
gcgaaaaggcttaatacatattaagctcccttaactaagcaacttttatcacatgacgcctccgatttctggatgtctcaacttggggaaagtattcactacagacccctactcaatatcacttgtcaccttttgattagagggaggaaaataaaaaattttgacatctttgaaaagtcaaatcaatcacaaggtgacac

SEQ ID NO: 465 - DNA, 200 bp, Euphorbia heterophylla
cttaatatgtattaagccttttcgctaccttgccatttaaatccttagtccaagcaaaatatacaaaaattagttagaactttatgattcagaacctacgtaactttaatttgaccagtagttctatcttatgcatgccagccatgttgactggtgaacttatattcaattttgctattcaagtaaatccttatccatca

SEQ ID NO: 466 - DNA, 200 bp, Euphorbia heterophylla
tgatggataaggatttacttgaatagcaaaattgaatataagttcaccagtcaacatggctggcatgcataagatagaactactggtcaaattaaagttacgtaggttctgaatcataaagttctaactaatttttgtatattttgcttggactaaggatttaaatggcaaggtagcgaaaaggcttaatacatattaag

SEQ ID NO: 467 - DNA, 200 bp, Euphorbia heterophylla
tattcaagtaaatccttatccatcaatccgaatcaaaagattttaatgttaggttaaacttgtacgattcaaaacttaggttgtactttttgactagtagttctattatgtatgtgtatatataatggggttttttttctgtaggccaacgattttcgtagagataatacagagagtagggtgtatgctaaaggacgaag

SEQ ID NO: 468 - DNA, 200 bp, Euphorbia heterophylla
cttcgtcctttagcatacaccctactctctgtattatctctacgaaaatcgttggcctacagaaaaaaaaccccattatatatacacatacataatagaactactagtcaaaaagtacaacctaagttttgaatcgtacaagtttaacctaacattaaaatcttttgattcggattgatggataaggatttacttgaata

SEQ ID NO: 469 - DNA, 200 bp, Euphorbia heterophylla
gtagggtgtatgctaaaggacgaagcagggagagaataccagaagggtggatgtggcggttttggtaagggaaacttttcggagttattcaaatccattgaagaatacgagaaaacactggaagccaaacgaactgcacaagcatagaggttagtagggaatttgatgatacccataaataatggttgttacttgttgcc

SEQ ID NO: 470 - DNA, 200 bp, Euphorbia heterophylla
ggcaacaagtaacaaccattatttatgggtatcatcaaattccctactaacctctatgcttgtgcagttcgtttggcttccagtgttttctcgtattcttcaatggatttgaataactccgaaaagtttcccttaccaaaaccgccacatccacccttctggtattctctccctgcttcgtcctttagcatacaccctac

SEQ ID NO: 471 - DNA, 200 bp, Euphorbia heterophylla
tgactgaaattaaattttaattatttgcaaagcttacggtgaaattaaactctaaccattaaaattttaatgagtcaaaattgaccgaaatgttcatgtcataaatttagataatcctttcagatatgtacgtgaaaccatgtacctaaattgtagtcagattgctaaacccccttcgaaaatgaatgaaatgatatgat

SEQ ID NO: 472 - DNA, 200 bp, Euphorbia heterophylla
atcatatcatttcattcattttcgaagggggtttagcaatctgactacaatttaggtacatggtttcacgtacatatctgaaaggattatctaaatttatgacatgaacatttcggtcaattttgactcattaaaattttaatggttagagtttaatttcaccgtaagctttgcaaataattaaaatttaatttcagtca

SEQ ID NO: 473 - DNA, 200 bp, Euphorbia heterophylla
tcgaaaatgaatgaaatgatatgatatgctatgctatgtattttaaggattattttatagtaactttgtttataatttactttatatgatcatcatccaattaactttcacctcactaattcaatgattgaaatggactaagtaattttacttaataaaaaaataaaatcactataacctacacatatttttaaaaataa

SEQ ID NO: 474 - DNA, 200 bp, Euphorbia heterophylla
ttatttttaaaaatatgtgtaggttatagtgattttatttttttattaagtaaaattacttagtccatttcaatcattgaattagtgaggtgaaagttaattggatgatgatcatataaagtaaattataaacaaagttactataaaataatccttaaaatacatagcatagcatatcatatcatttcattcattttcga

SEQ ID NO: 475 - DNA, 200 bp, Euphorbia heterophylla
aacctacacatatttttaaaaataacaccataatttattaaaggacatttaattgaagtagaaaattatataattactcgctaataaattttcaattggaggagaggaaaacaccacgtttaaaaatcccgttgttatccgattgacgaaaaaaaagattaacacgtcacgacttctccattcaataatccattttcttt

SEQ ID NO: 476 - DNA, 200 bp, Euphorbia heterophylla
aaagaaaatggattattgaatggagaagtcgtgacgtgttaatcttttttttcgtcaatcggataacaacgggatttttaaacgtggtgttttcctctcctccaattgaaaatttattagcgagtaattatataattttctacttcaattaaatgtcctttaataaattatggtgttatttttaaaaatatgtgtaggtt

SEQ ID NO: 477 - DNA, 200 bp, Euphorbia heterophylla
ctccattcaataatccattttctttatcttataaataatttgaaatcccatcctcctcgttctccgttcaccagaaaaaacagaaatgggaaaagacacgtcagccgctgccgaagcattcaagctcgtcggattttccaatttcgtcagaatcaatccccggtccgaccgtttcccggtcaagcgcttccaccacgtcg

SEQ ID NO: 478 - DNA, 200 bp, Euphorbia heterophylla
cgacgtggtggaagcgcttgaccgggaaacggtcggaccggggattgattctgacgaaattggaaaatccgacgagcttgaatgcttcggcagcggctgacgtgtcttttcccatttctgttttttctggtgaacggagaacgaggaggatgggatttcaaattatttataagataaagaaaatggattattgaatggag

SEQ ID NO: 479 - DNA, 200 bp, Euphorbia heterophylla
ccggtcaagcgcttccaccacgtcgagttcaggtgctccgacgcaaccaacaccgctcgccgcttctcatggggcctcgggatgccgttcgtcgccaaatcggatctctccaccggcaacgtcacccacgcctcctacctcctccgctccggcgacctcaatttcctcttcacagctccctactctccctccatagccgc

SEQ ID NO: 480 - DNA, 200 bp, Euphorbia heterophylla
gcggctatggagggagagtagggagctgtgaagaggaaattgaggtcgccggagcggaggaggtaggaggcgtgggtgacgttgccggtggagagatccgatttggcgacgaacggcatcccgaggccccatgagaagcggcgagcggtgttggttgcgtcggagcacctgaactcgacgtggtggaagcgcttgaccgg

SEQ ID NO: 481 - DNA, 200 bp, Euphorbia heterophylla
ctccctactctccctccatagccgccatggagaatctctccgatactgccaccgcatcaatccctactttctcccgcgacgttttcctcgatttctccgccaaacacggcctcgccgtccgagctatagccattgaagtggaagatgctgcgattgctttcactaccagcgttgctcaaggcgcgattccggtggccgga

SEQ ID NO: 482 - DNA, 200 bp, Euphorbia heterophylla
tccggccaccggaatcgcgccttgagcaacgctggtagtgaaagcaatcgcagcatcttccacttcaatggctatagctcggacggcgaggccgtgtttggcggagaaatcgaggaaaacgtcgcgggagaaagtagggattgatgcggtggcagtatcggagagattctccatggcggctatggagggagagtagggag

SEQ ID NO: 483 - DNA, 200 bp, Euphorbia heterophylla
tcaaggcgcgattccggtggccggacctattgtgctcgataatcgtgcttcagttgcggaggtccacttgtacggcgacgtcgttttgcggtacgtcagttatctaaactccgatagttgcttgtttctgccgaaattcgagccggtagatgaggcgtcgtctttcccgttggattacgggattcgacggctagatcacg

SEQ ID NO: 484 - DNA, 200 bp, Euphorbia heterophylla
cgtgatctagccgtcgaatcccgtaatccaacgggaaagacgacgcctcatctaccggctcgaatttcggcagaaacaagcaactatcggagtttagataactgacgtaccgcaaaacgacgtcgccgtacaagtggacctccgcaactgaagcacgattatcgagcacaataggtccggccaccggaatcgcgccttga

SEQ ID NO: 485 - DNA, 200 bp, Euphorbia heterophylla
tacgggattcgacggctagatcacgcggttggaaatgtgccggaattatctccggcggtttcgtatgtgaaacagttcaccggattccatgagttcgccgagttcacggcggaggatgtggggacgagtgagagcggattgaactcggtggtgttggctaataacgaagaaacggttttactaccgttgaatgaaccggt

SEQ ID NO: 486 - DNA, 200 bp, Euphorbia heterophylla
accggttcattcaacggtagtaaaaccgtttcttcgttattagccaacaccaccgagttcaatccgctctcactcgtccccacatcctccgccgtgaactcggcgaactcatggaatccggtgaactgtttcacatacgaaaccgccggagataattccggcacatttccaaccgcgtgatctagccgtcgaatcccgta

SEQ ID NO: 487 - DNA, 200 bp, Euphorbia heterophylla
ttttactaccgttgaatgaaccggtgtatggcacaaagaggaaaagtcagatacaaacgtatttggagcacaacgaaggggctggagtacagcatttagcacttgtgagtgcggatatatttaacactttgagagagatgaggaagaggagtggcgttgggggatttgagtttatgccgtctcctccgcccacatattac

SEQ ID NO: 488 - DNA, 200 bp, Euphorbia heterophylla
gtaatatgtgggcggaggagacggcataaactcaaatcccccaacgccactcctcttcctcatctctctcaaagtgttaaatatatccgcactcacaagtgctaaatgctgtactccagccccttcgttgtgctccaaatacgtttgtatctgacttttcctctttgtgccatacaccggttcattcaacggtagtaaaa

SEQ ID NO: 489 - DNA, 200 bp, Euphorbia heterophylla
gccgtctcctccgcccacatattaccggaatttgaagaagagagtcggggatattttgagcgatgaacagattaaggaatgtgaagaattagggattttggtggatagggatgatcaagggaccttgcttcagattttcactaaacctgtgggagataggtatgtcaatcaatcttgtgaaatgattagttgttctataa

SEQ ID NO: 490 - DNA, 200 bp, Euphorbia heterophylla
ttatagaacaactaatcatttcacaagattgattgacatacctatctcccacaggtttagtgaaaatctgaagcaaggtcccttgatcatccctatccaccaaaatccctaattcttcacattccttaatctgttcatcgctcaaaatatccccgactctcttcttcaaattccggtaatatgtgggcggaggagacggc

SEQ ID NO: 491 - DNA, 200 bp, Euphorbia heterophylla
tgtgaaatgattagttgttctataattacttatgtgatttacattcttagagtctgtaaattcttgtgatcataaatttgttggcttggataccatattaatattatccttaatttagaatgtcaaagttatgttctgtctttacaatgccgttgaaatcgttatttatcaagtccaagcaaacggtcctaaaatgtcct

SEQ ID NO: 492 - DNA, 200 bp, Euphorbia heterophylla
aggacattttaggaccgtttgcttggacttgataaataacgatttcaacggcattgtaaagacagaacataactttgacattctaaattaaggataatattaatatggtatccaagccaacaaatttatgatcacaagaatttacagactctaagaatgtaaatcacataagtaattatagaacaactaatcatttcaca

SEQ ID NO: 493 - DNA, 200 bp, Euphorbia heterophylla
caagcaaacggtcctaaaatgtcctttaacttactggttcggaatctcatctgttcaatttgattagtagttccatcttttagtatatacgccaggcgccagacatgttgatttgtgagtttttgttatttggaatctcaaagttgagttttttctatctataaaaagtagctcaaatccttagttatcaagtcaaaacc

SEQ ID NO: 494 - DNA, 200 bp, Euphorbia heterophylla
ggttttgacttgataactaaggatttgagctactttttatagatagaaaaaactcaactttgagattccaaataacaaaaactcacaaatcaacatgtctggcgcctggcgtatatactaaaagatggaactactaatcaaattgaacagatgagattccgaaccagtaagttaaaggacattttaggaccgtttgcttg

SEQ ID NO: 495 - DNA, 200 bp, Euphorbia heterophylla
aatccttagttatcaagtcaaaacccaaagatcctattattaacttggatctgaaattatatcgctttctggtttatttctaaatttggtggtcacttgatttctcatcttgacaagtgaattcatctggacatagttggtcttttatgggatgaatggatgtgctgtttttgcaggccaaccattttcgttgaaataat

SEQ ID NO: 496 - DNA, 200 bp, Euphorbia heterophylla
attatttcaacgaaaatggttggcctgcaaaaacagcacatccattcatcccataaaagaccaactatgtccagatgaattcacttgtcaagatgagaaatcaagtgaccaccaaatttagaaataaaccagaaagcgatataatttcagatccaagttaataataggatctttgggttttgacttgataactaaggatt

SEQ ID NO: 497 - DNA, 200 bp, Euphorbia heterophylla
ggccaaccattttcgttgaaataattcagcgagtcgggtgtatggtcaaggatgaagcagggagagaataccagaagggtgggtgtggcggtttcggtaagggaaacttctcggagttattcaaatcaatcgaggaatatgagaaaacattggaggccaaacgaactgcagaagcagctcgagcataaaataaaggttag

SEQ ID NO: 498 - DNA, 200 bp, Euphorbia heterophylla
ctaacctttattttatgctcgagctgcttctgcagttcgtttggcctccaatgttttctcatattcctcgattgatttgaataactccgagaagtttcccttaccgaaaccgccacacccacccttctggtattctctccctgcttcatccttgaccatacacccgactcgctgaattatttcaacgaaaatggttggcc

SEQ ID NO: 499 - DNA, 200 bp, Euphorbia heterophylla
agctcgagcataaaataaaggttagtatggaagatgatatgcatacataaataaaggttggctgttgtttattacgttgtaatattctactccaatgatgctgttattgtctgaaaataccagtgcctgcgaattggaatgttctctaattgttaggaacaactggaatcattgtatatcgtaaactatggttcattaaa

SEQ ID NO: 500 - DNA, 200 bp, Euphorbia heterophylla
tttaatgaaccatagtttacgatatacaatgattccagttgttcctaacaattagagaacattccaattcgcaggcactggtattttcagacaataacagcatcattggagtagaatattacaacgtaataaacaacagccaacctttatttatgtatgcatatcatcttccatactaacctttattttatgctcgagct

SEQ ID NO: 501 - DNA, 200 bp, Euphorbia heterophylla
atatcgtaaactatggttcattaaaagttaaaaacatatatatgatatgattaatatagaattactccctttatttggaatctaaagattcatttctcagtttctttctgggcgattttgagcatttctgtacttaaattctgcgcgaatacacccaccaagccctagctagcttggtggttaccttttaagggtgttgg

SEQ ID NO: 502 - DNA, 200 bp, Euphorbia heterophylla
ccaacacccttaaaaggtaaccaccaagctagctagggcttggtgggtgtattcgcgcagaatttaagtacagaaatgctcaaaatcgcccagaaagaaactgagaaatgaatctttagattccaaataaagggagtaattctatattaatcatatcatatatatgtttttaacttttaatgaaccatagtttacgatat

SEQ ID NO: 503 - DNA, 200 bp, Euphorbia heterophylla
ggtggttaccttttaagggtgttggaaacctaaattagaaaagatttcagtttaatttatgtcattttatatataaaagtttgaaacttttttattcaaaataagttgattgataaattgaaatcaattagctttgtagcagatgttcacccaaatctaatataaatatattataaatctaatatatttctgttaacaaa

SEQ ID NO: 504 - DNA, 200 bp, Euphorbia heterophylla
tttgttaacagaaatatattagatttataatatatttatattagatttgggtgaacatctgctacaaagctaattgatttcaatttatcaatcaacttattttgaataaaaaagtttcaaacttttatatataaaatgacataaattaaactgaaatcttttctaatttaggtttccaacacccttaaaaggtaaccacc

SEQ ID NO: 505 - DNA, 200 bp, Euphorbia heterophylla
aatctaatatatttctgttaacaaacttataatatattaataaatacaattgcaaagacaaagagacttatattagaattataacataattttttataaatatattagatacgtatatcttataattgtaacatataaaagattttatagtaaatatgtgttataagtctaaaataagcctttttaagtttaatacaagt

SEQ ID NO: 506 - DNA, 200 bp, Euphorbia heterophylla
acttgtattaaacttaaaaaggcttattttagacttataacacatatttactataaaatcttttatatgttacaattataagatatacgtatctaatatatttataaaaaattatgttataattctaatataagtctctttgtctttgcaattgtatttattaatatattataagtttgttaacagaaatatattagatt

SEQ ID NO: 507 - DNA, 200 bp, Euphorbia heterophylla
aagcctttttaagtttaatacaagtctctttgtgtaacaccccgggttcgaccgattgacccccacaaaccaacacaagtctttctagtaagctttgccttgagattgttccaagtcaaggacgctcaactttggagttctcccatataagctcctgaaaagaaggtgtaacttgttggtataggcagcagtaccaatca

SEQ ID NO: 508 - DNA, 200 bp, Euphorbia heterophylla
tgattggtactgctgcctataccaacaagttacaccttcttttcaggagcttatatgggagaactccaaagttgagcgtccttgacttggaacaatctcaaggcaaagcttactagaaagacttgtgttggtttgtgggggtcaatcggtcgaacccggggtgttacacaaagagacttgtattaaacttaaaaaggctt

SEQ ID NO: 509 - DNA, 200 bp, Euphorbia heterophylla
ttggtataggcagcagtaccaatcaaacctttttaagtccctttccactttaatgtccctttctattgatataattttattgactttatgtaatcaaatgtttattcatcccgtgtttttttattcattatgttttaattttttaattagtcaaaatataatatctaaaaaatctatcaaatttgggctcggctcggctt

SEQ ID NO: 510 - DNA, 200 bp, Euphorbia heterophylla
aagccgagccgagcccaaatttgatagattttttagatattatattttgactaattaaaaaattaaaacataatgaataaaaaaacacgggatgaataaacatttgattacataaagtcaataaaattatatcaatagaaagggacattaaagtggaaagggacttaaaaaggtttgattggtactgctgcctataccaa

SEQ ID NO: 511 - DNA, 200 bp, Euphorbia heterophylla
atcaaatttgggctcggctcggcttgactcattaacatccgactctctacctcttacttttattaaatacatttgtgtttttcagttgtacgttccttctcatatttacatttcccttttttcttttctttttaatttattttttatttatttcgcaaatcgttgattttctcggtatgctgctttataattttttgttc

SEQ ID NO: 512 - DNA, 200 bp, Euphorbia heterophylla
gaacaaaaaattataaagcagcataccgagaaaatcaacgatttgcgaaataaataaaaaataaattaaaaagaaaagaaaaaagggaaatgtaaatatgagaaggaacgtacaactgaaaaacacaaatgtatttaataaaagtaagaggtagagagtcggatgttaatgagtcaagccgagccgagcccaaatttgat

SEQ ID NO: 513 - DNA, 200 bp, Euphorbia heterophylla
tatgctgctttataattttttgttcgtctcgtttttatcttcgaaagtatattcatgttgatatactttttcaagttcttattttcaaagatatgtaacaaatgacataaacattatccagcaaaaatcagtaaattactagcatttcaccaacaaaatactagcattttacccgtgacgaaaagtttgaaaaaaatata

SEQ ID NO: 514 - DNA, 200 bp, Euphorbia heterophylla
tatatttttttcaaacttttcgtcacgggtaaaatgctagtattttgttggtgaaatgctagtaatttactgatttttgctggataatgtttatgtcatttgttacatatctttgaaaataagaacttgaaaaagtatatcaacatgaatatactttcgaagataaaaacgagacgaacaaaaaattataaagcagcata

SEQ ID NO: 515 - DNA, 200 bp, Euphorbia heterophylla
tgacgaaaagtttgaaaaaaatatataaaaaaaatcaaacaaaaatcatttttataccaacattataccatgtatgaaaaaataaaactgatcataaacataaagaaatcaaacaaaattcaccttatcttaatattttaccaagagtgaaggaccaaacataaacatgttttacgaaaaaaatcaaacaaaaatcattt

SEQ ID NO: 516 - DNA, 200 bp, Euphorbia heterophylla
aaatgatttttgtttgatttttttcgtaaaacatgtttatgtttggtccttcactcttggtaaaatattaagataaggtgaattttgtttgatttctttatgtttatgatcagttttattttttcatacatggtataatgttggtataaaaatgatttttgtttgattttttttatatatttttttcaaacttttcgtca

SEQ ID NO: 517 - DNA, 200 bp, Euphorbia heterophylla
gaaaaaaatcaaacaaaaatcatttcacactagcattttattaacaaaatctcgatatattatcagtaaaataccaacattttatcaacaaaataccatcatttgagaggaggttcgagtgcgccttcgccgttctgcggcggcttctttgacggtggagaaagcatagctgcgtgatggaggcagaagaggggttgatg

SEQ ID NO: 518 - DNA, 200 bp, Euphorbia heterophylla
catcaacccctcttctgcctccatcacgcagctatgctttctccaccgtcaaagaagccgccgcagaacggcgaaggcgcactcgaacctcctctcaaatgatggtattttgttgataaaatgttggtattttactgataatatatcgagattttgttaataaaatgctagtgtgaaatgatttttgtttgatttttttc

SEQ ID NO: 519 - DNA, 200 bp, Euphorbia heterophylla
gatggaggcagaagaggggttgatgaaattgggttggattgaagtggaattgagtcaatgaggctaggtgatagggtgagatgatgcatgactcgaattggtggttgatcgcagaggtggaggtggcgattggccggcggagaagacggcaaatagatgtttttgggcggagtacaaagcgccttaaatcctagcaaagg

SEQ ID NO: 520 - DNA, 200 bp, Euphorbia heterophylla
cctttgctaggatttaaggcgctttgtactccgcccaaaaacatctatttgccgtcttctccgccggccaatcgccacctccacctctgcgatcaaccaccaattcgagtcatgcatcatctcaccctatcacctagcctcattgactcaattccacttcaatccaacccaatttcatcaacccctcttctgcctccatc

SEQ ID NO: 521 - DNA, 200 bp, Euphorbia heterophylla
aaagcgccttaaatcctagcaaaggctgatgaaaataaaaaagggtaatgctgcacttttatctactttgtgcaatatctatatacagtatttattatcatcttattccttttatttatttctttcatcttttatgggagttgaaattctttctagatattcttttctttttgctaaatttttctgctcattgtcataca

SEQ ID NO: 522 - DNA, 200 bp, Euphorbia heterophylla
tgtatgacaatgagcagaaaaatttagcaaaaagaaaagaatatctagaaagaatttcaactcccataaaagatgaaagaaataaataaaaggaataagatgataataaatactgtatatagatattgcacaaagtagataaaagtgcagcattacccttttttattttcatcagcctttgctaggatttaaggcgcttt

SEQ ID NO: 523 - DNA, 200 bp, Euphorbia heterophylla
aaatttttctgctcattgtcatacacttgcttggtaaactgaatctcaaaccaaataatgaaatctgaagtatgtatccacagaatcgcaatgctcatcgacggaagtagaaggggaaaactgctgttggagttgaggtatataatctgcagttgaaatcgacgttgtgtatgtaattttgaattgaacaagagcgaaac

SEQ ID NO: 524 - DNA, 200 bp, Euphorbia heterophylla
gtttcgctcttgttcaattcaaaattacatacacaacgtcgatttcaactgcagattatatacctcaactccaacagcagttttccccttctacttccgtcgatgagcattgcgattctgtggatacatacttcagatttcattatttggtttgagattcagtttaccaagcaagtgtatgacaatgagcagaaaaattt

SEQ ID NO: 525 - DNA, 200 bp, Euphorbia heterophylla
attttgaattgaacaagagcgaaacctagttttctatgatctgtacctggcgtataagaattccatcaatatcggcagggcgatgcgttaatattgcattaacaccggtggtgagaagggagctaattgcttgaaatccgctggcatgccgcaatgttgatatgtcacataaatgcgactaagtgtgacttatcttgaag

SEQ ID NO: 526 - DNA, 200 bp, Euphorbia heterophylla
cttcaagataagtcacacttagtcgcatttatgtgacatatcaacattgcggcatgccagcggatttcaagcaattagctcccttctcaccaccggtgttaatgcaatattaacgcatcgccctgccgatattgatggaattcttatacgccaggtacagatcatagaaaactaggtttcgctcttgttcaattcaaaat

SEQ ID NO: 527 - DNA, 200 bp, Euphorbia heterophylla
cgactaagtgtgacttatcttgaagaggagtgaaattggggaaacgactaagagtgacttatcaagaagtgtgattaccatgcaactaagtgcgacacggtgaaacgactcgactcctgcagccaccaccaaatttcacaatttcatataggtaaaagggagacgatcagaaagaggggaaacaaaacacaaagagaggg

SEQ ID NO: 528 - DNA, 200 bp, Euphorbia heterophylla
ccctctctttgtgttttgtttcccctctttctgatcgtctcccttttacctatatgaaattgtgaaatttggtggtggctgcaggagtcgagtcgtttcaccgtgtcgcacttagttgcatggtaatcacacttcttgataagtcactcttagtcgtttccccaatttcactcctcttcaagataagtcacacttagtcg

SEQ ID NO: 529 - DNA, 200 bp, Euphorbia heterophylla
ggggaaacaaaacacaaagagagggaaaagaaatgttggagggactgaaattatagaaggggtaaaattagaaacaaaaaaaattataaggtgttgtaagtagaaattaaattacgagcaaaagttggttgaaaataaaaatgaagatattgtgataaattatttctttttttttatatttgaccaacttacattgtaaa

SEQ ID NO: 530 - DNA, 200 bp, Euphorbia heterophylla
tttacaatgtaagttggtcaaatataaaaaaaaagaaataatttatcacaatatcttcatttttattttcaaccaacttttgctcgtaatttaatttctacttacaacaccttataattttttttgtttctaattttaccccttctataatttcagtccctccaacatttcttttccctctctttgtgttttgtttcccc

SEQ ID NO: 531 - DNA, 200 bp, Euphorbia heterophylla
atatttgaccaacttacattgtaaagcctgtaatttttattaatttcgcattatagccataaaaagttattccaatctatgaagttgtgactttgcggaacatattttccgaaaaacttatttttcaagttttttaaatagtaagaaaaaggaaattttaaattataataacataataggtagtaaatataaatagtata

SEQ ID NO: 532 - DNA, 200 bp, Euphorbia heterophylla
tatactatttatatttactacctattatgttattataatttaaaatttcctttttcttactatttaaaaaacttgaaaaataagtttttcggaaaatatgttccgcaaagtcacaacttcatagattggaataactttttatggctataatgcgaaattaataaaaattacaggctttacaatgtaagttggtcaaatat

SEQ ID NO: 533 - DNA, 200 bp, Euphorbia heterophylla
ataggtagtaaatataaatagtatatatttataattcaaataattatcaggaaatcttatttattttatgaaatttctaggtaaaattaatttagtcatgactaagtgttgtttctcacaaaattgtaatcataatcaaactaaattttgatatcgagaaacacaaaatgagcatttgttgaaagagacttataggtcga

SEQ ID NO: 534 - DNA, 200 bp, Euphorbia heterophylla
tcgacctataagtctctttcaacaaatgctcattttgtgtttctcgatatcaaaatttagtttgattatgattacaattttgtgagaaacaacacttagtcatgactaaattaattttacctagaaatttcataaaataaataagatttcctgataattatttgaattataaatatatactatttatatttactacctat

SEQ ID NO: 535 - DNA, 200 bp, Euphorbia heterophylla
ttgttgaaagagacttataggtcgagtcttaacaaacttgataagattataaaattgtctagcataaactcctcatgcagcgaaacaataatgtatgattacaatcataagccaatgttcatgttggataaaccacacgataactaattctagaatctctcaagagtctaaataatttctatgcacacttttaaatttat

SEQ ID NO: 536 - DNA, 200 bp, Euphorbia heterophylla
ataaatttaaaagtgtgcatagaaattatttagactcttgagagattctagaattagttatcgtgtggtttatccaacatgaacattggcttatgattgtaatcatacattattgtttcgctgcatgaggagtttatgctagacaattttataatcttatcaagtttgttaagactcgacctataagtctctttcaacaa

SEQ ID NO: 537 - DNA, 200 bp, Euphorbia heterophylla
tttctatgcacacttttaaatttatttaagcttcatagtgaaatattagcttgtgctacactaatttacattgtacgttattttcaatggaatgacaactttattttgtgcccatagtgcataacagtaaaatttcagttcaatgatttcatatattattttttatattacgaagttttaggtttttggattttagatta

SEQ ID NO: 538 - DNA, 200 bp, Euphorbia heterophylla
taatctaaaatccaaaaacctaaaacttcgtaatataaaaaataatatatgaaatcattgaactgaaattttactgttatgcactatgggcacaaaataaagttgtcattccattgaaaataacgtacaatgtaaattagtgtagcacaagctaatatttcactatgaagcttaaataaatttaaaagtgtgcatagaaa

SEQ ID NO: 539 - DNA, 200 bp, Euphorbia heterophylla
ttttaggtttttggattttagattacaatcgagaccatagcaagagttgaaacatgcaaatgagatttaagcttaaaaaaatgttataaaattattaacatttagcacttatagtagctacaaaaatttgaaggattgaatatttctaaatattacaaagtataaacttaaatatcaacaaatatgattcatggagtttg

SEQ ID NO: 540 - DNA, 200 bp, Euphorbia heterophylla
caaactccatgaatcatatttgttgatatttaagtttatactttgtaatatttagaaatattcaatccttcaaatttttgtagctactataagtgctaaatgttaataattttataacatttttttaagcttaaatctcatttgcatgtttcaactcttgctatggtctcgattgtaatctaaaatccaaaaacctaaaa

SEQ ID NO: 541 - DNA, 200 bp, Euphorbia heterophylla
caacaaatatgattcatggagtttgagttaagtataatttaacaatatatcaatgatcgagttttttgcaaaaatcataaatattagctcaatttgaagaacttaacaaagcactcaaaactttaattgaaaacaaaaatttaaggttaagggcttatatatataaaaaaaaattcaaagtatgaaagcaatttcggact

SEQ ID NO: 542 - DNA, 200 bp, Euphorbia heterophylla
agtccgaaattgctttcatactttgaatttttttttatatatataagcccttaaccttaaatttttgttttcaattaaagttttgagtgctttgttaagttcttcaaattgagctaatatttatgatttttgcaaaaaactcgatcattgatatattgttaaattatacttaactcaaactccatgaatcatatttgttg

SEQ ID NO: 543 - DNA, 200 bp, Euphorbia heterophylla
caaagtatgaaagcaatttcggacttcaatttttataatttttgattcataacgaactattaaattattgataaatatacttctgcatagagtccaaaaaattataaggccggcctaataaagcctatgatagaatcctatagaaacattgttgacccaagcacaggccgagtgaactcgtggctgagtttctatgctaa

SEQ ID NO: 544 - DNA, 200 bp, Euphorbia heterophylla
ttagcatagaaactcagccacgagttcactcggcctgtgcttgggtcaacaatgtttctataggattctatcataggctttattaggccggccttataattttttggactctatgcagaagtatatttatcaataatttaatagttcgttatgaatcaaaaattataaaaattgaagtccgaaattgctttcatactttg

SEQ ID NO: 545 - DNA, 200 bp, Euphorbia heterophylla
ttgtttctgccgaaattcgagccggtagatgaggcgtcgtcgtttccgttggattacgggattcgacggctagatcacgcggttggaaatgtaccggatcttgctccggcggtttcgtatgtgaagaagttcaccggattccacgagttcgctgagttcacggcggaggatgtagggacgagtgagagcggattgaactc

SEQ ID NO: 546 - DNA, 200 bp, Euphorbia heterophylla
gagttcaatccgctctcactcgtccctacatcctccgccgtgaactcagcgaactcgtggaatccggtgaacttcttcacatacgaaaccgccggagcaagatccggtacatttccaaccgcgtgatctagccgtcgaatcccgtaatccaacggaaacgacgacgcctcatctaccggctcgaatttcggcagaaacaa

SEQ ID NO: 547 - DNA, 200 bp, Euphorbia heterophylla
ggacgagtgagagcggattgaactcggtggtgttggctaacaacgaagaaacggtactgctgccgatgaatgaaccggtgtttgggacgaagaggaaaagtcagatacagacgtatttggagcacaatgaaggagctggagtacagcatttggcgcttgtgagtgctgatattttcaatactttgagagagatgaggagg

SEQ ID NO: 548 - DNA, 200 bp, Euphorbia heterophylla
cctcctcatctctctcaaagtattgaaaatatcagcactcacaagcgccaaatgctgtactccagctccttcattgtgctccaaatacgtctgtatctgacttttcctcttcgtcccaaacaccggttcattcatcggcagcagtaccgtttcttcgttgttagccaacaccaccgagttcaatccgctctcactcgtcc

SEQ ID NO: 549 - DNA, 200 bp, Kochia scoparia
accaccgcctcaaccgagttcaagctggtgggttactccaacttcgtccgagttaaccccatgtccgacctcttctccgtcaaaaaattccaccacatcgagttctggtgcggcgacgcgaccaacgtcagccgtcgcttctcatggggtctaggcatgccggcggtcgctaaatccgacctctccaccggaaactccgt

SEQ ID NO: 550 - DNA, 200 bp, Kochia scoparia
acggagtttccggtggagaggtcggatttagcgaccgccggcatgcctagaccccatgagaagcgacggctgacgttggtcgcgtcgccgcaccagaactcgatgtggtggaattttttgacggagaagaggtcggacatggggttaactcggacgaagttggagtaacccaccagcttgaactcggttgaggcggtggt

SEQ ID NO: 551 - DNA, 200 bp, Kochia scoparia
ccgacctctccaccggaaactccgtacacgcttcgtacctccttcgctcaggcgacctctccttcctcttcacctccccgtactccccttctctctcctccccctcttccgctgcaatacccacgtttgatttctctctcttctcctcttttatcacctcccacggcatcggggttcgcgccgttgcactcgaggtcgac

SEQ ID NO: 552 - DNA, 200 bp, Kochia scoparia
gtcgacctcgagtgcaacggcgcgaaccccgatgccgtgggaggtgataaaagaggagaagagagagaaatcaaacgtgggtattgcagcggaagagggggaggagagagaaggggagtacggggaggtgaagaggaaggagaggtcgcctgagcgaaggaggtacgaagcgtgtacggagtttccggtggagaggtcgg

SEQ ID NO: 553 - DNA, 200 bp, Kochia scoparia
tcgcgccgttgcactcgaggtcgacgatgccgaggttgctttcaatacgagcgtctcccacggcgcaatccctggttctcccccgattaagctcggaaacggcgtcgttttgtccgaggtcagcctctacggcgacgtcgttcttcgctacgtgagctacggaggtgagacagagaaagcggatacgaattcgaattcat

SEQ ID NO: 554 - DNA, 200 bp, Kochia scoparia
atgaattcgaattcgtatccgctttctctgtctcacctccgtagctcacgtagcgaagaacgacgtcgccgtagaggctgacctcggacaaaacgacgccgtttccgagcttaatcgggggagaaccagggattgcgccgtgggagacgctcgtattgaaagcaacctcggcatcgtcgacctcgagtgcaacggcgcga

SEQ ID NO: 555 - DNA, 200 bp, Kochia scoparia
aaagcggatacgaattcgaattcatggtttcttcctgggttcgaggaaatgccggaggaatcgtcgtttcgagggctcgatttcgggctccggagactagaccatgcggtggggaatgtcccggagctagggaaagcaatcgagtatgtgaagaagttcactgggtttcacgaatttgctgagtttacagctgacgatgt

SEQ ID NO: 556 - DNA, 200 bp, Kochia scoparia
acatcgtcagctgtaaactcagcaaattcgtgaaacccagtgaacttcttcacatactcgattgctttccctagctccgggacattccccaccgcatggtctagtctccggagcccgaaatcgagccctcgaaacgacgattcctccggcatttcctcgaacccaggaagaaaccatgaattcgaattcgtatccgcttt

SEQ ID NO: 557 - DNA, 200 bp, Kochia scoparia
ttgctgagtttacagctgacgatgttgggacgagtgaaagtgggctgaattcggctgtgctggcgaacaactcggagacggtgttgattccgatgaacgagccggtttacgggacgaagaggaagagtcaaattcagacatacttggagcacaatgaaggagctggggttcagcatttggcattgatgagtgaggatata

SEQ ID NO: 558 - DNA, 200 bp, Kochia scoparia
tatatcctcactcatcaatgccaaatgctgaaccccagctccttcattgtgctccaagtatgtctgaatttgactcttcctcttcgtcccgtaaaccggctcgttcatcggaatcaacaccgtctccgagttgttcgccagcacagccgaattcagcccactttcactcgtcccaacatcgtcagctgtaaactcagcaa

SEQ ID NO: 559 - DNA, 200 bp, Kochia scoparia
tttggcattgatgagtgaggatatattctggactcttagggagatgaggaagcggagcgggctcggtgggttcgagtttatgccagcaccgccgcctacgtattatcggaacctgaggaatcgtgtcggggatgtgttgagtgaagaacagatgaaggagtgtgaagagttggggatattggttgataaggatgatcagg

SEQ ID NO: 560 - DNA, 200 bp, Kochia scoparia
cctgatcatccttatcaaccaatatccccaactcttcacactccttcatctgttcttcactcaacacatccccgacacgattcctcaggttccgataatacgtaggcggcggtgctggcataaactcgaacccaccgagcccgctccgcttcctcatctccctaagagtccagaatatatcctcactcatcaatgccaaa

SEQ ID NO: 561 - DNA, 200 bp, Kochia scoparia
atattggttgataaggatgatcagggcactctgctccagatttttactaagcatattggtgacccaactatgttcattgagattatccaaagaattggctgcatgatgaaagatgaagaaggcaaggtgtaccaaaagggaggctgtggaggatttgggaagggaaacttttcagagcttttcaaatctatcgaagagta

SEQ ID NO: 562 - DNA, 200 bp, Kochia scoparia
tactcttcgatagatttgaaaagctctgaaaagtttcccttcccaaatcctccacagcctcccttttggtacaccttgccttcttcatctttcatcatgcagccaattctttggataatctcaatgaacatagttgggtcaccaatatgcttagtaaaaatctggagcagagtgccctgatcatccttatcaaccaatat

SEQ ID NO: 563 - DNA, 200 bp, Lolium multiflorum
accgatcgcttccacgtgatggatttccaccacgtcgagttttggtgcgccgatgccgcctcggccgccgcacggttctccttcgggctcggcgtgccactcgccgcgcagtccgacctctccacggggaacactgcgcacgcctcacgcctactacgcgcacgctcgggctctctctcgttcctcttcaccgcgccgta

SEQ ID NO: 564 - DNA, 200 bp, Lolium multiflorum
tacggcgcggtgaagaggaacgagagagagcccgagcgtgcgcgtagtaggcgtgaggcgtgcgcagtgttccccgtggagaggtcggactgcgcggcgagtggcacgccgagcccgaaggagaaccgtgcggcggccgaggcggcatcggcgcaccaaaactcgacgtggtggaaatccatcacgtggaagcgatcggt

SEQ ID NO: 565 - DNA, 200 bp, Lolium multiflorum
tctcgttcctcttcaccgcgccgtacgcgccgcacgtcgccgactcggcgaccaccgcgtccctgccctccttctcggcggacgccgcgcggcgcttcacgggcacccacggcggcctggccgtgcgtgccgtggccgtccgcgtcgctgacgcggccgaggccttcgtcgcgagcgtggacgccggagcgcggccagcc

SEQ ID NO: 566 - DNA, 200 bp, Lolium multiflorum
ggctggccgcgctccggcgtccacgctcgcgacgaaggcctcggccgcgtcagcgacgcggacggccacggcacgcacggccaggccgccgtgggtgcccgtgaagcgccgcgcggcgtccgccgagaaggagggcagggacgcggtggtcgccgagtcggcgacgtgcggcgcgtacggcgcggtgaagaggaacgaga

SEQ ID NO: 567 - DNA, 200 bp, Lolium multiflorum
cgtggacgccggagcgcggccagcctgcgccccgactgatctcggccacgggtttggcttcgcggaggtggagctagccggggacagcgttctccgcttcgtgagctacccggacggcaccgacgtgtccttcctgccggggttccaggacgtggcgagcgccggcggggcgccggacttcgggctcacccggtttgacc

SEQ ID NO: 568 - DNA, 200 bp, Lolium multiflorum
ggtcaaaccgggtgagcccgaagtccggcgccccgccggcgctcgccacgtcctggaaccccggcaggaaggacacgtcggtgccgtccgggtagctcacgaagcggagaacgctgtccccggctagctccacctccgcgaagccaaacccgtggccgagatcagtcggggcgcaggctggccgcgctccggcgtccacg

SEQ ID NO: 569 - DNA, 200 bp, Lolium multiflorum
gacttcgggctcacccggtttgaccacgtcgacgttaacatcccggagctggcacccgtcgccgccaatgttgccggcttcaccgggttccacaaattctgggagttcaccgcggacgacgtgtgcccggaagagagcggggtgaacggcgtggtgatcgccaacaactcagagaacgtgctgctcagtatcttggagcc

SEQ ID NO: 570 - DNA, 200 bp, Lolium multiflorum
ggctccaagatactgagcagcacgttctctgagttgttggcgatcaccacgccgttcaccccgctctcttccgggcacacgtcgtccgcggtgaactcccagaatttgtggaacccggtgaagccggcaacattggcggcgacgggtgccagctccgggatgttaacgtcgacgtggtcaaaccgggtgagcccgaagtc

SEQ ID NO: 571 - DNA, 200 bp, Lolium multiflorum
acgtgctgctcagtatcttggagccggtgttcggcaccaagctgcggagccatgtcgagacgttcctggaccaccacggtggcccgggcatacagcacctggcaatgaccagccacgacatccttggcgcgctcaggaaaatccgagctcggtcctccatgggcgggtttgagctcctgccgccgccgccggccagctac

SEQ ID NO: 572 - DNA, 200 bp, Lolium multiflorum
gtagctggccggcggcggcggcaggagctcaaacccgcccatggaggaccgagctcggattttcctgagcgcgccaaggatgtcgtggctggtcattgccaggtgctgtatgcccgggccaccgtggtggtccaggaacgtctcgacatggctccgcagcttggtgccgaacaccggctccaagatactgagcagcacgt

SEQ ID NO: 573 - DNA, 200 bp, Lolium multiflorum
ctcggccacgggtttggcttcgcggaggtggagctagccggggacagcgttctccgcttcgtgagctacccggacggcaccgacgtgtccttcctgccggggttccaggacgtggcgagcgccggcggggcgccggacttcgggctcacccggtttgaccacgtcgacgttaacatcccggagctggcacccgtcgccgc

SEQ ID NO: 574 - DNA, 200 bp, Lolium multiflorum
gcggcgacgggtgccagctccgggatgttaacgtcgacgtggtcaaaccgggtgagcccgaagtccggcgccccgccggcgctcgccacgtcctggaaccccggcaggaaggacacgtcggtgccgtccgggtagctcacgaagcggagaacgctgtccccggctagctccacctccgcgaagccaaacccgtggccgag

SEQ ID NO: 575 - DNA, 200 bp, Lolium multiflorum
tcccggagctggcacccgtcgccgccaatgttgccggcttcaccgggttccacaaattctgggagttcaccgcggacgacgtgtgcccggaagagagcggggtgaacggcgtggtgatcgccaacaactcagagaacgtgctgctcagtatcttggagccggtgttcggcaccaagctgcggagccatgtcgagacgttc

SEQ ID NO: 576 - DNA, 200 bp, Lolium multiflorum
gaacgtctcgacatggctccgcagcttggtgccgaacaccggctccaagatactgagcagcacgttctctgagttgttggcgatcaccacgccgttcaccccgctctcttccgggcacacgtcgtccgcggtgaactcccagaatttgtggaacccggtgaagccggcaacattggcggcgacgggtgccagctccggga

SEQ ID NO: 577 - DNA, 200 bp, Lolium multiflorum
gctgcggagccatgtcgagacgttcctggaccaccacggtggcccgggcatacagcacctggcaatgaccagccacgacatccttggcgcgctcaggaaaatccgagctcggtcctccatgggcgggtttgagctcctgccgccgccgccggccagctactatgacggtgtaaggcagcgcgccggggacgtgctgtcgg

SEQ ID NO: 578 - DNA, 200 bp, Lolium multiflorum
ccgacagcacgtccccggcgcgctgccttacaccgtcatagtagctggccggcggcggcggcaggagctcaaacccgcccatggaggaccgagctcggattttcctgagcgcgccaaggatgtcgtggctggtcattgccaggtgctgtatgcccgggccaccgtggtggtccaggaacgtctcgacatggctccgcagc

SEQ ID NO: 579 - DNA, 200 bp, Lolium multiflorum
cagcgcgccggggacgtgctgtcggaagaacagatcaaggagtgccaagagctgggcgtgcgggtggacagagggtatgaggacggagttgtgctccaagtcttcaccaaaccggcgggagacccaaccttactgttagagtttatccaaagaatcgggtgcatggtcaaggacgagaaccagcaggaataccagagagg

SEQ ID NO: 580 - DNA, 200 bp, Lolium multiflorum
cctctctggtattcctgctggttctcgtccttgaccatgcacccgattctttggataaactctaacagtaaggttgggtctcccgccggtttggtgaagacttggagcacaactccgtcctcataccctctgtccacccgcacgcccagctcttggcactccttgatctgttcttccgacagcacgtccccggcgcgctg

SEQ ID NO: 581 - DNA, 200 bp, Xanthium strumarium
agaaataaaagacacatatgttcgtgaggttatgacacctttggttgatgttgtagccagtgattctagtgcaacactaatcgatttccacaccttgtgggtaagtcatcaatattcaagggtacctgtttttgaacaacgtgttgataatatagtcggtattgcatatgcaatggatctattagactatgttcaaaagg

SEQ ID NO: 582 - DNA, 200 bp, Xanthium strumarium
ccttttgaacatagtctaatagatccattgcatatgcaataccgactatattatcaacacgttgttcaaaaacaggtacccttgaatattgatgacttacccacaaggtgtggaaatcgattagtgttgcactagaatcactggctacaacatcaaccaaaggtgtcataacctcacgaacatatgtgtcttttatttct

SEQ ID NO: 583 - DNA, 200 bp, Xanthium strumarium
gatctattagactatgttcaaaagggagaccttttagaaagtaccacggtgggggatatggctcataaacctgcttattttgtgcctgattcaatgtcggtatggaatcttcttagagaatttcgcattaggaaggtacacatggctgttgtcctgaacgaatacggtgggacagttggaattgtaacactagaagacgt

SEQ ID NO: 584 - DNA, 200 bp, Xanthium strumarium
acgtcttctagtgttacaattccaactgtcccaccgtattcgttcaggacaacagccatgtgtaccttcctaatgcgaaattctctaagaagattccataccgacattgaatcaggcacaaaataagcaggtttatgagccatatcccccaccgtggtactttctaaaaggtctcccttttgaacatagtctaatagatc

SEQ ID NO: 585 - DNA, 200 bp, Xanthium strumarium
ttggaattgtaacactagaagacgtggttgaagaaattgttggtgaaatttcttcaacccacgcctcttatctccttaaatccggccaacttaacttcctcttcaccgccccttattccccttccatctcatccaccacaaccaccgcctccatcccttccttttctcactccacctgtcgccacttcacctcctcccac

SEQ ID NO: 586 - DNA, 200 bp, Xanthium strumarium
gtgggaggaggtgaagtggcgacaggtggagtgagaaaaggaagggatggaggcggtggttgtggtggatgagatggaaggggaataaggggcggtgaagaggaagttaagttggccggatttaaggagataagaggcgtgggttgaagaaatttcaccaacaatttcttcaaccacgtcttctagtgttacaattccaa

SEQ ID NO: 587 - DNA, 200 bp, Xanthium strumarium
ctgtcgccacttcacctcctcccacggcctcgctgttcgtgccatcgccgttgaagtccatgacgccgaactcgctttctccgttagcgtcgctcacggcgctaaagcctccggataccataatccagctcttcaaacgacgacgtttctccacagcttcaaacccaggcaaaacatattgttattattattattgtttg

SEQ ID NO: 588 - DNA, 200 bp, Xanthium strumarium
caaacaataataataataacaatatgttttgcctgggtttgaagctgtggagaaacgtcgtcgtttgaagagctggattatggtatccggaggctttagcgccgtgagcgacgctaacggagaaagcgagttcggcgtcatggacttcaacggcgatggcacgaacagcgaggccgtgggaggaggtgaagtggcgacag

SEQ ID NO: 589 - DNA, 200 bp, Xanthium strumarium
atattgttattattattattgtttgtaactaatataacgcaaaacgacgtcgccgtagagttgaacttctgataagacgacgtcgttatgaccgagggtgataatccggagggttgaccacgcagtggggaacgtgccggagttagcaccggcagtggaatatataaaatcatttactggatttcacgagtttgctgagt

SEQ ID NO: 590 - DNA, 200 bp, Xanthium strumarium
actcagcaaactcgtgaaatccagtaaatgattttatatattccactgccggtgctaactccggcacgttccccactgcgtggtcaaccctccggattatcaccctcggtcataacgacgtcgtcttatcagaagttcaactctacggcgacgtcgttttgcgttatattagttacaaacaataataataataacaatat

SEQ ID NO: 591 - DNA, 200 bp, Xanthium strumarium
actggatttcacgagtttgctgagtttacggcggaggatgtgggaacgagtgagagtggactaaactcggtggttttggcttgcaatagtgagatggtattgataccgatgaatgaaccggtttacgggacgaagaggaagagtcagatacagacgtatttggaacataatgaaggggcgggggttcagcatttggcgtt

SEQ ID NO: 592 - DNA, 200 bp, Xanthium strumarium
aacgccaaatgctgaacccccgccccttcattatgttccaaatacgtctgtatctgactcttcctcttcgtcccgtaaaccggttcattcatcggtatcaataccatctcactattgcaagccaaaaccaccgagtttagtccactctcactcgttcccacatcctccgccgtaaactcagcaaactcgtgaaatccagt

SEQ ID NO: 593 - DNA, 200 bp, Xanthium strumarium
gggcgggggttcagcatttggcgttggctagtgaggatatatttaggacgttaagggagatgaggaaaaggagtggggttggtggctttgagtttatgccgtctccccctcctacttattataggaatttgaagaatagggtgggcgatgtgttgtctgatgaacagattaaggagtgtgaagaattggggatattggtt

SEQ ID NO: 594 - DNA, 200 bp, Xanthium strumarium
aaccaatatccccaattcttcacactccttaatctgttcatcagacaacacatcgcccaccctattcttcaaattcctataataagtaggagggggagacggcataaactcaaagccaccaaccccactccttttcctcatctcccttaacgtcctaaatatatcctcactagccaacgccaaatgctgaacccccgccc

SEQ ID NO: 595 - DNA, 200 bp, Xanthium strumarium
gtgtgaagaattggggatattggttgatagagatgatcaggggactttgcttcagatttttaccaagcctgttggtgacaggccgacgatattcatagagataattcagagagtagggtgtatggtgaaggataatgaaggaaaggagcagcagaaggcagggtgtggagggtttggcaaagggaacttctcagagcttt

SEQ ID NO: 596 - DNA, 200 bp, Xanthium strumarium
aaagctctgagaagttccctttgccaaaccctccacaccctgccttctgctgctcctttccttcattatccttcaccatacaccctactctctgaattatctctatgaatatcgtcggcctgtcaccaacaggcttggtaaaaatctgaagcaaagtcccctgatcatctctatcaaccaatatccccaattcttcacac

SEQ ID NO: 597 - DNA, 21 bp, Unknown
  Found in multiple species
tccccaactcttcacactcct

SEQ ID NO: 598 - DNA, 21 bp, Unknown
  Found in multiple species
ggagtgtgaagagttggggat

SEQ ID NO: 599 - DNA, 21 bp, Unknown
  Found in multiple species
ccccaactcttcacactcctt

SEQ ID NO: 600 - DNA, 21 bp, Unknown
  Found in multiple species
atccccaactcttcacactcc

SEQ ID NO: 601 - DNA, 21 bp, Unknown
  Found in multiple species
aggagtgtgaagagttgggga

SEQ ID NO: 602 - DNA, 21 bp, Unknown
  Found in multiple species
aaggagtgtgaagagttgggg

SEQ ID NO: 603 - DNA, 21 bp, Unknown
  Found in multiple species
tgtgaagagttggggattttg

SEQ ID NO: 604 - DNA, 21 bp, Unknown
  Found in multiple species
tgggtttgagtttatgccgtc

SEQ ID NO: 605 - DNA, 21 bp, Unknown
  Found in multiple species
tgaaggagtgtgaagagttgg

SEQ ID NO: 606 - DNA, 21 bp, Unknown
  Found in multiple species
tgaagagttggggattttggt

SEQ ID NO: 607 - DNA, 21 bp, Unknown
  Found in multiple species
gtgtgaagagttggggatttt

SEQ ID NO: 608 - DNA, 21 bp, Unknown
  Found in multiple species
gtgggtttgagtttatgccgt

SEQ ID NO: 609 - DNA, 21 bp, Unknown
  Found in multiple species
gtgaagagttggggattttgg

SEQ ID NO: 610 - DNA, 21 bp, Unknown
  Found in multiple species
ggtgggtttgagtttatgccg

SEQ ID NO: 611 - DNA, 21 bp, Unknown
  Found in multiple species
gatgaaggagtgtgaagagtt

SEQ ID NO: 612 - DNA, 21 bp, Unknown
  Found in multiple species
gagtgtgaagagttggggatt

SEQ ID NO: 613 - DNA, 21 bp, Unknown
  Found in multiple species
gacggcataaactcaaaccca

SEQ ID NO: 614 - DNA, 21 bp, Unknown
  Found in multiple species
gaaggagtgtgaagagttggg

SEQ ID NO: 615 - DNA, 21 bp, Unknown
  Found in multiple species
ctcttcacactccttcatctg

SEQ ID NO: 616 - DNA, 21 bp, Unknown
  Found in multiple species
cggcataaactcaaacccacc

SEQ ID NO: 617 - DNA, 21 bp, Unknown
  Found in multiple species
cccaactcttcacactccttc

SEQ ID NO: 618 - DNA, 21 bp, Unknown
  Found in multiple species
ccaactcttcacactccttca

SEQ ID NO: 619 - DNA, 21 bp, Unknown
  Found in multiple species
ccaaaatccccaactcttcac

SEQ ID NO: 620 - DNA, 21 bp, Unknown
  Found in multiple species
cagatgaaggagtgtgaagag

SEQ ID NO: 621 - DNA, 21 bp, Unknown
  Found in multiple species
caactcttcacactccttcat

SEQ ID NO: 622 - DNA, 21 bp, Unknown
  Found in multiple species
caaaatccccaactcttcaca

SEQ ID NO: 623 - DNA, 21 bp, Unknown
  Found in multiple species
atgaaggagtgtgaagagttg

SEQ ID NO: 624 - DNA, 21 bp, Unknown
  Found in multiple species
agtgtgaagagttggggattt

SEQ ID NO: 625 - DNA, 21 bp, Unknown
  Found in multiple species
agatgaaggagtgtgaagagt

SEQ ID NO: 626 - DNA, 21 bp, Unknown
  Found in multiple species
actcttcacactccttcatct

SEQ ID NO: 627 - DNA, 21 bp, Unknown
  Found in multiple species
acggcataaactcaaacccac

SEQ ID NO: 628 - DNA, 21 bp, Unknown
  Found in multiple species
accaaaatccccaactcttca

SEQ ID NO: 629 - DNA, 21 bp, Unknown
  Found in multiple species
aatccccaactcttcacactc

SEQ ID NO: 630 - DNA, 21 bp, Unknown
  Found in multiple species
aactcttcacactccttcatc

SEQ ID NO: 631 - DNA, 21 bp, Unknown
  Found in multiple species
aaatccccaactcttcacact

SEQ ID NO: 632 - DNA, 21 bp, Unknown
  Found in multiple species
aaaatccccaactcttcacac

SEQ ID NO: 633 - DNA, 21 bp, Unknown
  Found in multiple species
tttttgtcccatacacaggtt

SEQ ID NO: 634 - DNA, 21 bp, Unknown
  Found in multiple species
ttttgtcccatacacaggttc

SEQ ID NO: 635 - DNA, 21 bp, Unknown
  Found in multiple species
ttttggactttaagggagatg

SEQ ID NO: 636 - DNA, 21 bp, Unknown
  Found in multiple species
tttgtcccatacacaggttca

SEQ ID NO: 637 - DNA, 21 bp, Unknown
  Found in multiple species
tttggctttgatgagtgaaga

SEQ ID NO: 638 - DNA, 21 bp, Unknown
  Found in multiple species
tttggagcataatgaaggagc

SEQ ID NO: 639 - DNA, 21 bp, Unknown
  Found in multiple species
tttggactttaagggagatga

SEQ ID NO: 640 - DNA, 21 bp, Unknown
  Found in multiple species
tttgctgagtttacagctgaa

SEQ ID NO: 641 - DNA, 21 bp, Unknown
  Found in multiple species
tttgccaatacggctgaattc

SEQ ID NO: 642 - DNA, 21 bp, Unknown
  Found in multiple species
tttgagtttatgccgtcgccg

SEQ ID NO: 643 - DNA, 21 bp, Unknown
  Found in multiple species
tttccaatgaatgaacctgtg

SEQ ID NO: 644 - DNA, 21 bp, Unknown
  Found in multiple species
tttcatgagtttgctgagttt

SEQ ID NO: 645 - DNA, 21 bp, Unknown
  Found in multiple species
tttatgccgtcgccgcctccg

SEQ ID NO: 646 - DNA, 21 bp, Unknown
  Found in multiple species
tttactgggtttcatgagttt

SEQ ID NO: 647 - DNA, 21 bp, Unknown
  Found in multiple species
tttaagggagatgaggaagag

SEQ ID NO: 648 - DNA, 21 bp, Unknown
  Found in multiple species
ttgtttccaatgaatgaacct

SEQ ID NO: 649 - DNA, 21 bp, Unknown
  Found in multiple species
ttgtcccatacacaggttcat

SEQ ID NO: 650 - DNA, 21 bp, Unknown
  Found in multiple species
ttggtgggtttgagtttatgc

SEQ ID NO: 651 - DNA, 21 bp, Unknown
  Found in multiple species
ttggctcctgcaattgcttat

SEQ ID NO: 652 - DNA, 21 bp, Unknown
  Found in multiple species
ttggagcataatgaaggagct

SEQ ID NO: 653 - DNA, 21 bp, Unknown
  Found in multiple species
ttggactttaagggagatgag

SEQ ID NO: 654 - DNA, 21 bp, Unknown
  Found in multiple species
ttggaaacaacaccatttcat

SEQ ID NO: 655 - DNA, 21 bp, Unknown
  Found in multiple species
ttgctgagtttacagctgaag

SEQ ID NO: 656 - DNA, 21 bp, Unknown
  Found in multiple species
ttgccaatacggctgaattca

SEQ ID NO: 657 - DNA, 21 bp, Unknown
  Found in multiple species
ttgagtttatgccgtcgccgc

SEQ ID NO: 658 - DNA, 21 bp, Unknown
  Found in multiple species
ttgagtgaggagcagatgaag

SEQ ID NO: 659 - DNA, 21 bp, Unknown
  Found in multiple species
ttgaattcagccgtattggca

SEQ ID NO: 660 - DNA, 21 bp, Unknown
  Found in multiple species
ttctcttcctcatctccctta

SEQ ID NO: 661 - DNA, 21 bp, Unknown
  Found in multiple species
ttcctttttgtcccatacaca

SEQ ID NO: 662 - DNA, 21 bp, Unknown
  Found in multiple species
ttcctcatctcccttaaagtc

SEQ ID NO: 663 - DNA, 21 bp, Unknown
  Found in multiple species
ttcctcaaattccggtaataa

SEQ ID NO: 664 - DNA, 21 bp, Unknown
  Found in multiple species
ttccggtaataagtcggaggc

SEQ ID NO: 665 - DNA, 21 bp, Unknown
  Found in multiple species
ttccaatgaatgaacctgtgt

SEQ ID NO: 666 - DNA, 21 bp, Unknown
  Found in multiple species
ttcattggaaacaacaccatt

SEQ ID NO: 667 - DNA, 21 bp, Unknown
  Found in multiple species
ttcattcattggaaacaacac

SEQ ID NO: 668 - DNA, 21 bp, Unknown
  Found in multiple species
ttcattatgctccaaataagt

SEQ ID NO: 669 - DNA, 21 bp, Unknown
  Found in multiple species
ttcatgagtttgctgagttta

SEQ ID NO: 670 - DNA, 21 bp, Unknown
  Found in multiple species
ttcatctgctcctcactcaat

SEQ ID NO: 671 - DNA, 21 bp, Unknown
  Found in multiple species
ttcagctgtaaactcagcaaa

SEQ ID NO: 672 - DNA, 21 bp, Unknown
  Found in multiple species
ttcactcatcaaagccaaatg

SEQ ID NO: 673 - DNA, 21 bp, Unknown
  Found in multiple species
ttcacactccttcatctgctc

SEQ ID NO: 674 - DNA, 21 bp, Unknown
  Found in multiple species
ttcaaataagcaattgcagga

SEQ ID NO: 675 - DNA, 21 bp, Unknown
  Found in multiple species
ttcaaacttatttggagcata

SEQ ID NO: 676 - DNA, 21 bp, Unknown
  Found in multiple species
ttatttggagcataatgaagg

SEQ ID NO: 677 - DNA, 21 bp, Unknown
  Found in multiple species
ttattaccggaatttgaggaa

SEQ ID NO: 678 - DNA, 21 bp, Unknown
  Found in multiple species
ttatgctccaaataagtttga

SEQ ID NO: 679 - DNA, 21 bp, Unknown
  Found in multiple species
ttatgccgtcgccgcctccga

SEQ ID NO: 680 - DNA, 21 bp, Unknown
  Found in multiple species
ttactgggtttcatgagtttg

SEQ ID NO: 681 - DNA, 21 bp, Unknown
  Found in multiple species
ttaagggagatgaggaagaga

SEQ ID NO: 682 - DNA, 21 bp, Unknown
  Found in multiple species
tgtttccaatgaatgaacctg

SEQ ID NO: 683 - DNA, 21 bp, Unknown
  Found in multiple species
tgttgtttccaatgaatgaac

SEQ ID NO: 684 - DNA, 21 bp, Unknown
  Found in multiple species
tgtgtatgggacaaaaaggaa

SEQ ID NO: 685 - DNA, 21 bp, Unknown
  Found in multiple species
tgtcccatacacaggttcatt

SEQ ID NO: 686 - DNA, 21 bp, Unknown
  Found in multiple species
tgtattgagtgaggagcagat

SEQ ID NO: 687 - DNA, 21 bp, Unknown
  Found in multiple species
tgtatgggacaaaaaggaaga

SEQ ID NO: 688 - DNA, 21 bp, Unknown
  Found in multiple species
tgtacaccagctccttcatta

SEQ ID NO: 689 - DNA, 21 bp, Unknown
  Found in multiple species
tgtaaactcagcaaactcatg

SEQ ID NO: 690 - DNA, 21 bp, Unknown
  Found in multiple species
tggtgttgtttccaatgaatg

SEQ ID NO: 691 - DNA, 21 bp, Unknown
  Found in multiple species
tggtgggtttgagtttatgcc

SEQ ID NO: 692 - DNA, 21 bp, Unknown
  Found in multiple species
tggtcttggtgggtttgagtt

SEQ ID NO: 693 - DNA, 21 bp, Unknown
  Found in multiple species
tgggtttcatgagtttgctga

SEQ ID NO: 694 - DNA, 21 bp, Unknown
  Found in multiple species
tggctcctgcaattgcttatt

SEQ ID NO: 695 - DNA, 21 bp, Unknown
  Found in multiple species
tggagcataatgaaggagctg

SEQ ID NO: 696 - DNA, 21 bp, Unknown
  Found in multiple species
tggactttaagggagatgagg

SEQ ID NO: 697 - DNA, 21 bp, Unknown
  Found in multiple species
tggaaacaacaccatttcatc

SEQ ID NO: 698 - DNA, 21 bp, Unknown
  Found in multiple species
tgctgatgtattgagtgagga

SEQ ID NO: 699 - DNA, 21 bp, Unknown
  Found in multiple species
tgctgagtttacagctgaaga

SEQ ID NO: 700 - DNA, 21 bp, Unknown
  Found in multiple species
tgctcctcactcaatacatca

SEQ ID NO: 701 - DNA, 21 bp, Unknown
  Found in multiple species
tgctccaaataagtttgaatt

SEQ ID NO: 702 - DNA, 21 bp, Unknown
  Found in multiple species
tgccgtcgccgcctccgactt

SEQ ID NO: 703 - DNA, 21 bp, Unknown
  Found in multiple species
tgccaatacggctgaattcaa

SEQ ID NO: 704 - DNA, 21 bp, Unknown
  Found in multiple species
tgatgtattgagtgaggagca

SEQ ID NO: 705 - DNA, 21 bp, Unknown
  Found in multiple species
tgatgaaatggtgttgtttcc

SEQ ID NO: 706 - DNA, 21 bp, Unknown
  Found in multiple species
tgagtttgctgagtttacagc

SEQ ID NO: 707 - DNA, 21 bp, Unknown
  Found in multiple species
tgagtttatgccgtcgccgcc

SEQ ID NO: 708 - DNA, 21 bp, Unknown
  Found in multiple species
tgagtttacagctgaagatgt

SEQ ID NO: 709 - DNA, 21 bp, Unknown
  Found in multiple species
tgagtgaggagcagatgaagg

SEQ ID NO: 710 - DNA, 21 bp, Unknown
  Found in multiple species
tgaggagcagatgaaggagtg

SEQ ID NO: 711 - DNA, 21 bp, Unknown
  Found in multiple species
tgaggaagagaagtggtcttg

SEQ ID NO: 712 - DNA, 21 bp, Unknown
  Found in multiple species
tgaattcagccgtattggcaa

SEQ ID NO: 713 - DNA, 21 bp, Unknown
  Found in multiple species
tgaatgaacctgtgtatggga

SEQ ID NO: 714 - DNA, 21 bp, Unknown
  Found in multiple species
tgaacctgtgtatgggacaaa

SEQ ID NO: 715 - DNA, 21 bp, Unknown
  Found in multiple species
tgaaatggtgttgtttccaat

SEQ ID NO: 716 - DNA, 21 bp, Unknown
  Found in multiple species
tcttggtgggtttgagtttat

SEQ ID NO: 717 - DNA, 21 bp, Unknown
  Found in multiple species
tcttcctttttgtcccataca

SEQ ID NO: 718 - DNA, 21 bp, Unknown
  Found in multiple species
tcttcctcatctcccttaaag

SEQ ID NO: 719 - DNA, 21 bp, Unknown
  Found in multiple species
tcttcagctgtaaactcagca

SEQ ID NO: 720 - DNA, 21 bp, Unknown
  Found in multiple species
tcttcactcatcaaagccaaa

SEQ ID NO: 721 - DNA, 21 bp, Unknown
  Found in multiple species
tcttcacactccttcatctgc

SEQ ID NO: 722 - DNA, 21 bp, Unknown
  Found in multiple species
tctgctcctcactcaatacat

SEQ ID NO: 723 - DNA, 21 bp, Unknown
  Found in multiple species
tctcttcctcatctcccttaa

SEQ ID NO: 724 - DNA, 21 bp, Unknown
  Found in multiple species
tctcccttaaagtccaaaata

SEQ ID NO: 725 - DNA, 21 bp, Unknown
  Found in multiple species
tcggaggcggcgacggcataa

SEQ ID NO: 726 - DNA, 21 bp, Unknown
  Found in multiple species
tcgccgcctccgacttattac

SEQ ID NO: 727 - DNA, 21 bp, Unknown
  Found in multiple species
tcctttttgtcccatacacag

SEQ ID NO: 728 - DNA, 21 bp, Unknown
  Found in multiple species
tccttcattatgctccaaata

SEQ ID NO: 729 - DNA, 21 bp, Unknown
  Found in multiple species
tccttcatctgctcctcactc

SEQ ID NO: 730 - DNA, 21 bp, Unknown
  Found in multiple species
tcctgcaattgcttatttgaa

SEQ ID NO: 731 - DNA, 21 bp, Unknown
  Found in multiple species
tcctcatctcccttaaagtcc

SEQ ID NO: 732 - DNA, 21 bp, Unknown
  Found in multiple species
tcctcactcaatacatcagca

SEQ ID NO: 733 - DNA, 21 bp, Unknown
  Found in multiple species
tcctcaaattccggtaataag

SEQ ID NO: 734 - DNA, 21 bp, Unknown
  Found in multiple species
tccggtaataagtcggaggcg

SEQ ID NO: 735 - DNA, 21 bp, Unknown
  Found in multiple species
tccgacttattaccggaattt

SEQ ID NO: 736 - DNA, 21 bp, Unknown
  Found in multiple species
tcccatacacaggttcattca

SEQ ID NO: 737 - DNA, 21 bp, Unknown
  Found in multiple species
tccaatgaatgaacctgtgta

SEQ ID NO: 738 - DNA, 21 bp, Unknown
  Found in multiple species
tcattggaaacaacaccattt

SEQ ID NO: 739 - DNA, 21 bp, Unknown
  Found in multiple species
tcattcattggaaacaacacc

SEQ ID NO: 740 - DNA, 21 bp, Unknown
  Found in multiple species
tcattatgctccaaataagtt

SEQ ID NO: 741 - DNA, 21 bp, Unknown
  Found in multiple species
tcatgagtttgctgagtttac

SEQ ID NO: 742 - DNA, 21 bp, Unknown
  Found in multiple species
tcatctgctcctcactcaata

SEQ ID NO: 743 - DNA, 21 bp, Unknown
  Found in multiple species
tcatctcccttaaagtccaaa

SEQ ID NO: 744 - DNA, 21 bp, Unknown
  Found in multiple species
tcagctgtaaactcagcaaac

SEQ ID NO: 745 - DNA, 21 bp, Unknown
  Found in multiple species
tcagcaaactcatgaaaccca

SEQ ID NO: 746 - DNA, 21 bp, Unknown
  Found in multiple species
tcactcaatacatcagcagct

SEQ ID NO: 747 - DNA, 21 bp, Unknown
  Found in multiple species
tcacactccttcatctgctcc

SEQ ID NO: 748 - DNA, 21 bp, Unknown
  Found in multiple species
tcaaattccggtaataagtcg

SEQ ID NO: 749 - DNA, 21 bp, Unknown
  Found in multiple species
tcaaataagcaattgcaggag

SEQ ID NO: 750 - DNA, 21 bp, Unknown
  Found in multiple species
tcaaacttatttggagcataa

SEQ ID NO: 751 - DNA, 21 bp, Unknown
  Found in multiple species
tcaaacccaccaagaccactt

SEQ ID NO: 752 - DNA, 21 bp, Unknown
  Found in multiple species
tattttggactttaagggaga

SEQ ID NO: 753 - DNA, 21 bp, Unknown
  Found in multiple species
tatttggagcataatgaagga

SEQ ID NO: 754 - DNA, 21 bp, Unknown
  Found in multiple species
tattgagtgaggagcagatga

SEQ ID NO: 755 - DNA, 21 bp, Unknown
  Found in multiple species
tatgctccaaataagtttgaa

SEQ ID NO: 756 - DNA, 21 bp, Unknown
  Found in multiple species
tatgccgtcgccgcctccgac

SEQ ID NO: 757 - DNA, 21 bp, Unknown
  Found in multiple species
tactgggtttcatgagtttgc

SEQ ID NO: 758 - DNA, 21 bp, Unknown
  Found in multiple species
tacaccagctccttcattatg

SEQ ID NO: 759 - DNA, 21 bp, Unknown
  Found in multiple species
tacacaggttcattcattgga

SEQ ID NO: 760 - DNA, 21 bp, Unknown
  Found in multiple species
taatgaaggagctggtgtaca

SEQ ID NO: 761 - DNA, 21 bp, Unknown
  Found in multiple species
taataagtcggaggcggcgac

SEQ ID NO: 762 - DNA, 21 bp, Unknown
  Found in multiple species
taagtcggaggcggcgacggc

SEQ ID NO: 763 - DNA, 21 bp, Unknown
  Found in multiple species
taagggagatgaggaagagaa

SEQ ID NO: 764 - DNA, 21 bp, Unknown
  Found in multiple species
taagcaattgcaggagccaac

SEQ ID NO: 765 - DNA, 21 bp, Unknown
  Found in multiple species
taaactcagcaaactcatgaa

SEQ ID NO: 766 - DNA, 21 bp, Unknown
  Found in multiple species
taaactcaaacccaccaagac

SEQ ID NO: 767 - DNA, 21 bp, Unknown
  Found in multiple species
gtttgctgagtttacagctga

SEQ ID NO: 768 - DNA, 21 bp, Unknown
  Found in multiple species
gtttgagtttatgccgtcgcc

SEQ ID NO: 769 - DNA, 21 bp, Unknown
  Found in multiple species
gtttccaatgaatgaacctgt

SEQ ID NO: 770 - DNA, 21 bp, Unknown
  Found in multiple species
gtttcatgagtttgctgagtt

SEQ ID NO: 771 - DNA, 21 bp, Unknown
  Found in multiple species
gtttatgccgtcgccgcctcc

SEQ ID NO: 772 - DNA, 21 bp, Unknown
  Found in multiple species
gtttactgggtttcatgagtt

SEQ ID NO: 773 - DNA, 21 bp, Unknown
  Found in multiple species
gtttacagctgaagatgttgg

SEQ ID NO: 774 - DNA, 21 bp, Unknown
  Found in multiple species
gttgtttccaatgaatgaacc

SEQ ID NO: 775 - DNA, 21 bp, Unknown
  Found in multiple species
gttggctcctgcaattgctta

SEQ ID NO: 776 - DNA, 21 bp, Unknown
  Found in multiple species
gttcattcattggaaacaaca

SEQ ID NO: 777 - DNA, 21 bp, Unknown
  Found in multiple species
gtgttgtttccaatgaatgaa

SEQ ID NO: 778 - DNA, 21 bp, Unknown
  Found in multiple species
gtgtatgggacaaaaaggaag

SEQ ID NO: 779 - DNA, 21 bp, Unknown
  Found in multiple species
gtggtcttggtgggtttgagt

SEQ ID NO: 780 - DNA, 21 bp, Unknown
  Found in multiple species
gtgaggagcagatgaaggagt

SEQ ID NO: 781 - DNA, 21 bp, Unknown
  Found in multiple species
gtcttggtgggtttgagttta

SEQ ID NO: 782 - DNA, 21 bp, Unknown
  Found in multiple species
gtcggaggcggcgacggcata

SEQ ID NO: 783 - DNA, 21 bp, Unknown
  Found in multiple species
gtcgccgcctccgacttatta

SEQ ID NO: 784 - DNA, 21 bp, Unknown
  Found in multiple species
gtcccatacacaggttcattc

SEQ ID NO: 785 - DNA, 21 bp, Unknown
  Found in multiple species
gtattgagtgaggagcagatg

SEQ ID NO: 786 - DNA, 21 bp, Unknown
  Found in multiple species
gtatgggacaaaaaggaagag

SEQ ID NO: 787 - DNA, 21 bp, Unknown
  Found in multiple species
gtacaccagctccttcattat

SEQ ID NO: 788 - DNA, 21 bp, Unknown
  Found in multiple species
gtaataagtcggaggcggcga

SEQ ID NO: 789 - DNA, 21 bp, Unknown
  Found in multiple species
gtaaactcagcaaactcatga

SEQ ID NO: 790 - DNA, 21 bp, Unknown
  Found in multiple species
ggtttgagtttatgccgtcgc

SEQ ID NO: 791 - DNA, 21 bp, Unknown
  Found in multiple species
ggtttcatgagtttgctgagt

SEQ ID NO: 792 - DNA, 21 bp, Unknown
  Found in multiple species
ggttcattcattggaaacaac

SEQ ID NO: 793 - DNA, 21 bp, Unknown
  Found in multiple species
ggtgttgtttccaatgaatga

SEQ ID NO: 794 - DNA, 21 bp, Unknown
  Found in multiple species
ggtcttggtgggtttgagttt

SEQ ID NO: 795 - DNA, 21 bp, Unknown
  Found in multiple species
ggtaataagtcggaggcggcg

SEQ ID NO: 796 - DNA, 21 bp, Unknown
  Found in multiple species
gggtttgagtttatgccgtcg

SEQ ID NO: 797 - DNA, 21 bp, Unknown
  Found in multiple species
gggtttcatgagtttgctgag

SEQ ID NO: 798 - DNA, 21 bp, Unknown
  Found in multiple species
gggagatgaggaagagaagtg

SEQ ID NO: 799 - DNA, 21 bp, Unknown
  Found in multiple species
ggctcctgcaattgcttattt

SEQ ID NO: 800 - DNA, 21 bp, Unknown
  Found in multiple species
ggcggcgacggcataaactca

SEQ ID NO: 801 - DNA, 21 bp, Unknown
  Found in multiple species
ggcgacggcataaactcaaac

SEQ ID NO: 802 - DNA, 21 bp, Unknown
  Found in multiple species
ggcataaactcaaacccacca

SEQ ID NO: 803 - DNA, 21 bp, Unknown
  Found in multiple species
ggaggcggcgacggcataaac

SEQ ID NO: 804 - DNA, 21 bp, Unknown
  Found in multiple species
ggagcataatgaaggagctgg

SEQ ID NO: 805 - DNA, 21 bp, Unknown
  Found in multiple species
ggagcagatgaaggagtgtga

SEQ ID NO: 806 - DNA, 21 bp, Unknown
  Found in multiple species
ggagatgaggaagagaagtgg

SEQ ID NO: 807 - DNA, 21 bp, Unknown
  Found in multiple species
ggactttaagggagatgagga

SEQ ID NO: 808 - DNA, 21 bp, Unknown
  Found in multiple species
ggaagagaagtggtcttggtg

SEQ ID NO: 809 - DNA, 21 bp, Unknown
  Found in multiple species
ggaaacaacaccatttcatca

SEQ ID NO: 810 - DNA, 21 bp, Unknown
  Found in multiple species
gctgtaaactcagcaaactca

SEQ ID NO: 811 - DNA, 21 bp, Unknown
  Found in multiple species
gctgctgatgtattgagtgag

SEQ ID NO: 812 - DNA, 21 bp, Unknown
  Found in multiple species
gctgatgtattgagtgaggag

SEQ ID NO: 813 - DNA, 21 bp, Unknown
  Found in multiple species
gctgagtttacagctgaagat

SEQ ID NO: 814 - DNA, 21 bp, Unknown
  Found in multiple species
gctccttcattatgctccaaa

SEQ ID NO: 815 - DNA, 21 bp, Unknown
  Found in multiple species
gctcctgcaattgcttatttg

SEQ ID NO: 816 - DNA, 21 bp, Unknown
  Found in multiple species
gctcctcactcaatacatcag

SEQ ID NO: 817 - DNA, 21 bp, Unknown
  Found in multiple species
gctccaaataagtttgaattt

SEQ ID NO: 818 - DNA, 21 bp, Unknown
  Found in multiple species
gcggcgacggcataaactcaa

SEQ ID NO: 819 - DNA, 21 bp, Unknown
  Found in multiple species
gcgacggcataaactcaaacc

SEQ ID NO: 820 - DNA, 21 bp, Unknown
  Found in multiple species
gcctccgacttattaccggaa

SEQ ID NO: 821 - DNA, 21 bp, Unknown
  Found in multiple species
gccgtcgccgcctccgactta

SEQ ID NO: 822 - DNA, 21 bp, Unknown
  Found in multiple species
gccgcctccgacttattaccg

SEQ ID NO: 823 - DNA, 21 bp, Unknown
  Found in multiple species
gcataatgaaggagctggtgt

SEQ ID NO: 824 - DNA, 21 bp, Unknown
  Found in multiple species
gcataaactcaaacccaccaa

SEQ ID NO: 825 - DNA, 21 bp, Unknown
  Found in multiple species
gcagatgaaggagtgtgaaga

SEQ ID NO: 826 - DNA, 21 bp, Unknown
  Found in multiple species
gcaaactcatgaaacccagta

SEQ ID NO: 827 - DNA, 21 bp, Unknown
  Found in multiple species
gatgtattgagtgaggagcag

SEQ ID NO: 828 - DNA, 21 bp, Unknown
  Found in multiple species
gatgaggaagagaagtggtct

SEQ ID NO: 829 - DNA, 21 bp, Unknown
  Found in multiple species
gatgaaatggtgttgtttcca

SEQ ID NO: 830 - DNA, 21 bp, Unknown
  Found in multiple species
gagtttgctgagtttacagct

SEQ ID NO: 831 - DNA, 21 bp, Unknown
  Found in multiple species
gagtttatgccgtcgccgcct

SEQ ID NO: 832 - DNA, 21 bp, Unknown
  Found in multiple species
gagtttacagctgaagatgtt

SEQ ID NO: 833 - DNA, 21 bp, Unknown
  Found in multiple species
gagttggctcctgcaattgct

SEQ ID NO: 834 - DNA, 21 bp, Unknown
  Found in multiple species
gagtgaggagcagatgaagga

SEQ ID NO: 835 - DNA, 21 bp, Unknown
  Found in multiple species
gaggcggcgacggcataaact

SEQ ID NO: 836 - DNA, 21 bp, Unknown
  Found in multiple species
gaggagcagatgaaggagtgt

SEQ ID NO: 837 - DNA, 21 bp, Unknown
  Found in multiple species
gaggaagagaagtggtcttgg

SEQ ID NO: 838 - DNA, 21 bp, Unknown
  Found in multiple species
gagctgctgatgtattgagtg

SEQ ID NO: 839 - DNA, 21 bp, Unknown
  Found in multiple species
gagcataatgaaggagctggt

SEQ ID NO: 840 - DNA, 21 bp, Unknown
  Found in multiple species
gagcagatgaaggagtgtgaa

SEQ ID NO: 841 - DNA, 21 bp, Unknown
  Found in multiple species
gagatgaggaagagaagtggt

SEQ ID NO: 842 - DNA, 21 bp, Unknown
  Found in multiple species
gagaagtggtcttggtgggtt

SEQ ID NO: 843 - DNA, 21 bp, Unknown
  Found in multiple species
gactttaagggagatgaggaa

SEQ ID NO: 844 - DNA, 21 bp, Unknown
  Found in multiple species
gacttattaccggaatttgag

SEQ ID NO: 845 - DNA, 21 bp, Unknown
  Found in multiple species
gaccacttctcttcctcatct

SEQ ID NO: 846 - DNA, 21 bp, Unknown
  Found in multiple species
gaattcagccgtattggcaaa

SEQ ID NO: 847 - DNA, 21 bp, Unknown
  Found in multiple species
gaatgaacctgtgtatgggac

SEQ ID NO: 848 - DNA, 21 bp, Unknown
  Found in multiple species
gaagtggtcttggtgggtttg

SEQ ID NO: 849 - DNA, 21 bp, Unknown
  Found in multiple species
gaagagaagtggtcttggtgg

SEQ ID NO: 850 - DNA, 21 bp, Unknown
  Found in multiple species
gaacctgtgtatgggacaaaa

SEQ ID NO: 851 - DNA, 21 bp, Unknown
  Found in multiple species
gaaatggtgttgtttccaatg

SEQ ID NO: 852 - DNA, 21 bp, Unknown
  Found in multiple species
gaaacaacaccatttcatcat

SEQ ID NO: 853 - DNA, 21 bp, Unknown
  Found in multiple species
ctttttgtcccatacacaggt

SEQ ID NO: 854 - DNA, 21 bp, Unknown
  Found in multiple species
ctttaagggagatgaggaaga

SEQ ID NO: 855 - DNA, 21 bp, Unknown
  Found in multiple species
cttggtgggtttgagtttatg

SEQ ID NO: 856 - DNA, 21 bp, Unknown
  Found in multiple species
cttctcttcctcatctccctt

SEQ ID NO: 857 - DNA, 21 bp, Unknown
  Found in multiple species
cttcctttttgtcccatacac

SEQ ID NO: 858 - DNA, 21 bp, Unknown
  Found in multiple species
cttcctcatctcccttaaagt

SEQ ID NO: 859 - DNA, 21 bp, Unknown
  Found in multiple species
cttcattatgctccaaataag

SEQ ID NO: 860 - DNA, 21 bp, Unknown
  Found in multiple species
cttcatctgctcctcactcaa

SEQ ID NO: 861 - DNA, 21 bp, Unknown
  Found in multiple species
cttcagctgtaaactcagcaa

SEQ ID NO: 862 - DNA, 21 bp, Unknown
  Found in multiple species
cttcactcatcaaagccaaat

SEQ ID NO: 863 - DNA, 21 bp, Unknown
  Found in multiple species
cttcacactccttcatctgct

SEQ ID NO: 864 - DNA, 21 bp, Unknown
  Found in multiple species
cttatttggagcataatgaag

SEQ ID NO: 865 - DNA, 21 bp, Unknown
  Found in multiple species
cttattaccggaatttgagga

SEQ ID NO: 866 - DNA, 21 bp, Unknown
  Found in multiple species
ctgtgtatgggacaaaaagga

SEQ ID NO: 867 - DNA, 21 bp, Unknown
  Found in multiple species
ctgtaaactcagcaaactcat

SEQ ID NO: 868 - DNA, 21 bp, Unknown
  Found in multiple species
ctgggtttcatgagtttgctg

SEQ ID NO: 869 - DNA, 21 bp, Unknown
  Found in multiple species
ctgctgatgtattgagtgagg

SEQ ID NO: 870 - DNA, 21 bp, Unknown
  Found in multiple species
ctgctcctcactcaatacatc

SEQ ID NO: 871 - DNA, 21 bp, Unknown
  Found in multiple species
ctgatgtattgagtgaggagc

SEQ ID NO: 872 - DNA, 21 bp, Unknown
  Found in multiple species
ctgagtttacagctgaagatg

SEQ ID NO: 873 - DNA, 21 bp, Unknown
  Found in multiple species
ctcttcctttttgtcccatac

SEQ ID NO: 874 - DNA, 21 bp, Unknown
  Found in multiple species
ctcttcctcatctcccttaaa

SEQ ID NO: 875 - DNA, 21 bp, Unknown
  Found in multiple species
ctccttcattatgctccaaat

SEQ ID NO: 876 - DNA, 21 bp, Unknown
  Found in multiple species
ctccttcatctgctcctcact

SEQ ID NO: 877 - DNA, 21 bp, Unknown
  Found in multiple species
ctcctgcaattgcttatttga

SEQ ID NO: 878 - DNA, 21 bp, Unknown
  Found in multiple species
ctcctcactcaatacatcagc

SEQ ID NO: 879 - DNA, 21 bp, Unknown
  Found in multiple species
ctccgacttattaccggaatt

SEQ ID NO: 880 - DNA, 21 bp, Unknown
  Found in multiple species
ctcccttaaagtccaaaatat

SEQ ID NO: 881 - DNA, 21 bp, Unknown
  Found in multiple species
ctccaaataagtttgaatttg

SEQ ID NO: 882 - DNA, 21 bp, Unknown
  Found in multiple species
ctcatgaaacccagtaaactt

SEQ ID NO: 883 - DNA, 21 bp, Unknown
  Found in multiple species
ctcatctcccttaaagtccaa

SEQ ID NO: 884 - DNA, 21 bp, Unknown
  Found in multiple species
ctcagcaaactcatgaaaccc

SEQ ID NO: 885 - DNA, 21 bp, Unknown
  Found in multiple species
ctcactcaatacatcagcagc

SEQ ID NO: 886 - DNA, 21 bp, Unknown
  Found in multiple species
ctcaaattccggtaataagtc

SEQ ID NO: 887 - DNA, 21 bp, Unknown
  Found in multiple species
ctcaaacccaccaagaccact

SEQ ID NO: 888 - DNA, 21 bp, Unknown
  Found in multiple species
cgtcgccgcctccgacttatt

SEQ ID NO: 889 - DNA, 21 bp, Unknown
  Found in multiple species
cggtaataagtcggaggcggc

SEQ ID NO: 890 - DNA, 21 bp, Unknown
  Found in multiple species
cggcgacggcataaactcaaa

SEQ ID NO: 891 - DNA, 21 bp, Unknown
  Found in multiple species
cggaggcggcgacggcataaa

SEQ ID NO: 892 - DNA, 21 bp, Unknown
  Found in multiple species
cgcctccgacttattaccgga

SEQ ID NO: 893 - DNA, 21 bp, Unknown
  Found in multiple species
cgccgcctccgacttattacc

SEQ ID NO: 894 - DNA, 21 bp, Unknown
  Found in multiple species
cgacttattaccggaatttga

SEQ ID NO: 895 - DNA, 21 bp, Unknown
  Found in multiple species
cgacggcataaactcaaaccc

SEQ ID NO: 896 - DNA, 21 bp, Unknown
  Found in multiple species
cctttttgtcccatacacagg

SEQ ID NO: 897 - DNA, 21 bp, Unknown
  Found in multiple species
ccttcattatgctccaaataa

SEQ ID NO: 898 - DNA, 21 bp, Unknown
  Found in multiple species
ccttcatctgctcctcactca

SEQ ID NO: 899 - DNA, 21 bp, Unknown
  Found in multiple species
cctgtgtatgggacaaaaagg

SEQ ID NO: 900 - DNA, 21 bp, Unknown
  Found in multiple species
cctccgacttattaccggaat

SEQ ID NO: 901 - DNA, 21 bp, Unknown
  Found in multiple species
cctcatctcccttaaagtcca

SEQ ID NO: 902 - DNA, 21 bp, Unknown
  Found in multiple species
cctcactcaatacatcagcag

SEQ ID NO: 903 - DNA, 21 bp, Unknown
  Found in multiple species
cctcaaattccggtaataagt

SEQ ID NO: 904 - DNA, 21 bp, Unknown
  Found in multiple species
ccgtcgccgcctccgacttat

SEQ ID NO: 905 - DNA, 21 bp, Unknown
  Found in multiple species
ccggtaataagtcggaggcgg

SEQ ID NO: 906 - DNA, 21 bp, Unknown
  Found in multiple species
ccgcctccgacttattaccgg

SEQ ID NO: 907 - DNA, 21 bp, Unknown
  Found in multiple species
ccgacttattaccggaatttg

SEQ ID NO: 908 - DNA, 21 bp, Unknown
  Found in multiple species
cccatacacaggttcattcat

SEQ ID NO: 909 - DNA, 21 bp, Unknown
  Found in multiple species
cccaccaagaccacttctctt

SEQ ID NO: 910 - DNA, 21 bp, Unknown
  Found in multiple species
ccatacacaggttcattcatt

SEQ ID NO: 911 - DNA, 21 bp, Unknown
  Found in multiple species
ccagctccttcattatgctcc

SEQ ID NO: 912 - DNA, 21 bp, Unknown
  Found in multiple species
ccacttctcttcctcatctcc

SEQ ID NO: 913 - DNA, 21 bp, Unknown
  Found in multiple species
ccaccaagaccacttctcttc

SEQ ID NO: 914 - DNA, 21 bp, Unknown
  Found in multiple species
ccaatgaatgaacctgtgtat

SEQ ID NO: 915 - DNA, 21 bp, Unknown
  Found in multiple species
ccaagaccacttctcttcctc

SEQ ID NO: 916 - DNA, 21 bp, Unknown
  Found in multiple species
ccaacatcttcagctgtaaac

SEQ ID NO: 917 - DNA, 21 bp, Unknown
  Found in multiple species
catttggctttgatgagtgaa

SEQ ID NO: 918 - DNA, 21 bp, Unknown
  Found in multiple species
cattggaaacaacaccatttc

SEQ ID NO: 919 - DNA, 21 bp, Unknown
  Found in multiple species
cattcattggaaacaacacca

SEQ ID NO: 920 - DNA, 21 bp, Unknown
  Found in multiple species
cattatgctccaaataagttt

SEQ ID NO: 921 - DNA, 21 bp, Unknown
  Found in multiple species
catgagtttgctgagtttaca

SEQ ID NO: 922 - DNA, 21 bp, Unknown
  Found in multiple species
catcttcagctgtaaactcag

SEQ ID NO: 923 - DNA, 21 bp, Unknown
  Found in multiple species
catctgctcctcactcaatac

SEQ ID NO: 924 - DNA, 21 bp, Unknown
  Found in multiple species
catctcccttaaagtccaaaa

SEQ ID NO: 925 - DNA, 21 bp, Unknown
  Found in multiple species
catacacaggttcattcattg

SEQ ID NO: 926 - DNA, 21 bp, Unknown
  Found in multiple species
cataatgaaggagctggtgta

SEQ ID NO: 927 - DNA, 21 bp, Unknown
  Found in multiple species
cataaactcaaacccaccaag

SEQ ID NO: 928 - DNA, 21 bp, Unknown
  Found in multiple species
caggttcattcattggaaaca

SEQ ID NO: 929 - DNA, 21 bp, Unknown
  Found in multiple species
cagctgtaaactcagcaaact

SEQ ID NO: 930 - DNA, 21 bp, Unknown
  Found in multiple species
cagctccttcattatgctcca

SEQ ID NO: 931 - DNA, 21 bp, Unknown
  Found in multiple species
cagcaaactcatgaaacccag

SEQ ID NO: 932 - DNA, 21 bp, Unknown
  Found in multiple species
cacttctcttcctcatctccc

SEQ ID NO: 933 - DNA, 21 bp, Unknown
  Found in multiple species
cactccttcatctgctcctca

SEQ ID NO: 934 - DNA, 21 bp, Unknown
  Found in multiple species
cactcaatacatcagcagctc

SEQ ID NO: 935 - DNA, 21 bp, Unknown
  Found in multiple species
caccagctccttcattatgct

SEQ ID NO: 936 - DNA, 21 bp, Unknown
  Found in multiple species
caccaagaccacttctcttcc

SEQ ID NO: 937 - DNA, 21 bp, Unknown
  Found in multiple species
cacaggttcattcattggaaa

SEQ ID NO: 938 - DNA, 21 bp, Unknown
  Found in multiple species
cacactccttcatctgctcct

SEQ ID NO: 939 - DNA, 21 bp, Unknown
  Found in multiple species
caatgaatgaacctgtgtatg

SEQ ID NO: 940 - DNA, 21 bp, Unknown
  Found in multiple species
caagaccacttctcttcctca

SEQ ID NO: 941 - DNA, 21 bp, Unknown
  Found in multiple species
caacatcttcagctgtaaact

SEQ ID NO: 942 - DNA, 21 bp, Unknown
  Found in multiple species
caaattccggtaataagtcgg

SEQ ID NO: 943 - DNA, 21 bp, Unknown
  Found in multiple species
caaattcaaacttatttggag

SEQ ID NO: 944 - DNA, 21 bp, Unknown
  Found in multiple species
caaataagcaattgcaggagc

SEQ ID NO: 945 - DNA, 21 bp, Unknown
  Found in multiple species
caaacttatttggagcataat

SEQ ID NO: 946 - DNA, 21 bp, Unknown
  Found in multiple species
caaactcatgaaacccagtaa

SEQ ID NO: 947 - DNA, 21 bp, Unknown
  Found in multiple species
caaacccaccaagaccacttc

SEQ ID NO: 948 - DNA, 21 bp, Unknown
  Found in multiple species
attttggactttaagggagat

SEQ ID NO: 949 - DNA, 21 bp, Unknown
  Found in multiple species
atttggctttgatgagtgaag

SEQ ID NO: 950 - DNA, 21 bp, Unknown
  Found in multiple species
atttggagcataatgaaggag

SEQ ID NO: 951 - DNA, 21 bp, Unknown
  Found in multiple species
attggaaacaacaccatttca

SEQ ID NO: 952 - DNA, 21 bp, Unknown
  Found in multiple species
attgagtgaggagcagatgaa

SEQ ID NO: 953 - DNA, 21 bp, Unknown
  Found in multiple species
attccggtaataagtcggagg

SEQ ID NO: 954 - DNA, 21 bp, Unknown
  Found in multiple species
attcattggaaacaacaccat

SEQ ID NO: 955 - DNA, 21 bp, Unknown
  Found in multiple species
attcaaacttatttggagcat

SEQ ID NO: 956 - DNA, 21 bp, Unknown
  Found in multiple species
attatgctccaaataagtttg

SEQ ID NO: 957 - DNA, 21 bp, Unknown
  Found in multiple species
atgtattgagtgaggagcaga

SEQ ID NO: 958 - DNA, 21 bp, Unknown
  Found in multiple species
atggtgttgtttccaatgaat

SEQ ID NO: 959 - DNA, 21 bp, Unknown
  Found in multiple species
atgctccaaataagtttgaat

SEQ ID NO: 960 - DNA, 21 bp, Unknown
  Found in multiple species
atgccgtcgccgcctccgact

SEQ ID NO: 961 - DNA, 21 bp, Unknown
  Found in multiple species
atgatgaaatggtgttgtttc

SEQ ID NO: 962 - DNA, 21 bp, Unknown
  Found in multiple species
atgagtttgctgagtttacag

SEQ ID NO: 963 - DNA, 21 bp, Unknown
  Found in multiple species
atgaggaagagaagtggtctt

SEQ ID NO: 964 - DNA, 21 bp, Unknown
  Found in multiple species
atgaatgaacctgtgtatggg

SEQ ID NO: 965 - DNA, 21 bp, Unknown
  Found in multiple species
atgaacctgtgtatgggacaa

SEQ ID NO: 966 - DNA, 21 bp, Unknown
  Found in multiple species
atgaaatggtgttgtttccaa

SEQ ID NO: 967 - DNA, 21 bp, Unknown
  Found in multiple species
atcttcagctgtaaactcagc

SEQ ID NO: 968 - DNA, 21 bp, Unknown
  Found in multiple species
atctgctcctcactcaataca

SEQ ID NO: 969 - DNA, 21 bp, Unknown
  Found in multiple species
atctcccttaaagtccaaaat

SEQ ID NO: 970 - DNA, 21 bp, Unknown
  Found in multiple species
atattttggactttaagggag

SEQ ID NO: 971 - DNA, 21 bp, Unknown
  Found in multiple species
atacacaggttcattcattgg

SEQ ID NO: 972 - DNA, 21 bp, Unknown
  Found in multiple species
ataatgaaggagctggtgtac

SEQ ID NO: 973 - DNA, 21 bp, Unknown
  Found in multiple species
ataagtcggaggcggcgacgg

SEQ ID NO: 974 - DNA, 21 bp, Unknown
  Found in multiple species
ataagcaattgcaggagccaa

SEQ ID NO: 975 - DNA, 21 bp, Unknown
  Found in multiple species
ataaactcaaacccaccaaga

SEQ ID NO: 976 - DNA, 21 bp, Unknown
  Found in multiple species
agtttgctgagtttacagctg

SEQ ID NO: 977 - DNA, 21 bp, Unknown
  Found in multiple species
agtttatgccgtcgccgcctc

SEQ ID NO: 978 - DNA, 21 bp, Unknown
  Found in multiple species
agtttactgggtttcatgagt

SEQ ID NO: 979 - DNA, 21 bp, Unknown
  Found in multiple species
agtttacagctgaagatgttg

SEQ ID NO: 980 - DNA, 21 bp, Unknown
  Found in multiple species
agttggctcctgcaattgctt

SEQ ID NO: 981 - DNA, 21 bp, Unknown
  Found in multiple species
agtggtcttggtgggtttgag

SEQ ID NO: 982 - DNA, 21 bp, Unknown
  Found in multiple species
agtgaggagcagatgaaggag

SEQ ID NO: 983 - DNA, 21 bp, Unknown
  Found in multiple species
agtcggaggcggcgacggcat

SEQ ID NO: 984 - DNA, 21 bp, Unknown
  Found in multiple species
aggttcattcattggaaacaa

SEQ ID NO: 985 - DNA, 21 bp, Unknown
  Found in multiple species
agggagatgaggaagagaagt

SEQ ID NO: 986 - DNA, 21 bp, Unknown
  Found in multiple species
aggcggcgacggcataaactc

SEQ ID NO: 987 - DNA, 21 bp, Unknown
  Found in multiple species
aggagcagatgaaggagtgtg

SEQ ID NO: 988 - DNA, 21 bp, Unknown
  Found in multiple species
aggaagagaagtggtcttggt

SEQ ID NO: 989 - DNA, 21 bp, Unknown
  Found in multiple species
agctgtaaactcagcaaactc

SEQ ID NO: 990 - DNA, 21 bp, Unknown
  Found in multiple species
agctgctgatgtattgagtga

SEQ ID NO: 991 - DNA, 21 bp, Unknown
  Found in multiple species
agctccttcattatgctccaa

SEQ ID NO: 992 - DNA, 21 bp, Unknown
  Found in multiple species
agcataatgaaggagctggtg

SEQ ID NO: 993 - DNA, 21 bp, Unknown
  Found in multiple species
agcagatgaaggagtgtgaag

SEQ ID NO: 994 - DNA, 21 bp, Unknown
  Found in multiple species
agcaattgcaggagccaactc

SEQ ID NO: 995 - DNA, 21 bp, Unknown
  Found in multiple species
agcaaactcatgaaacccagt

SEQ ID NO: 996 - DNA, 21 bp, Unknown
  Found in multiple species
agatgaggaagagaagtggtc

SEQ ID NO: 997 - DNA, 21 bp, Unknown
  Found in multiple species
agagctgctgatgtattgagt

SEQ ID NO: 998 - DNA, 21 bp, Unknown
  Found in multiple species
agagaagtggtcttggtgggt

SEQ ID NO: 999 - DNA, 21 bp, Unknown
  Found in multiple species
agaccacttctcttcctcatc

SEQ ID NO: 1000 - DNA, 21 bp, Unknown
  Found in multiple species
agaagtggtcttggtgggttt

SEQ ID NO: 1001 - DNA, 21 bp, Unknown
  Found in multiple species
actttaagggagatgaggaag

SEQ ID NO: 1002 - DNA, 21 bp, Unknown
  Found in multiple species
acttctcttcctcatctccct

SEQ ID NO: 1003 - DNA, 21 bp, Unknown
  Found in multiple species
acttatttggagcataatgaa

SEQ ID NO: 1004 - DNA, 21 bp, Unknown
  Found in multiple species
acttattaccggaatttgagg

SEQ ID NO: 1005 - DNA, 21 bp, Unknown
  Found in multiple species
actgggtttcatgagtttgct

SEQ ID NO: 1006 - DNA, 21 bp, Unknown
  Found in multiple species
actccttcatctgctcctcac

SEQ ID NO: 1007 - DNA, 21 bp, Unknown
  Found in multiple species
actcatgaaacccagtaaact

SEQ ID NO: 1008 - DNA, 21 bp, Unknown
  Found in multiple species
actcagcaaactcatgaaacc

SEQ ID NO: 1009 - DNA, 21 bp, Unknown
  Found in multiple species
actcaatacatcagcagctct

SEQ ID NO: 1010 - DNA, 21 bp, Unknown
  Found in multiple species
actcaaacccaccaagaccac

SEQ ID NO: 1011 - DNA, 21 bp, Unknown
  Found in multiple species
acctgtgtatgggacaaaaag

SEQ ID NO: 1012 - DNA, 21 bp, Unknown
  Found in multiple species
acccaccaagaccacttctct

SEQ ID NO: 1013 - DNA, 21 bp, Unknown
  Found in multiple species
accagctccttcattatgctc

SEQ ID NO: 1014 - DNA, 21 bp, Unknown
  Found in multiple species
accacttctcttcctcatctc

SEQ ID NO: 1015 - DNA, 21 bp, Unknown
  Found in multiple species
accaagaccacttctcttcct

SEQ ID NO: 1016 - DNA, 21 bp, Unknown
  Found in multiple species
acatcttcagctgtaaactca

SEQ ID NO: 1017 - DNA, 21 bp, Unknown
  Found in multiple species
acaggttcattcattggaaac

SEQ ID NO: 1018 - DNA, 21 bp, Unknown
  Found in multiple species
acactccttcatctgctcctc

SEQ ID NO: 1019 - DNA, 21 bp, Unknown
  Found in multiple species
acaccagctccttcattatgc

SEQ ID NO: 1020 - DNA, 21 bp, Unknown
  Found in multiple species
acacaggttcattcattggaa

SEQ ID NO: 1021 - DNA, 21 bp, Unknown
  Found in multiple species
aattccggtaataagtcggag

SEQ ID NO: 1022 - DNA, 21 bp, Unknown
  Found in multiple species
aattcaaacttatttggagca

SEQ ID NO: 1023 - DNA, 21 bp, Unknown
  Found in multiple species
aatggtgttgtttccaatgaa

SEQ ID NO: 1024 - DNA, 21 bp, Unknown
  Found in multiple species
aatgatgaaatggtgttgttt

SEQ ID NO: 1025 - DNA, 21 bp, Unknown
  Found in multiple species
aatgaatgaacctgtgtatgg

SEQ ID NO: 1026 - DNA, 21 bp, Unknown
  Found in multiple species
aatgaacctgtgtatgggaca

SEQ ID NO: 1027 - DNA, 21 bp, Unknown
  Found in multiple species
aataagtcggaggcggcgacg

SEQ ID NO: 1028 - DNA, 21 bp, Unknown
  Found in multiple species
aataagcaattgcaggagcca

SEQ ID NO: 1029 - DNA, 21 bp, Unknown
  Found in multiple species
aagtttactgggtttcatgag

SEQ ID NO: 1030 - DNA, 21 bp, Unknown
  Found in multiple species
aagtggtcttggtgggtttga

SEQ ID NO: 1031 - DNA, 21 bp, Unknown
  Found in multiple species
aagtcggaggcggcgacggca

SEQ ID NO: 1032 - DNA, 21 bp, Unknown
  Found in multiple species
aagggagatgaggaagagaag

SEQ ID NO: 1033 - DNA, 21 bp, Unknown
  Found in multiple species
aagcaattgcaggagccaact

SEQ ID NO: 1034 - DNA, 21 bp, Unknown
  Found in multiple species
aagagaagtggtcttggtggg

SEQ ID NO: 1035 - DNA, 21 bp, Unknown
  Found in multiple species
aagaccacttctcttcctcat

SEQ ID NO: 1036 - DNA, 21 bp, Unknown
  Found in multiple species
aacttatttggagcataatga

SEQ ID NO: 1037 - DNA, 21 bp, Unknown
  Found in multiple species
aactcatgaaacccagtaaac

SEQ ID NO: 1038 - DNA, 21 bp, Unknown
  Found in multiple species
aactcagcaaactcatgaaac

SEQ ID NO: 1039 - DNA, 21 bp, Unknown
  Found in multiple species
aactcaaacccaccaagacca

SEQ ID NO: 1040 - DNA, 21 bp, Unknown
  Found in multiple species
aacctgtgtatgggacaaaaa

SEQ ID NO: 1041 - DNA, 21 bp, Unknown
  Found in multiple species
aacccaccaagaccacttctc

SEQ ID NO: 1042 - DNA, 21 bp, Unknown
  Found in multiple species
aacatcttcagctgtaaactc

SEQ ID NO: 1043 - DNA, 21 bp, Unknown
  Found in multiple species
aaattccggtaataagtcgga

SEQ ID NO: 1044 - DNA, 21 bp, Unknown
  Found in multiple species
aaattcaaacttatttggagc

SEQ ID NO: 1045 - DNA, 21 bp, Unknown
  Found in multiple species
aaatggtgttgtttccaatga

SEQ ID NO: 1046 - DNA, 21 bp, Unknown
  Found in multiple species
aaataagcaattgcaggagcc

SEQ ID NO: 1047 - DNA, 21 bp, Unknown
  Found in multiple species
aaacttatttggagcataatg

SEQ ID NO: 1048 - DNA, 21 bp, Unknown
  Found in multiple species
aaactcatgaaacccagtaaa

SEQ ID NO: 1049 - DNA, 21 bp, Unknown
  Found in multiple species
aaactcagcaaactcatgaaa

SEQ ID NO: 1050 - DNA, 21 bp, Unknown
  Found in multiple species
aaactcaaacccaccaagacc

SEQ ID NO: 1051 - DNA, 21 bp, Unknown
  Found in multiple species
aaacccaccaagaccacttct

SEQ ID NO: 1052 - DNA, 21 bp, Unknown
  Found in multiple species
aaacaacaccatttcatcatt

SEQ ID NO: 1053 - DNA, 21 bp, Unknown
  Found in multiple species
tgggttgaattcagccgtatt

SEQ ID NO: 1054 - DNA, 21 bp, Unknown
  Found in multiple species
tgaattcaacccactttcact

SEQ ID NO: 1055 - DNA, 21 bp, Unknown
  Found in multiple species
tgaaagtgggttgaattcagc

SEQ ID NO: 1056 - DNA, 21 bp, Unknown
  Found in multiple species
tacggctgaattcaacccact

SEQ ID NO: 1057 - DNA, 21 bp, Unknown
  Found in multiple species
gttgaattcagccgtattggc

SEQ ID NO: 1058 - DNA, 21 bp, Unknown
  Found in multiple species
gtgggttgaattcagccgtat

SEQ ID NO: 1059 - DNA, 21 bp, Unknown
  Found in multiple species
gtgaaagtgggttgaattcag

SEQ ID NO: 1060 - DNA, 21 bp, Unknown
  Found in multiple species
ggttgaattcagccgtattgg

SEQ ID NO: 1061 - DNA, 21 bp, Unknown
  Found in multiple species
gggttgaattcagccgtattg

SEQ ID NO: 1062 - DNA, 21 bp, Unknown
  Found in multiple species
ggctgaattcaacccactttc

SEQ ID NO: 1063 - DNA, 21 bp, Unknown
  Found in multiple species
gctgaattcaacccactttca

SEQ ID NO: 1064 - DNA, 21 bp, Unknown
  Found in multiple species
gccaatacggctgaattcaac

SEQ ID NO: 1065 - DNA, 21 bp, Unknown
  Found in multiple species
gagtgaaagtgggttgaattc

SEQ ID NO: 1066 - DNA, 21 bp, Unknown
  Found in multiple species
gaattcaacccactttcactc

SEQ ID NO: 1067 - DNA, 21 bp, Unknown
  Found in multiple species
gaaagtgggttgaattcagcc

SEQ ID NO: 1068 - DNA, 21 bp, Unknown
  Found in multiple species
ctgaattcaacccactttcac

SEQ ID NO: 1069 - DNA, 21 bp, Unknown
  Found in multiple species
cggctgaattcaacccacttt

SEQ ID NO: 1070 - DNA, 21 bp, Unknown
  Found in multiple species
cgagtgaaagtgggttgaatt

SEQ ID NO: 1071 - DNA, 21 bp, Unknown
  Found in multiple species
ccaatacggctgaattcaacc

SEQ ID NO: 1072 - DNA, 21 bp, Unknown
  Found in multiple species
caatacggctgaattcaaccc

SEQ ID NO: 1073 - DNA, 21 bp, Unknown
  Found in multiple species
attcaacccactttcactcgt

SEQ ID NO: 1074 - DNA, 21 bp, Unknown
  Found in multiple species
atacggctgaattcaacccac

SEQ ID NO: 1075 - DNA, 21 bp, Unknown
  Found in multiple species
agtgggttgaattcagccgta

SEQ ID NO: 1076 - DNA, 21 bp, Unknown
  Found in multiple species
agtgaaagtgggttgaattca

SEQ ID NO: 1077 - DNA, 21 bp, Unknown
  Found in multiple species
acggctgaattcaacccactt

SEQ ID NO: 1078 - DNA, 21 bp, Unknown
  Found in multiple species
acgagtgaaagtgggttgaat

SEQ ID NO: 1079 - DNA, 21 bp, Unknown
  Found in multiple species
aattcaacccactttcactcg

SEQ ID NO: 1080 - DNA, 21 bp, Unknown
  Found in multiple species
aatacggctgaattcaaccca

SEQ ID NO: 1081 - DNA, 21 bp, Unknown
  Found in multiple species
aagtgggttgaattcagccgt

SEQ ID NO: 1082 - DNA, 21 bp, Unknown
  Found in multiple species
aaagtgggttgaattcagccg

SEQ ID NO: 1083 - DNA, 21 bp, Amaranthus palmeri
tccgtagcttacataccgaag

SEQ ID NO: 1084 - DNA, 21 bp, Amaranthus palmeri
tccaagtgaataggagaaaca

SEQ ID NO: 1085 - DNA, 21 bp, Amaranthus palmeri
agcagcttctgcgtcttctac

SEQ ID NO: 1086 - DNA, 21 bp, Amaranthus palmeri
acagcacgcacgccaagaccg

SEQ ID NO: 1087 - DNA, 21 bp, Amaranthus palmeri
cgatgtaaggaatttggtaaa

SEQ ID NO: 1088 - DNA, 21 bp, Amaranthus palmeri
cgaggggattgcagcagaaga

SEQ ID NO: 1089 - DNA, 21 bp, Amaranthus palmeri
gtaggagaatacggtgaagta

SEQ ID NO: 1090 - DNA, 21 bp, Amaranthus palmeri
gaccccaagaaaatcgtctgc

SEQ ID NO: 1091 - DNA, 21 bp, Amaranthus palmeri
attgaggagtacgagaagact

SEQ ID NO: 1092 - DNA, 21 bp, Amaranthus palmeri
cttgaacgtaaacaggttcca

(19) United States
(12) Patent Application Publication
(10) Pub. No.: US 2013/0097726 A1
(43) Pub. Date: Apr. 18, 2013

(54) METHODS AND COMPOSITIONS FOR WEED CONTROL

(71) Applicants: DANIEL ADER, St. Louis, MO (US); John J. Finnessy, Des Peres, MO (US); Mahak Kapoor, Creve Coeur, MO (US); James D. Masucci, Manchester, MO (US); Zhaolong Li, St. Charles, MO (US); Ronak Hasmukh Shah, Chesterfield, MO (US); Nengbing Tao, O'Fallon, MO (US); Jennifer Chou Taylor, Ballwin, MO (US); Dafu Wang, Chesterfield, MO (US); Heping Yang, Chesterfield, MO (US)

(72) Inventors: DANIEL ADER, St. Louis, MO (US); John J. Finnessy, Des Peres, MO (US); Mahak Kapoor, Creve Coeur, MO (US); James D. Masucci, Manchester, MO (US); Zhaolong Li, St. Charles, MO (US); Ronak Hasmukh Shah, Chesterfield, MO (US); Nengbing Tao, O'Fallon, MO (US); Jennifer Chou Taylor, Ballwin, MO (US); Dafu Wang, Chesterfield, MO (US); Heping Yang, Chesterfield, MO (US)

(21) Appl. No.: 13/612,936

(22) Filed: Sep. 13, 2012

Related U.S. Application Data
(60) Provisional application No. 61/534,066, filed on Sep. 13, 2011.

(51) Int. Cl. C12N 15/82

(52) U.S. Cl. CPC C12N 15/8243
USPC 800/278; 435/320.1; 435/91.1; 504/348; 504/118; 504/196; 504/234

(57) ABSTRACT

The present invention provides novel compositions for use to enhance weed control. Specifically, the present invention provides for methods and compositions that modulate 4-hydroxyphenyl-pyruvate-dioxygenase in weed species. The present invention also provides for combinations of compositions and methods that enhance weed control.

This application claims benefit under 35USC §119(e) of U.S. provisional application Ser. No. 61/534,066 filed Sep. 13, 2011, herein incorporated by reference in it's entirety. The sequence listing that is contained in the file named “40_{—}21(58636)B seq listing.txt”, which is 400,732 bytes (measured in operating system MS-Windows) and was created on 7 Sep. 2012, is filed herewith and incorporated herein by reference.

### FIELD

The invention relates generally to the field of weed management. More specifically, the invention relates to 4-hydroxyphenyl-pyruvate-dioxygenase genes in weedy plants and compositions containing polynucleotide molecules for modulating their expression. The invention further provides methods and compositions useful for weed control.

### BACKGROUND

Weeds are plants that compete with cultivated plants in an agronomic environment and cost farmers billions of dollars annually in crop losses and the expense of efforts to keep weeds under control. Weeds also serve as hosts for crop diseases and insect pests. The losses caused by weeds in agricultural production environments include decreases in crop yield, reduced crop quality, increased irrigation costs, increased harvesting costs, reduced land value, injury to livestock, and crop damage from insects and diseases harbored by the weeds. The principal means by which weeds cause these effects are: 1) competing with crop plants for water, nutrients, sunlight and other essentials for growth and development, 2) production of toxic or irritant chemicals that cause human or animal health problem, 3) production of immense quantities of seed or vegetative reproductive parts or both that contaminate agricultural products and perpetuate the species in agricultural lands, and 4) production on agricultural and nonagricultural lands of vast amounts of vegetation that must be disposed of. Herbicide tolerant weeds are a problem with nearly all herbicides in use, there is a need to effectively manage these weeds. There are over 365 weed biotypes currently identified as being herbicide resistant to one or more herbicides by the Herbicide Resistance Action Committee (HRAC), the North American Herbicide Resistance Action Committee (NAHRAC), and the Weed Science Society of America (WSSA).

The 4-hydroxyphenyl-pyruvate-dioxygenase (HPPD) is an Fe-containing enzyme, that catalyzes the second reaction in the catabolism of tyrosine, the conversion of 4-hydroxyphenylpyruvate to homogentisate. This enzyme is the target of many herbicides that include members of the chemical families of Triketones, Isoxazoles, and Pyrazoles.

### SUMMARY OF THE INVENTION

In one aspect, the invention provides a method of weedy plant control comprising an external application to a weedy plant of a composition comprising a polynucleotide and a transfer agent, wherein the polynucleotide is essentially identical or essentially complementary to an HPPD gene sequence or fragment thereof, or to the RNA transcript of said HPPD gene sequence or fragment thereof, wherein said HPPD gene sequence is selected from the group consisting of SEQ ID NO:1-32 or a polynucleotide fragment thereof, whereby the weedy plant growth or development or reproductive ability is reduced or the weedy plant is made more sensitive to an HPPD inhibitor herbicide relative to a weedy plant not treated with said composition. In this manner, plants that have become resistant to the application of glyphosate containing herbicides may be made more susceptible to the herbicidal effects of a glyphosate containing herbicide, thus potentiating the effect of the herbicide. The polynucleotide fragment is at least 18 contiguous nucleotides, at least 19 contiguous nucleotides, at least 20 contiguous nucleotides or at least 21 contiguous nucleotides in length and at least 85 percent identical to an HPPD gene sequence selected from the group consisting of SEQ ID NO:1-32 and the transfer agent is an organosilicone composition or compound. The polynucleotide fragment can also be sense or anti-sense ssDNA or ssRNA, dsRNA, or dsDNA, or dsDNA/RNA hybrids. The composition can include more than one polynucleotide fragments, and the composition can include an HPPD inhibitor herbicide and/or other herbicides that enhance the weed control activity of the composition.

In another aspect of the invention, polynucleotide molecules and methods for modulating HPPD gene expression in weedy plant species are provided. The method reduces, represses or otherwise delays expression of an HPPD gene in a plant comprising an external application to a weedy plant of a composition comprising a polynucleotide and a transfer agent, wherein the polynucleotide is essentially identical or essentially complementary to an HPPD gene sequence or fragment thereof, or to the RNA transcript of the HPPD gene sequence or fragment thereof, wherein the HPPD gene sequence is selected from the group consisting of SEQ ID NO:1-32 or a polynucleotide fragment thereof. The polynucleotide fragment is at least 18 contiguous nucleotides, at least 19 contiguous nucleotides, at least 20 contiguous nucleotides at least 21 contiguous nucleotides in length and at least 85 percent identical to an HPPD gene sequence selected from the group consisting of SEQ ID NO:1-32 and the transfer agent is an organosilicone compound. The polynucleotide fragment can also be sense or anti-sense ssDNA or ssRNA, dsRNA, or dsDNA, or dsDNA/RNA hybrids.

In a further aspect of the invention, the polynucleotide molecule containing composition of the invention may be combined with other herbicidal (co-herbicides) compounds to provide additional control of unwanted plants in a field of cultivated plants.

In a further aspect, the polynucleotide molecule composition may be combined with any one or more additional agricultural chemicals, such as, insecticides, fungicides, nematocides, bactericides, acaricides, growth regulators, chemosterilants, semiochemicals, repellents, attractants, pheromones, feeding stimulants, biopesticides, microbial pesticides or other biologically active compounds to form a multi-component pesticide giving an even broader spectrum of agricultural protection.

### BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein. The invention can be more fully understood from the following description of the figures:

FIG. 1. Treatment of Amaranthus palmeri plants with ssDNA trigger polynucleotides and HPPD inhibitor herbicide, Mesotrione.

### DETAILED DESCRIPTION

Provided are methods and compositions containing a polynucleotide that provide for regulation, repression or delay of HPPD (4-hydroxyphenyl-pyruvate-dioxygenase) gene expression and enhanced control of weedy plant species amd importantly HPPD inhibitor herbicide resistant weed biotypes. Aspects of the method can be applied to manage various weedy plants in agronomic and other cultivated environments.

The following definitions and methods are provided to better define the present invention and to guide those of ordinary skill in the art in the practice of the present invention. Unless otherwise noted, terms are to be understood according to conventional usage by those of ordinary skill in the relevant art. Where a term is provided in the singular, the inventors also contemplate aspects of the invention described by the plural of that term.

By “non-transcribable” polynucleotides is meant that the polynucleotides do not comprise a complete polymerase II transcription unit.

As used herein “solution” refers to homogeneous mixtures and non-homogeneous mixtures such as suspensions, colloids, micelles, and emulsions.

Weedy plants are plants that compete with cultivated plants, those of particular importance include, but are not limited to important invasive and noxious weeds and herbicide resistant biotypes in crop production, such as, Amaranthus species—A. albus, A. blitoides, A. hybridus, A. palmeri, A. powellii, A. retroflexus, A. spinosus, A. tuberculatus, and A. viridis; Ambrosia species—A. trifida, A. artemisifolia; Lolium species—L. multiflorum, L. rigidium, L perenne; Digitaria species—D. insularis; Euphorbia species—E. heterophylla; Kochia species—K. scoparia; Sorghum species—S. halepense; Conyza species—C. bonariensis, C. canadensis, C. sumatrensis; Chloris species—C. truncate; Echinochola species—E. colona, E. crus-galli; Eleusine species—E. indica; Poa species—P. annua; Plantago species—P. lanceolata; Avena species—A. fatua; Chenopodium species—C. album; Setaria species—S. viridis, Abutilon theophrasti, Ipomoea species, Sesbania, species, Cassia species, Sida species, Brachiaria, species and Solanum species.

Additional weedy plant species found in cultivated areas include Alopecurus myosuroides, Avena sterilis, Avena sterilis ludoviciana, Brachiaria plantaginea, Bromus diandrus, Bromus rigidus, Cynosurus echinatus, Digitaria ciliaris, Digitaria ischaemum, Digitaria sanguinalis, Echinochloa oryzicola, Echinochloa phyllopogon, Eriochloa punctata, Hordeum glaucum, Hordeum leporinum, Ischaemum rugosum, Leptochloa chinensis, Lolium persicum, Phalaris minor, Phalaris paradoxa, Rottboellia exalta, Setaria faberi, Setaria viridis var, robusta-alba schreiber, Setaria viridis var, robusta-purpurea, Snowdenia polystachea, Sorghum sudanese, Alisma plantago-aquatica, Amaranthus lividus, Amaranthus quitensis, Ammania auriculata, Ammania coccinea, Anthemis cotula, Apera spica-venti, Bacopa rotundifolia, Bidens pilosa, Bidens subalternans, Brassica tournefortii, Bromus tectorum, Camelina microcarpa, Chrysanthemum coronarium, Cuscuta campestris, Cyperus difformis, Damasonium minus, Descurainia sophia, Diplotaxis tenuifolia, Echium plantagineum, Elatine triandra var, pedicellate, Euphorbia heterophylla, Fallopia convolvulus, Fimbristylis miliacea, Galeopsis tetrahit, Galium spurium, Helianthus annuus, Iva xanthifolia, Ixophorus unisetus, Ipomoea indica, Ipomoea purpurea, Ipomoea sepiaria, Ipomoea aquatic, Ipomoea triloba, Lactuca serriola, Limnocharis flava, Limnophila erecta, Limnophila sessiliflora, Lindernia dubia, Lindernia dubia var, major, Lindernia micrantha, Lindernia procumbens, Mesembryanthemum crystallinum, Monochoria korsakowii, Monochoria vaginalis, Neslia paniculata, Papaver rhoeas, Parthenium hysterophorus, Pentzia suffruticosa, Phalaris minor, Raphanus raphanistrum, Raphanus sativus, Rapistrum rugosum, Rotala indica var, uliginosa, Sagittaria guyanensis, Sagittaria montevidensis, Sagittaria pygmaea, Salsola iberica, Scirpus juncoides var, ohwianus, Scirpus mucronatus, Setaria lutescens, Sida spinosa, Sinapis arvensis, Sisymbrium orientale, Sisymbrium thellungii, Solanum ptycanthum, Sonchus aspen, Sonchus oleraceus, Sorghum bicolor, Stellaria media, Thlaspi arvense, Xanthium strumarium, Arctotheca calendula, Conyza sumatrensis, Crassocephalum crepidiodes, Cuphea carthagenenis, Epilobium adenocaulon, Erigeron philadelphicus, Landoltia punctata, Lepidium virginicum, Monochoria korsakowii, Solanum americanum, Solanum nigrum, Vulpia bromoides, Youngia japonica, Hydrilla verticillata, Carduus nutans, Carduus pycnocephalus, Centaurea solstitialis, Cirsium arvense, Commelina diffusa, Convolvulus arvensis, Daucus carota, Digitaria ischaemum, Echinochloa crus-pavonis, Fimbristylis miliacea, Galeopsis tetrahit, Galium spurium, Limnophila erecta, Matricaria perforate, Papaver rhoeas, Ranunculus acris, Soliva sessilis, Sphenoclea zeylanica, Stellaria media, Nassella trichotoma, Stipa neesiana, Agrostis stolonifera, Polygonum aviculare, Alopecurus japonicus, Beckmannia syzigachne, Bromus tectorum, Chloris inflate, Echinochloa erecta, Portulaca oleracea, and Senecio vulgaris. It is believed that all plants contain a phytoene desaturase gene in their genome, the sequence of which can be isolated and polynucleotides made according to the methods of the present invention that are useful for regulation, suppressing or delaying the expression of the target HPPD gene in the plants and the growth or development of the treated plants.

A cultivated plant may also be considered a weedy plant when they occur in unwanted environments. For example, corn plants growing in a soybean field. Transgenic crops with one or more herbicide tolerances will need specialized methods of management to control weeds and volunteer crop plants. The present invention enables the targeting of a transgene for herbicide tolerance to permit the treated plants to become sensitive to the herbicide. For example, transgene HPPD DNA sequences in transgenic events that include FG72.

A “trigger” or “trigger polynucleotide” is a polynucleotide molecule that is homologous or complementary to a target gene polynucleotide. The trigger polynucleotide molecules modulate expression of the target gene when topically applied to a plant surface with a transfer agent, whereby a plant treated with said composition has its growth or development or reproductive ability regulated, suppressed or delayed or said plant is more sensitive to a EPSPS inhibitor herbicide as a result of said polynucleotide containing composition relative to a plant not treated with a composition containing the trigger molecule. Trigger polynucleotides disclosed herein are generally described in relation to the target gene sequence and maybe used in the sense (homologous) or antisense (complementary) orientation as single stranded molecules or comprise both strands as double stranded molecules or nucleotide variants and modified nucleotides thereof depending on the various regions of a gene being targeted.

It is contemplated that the composition of the present invention will contain multiple polynucleotides and herbicides that include but not limited to HPPD gene trigger polynucleotides and an HPPD inhibitor herbicide and anyone or more additional herbicide target gene trigger polynucleotides and the related herbicides and anyone or more additional essential gene trigger polynucleotides. Essential genes are genes in a plant that provide key enzymes or other proteins, for example, a biosynthetic enzyme, metabolizing enzyme, receptor, signal transduction protein, structural gene product, transcription factor, or transport protein; or regulating RNAs, such as, microRNAs, that are essential to the growth or survival of the organism or cell or involved in the normal growth and development of the plant (Meinke, et al., Trends Plant Sci. 2008 September; 13(9):483-91). The suppression of an essential gene enhances the effect of a herbicide that affects the function of a gene product different than the suppressed essential gene. The compositions of the present invention can include various trigger polynucleotides that modulate the expression of an essential gene other than HPPD.

Herbicides for which transgenes for plant tolerance have been demonstrated and the method can be applied, include but are not limited to: auxin-like herbicides, glyphosate, glufosinate, sulfonylureas, imidazolinones, bromoxynil, delapon, dicamba, cyclohezanedione, protoporphyrionogen oxidase inhibitors, 4-hydroxyphenyl-pyruvate-dioxygenase inhibitors herbicides. For example, transgenes and their polynucleotide molecules that encode proteins involved in herbicide tolerance are known in the art, and include, but are not limited to an 5-enolpyruvylshikimate-3-phosphate synthase (EPSPS), for example, as more fully described in U.S. Pat. Nos. 7,807,791 (SEQ ID NO:5); 6,248,876 B1; 5,627,061; 5,804,425; 5,633,435; 5,145,783; 4,971,908; 5,312,910; 5,188,642; 4,940,835; 5,866,775; 6,225,114 B1; 6,130,366; 5,310,667; 4,535,060; 4,769,061; 5,633,448; 5,510,471; U.S. Pat. No. Re. 36,449; U.S. Pat. Nos. RE 37,287 E; and 5,491,288; tolerance to sulfonylurea and/or imidazolinone, for example, as described more fully in U.S. Pat. Nos. 5,605,011; 5,013,659; 5,141,870; 5,767,361; 5,731,180; 5,304,732; 4,761,373; 5,331,107; 5,928,937; and 5,378,824; and international publication WO 96/33270; tolerance to hydroxyphenylpyruvatedioxygenases inhibitiong herbicides in plants are described in U.S. Pat. Nos. 6,245,968 B1; 6,268,549; and 6,069,115; US Pat. Pub. 20110191897 and U.S. Pat. No. 7,312,379 SEQ ID NO:3; U.S. Pat. No. 7,935,869; U.S. Pat. No. 7,304,209, SEQ ID NO:1, 3, 5 and 15; aryloxyalkanoate dioxygenase polynucleotides, which confer tolerance to 2,4-D and other phenoxy auxin herbicides as well as to aryloxyphenoxypropionate herbicides as described, for example, in WO2005/107437; U.S. Pat. No. 7,838,733 SEQ ID NO:5;) and dicamba-tolerance polynucleotides as described, for example, in Herman et al. (2005) J. Biol. Chem. 280: 24759-24767. Other examples of herbicide-tolerance traits include those conferred by polynucleotides encoding an exogenous phosphinothricin acetyltransferase, as described in U.S. Pat. Nos. 5,969,213; 5,489,520; 5,550,318; 5,874,265; 5,919,675; 5,561,236; 5,648,477; 5,646,024; 6,177,616; and 5,879,903. Plants containing an exogenous phosphinothricin acetyltransferase can exhibit improved tolerance to glufosinate herbicides, which inhibit the enzyme glutamine synthase. Additionally, herbicide-tolerance polynucleotides include those conferred by polynucleotides conferring altered protoporphyrinogen oxidase (protox) activity, as described in U.S. Pat. Nos. 6,288,306 B1; 6,282,837 B1; and 5,767,373; and WO 01/12825. Plants containing such polynucleotides can exhibit improved tolerance to any of a variety of herbicides which target the protox enzyme (also referred to as protox inhibitors). Polynucleotides encoding a glyphosate oxidoreductase and a glyphosate-N-acetyl transferase (GOX described in U.S. Pat. No. 5,463,175 and GAT described in U.S. Patent publication 20030083480, dicamba monooxygenase U.S. Patent publication 20030135879, all of which are incorporated herein by reference); a polynucleotide molecule encoding bromoxynil nitrilase (Bxn described in U.S. Pat. No. 4,810,648 for Bromoxynil tolerance, which is incorporated herein by reference); a polynucleotide molecule encoding phytoene desaturase (crtl) described in Misawa et al, (1993) Plant J. 4:833-840 and Misawa et al, (1994) Plant J. 6:481-489 for norflurazon tolerance; a polynucleotide molecule encoding acetohydroxyacid synthase (AHAS, aka ALS) described in Sathasiivan et al. (1990) Nucl. Acids Res. 18:318-2193 for tolerance to sulfonylurea herbicides; and the bar gene described in DeBlock, et al. (1987) EMBO J. 6:2513-2519 for glufosinate and bialaphos tolerance. The transgenic coding regions and regulatory elements of the herbicide tolerance genes are targets in which polynucleotide triggers and herbicides can be included in the composition of the present invention.

The composition of the present invention include a component that is an HPPD inhibitor herbicide which includes but are not limited to Triketones, such as, mesotrione, tefuryltrione, tembotrione, and sulcotrione; Isoxazoles, such as, isoxachlortole, pyrasulfotole, and isoxaflutole; Pyrazoles, such as, benzofenap, pyrazolynate, topramezone and pyrazoxyfen. Additional HPPD inhibitors include benzobicyclon and bicyclopyrone,

Numerous herbicides with similar or different modes of action (herein referred to as co-herbicides) are available that can be added to the composition, for example, members of the herbicide families that include but are not limited to amide herbicides, aromatic acid herbicides, arsenical herbicides, benzothiazole herbicides, benzoylcyclohexanedione herbicides, benzofuranyl alkylsulfonate herbicides, carbamate herbicides, cyclohexene oxime herbicides, cyclopropylisoxazole herbicides, dicarboximide herbicides, dinitroaniline herbicides, dinitrophenol herbicides, diphenyl ether herbicides, dithiocarbamate herbicides, halogenated aliphatic herbicides, imidazolinone herbicides, inorganic herbicides, nitrile herbicides, organophosphorus herbicides, oxadiazolone herbicides, oxazole herbicides, phenoxy herbicides, phenylenediamine herbicides, pyrazole herbicides, pyridazine herbicides, pyridazinone herbicides, pyridine herbicides, pyrimidinediamine herbicides, pyrimidinyloxybenzylamine herbicides, quaternary ammonium herbicides, thiocarbamate herbicides, thiocarbonate herbicides, thiourea herbicides, triazine herbicides, triazinone herbicides, triazole herbicides, triazolone herbicides, triazolopyrimidine herbicides, uracil herbicides, and urea herbicides. In particular, the rates of use of the added herbicides can be reduced in compositions comprising the polynucleotides of the invention. Use rate reductions of the additional added herbicides can be 10-25 percent, 26-50 percent, 51-75 percent or more can be achieved that enhance the activity of the polynucleotides and herbicide composition and is contemplated. Representative herbicides of the families include but are not limited to acetochlor, acifluorfen, acifluorfen-sodium, aclonifen, acrolein, alachlor, alloxydim, allyl alcohol, ametryn, amicarbazone, amidosulfuron, aminopyralid, amitrole, ammonium sulfamate, anilofos, asulam, atraton, atrazine, azimsulfuron, BCPC, beflubutamid, benazolin, benfluralin, benfuresate, bensulfuron, bensulfuron-methyl, bensulide, bentazone, benzfendizone, benzobicyclon, benzofenap, bifenox, bilanafos, bispyribac, bispyribac-sodium, borax, bromacil, bromobutide, bromoxynil, butachlor, butafenacil, butamifos, butralin, butroxydim, butylate, cacodylic acid, calcium chlorate, cafenstrole, carbetamide, carfentrazone, carfentrazone-ethyl, CDEA, CEPC, chlorflurenol, chlorflurenol-methyl, chloridazon, chlorimuron, chlorimuron-ethyl, chloroacetic acid, chlorotoluron, chlorpropham, chlorsulfuron, chlorthal, chlorthal-dimethyl, cinidon-ethyl, cinmethylin, cinosulfuron, cisanilide, clethodim, clodinafop, clodinafop-propargyl, clomazone, clomeprop, clopyralid, cloransulam, cloransulam-methyl, CMA, 4-CPB, CPMF, 4-CPP, CPPC, cresol, cumyluron, cyanamide, cyanazine, cycloate, cyclosulfamuron, cycloxydim, cyhalofop, cyhalofop-butyl, 2,4-D, 3,4-DA, daimuron, dalapon, dazomet, 2,4-DB, 3,4-DB, 2,4-DEB, desmedipham, dicamba, dichlobenil, ortho-dichlorobenzene, para-dichlorobenzene, dichlorprop, dichlorprop-P, diclofop, diclofop-methyl, diclosulam, difenzoquat, difenzoquat metilsulfate, diflufenican, diflufenzopyr, dimefuron, dimepiperate, dimethachlor, dimethametryn, dimethenamid, dimethenamid-P, dimethipin, dimethylarsinic acid, dinitramine, dinoterb, diphenamid, diquat, diquat dibromide, dithiopyr, diuron, DNOC, 3,4-DP, DSMA, EBEP, endothal, EPTC, esprocarb, ethalfluralin, ethametsulfuron, ethametsulfuron-methyl, ethofumesate, ethoxyfen, ethoxysulfuron, etobenzanid, fenoxaprop-P, fenoxaprop-P-ethyl, fentrazamide, ferrous sulfate, flamprop-M, flazasulfuron, florasulam, fluazifop, fluazifop-butyl, fluazifop-P, fluazifop-P-butyl, flucarbazone, flucarbazone-sodium, flucetosulfuron, fluchloralin, flufenacet, flufenpyr, flufenpyr-ethyl, flumetsulam, flumiclorac, flumiclorac-pentyl, flumioxazin, fluometuron, fluoroglycofen, fluoroglycofen-ethyl, flupropanate, flupyrsulfuron, flupyrsulfuron-methyl-sodium, flurenol, fluridone, fluorochloridone, fluoroxypyr, flurtamone, fluthiacet, fluthiacet-methyl, fomesafen, foramsulfuron, fosamine, glufosinate, glufosinate-ammonium, glyphosate, halosulfuron, halosulfuron-methyl, haloxyfop, haloxyfop-P, HC-252, hexazinone, imazamethabenz, imazamethabenz-methyl, imazamox, imazapic, imazapyr, imazaquin, imazethapyr, imazosulfuron, indanofan, iodomethane, iodosulfuron, iodosulfuron-methyl-sodium, ioxynil, isoproturon, isouron, isoxaben, isoxachlortole, isoxaflutole, karbutilate, lactofen, lenacil, linuron, MAA, MAMA, MCPA, MCPA-thioethyl, MCPB, mecoprop, mecoprop-P, mefenacet, mefluidide, mesosulfuron, mesosulfuron-methyl, mesotrione, metam, metamifop, metamitron, metazachlor, methabenzthiazuron, methylarsonic acid, methyldymron, methyl isothiocyanate, metobenzuron, metolachlor, S-metolachlor, metosulam, metoxuron, metribuzin, metsulfuron, metsulfuron-methyl, MK-66, molinate, monolinuron, MSMA, naproanilide, napropamide, naptalam, neburon, nicosulfuron, nonanoic acid, norflurazon, oleic acid (fatty acids), orbencarb, orthosulfamuron, oryzalin, oxadiargyl, oxadiazon, oxasulfuron, oxaziclomefone, oxyfluorfen, paraquat, paraquat dichloride, pebulate, pendimethalin, penoxsulam, pentachlorophenol, pentanochlor, pentoxazone, pethoxamid, petrolium oils, phenmedipham, phenmedipham-ethyl, picloram, picolinafen, pinoxaden, piperophos, potassium arsenite, potassium azide, pretilachlor, primisulfuron, primisulfuron-methyl, prodiamine, profluazol, profoxydim, prometon, prometryn, propachlor, propanil, propaquizafop, propazine, propham, propisochlor, propoxycarbazone, propoxycarbazone-sodium, propyzamide, prosulfocarb, prosulfuron, pyraclonil, pyraflufen, pyraflufen-ethyl, pyrazolynate, pyrazosulfuron, pyrazosulfuron-ethyl, pyrazoxyfen, pyribenzoxim, pyributicarb, pyridafol, pyridate, pyriftalid, pyriminobac, pyriminobac-methyl, pyrimisulfan, pyrithiobac, pyrithiobac-sodium, quinclorac, quinmerac, quinoclamine, quizalofop, quizalofop-P, rimsulfuron, sethoxydim, siduron, simazine, simetryn, SMA, sodium arsenite, sodium azide, sodium chlorate, sulcotrione, sulfentrazone, sulfometuron, sulfometuron-methyl, sulfosate, sulfosulfuron, sulfuric acid, tar oils, 2,3,6-TBA, TCA, TCA-sodium, tebuthiuron, tepraloxydim, terbacil, terbumeton, terbuthylazine, terbutryn, thenylchlor, thiazopyr, thifensulfuron, thifensulfuron-methyl, thiobencarb, tiocarbazil, topramezone, tralkoxydim, tri-allate, triasulfuron, triaziflam, tribenuron, tribenuron-methyl, tricamba, triclopyr, trietazine, trifloxysulfuron, trifloxysulfuron-sodium, trifluralin, triflusulfuron, triflusulfuron-methyl, trihydroxytriazine, tritosulfuron, [3-[2-chloro-4-fluoro-5-(-methyl-6-trifluoromethyl-2,4-dioxo-,2,3,4-t-etrahydropyrimidin-3-yl)phenoxy]-2-pyridyloxy]acetic acid ethyl ester (CAS RN 353292-3-6), 4-[(4,5-dihydro-3-methoxy-4-methyl-5-oxo)-H—,2,4-triazol-1-ylcarbonyl-sulfamoyl]-5-methylthiophene-3-carboxylic acid (BAY636), BAY747 (CAS RN 33504-84-2), topramezone (CAS RN 2063-68-8), 4-hydroxy-3-[[2-[(2-methoxyethoxy)methyl]-6-(trifluoro-methyl)-3-pyridi-nyl]carbonyl]-bicyclo[3.2.]oct-3-en-2-one (CAS RN 35200-68-5), and 4-hydroxy-3-[[2-(3-methoxypropyl)-6-(difluoromethyl)-3-pyridinyl]carbon-yl]-bicyclo[3,2.]oct-3-en-2-one. Additionally, including herbicidal compounds of unspecified modes of action as described in CN101279950A, CN101279951A, DE10000600A1, DE10116399A1, DE102004054666A1, DE102005014638A1, DE102005014906A1, DE102007012168A1, DE102010042866A1, DE10204951A1, DE10234875A1, DE10234876A1, DE10256353A1, DE10256354A1, DE10256367A1, EP1157991A2, EP1238586A1, EP2147919A1, EP2160098A2, JP03968012B2, JP2001253874A, JP2002080454A, JP2002138075A, JP2002145707A, JP2002220389A, JP2003064059A, JP2003096059A, JP2004051628A, JP2004107228A, JP2005008583A, JP2005239675A, JP2005314407A, JP2006232824A, JP2006282552A, JP2007153847A, JP2007161701A, JP2007182404A, JP2008074840A, JP2008074841A, JP2008133207A, JP2008133218A, JP2008169121A, JP2009067739A, JP2009114128A, JP2009126792A, JP2009137851A, US20060111241A1, US20090036311A1, US20090054240A1, US20090215628A1, US20100099561A1, US20100152443A1, US20110105329A1, US20110201501A1, WO2001055066A2, WO2001056975A1, WO2001056979A1, WO2001090071A2, WO2001090080A1, WO2002002540A1, WO2002028182A1, WO2002040473A1, WO2002044173A2, WO2003000679A2, WO2003006422A1, WO2003013247A1, WO2003016308A1, WO2003020704A1, WO2003022051A1, WO2003022831A1, WO2003022843A1, WO2003029243A2, WO2003037085A1, WO2003037878A1, WO2003045878A2, WO2003050087A2, WO2003051823A1, WO2003051824A1, WO2003051846A2, WO2003076409A1, WO2003087067A1, WO2003090539A1, WO2003091217A1, WO2003093269A2, WO2003104206A2, WO2004002947A1, WO2004002981A2, WO2004011429A1, WO2004029060A1, WO2004035545A2, WO2004035563A1, WO2004035564A1, WO2004037787A1, WO2004067518A1, WO2004067527A1, WO2004077950A1, WO2005000824A1, WO2005007627A1, WO2005040152A1, WO2005047233A1, WO2005047281A1, WO2005061443A2, WO2005061464A1, WO2005068434A1, WO2005070889A1, WO2005089551A1, WO2005095335A1, WO2006006569A1, WO2006024820A1, WO2006029828A1, WO2006029829A1, WO2006037945A1, WO2006050803A1, WO2006090792A1, WO2006123088A2, WO2006125687A1, WO2006125688A1, WO2007003294A1, WO2007026834A1, WO2007071900A1, WO2007077201A1, WO2007077247A1, WO2007096576A1, WO2007119434A1, WO2007134984A1, WO2008009908A1, WO2008029084A1, WO2008059948A1, WO2008071918A1, WO2008074991A1, WO2008084073A1, WO2008100426A2, WO2008102908A1, WO2008152072A2, WO2008152073A2, WO2009000757A1, WO2009005297A2, WO2009035150A2, WO2009063180A1, WO2009068170A2, WO2009068171A2, WO2009086041A1, WO2009090401A2, WO2009090402A2, WO2009115788A1, WO2009116558A1, WO2009152995A1, WO2009158258A1, WO2010012649A1, WO2010012649A1, WO2010026989A1, WO2010034153A1, WO2010049270A1, WO2010049369A1, WO2010049405A1, WO2010049414A1, WO2010063422A1, WO2010069802A1, WO2010078906A2, WO2010078912A1, WO2010104217A1, WO2010108611A1, WO2010112826A3, WO2010116122A3, WO2010119906A1, WO2010130970A1, WO2011003776A2, WO2011035874A1, WO2011065451A1, all of which are incorporated herein by reference.

The trigger polynucleotide and oligonucleotide molecule compositions are useful in compositions, such as liquids that comprise the polynucleotide molecules at low concentrations, alone or in combination with other components, for example one or more herbicide molecules, either in the same solution or in separately applied liquids that also provide a transfer agent. While there is no upper limit on the concentrations and dosages of polynucleotide molecules that can useful in the methods, lower effective concentrations and dosages will generally be sought for efficiency. The concentrations can be adjusted in consideration of the volume of spray or treatment applied to plant leaves or other plant part surfaces, such as flower petals, stems, tubers, fruit, anthers, pollen, or seed. In one embodiment, a useful treatment for herbaceous plants using 25-mer oligonucleotide molecules is about 1 nanomole (nmol) of oligonucleotide molecules per plant, for example, from about 0.05 to 1 nmol per plant. Other embodiments for herbaceous plants include useful ranges of about 0.05 to about 100 nmol, or about 0.1 to about 20 nmol, or about 1 nmol to about 10 nmol of polynucleotides per plant. Very large plants, trees, or vines may require correspondingly larger amounts of polynucleotides. When using long dsRNA molecules that can be processed into multiple oligonucleotides, lower concentrations can be used. To illustrate embodiments, the factor 1×, when applied to oligonucleotide molecules is arbitrarily used to denote a treatment of 0.8 nmol of polynucleotide molecule per plant; 10×, 8 nmol of polynucleotide molecule per plant; and 100×, 80 nmol of polynucleotide molecule per plant.

The polynucleotide compositions are useful in compositions, such as liquids that comprise polynucleotide molecules, alone or in combination with other components either in the same liquid or in separately applied liquids that provide a transfer agent. As used herein, a transfer agent is an agent that, when combined with a polynucleotide in a composition that is topically applied to a target plant surface, enables the polynucleotide to enter a plant cell. In certain embodiments, a transfer agent is an agent that conditions the surface of plant tissue, e.g., leaves, stems, roots, flowers, or fruits, to permeation by the polynucleotide molecules into plant cells. The transfer of polynucleotides into plant cells can be facilitated by the prior or contemporaneous application of a polynucleotide-transferring agent to the plant tissue. In some embodiments the transferring agent is applied subsequent to the application of the polynucleotide composition. The polynucleotide transfer agent enables a pathway for polynucleotides through cuticle wax barriers, stomata and/or cell wall or membrane barriers into plant cells. Suitable transfer agents to facilitate transfer of the polynucleotide into a plant cell include agents that increase permeability of the exterior of the plant or that increase permeability of plant cells to oligonucleotides or polynucleotides. Such agents to facilitate transfer of the composition into a plant cell include a chemical agent, or a physical agent, or combinations thereof. Chemical agents for conditioning or transfer include (a) surfactants, (b) an organic solvent or an aqueous solution or aqueous mixtures of organic solvents, (c) oxidizing agents, (d) acids, (e) bases, (f) oils, (g) enzymes, or combinations thereof. Embodiments of the method can optionally include an incubation step, a neutralization step (e.g., to neutralize an acid, base, or oxidizing agent, or to inactivate an enzyme), a rinsing step, or combinations thereof. Embodiments of agents or treatments for conditioning of a plant to permeation by polynucleotides include emulsions, reverse emulsions, liposomes, and other micellar-like compositions. Embodiments of agents or treatments for conditioning of a plant to permeation by polynucleotides include counter-ions or other molecules that are known to associate with nucleic acid molecules, e.g., inorganic ammonium ions, alkyl ammonium ions, lithium ions, polyamines such as spermine, spermidine, or putrescine, and other cations. Organic solvents useful in conditioning a plant to permeation by polynucleotides include DMSO, DMF, pyridine, N-pyrrolidine, hexamethylphosphoramide, acetonitrile, dioxane, polypropylene glycol, other solvents miscible with water or that will dissolve phosphonucleotides in non-aqueous systems (such as is used in synthetic reactions). Naturally derived or synthetic oils with or without surfactants or emulsifiers can be used, e.g., plant-sourced oils, crop oils (such as those listed in the 9^{th} Compendium of Herbicide Adjuvants, publicly available on the worldwide web (internet) at herbicide.adjuvants.com can be used, e.g., paraffinic oils, polyol fatty acid esters, or oils with short-chain molecules modified with amides or polyamines such as polyethyleneimine or N-pyrrolidine. Transfer agents include, but are not limited to, organosilicone preparations.

An agronomic field in need of plant control is treated by application of the composition directly to the surface of the growing plants, such as by a spray. For example, the method is applied to control weeds in a field of crop plants by spraying the field with the composition. The composition can be provided as a tank mix, a sequential treatment of components (generally the polynucleotide containing composition followed by the herbicide), or a simultaneous treatment or mixing of one or more of the components of the composition from separate containers. Treatment of the field can occur as often as needed to provide weed control and the components of the composition can be adjusted to target specific weed species or weed families through utilization of specific polynucleotides or polynucleotide compositions capable of selectively targeting the specific species or plant family to be controlled. The composition can be applied at effective use rates according to the time of application to the field, for example, preplant, at planting, post planting, post harvest. HPPD inhibitor herbicides can be applied to a field at rates of 1 to 2000 g ai/ha (active ingredient per hectare or more. The polynucleotides of the composition can be applied at rates of 1 to 30 grams per acre depending on the number of trigger molecules needed for the scope of weeds in the field.

Crop plants in which weed control is needed include but are not limited to, i) corn, soybean, cotton, canola, sugar beet, alfalfa, sugarcane, rice, and wheat; ii) vegetable plants including, but not limited to, tomato, sweet pepper, hot pepper, melon, watermelon, cucumber, eggplant, cauliflower, broccoli, lettuce, spinach, onion, peas, carrots, sweet corn, Chinese cabbage, leek, fennel, pumpkin, squash or gourd, radish, Brussels sprouts, tomatillo, garden beans, dry beans, or okra; iii) culinary plants including, but not limited to, basil, parsley, coffee, or tea; or, iv) fruit plants including but not limited to apple, pear, cherry, peach, plum, apricot, banana, plantain, table grape, wine grape, citrus, avocado, mango, or berry; v) a tree grown for ornamental or commercial use, including, but not limited to, a fruit or nut tree; or, yl) an ornamental plant (e.g., an ornamental flowering plant or shrub or turf grass). The methods and compositions provided herein can also be applied to plants produced by a cutting, cloning, or grafting process (i.e., a plant not grown from a seed) include fruit trees and plants that include, but are not limited to, citrus, apples, avocados, tomatoes, eggplant, cucumber, melons, watermelons, and grapes as well as various ornamental plants.

### Pesticidal Mixtures

The polynucleotide compositions may also be used as mixtures with various agricultural chemicals and/or insecticides, miticides and fungicides, pesticidal and biopesticidal agents. Examples include but are not limited to azinphos-methyl, acephate, isoxathion, isofenphos, ethion, etrimfos, oxydemeton-methyl, oxydeprofos, quinalphos, chlorpyrifos, chlorpyrifos-methyl, chlorfenvinphos, cyanophos, dioxabenzofos, dichlorvos, disulfoton, dimethylvinphos, dimethoate, sulprofos, diazinon, thiometon, tetrachlorvinphos, temephos, tebupirimfos, terbufos, naled, vamidothion, pyraclofos, pyridafenthion, pirimiphos-methyl, fenitrothion, fenthion, phenthoate, flupyrazophos, prothiofos, propaphos, profenofos, phoxime, phosalone, phosmet, formothion, phorate, malathion, mecarbam, mesulfenfos, methamidophos, methidathion, parathion, methyl parathion, monocrotophos, trichlorphon, EPN, isazophos, isamidofos, cadusafos, diamidaphos, dichlofenthion, thionazin, fenamiphos, fosthiazate, fosthietan, phosphocarb, DSP, ethoprophos, alanycarb, aldicarb, isoprocarb, ethiofencarb, carbaryl, carbosulfan, xylylcarb, thiodicarb, pirimicarb, fenobucarb, furathiocarb, propoxur, bendiocarb, benfuracarb, methomyl, metolcarb, XMC, carbofuran, aldoxycarb, oxamyl, acrinathrin, allethrin, esfenvalerate, empenthrin, cycloprothrin, cyhalothrin, gamma-cyhalothrin, lambda-cyhalothrin, cyfluthrin, beta-cyfluthrin, cypermethrin, alpha-cypermethrin, zeta-cypermethrin, silafluofen, tetramethrin, tefluthrin, deltamethrin, tralomethrin, bifenthrin, phenothrin, fenvalerate, fenpropathrin, furamethrin, prallethrin, flucythrinate, fluvalinate, flubrocythrinate, permethrin, resmethrin, ethofenprox, cartap, thiocyclam, bensultap, acetamiprid, imidacloprid, clothianidin, dinotefuran, thiacloprid, thiamethoxam, nitenpyram, chlorfluazuron, diflubenzuron, teflubenzuron, triflumuron, novaluron, noviflumuron, bistrifluoron, fluazuron, flucycloxuron, flufenoxuron, hexaflumuron, lufenuron, chromafenozide, tebufenozide, halofenozide, methoxyfenozide, diofenolan, cyromazine, pyriproxyfen, buprofezin, methoprene, hydroprene, kinoprene, triazamate, endosulfan, chlorfenson, chlorobenzilate, dicofol, bromopropylate, acetoprole, fipronil, ethiprole, pyrethrin, rotenone, nicotine sulphate, BT (Bacillus Thuringiensis) agent, spinosad, abamectin, acequinocyl, amidoflumet, amitraz, etoxazole, chinomethionat, clofentezine, fenbutatin oxide, dienochlor, cyhexatin, spirodiclofen, spiromesifen, tetradifon, tebufenpyrad, binapacryl, bifenazate, pyridaben, pyrimidifen, fenazaquin, fenothiocarb, fenpyroximate, fluacrypyrim, fluazinam, flufenzin, hexythiazox, propargite, benzomate, polynactin complex, milbemectin, lufenuron, mecarbam, methiocarb, mevinphos, halfenprox, azadirachtin, diafenthiuron, indoxacarb, emamectin benzoate, potassium oleate, sodium oleate, chlorfenapyr, tolfenpyrad, pymetrozine, fenoxycarb, hydramethylnon, hydroxy propyl starch, pyridalyl, flufenerim, flubendiamide, flonicamid, metaflumizole, lepimectin, TPIC, albendazole, oxibendazole, oxfendazole, trichlamide, fensulfothion, fenbendazole, levamisole hydrochloride, morantel tartrate, dazomet, metam-sodium, triadimefon, hexaconazole, propiconazole, ipconazole, prochloraz, triflumizole, tebuconazole, epoxiconazole, difenoconazole, flusilazole, triadimenol, cyproconazole, metconazole, fluquinconazole, bitertanol, tetraconazole, triticonazole, flutriafol, penconazole, diniconazole, fenbuconazole, bromuconazole, imibenconazole, simeconazole, myclobutanil, hymexazole, imazalil, furametpyr, thifluzamide, etridiazole, oxpoconazole, oxpoconazole fumarate, pefurazoate, prothioconazole, pyrifenox, fenarimol, nuarimol, bupirimate, mepanipyrim, cyprodinil, pyrimethanil, metalaxyl, mefenoxam, oxadixyl, benalaxyl, thiophanate, thiophanate-methyl, benomyl, carbendazim, fuberidazole, thiabendazole, manzeb, propineb, zineb, metiram, maneb, ziram, thiuram, chlorothalonil, ethaboxam, oxycarboxin, carboxin, flutolanil, silthiofam, mepronil, dimethomorph, fenpropidin, fenpropimorph, spiroxamine, tridemorph, dodemorph, flumorph, azoxystrobin, kresoxim-methyl, metominostrobin, orysastrobin, fluoxastrobin, trifloxystrobin, dimoxystrobin, pyraclostrobin, picoxystrobin, iprodione, procymidone, vinclozolin, chlozolinate, flusulfamide, dazomet, methyl isothiocyanate, chloropicrin, methasulfocarb, hydroxyisoxazole, potassium hydroxyisoxazole, echlomezol, D-D, carbam, basic copper chloride, basic copper sulfate, copper nonylphenolsulfonate, oxine copper, DBEDC, anhydrous copper sulfate, copper sulfate pentahydrate, cupric hydroxide, inorganic sulfur, wettable sulfur, lime sulfur, zinc sulfate, fentin, sodium hydrogen carbonate, potassium hydrogen carbonate, sodium hypochlorite, silver, edifenphos, tolclofos-methyl, fosetyl, iprobenfos, dinocap, pyrazophos, carpropamid, fthalide, tricyclazole, pyroquilon, diclocymet, fenoxanil, kasugamycin, validamycin, polyoxins, blasticiden S, oxytetracycline, mildiomycin, streptomycin, rape seed oil, machine oil, benthiavalicarbisopropyl, iprovalicarb, propamocarb, diethofencarb, fluoroimide, fludioxanil, fenpiclonil, quinoxyfen, oxolinic acid, chlorothalonil, captan, folpet, probenazole, acibenzolar-S-methyl, tiadinil, cyflufenamid, fenhexamid, diflumetorim, metrafenone, picobenzamide, proquinazid, famoxadone, cyazofamid, fenamidone, zoxamide, boscalid, cymoxanil, dithianon, fluazinam, dichlofluanide, triforine, isoprothiolane, ferimzone, diclomezine, tecloftalam, pencycuron, chinomethionat, iminoctadine acetate, iminoctadine albesilate, ambam, polycarbamate, thiadiazine, chloroneb, nickel dimethyldithiocarbamate, guazatine, dodecylguanidine-acetate, quintozene, tolylfluanid, anilazine, nitrothalisopropyl, fenitropan, dimethirimol, benthiazole, harpin protein, flumetover, mandipropamide and penthiopyrad.

### Polynucleotides

As used herein, the term “DNA”, “DNA molecule”, “DNA polynucleotide molecule” refers to a single-stranded DNA (ssDNA) or double-stranded DNA (dsDNA) molecule of genomic or synthetic origin, such as, a polymer of deoxyribonucleotide bases or a DNA polynucleotide molecule. As used herein, the term “DNA sequence”, “DNA nucleotide sequence” or “DNA polynucleotide sequence” refers to the nucleotide sequence of a DNA molecule. As used herein, the term “RNA”, “RNA molecule”, “RNA polynucleotide molecule” refers to a single-stranded RNA (ssRNA) or double-stranded RNA (dsRNA) molecule of genomic or synthetic origin, such as, a polymer of ribonucleotide bases that comprise single or double stranded regions. Unless otherwise stated, nucleotide sequences in the text of this specification are given, when read from left to right, in the 5′ to 3′ direction. The nomenclature used herein is that required by Title 37 of the United States Code of Federal Regulations §1.822 and set forth in the tables in WIPO Standard ST.25 (1998), Appendix 2, Tables 1 and 3.

As used herein, “polynucleotide” refers to a DNA or RNA molecule containing multiple nucleotides and generally refers both to “oligonucleotides” (a polynucleotide molecule of typically 50 or fewer nucleotides in length) and polynucleotides of 51 or more nucleotides. Embodiments of this invention include compositions including oligonucleotides having a length of 18-25 nucleotides (18-mers, 19-mers, 20-mers, 21-mers, 22-mers, 23-mers, 24-mers, or 25-mers) for example, oligonucleotides SEQ ID NO:597-1082 or fragments thereof, or medium-length polynucleotides having a length of 26 or more nucleotides (polynucleotides of 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290, or about 300 nucleotides), for example, oligonucleotides SEQ ID NO:33-596 or fragments thereof or long polynucleotides having a length greater than about 300 nucleotides (for example, polynucleotides of between about 300 to about 400 nucleotides, between about 400 to about 500 nucleotides, between about 500 to about 600 nucleotides, between about 600 to about 700 nucleotides, between about 700 to about 800 nucleotides, between about 800 to about 900 nucleotides, between about 900 to about 1000 nucleotides, between about 300 to about 500 nucleotides, between about 300 to about 600 nucleotides, between about 300 to about 700 nucleotides, between about 300 to about 800 nucleotides, between about 300 to about 900 nucleotides, or about 1000 nucleotides in length, or even greater than about 1000 nucleotides in length, for example up to the entire length of a target gene including coding or non-coding or both coding and non-coding portions of the target gene), for example, polynucleotides of Table 1 (SEQ ID NO:1-32), wherein the selected polynucleotides or fragments thereof are homologous or complementary to SEQ ID NO:1-32 and suppresses, represses or otherwise delay the expression of the target EPSPS gene. A target gene comprises any polynucleotide molecule in a plant cell or fragment thereof for which the modulation of the expression of the target gene is provided by the methods and compositions of the present invention. Where a polynucleotide is double-stranded, its length can be similarly described in terms of base pairs. Oligonucleotides and polynucleotides of the present invention can be made that are essentially identical or essentially complementary to adjacent genetic elements of a gene, for example, spanning the junction region of an intron and exon, the junction region of a promoter and a transcribed region, the junction region of a 5′ leader and a coding sequence, the junction of a 3′ untranslated region and a coding sequence.

Polynucleotide compositions used in the various embodiments of this invention include compositions including oligonucleotides or polynucleotides or a mixture of both, including RNA or DNA or RNA/DNA hybrids or chemically modified oligonucleotides or polynucleotides or a mixture thereof. In some embodiments, the polynucleotide may be a combination of ribonucleotides and deoxyribonucleotides, for example, synthetic polynucleotides consisting mainly of ribonucleotides but with one or more terminal deoxyribonucleotides or synthetic polynucleotides consisting mainly of deoxyribonucleotides but with one or more terminal dideoxyribonucleotides. In some embodiments, the polynucleotide includes non-canonical nucleotides such as inosine, thiouridine, or pseudouridine. In some embodiments, the polynucleotide includes chemically modified nucleotides. Examples of chemically modified oligonucleotides or polynucleotides are well known in the art; see, for example, US Patent Publication 20110171287, US Patent Publication 20110171176, and US Patent Publication 20110152353, US Patent Publication, 20110152346, US Patent Publication 20110160082, herein incorporated by reference. For example, including but not limited to the naturally occurring phosphodiester backbone of an oligonucleotide or polynucleotide can be partially or completely modified with phosphorothioate, phosphorodithioate, or methylphosphonate internucleotide linkage modifications, modified nucleoside bases or modified sugars can be used in oligonucleotide or polynucleotide synthesis, and oligonucleotides or polynucleotides can be labeled with a fluorescent moiety (for example, fluorescein or rhodamine) or other label (for example, biotin).

The polynucleotides can be single- or double-stranded RNA or single- or double-stranded DNA or double-stranded DNA/RNA hybrids or modified analogues thereof, and can be of oligonucleotide lengths or longer. In more specific embodiments of the invention the polynucleotides that provide single-stranded RNA in the plant cell are selected from the group consisting of (a) a single-stranded RNA molecule (ssRNA), (b) a single-stranded RNA molecule that self-hybridizes to form a double-stranded RNA molecule, (c) a double-stranded RNA molecule (dsRNA), (d) a single-stranded DNA molecule (ssDNA), (e) a single-stranded DNA molecule that self-hybridizes to form a double-stranded DNA molecule, and (f) a single-stranded DNA molecule including a modified Pol III gene that is transcribed to an RNA molecule, (g) a double-stranded DNA molecule (dsDNA), (h) a double-stranded DNA molecule including a modified Pol III gene that is transcribed to an RNA molecule, (i) a double-stranded, hybridized RNA/DNA molecule, or combinations thereof. In some embodiments these polynucleotides include chemically modified nucleotides or non-canonical nucleotides. In embodiments of the method the polynucleotides include double-stranded DNA formed by intramolecular hybridization, double-stranded DNA formed by intermolecular hybridization, double-stranded RNA formed by intramolecular hybridization, or double-stranded RNA formed by intermolecular hybridization. In some embodiments, the oligonucleotides may be blunt-ended or may comprise a 3′ overhang of from 1-5 nucleotides of at least one or both of the strands. Other configurations of the oligonucleotide are known in the field and are contemplated herein. In one embodiment the polynucleotides include single-stranded DNA or single-stranded RNA that self-hybridizes to form a hairpin structure having an at least partially double-stranded structure including at least one segment that will hybridize to RNA transcribed from the gene targeted for suppression. Not intending to be bound by any mechanism, it is believed that such polynucleotides are or will produce single-stranded RNA with at least one segment that will hybridize to RNA transcribed from the gene targeted for suppression. In certain other embodiments the polynucleotides further includes a promoter, generally a promoter functional in a plant, for example, a pol II promoter, a pol III promoter, a pol IV promoter, or a pol V promoter.

The term “gene” refers to chromosomal DNA, plasmid DNA, cDNA, intron and exon DNA, artificial DNA polynucleotide, or other DNA that encodes a peptide, polypeptide, protein, or RNA transcript molecule, and the genetic elements flanking the coding sequence that are involved in the regulation of expression, such as, promoter regions, 5′ leader regions, 3′ untranslated regions. Any of the components of the gene are potential targets for the oligonucleotides and polynucleotides of the present invention.

The trigger polynucleotide molecules are designed to modulate expression by inducing regulation or suppression of an endogenous HPPD gene in a plant and are designed to have a nucleotide sequence essentially identical or essentially complementary to the nucleotide sequence of an endogenous HPPD gene of a plant or to the sequence of RNA transcribed from an endogenous HPPD gene of a plant, including a transgene in a plant that provides for a herbicide resistant HPPD enzyme, which can be coding sequence or non-coding sequence. Effective molecules that modulate expression are referred to as “a trigger molecule, or trigger polynucleotide”. By “essentially identical” or “essentially complementary” is meant that the trigger polynucleotides (or at least one strand of a double-stranded polynucleotide or portion thereof, or a portion of a single strand polynucleotide) are designed to hybridize to the endogenous gene noncoding sequence or to RNA transcribed (known as messenger RNA or an RNA transcript) from the endogenous gene to effect regulation or suppression of expression of the endogenous gene. Trigger molecules are identified by “tiling” the gene targets with partially overlapping probes or non-overlapping probes of antisense or sense polynucleotides that are essentially identical or essentially complementary to the nucleotide sequence of an endogenous gene. Multiple target sequences can be aligned and sequence regions with homology in common, according to the methods of the present invention, are identified as potential trigger molecules for the multiple targets. Multiple trigger molecules of various lengths, for example 18-25 nucleotides, 26-50 nucleotides, 51-100 nucleotides, 101-200 nucleotides, 201-300 nucleotides or more can be pooled into a few treatments in order to investigate polynucleotide molecules that cover a portion of a gene sequence (for example, a portion of a coding versus a portion of a noncoding region, or a 5′ versus a 3′ portion of a gene) or an entire gene sequence including coding and noncoding regions of a target gene. Polynucleotide molecules of the pooled trigger molecules can be divided into smaller pools or single molecules in order to identify trigger molecules that provide the desired effect.

The target gene RNA and DNA polynucleotide molecules are (Table 1, SEQ ID NO: 1-32) sequenced by any number of available methods and equipment. Some of the sequencing technologies are available commercially, such as the sequencing-by-hybridization platform from Affymetrix Inc. (Sunnyvale, Calif.) and the sequencing-by-synthesis platforms from 454 Life Sciences (Bradford, Conn.), Illumina/Solexa (Hayward, Calif.) and Helicos Biosciences (Cambridge, Mass.), and the sequencing-by-ligation platform from Applied Biosystems (Foster City, Calif.), as described below. In addition to the single molecule sequencing performed using sequencing-by-synthesis of Helicos Biosciences, other single molecule sequencing technologies are encompassed by the method of the invention and include the SMRT™ technology of Pacific Biosciences, the Ion Torrent™ technology, and nanopore sequencing being developed for example, by Oxford Nanopore Technologies. A HPPD target gene comprising DNA or RNA can be isolated using primers or probes essentially complementary or essentially homologous to SEQ ID NO:1-32 or a fragment thereof. A polymerase chain reaction (PCR) gene fragment can be produced using primers essentially complementary or essentially homologous to SEQ ID NO:1-32 or a fragment thereof that is useful to isolate an HPPD gene from a plant genome. SEQ ID NO: 1-32 or fragments thereof can be used in various sequence capture technologies to isolate additional target gene sequences, for example, including but not limited to Roche NimbleGen®(Madison, Wis.) and Streptavdin-coupled Dynabeads® (Life Technologies, Grand Island, N.Y.) and US20110015084, herein incorporated by reference in its entirety.

Embodiments of single-stranded polynucleotides functional in this invention have sequence complementarity that need not be 100 percent, but is at least sufficient to permit hybridization to RNA transcribed from the target gene or DNA of the target gene to form a duplex to permit a gene silencing mechanism. Thus, in embodiments, a polynucleotide fragment is designed to be essentially identical to, or essentially complementary to, a sequence of 18 or more contiguous nucleotides in either the target HPPD gene sequence or messenger RNA transcribed from the target gene. By “essentially identical” is meant having 100 percent sequence identity or at least about 83, 84, 85, 86, 87, 88, 89, 90, 91, 92, 93, 94, 95, 96, 97, 98, or 99 percent sequence identity when compared to the sequence of 18 or more contiguous nucleotides in either the target gene or RNA transcribed from the target gene; by “essentially complementary” is meant having 100 percent sequence complementarity or at least about 83, 84, 85, 86, 87, 88, 89, 90, 91, 92, 93, 94, 95, 96, 97, 98, or 99 percent sequence complementarity when compared to the sequence of 18 or more contiguous nucleotides in either the target gene or RNA transcribed from the target gene. In some embodiments of this invention polynucleotide molecules are designed to have 100 percent sequence identity with or complementarity to one allele or one family member of a given target gene (coding or non-coding sequence of a gene for of the present invention); in other embodiments the polynucleotide molecules are designed to have 100 percent sequence identity with or complementarity to multiple alleles or family members of a given target gene.

In certain embodiments, the polynucleotides used in the compositions that are essentially identical or essentially complementary to the target gene or transcript will comprise the predominant nucleic acid in the composition. Thus in certain embodiments, the polynucleotides that are essentially identical or essentially complementary to the target gene or transcript will comprise at least about 50%, 75%, 95%, 98% or 100% of the nucleic acids provided in the composition by either mass or molar concentration. However, in certain embodiments, the polynucleotides that are essentially identical or essentially complementary to the target gene or transcript can comprise at least about 1% to about 50%, about 10% to about 50%, about 20% to about 50%, or about 30% to about 50% of the nucleic acids provided in the composition by either mass or molar concentration. Also provided are compositions where the polynucleotides that are essentially identical or essentially complementary to the target gene or transcript can comprise at least about 1% to 100%, about 10% to 100%, about 20% to about 100%, about 30% to about 50%, or about 50% to a 100% of the nucleic acids provided in the composition by either mass or molar concentration.

“Identity” refers to the degree of similarity between two polynucleic acid or protein sequences. An alignment of the two sequences is performed by a suitable computer program. A widely used and accepted computer program for performing sequence alignments is CLUSTALW v1.6 (Thompson, et al. Nucl. Acids Res., 22: 4673-4680, 1994). The number of matching bases or amino acids is divided by the total number of bases or amino acids, and multiplied by 100 to obtain a percent identity. For example, if two 580 base pair sequences had 145 matched bases, they would be 25 percent identical. If the two compared sequences are of different lengths, the number of matches is divided by the shorter of the two lengths. For example, if there are 100 matched amino acids between a 200 and a 400 amino acid protein, they are 50 percent identical with respect to the shorter sequence. If the shorter sequence is less than 150 bases or 50 amino acids in length, the number of matches are divided by 150 (for nucleic acid bases) or 50 (for amino acids), and multiplied by 100 to obtain a percent identity.

Trigger molecules for specific gene family members can be identified from coding and/or non-coding sequences of gene families of a plant or multiple plants, by aligning and selecting 200-300 polynucleotide fragments from the least homologous regions amongst the aligned sequences and evaluated using topically applied polynucleotides (as sense or anti-sense ssDNA or ssRNA, dsRNA, or dsDNA) to determine their relative effectiveness in inducing the herbicidal phenotype. The effective segments are further subdivided into 50-60 polynucleotide fragments, prioritized by least homology, and reevaluated using topically applied polynucleotides. The effective 50-60 polynucleotide fragments are subdivided into 19-30 polynucleotide fragments, prioritized by least homology, and again evaluated for induction of the yield/quality phenotype. Once relative effectiveness is determined, the fragments are utilized singly, or again evaluated in combination with one or more other fragments to determine the trigger composition or mixture of trigger polynucleotides for providing the yield/quality phenotype.

Trigger molecules for broad activity can be identified from coding and/or non-coding sequences of gene families of a plant or multiple plants, by aligning and selecting 200-300 polynucleotide fragments from the most homologous regions amongst the aligned sequences and evaluated using topically applied polynucleotides (as sense or anti-sense ssDNA or ssRNA, dsRNA, or dsDNA) to determine their relative effectiveness in inducing the yield/quality phenotype. The effective segments are subdivided into 50-60 polynucleotide fragments, prioritized by most homology, and reevaluated using topically applied polynucleotides. The effective 50-60 polynucleotide fragments are subdivided into 19-30 polynucleotide fragments, prioritized by most homology, and again evaluated for induction of the yield/quality phenotype. Once relative effectiveness is determined, the fragments may be utilized singly, or in combination with one or more other fragments to determine the trigger composition or mixture of trigger polynucleotides for providing the yield/quality phenotype.

Methods of making polynucleotides are well known in the art. Chemical synthesis, in vivo synthesis and in vitro synthesis methods and compositions are known in the art and include various viral elements, microbial cells, modified polymerases, and modified nucleotides. Commercial preparation of oligonucleotides often provides two deoxyribonucleotides on the 3′ end of the sense strand. Long polynucleotide molecules can be synthesized from commercially available kits, for example, kits from Applied Biosystems/Ambion (Austin, Tex.) have DNA ligated on the 5′ end in a microbial expression cassette that includes a bacterial T7 polymerase promoter that makes RNA strands that can be assembled into a dsRNA and kits provided by various manufacturers that include T7 RiboMax Express (Promega, Madison, Wis.), AmpliScribe T7-Flash (Epicentre, Madison, Wis.), and TranscriptAid T7 High Yield (Fermentas, Glen Burnie, Md.). dsRNA molecules can be produced from microbial expression cassettes in bacterial cells (Ongvarrasopone et al. ScienceAsia 33:35-39; Yin, Appl. Microbiol. Biotechnol 84:323-333, 2009; Liu et al., BMC Biotechnology 10:85, 2010) that have regulated or deficient RNase III enzyme activity or the use of various viral vectors to produce sufficient quantities of dsRNA. In the present invention, HPPD gene fragments are inserted into the microbial expression cassettes in a position in which the fragments are express to produce ssRNA or dsRNA useful in the methods described herein to regulate expression on a target HPPD gene. Long polynucleotide molecules can also be assembled from multiple RNA or DNA fragments. In some embodiments design parameters such as Reynolds score (Reynolds et al. Nature Biotechnology 22, 326-330 (2004), Tuschl rules (Pei and Tuschl, Nature Methods 3(9): 670-676, 2006), i-score (Nucleic Acids Res 35: e123, 2007), i-Score Designer tool and associated algorithms (Nucleic Acids Res 32: 936-948, 2004. Biochem Biophys Res Commun 316: 1050-1058, 2004, Nucleic Acids Res 32: 893-901, 2004, Cell Cycle 3: 790-5, 2004, Nat Biotechnol 23: 995-1001, 2005, Nucleic Acids Res 35: e27, 2007, BMC Bioinformatics 7: 520, 2006, Nucleic Acids Res 35: e123, 2007, Nat Biotechnol 22: 326-330, 2004) are known in the art and may be used in selecting polynucleotide sequences effective in gene silencing. In some embodiments the sequence of a polynucleotide is screened against the genomic DNA of the intended plant to minimize unintentional silencing of other genes.

Ligands can be tethered to a polynucleotide, for example a dsRNA, ssRNA, dsDNA or ssDNA. Ligands in general can include modifiers, e.g., for enhancing uptake; diagnostic compounds or reporter groups e.g., for monitoring distribution; cross-linking agents; nuclease-resistance conferring moieties; and natural or unusual nucleobases. General examples include lipophiles, lipids (e.g., cholesterol, a bile acid, or a fatty acid (e.g., lithocholic-oleyl, lauroyl, docosnyl, stearoyl, palmitoyl, myristoyl oleoyl, linoleoyl), steroids (e.g., uvaol, hecigenin, diosgenin), terpenes (e.g., triterpenes, e.g., sarsasapogenin, Friedelin, epifriedelanol derivatized lithocholic acid), vitamins (e.g., folic acid, vitamin A, biotin, pyridoxal), carbohydrates, proteins, protein binding agents, integrin targeting molecules, polycationics, peptides, polyamines, and peptide mimics. The ligand may also be a recombinant or synthetic molecule, such as a synthetic polymer, e.g., polyethylene glycol (PEG), PEG-40K, PEG-20K and PEG-5K. Other examples of ligands include lipophilic molecules, e.g, cholesterol, cholic acid, adamantane acetic acid, 1-pyrene butyric acid, dihydrotestosterone, glycerol (e.g., esters and ethers thereof, e.g., C.sub.10, C.sub.11, C.sub.12, C.sub.13, C.sub.14, C.sub.15, C.sub.16, C.sub.17, C.sub.18, C.sub.19, or C.sub.20 alkyl; e.g., lauroyl, docosnyl, stearoyl, oleoyl, linoleoyl 1,3-bis-O(hexadecyl)glycerol, 1,3-bis-O(octaadecyl)glycerol), geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dodecanoyl, lithocholyl, 5.beta.-cholanyl, N,N-distearyl-lithocholamide, 1,2-di-O-stearoylglyceride, dimethoxytrityl, or phenoxazine) and PEG (e.g., PEG-5K, PEG-20K, PEG-40K). Preferred lipophilic moieties include lipid, cholesterols, oleyl, retinyl, or cholesteryl residues.

Conjugating a ligand to a dsRNA can enhance its cellular absorption, lipophilic compounds that have been conjugated to oligonucleotides include 1-pyrene butyric acid, 1,3-bis-O-(hexadecyl)glycerol, and menthol. One example of a ligand for receptor-mediated endocytosis is folic acid. Folic acid enters the cell by folate-receptor-radiated endocytosis. dsRNA compounds bearing folic acid would be efficiently transported into the cell via the folate-receptor-mediated endocytosis. Other ligands that have been conjugated to oligonucleotides include polyethylene glycols, carbohydrate clusters, cross-linking agents, porphyrin conjugates, delivery peptides and lipids such as cholesterol. In certain instances, conjugation of a cationic ligand to oligonucleotides results in improved resistance to nucleases. Representative examples of cationic ligands are propylammonium and dimethylpropylammonium. Interestingly, antisense oligonucleotides were reported to retain their high binding affinity to mRNA when the cationic ligand was dispersed, throughout the oligonucleotide. See M. Manoharan Antisense & Nucleic Acid Drug Development 2002, 12, 103 and references therein.

A biologic delivery can be accomplished by a variety of methods including, without limitation, (1) loading liposomes with a dsRNA acid molecule provided herein and (2) complexing a dsRNA molecule with lipids or liposomes to form nucleic acid-lipid or nucleic acid-liposome complexes. The liposome can be composed of cationic and neutral lipids commonly used to transfect cells in vitro. Cationic lipids can complex (e.g., charge-associate) with negatively charged, nucleic acids to form liposomes. Examples of cationic liposomes include, without limitation, lipofectin, lipofectamine, lipofectace, and DOTAP. Procedures for forming liposomes are well known in the art. Liposome compositions can be formed, for example, from phosphatidylcholine, dimyristoyl phosphatidylcholine, dipalmitoyl phosphatidylcholine, dimyristoyl phosphatidyl glycerol, dioleoyl phosphatidylethanolamine or liposomes comprising dihydrosphingomyelin (DHSM) Numerous lipophilic agents are commercially available, including Lipofectin® (Invitrogen/Life Technologies, Carlsbad, Calif.) and Effectene™ (Qiagen, Valencia, Calif.), In addition, systemic delivery methods can be optimized using commercially available cationic lipids such as DDAB or DOTAP, each of which can be mixed with a neutral lipid such as DOPE or cholesterol. In some eases, liposomes such as those described by Templeton et al. (Nature Biotechnology, 15:647-652 (1997)) can be used. In other embodiments, polycations such as polyethyleneimine can be used to achieve delivery in vivo and ex vivo (Boletta et al., J. Am. Soc. Nephrol. 7:1728 (1996)). Additional information regarding the use of liposomes to deliver nucleic acids can be found in U.S. Pat. No. 6,271,359, PCT Publication WO 96/40964 and Morrissey, D. et al. 2005. Nat. Biotechnol. 23(8):1002-7.

In certain embodiments, an organosilicone preparation that is commercially available as Silwet® L-77 surfactant having CAS Number 27306-78-1 and EPA Number: CAL.REG.NO. 5905-50073-AA, and currently available from Momentive Performance Materials, Albany, N.Y. can be used to prepare a polynucleotide composition. In certain embodiments where a Silwet L-77 organosilicone preparation is used as a pre-spray treatment of plant leaves or other plant surfaces, freshly made concentrations in the range of about 0.015 to about 2 percent by weight (wt percent) (e.g., about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.5 wt percent) are efficacious in preparing a leaf or other plant surface for transfer of polynucleotide molecules into plant cells from a topical application on the surface. In certain embodiments of the methods and compositions provided herein, a composition that comprises a polynucleotide molecule and an organosilicone preparation comprising Silwet L-77 in the range of about 0.015 to about 2 percent by weight (wt percent) (e.g., about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.5 wt percent) is used or provided.

In certain embodiments, any of the commercially available organosilicone preparations provided such as the following Breakthru S 321, Breakthru S 200 Cat#67674-67-3, Breakthru OE 441 Cat#68937-55-3, Breakthru S 278 Cat #27306-78-1, Breakthru S 243, Breakthru S 233 Cat#134180-76-0, available from manufacturer Evonik Goldschmidt (Germany), Silwet® HS 429, Silwet® HS 312, Silwet® HS 508, Silwet® HS 604 (Momentive Performance Materials, Albany, N.Y.) can be used as transfer agents in a polynucleotide composition. In certain embodiments where an organosilicone preparation is used as a pre-spray treatment of plant leaves or other surfaces, freshly made concentrations in the range of about 0.015 to about 2 percent by weight (wt percent) (e.g., about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.5 wt percent) are efficacious in preparing a leaf or other plant surface for transfer of polynucleotide molecules into plant cells from a topical application on the surface. In certain embodiments of the methods and compositions provided herein, a composition that comprises a polynucleotide molecule and an organosilicone preparation in the range of about 0.015 to about 2 percent by weight (wt percent) (e.g., about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.5 wt percent) is used or provided.

Organosilicone preparations used in the methods and compositions provided herein can comprise one or more effective organosilicone compounds. As used herein, the phrase “effective organosilicone compound” is used to describe any organosilicone compound that is found in an organosilicone preparation that enables a polynucleotide to enter a plant cell. In certain embodiments, an effective organosilicone compound can enable a polynucleotide to enter a plant cell in a manner permitting a polynucleotide mediated suppression of a target gene expression in the plant cell. In general, effective organosilicone compounds include, but are not limited to, compounds that can comprise: i) a trisiloxane head group that is covalently linked to, ii) an alkyl linker including, but not limited to, an n-propyl linker, that is covalently linked to, iii) a poly glycol chain, that is covalently linked to, iv) a terminal group. Trisiloxane head groups of such effective organosilicone compounds include, but are not limited to, heptamethyltrisiloxane. Alkyl linkers can include, but are not limited to, an n-propyl linker Poly glycol chains include, but are not limited to, polyethylene glycol or polypropylene glycol. Poly glycol chains can comprise a mixture that provides an average chain length “n” of about “7.5”. In certain embodiments, the average chain length “n” can vary from about 5 to about 14. Terminal groups can include, but are not limited to, alkyl groups such as a methyl group. Effective organosilicone compounds are believed to include, but are not limited to, trisiloxane ethoxylate surfactants or polyalkylene oxide modified heptamethyl trisiloxane.

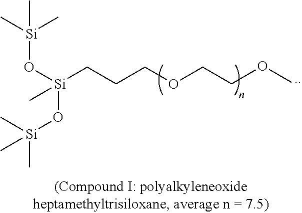

In certain embodiments, an organosilicone preparation that comprises an organosilicone compound comprising a trisiloxane head group is used in the methods and compositions provided herein. In certain embodiments, an organosilicone preparation that comprises an organosilicone compound comprising a heptamethyltrisiloxane head group is used in the methods and compositions provided herein. In certain embodiments, an organosilicone composition that comprises Compound I is used in the methods and compositions provided herein. In certain embodiments, an organosilicone composition that comprises Compound I is used in the methods and compositions provided herein. In certain embodiments of the methods and compositions provided herein, a composition that comprises a polynucleotide molecule and one or more effective organosilicone compound in the range of about 0.015 to about 2 percent by weight (wt percent) (e.g., about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.5 wt percent) is used or provided.

Compositions include but are not limited components that are one or more polynucleotides essentially identical to, or essentially complementary to an HPPD gene sequence (promoter, intron, exon, 5′ untranslated region, 3′ untranslated region), a transfer agent that provides for the polynucleotide to enter a plant cell, a herbicide that complements the action of the polynucleotide, one or more additional herbicides that further enhance the herbicide activity of the composition or provide an additional mode of action different from the complementing herbicide, various salts and stabilizing agents that enhance the utility of the composition as an admixture of the components of the composition.

In certain aspects, methods include one or more applications of a polynucleotide composition and one or more applications of a permeability-enhancing agent for conditioning of a plant to permeation by polynucleotides. When the agent for conditioning to permeation is an organosilicone composition or compound contained therein, embodiments of the polynucleotide molecules are double-stranded RNA oligonucleotides, single-stranded RNA oligonucleotides, double-stranded RNA polynucleotides, single-stranded RNA polynucleotides, double-stranded DNA oligonucleotides, single-stranded DNA oligonucleotides, double-stranded DNA polynucleotides, single-stranded DNA polynucleotides, chemically modified RNA or DNA oligonucleotides or polynucleotides or mixtures thereof.

Compositions and methods are useful for modulating the expression of an endogenous HPPD gene (for example, U.S. Pat. No. 7,297,541, U.S. Patent Publ. 20110185444, and 20110185445) or transgenic HPPD gene (for example,U.S. Pat. No. 7,312,379, U.S. Patent Publ. 20110191897) or HPPD inhibitor inactivating genes (U.S. Pat. Nos. 6,268,549; 6,768,044; 7,312,379; 7,304,209; WO 96/38567, WO 99/24585) in a plant cell. In various embodiments, an HPPD gene includes coding (protein-coding or translatable) sequence, non-coding (non-translatable) sequence, or both coding and non-coding sequence. Compositions can include polynucleotides and oligonucleotides designed to target multiple genes, or multiple segments of one or more genes. The target gene can include multiple consecutive segments of a target gene, multiple non-consecutive segments of a target gene, multiple alleles of a target gene, or multiple target genes from one or more species.

Provided is a method for modulating expression of an HPPD gene in a plant including (a) conditioning of a plant to permeation by polynucleotides and (b) treatment of the plant with the polynucleotide molecules, wherein the polynucleotide molecules include at least one segment of 18 or more contiguous nucleotides cloned from or otherwise identified from the target HPPD gene in either anti-sense or sense orientation, whereby the polynucleotide molecules permeate the interior of the plant and induce modulation of the target gene. The conditioning and polynucleotide application can be performed separately or in a single step. When the conditioning and polynucleotide application are performed in separate steps, the conditioning can precede or can follow the polynucleotide application within minutes, hours, or days. In some embodiments more than one conditioning step or more than one polynucleotide molecule application can be performed on the same plant. In embodiments of the method, the segment can be cloned or identified from (a) coding (protein-encoding), (b) non-coding (promoter and other gene related molecules), or (c) both coding and non-coding parts of the target gene. Non-coding parts include DNA, such as promoter regions or the RNA transcribed by the DNA that provide RNA regulatory molecules, including but not limited to: introns, 5′ or 3′ untranslated regions, and microRNAs (miRNA), trans-acting siRNAs, natural anti-sense siRNAs, and other small RNAs with regulatory function or RNAs having structural or enzymatic function including but not limited to: ribozymes, ribosomal RNAs, t-RNAs, aptamers, and riboswitches.

All publications, patents and patent applications are herein incorporated by reference to the same extent as if each individual publication or patent application was specifically and individually indicated to be incorporated by reference.

The following examples are included to demonstrate examples of certain preferred embodiments of the invention. It should be appreciated by those of skill in the art that the techniques disclosed in the examples that follow represent approaches the inventors have found function well in the practice of the invention, and thus can be considered to constitute examples of preferred modes for its practice. However, those of skill in the art should, in light of the present disclosure, appreciate that many changes can be made in the specific embodiments that are disclosed and still obtain a like or similar result without departing from the spirit and scope of the invention.

### EXAMPLES

### Example 1

### Polynucleotides Related to the HPPD Gene Sequences

The target HPPD gene polynucleotide molecules have been found that naturally occur in the genome of Amaranthus palmeri, Amaranthus rudis, Amaranthus thunbergii, Amaranthus graecizans, Amaranthus hybridus, Amaranthus viridis, Ambrosia trifida, Kochia scoparia, Abufilon theophrasti, Conyza candensis, Digitaria sanguinalis, Euphorbia heterophylla, Lolium multiflorum, and Xanthium strumarium and include molecules related to the expression of a polypeptide identified as an HPPD, that include regulatory molecules, cDNAs comprising coding and noncoding regions of an HPPD gene and fragments thereof as shown in Table 1.

Polynucleotide molecules were extracted from these plant species by methods standard in the field, for example, total RNA is extracted using Trizol Reagent (Invitrogen Corp, Carlsbad, Calif. Cat. No. 15596-018), following the manufacturer's protocol or modifications thereof by those skilled in the art of polynucleotide extraction that may enhance recover or purity of the extracted RNA. Briefly, start with 1 gram of ground plant tissue for extraction. Prealiquot 10 milliliters (mL) Trizol reagent to 15 mL conical tubes. Add ground powder to tubes and shake to homogenize. Incubate the homogenized samples for 5 minutes (min) at room temperature (RT) and then add 3 mL of chloroform. Shakes tubes vigorously by hand for 15-30 seconds(sec) and incubate at RT for 3 min. Centrifuge the tubes at 7,000 revolutions per minute (rpm) for 10 min at 4 degrees C. Transfer the aqueous phase to a new 1.5 mL tube and add 1 volume of cold isopropanol. Incubate the samples for 20-30 min at RT and centrifuge at 10,000 rpm for 10 min at 4 degrees C. Wash pellet with Sigma-grade 80 percent ethanol. Remove the supernatant and briefly air-dry the pellet. Dissolve the RNA pellet in approximately 200 microliters of DEPC treated water. Heat briefly at 65 C to dissolve pellet and vortex or pipet to resuspend RNA pellet. Adjust RNA concentration to 1-2 microgram/microliter.

DNA was extracted using EZNA SP Plant DNA Mini kit (Omega Biotek, Norcross Ga., Cat#D5511) and Lysing Matrix E tubes (Q-Biogen, Cat#6914), following the manufacturer's protocol or modifications thereof by those skilled in the art of polynucleotide extraction that may enhance recover or purity of the extracted DNA. Briefly, aliquot ground tissue to a Lysing Matrix E tube on dry ice, add 800 μl Buffer SP1 to each sample, homogenize in a bead beater for 35-45 sec, incubate on ice for 45-60 sec, centrifuge at ≧14000 rpm for 1 min at RT, add 10 microliter RNase A to the lysate, incubate at 65° C. for 10 min, centrifuge for 1 min at RT, add 280 μl Buffer SP2 and vortex to mix, incubate the samples on ice for 5 min, centrifuge at ≧10,000 g for 10 min at RT, transfer the supernatant to a homogenizer column in a 2 ml collection tube, centrifuge at 10,000 g for 2 min at RT, transfer the cleared lysate into a 1.5 ml microfuge tube, add 1.5 volumes Buffer SP3 to the cleared lysate, vortex immediately to obtain a homogeneous mixture, transfer up to 650 μl supernatant to the Hi-Bind column, centrifuge at 10,000 g for 1 min, repeat, apply 100 μl 65° C. Elution Buffer to the column, centrifuge at 10,000 g for 5 min at RT.

Next-generation DNA sequencers, such as the 454-FLX (Roche, Branford, Conn.), the SOLiD (Applied Biosystems,), and the Genome Analyzer (HiSeq2000, Illumina, San Diego, Calif.) are used to provide polynucleotide sequence from the DNA and RNA extracted from the plant tissues. Raw sequence data is assembled into contigs. The contig sequence is used to identify trigger molecules that can be applied to the plant to enable regulation of the gene expression. The target DNA sequence isolated from genomic (gDNA) and coding DNA (cDNA) from the various weedy plant species for the HPPD gene and the assembled contigs as set forth in SEQ ID NOs 1-32 and Table 1.

### Example 2

### Polynucleotides of the Invention Related to the Trigger Molecules

The gene sequences and fragments of Table 1 were divided into 200 polynucleotide (200-mer) lengths with 25 polynucleotide overlapping regions SEQ ID NO:33-596. These polynucleotides are tested to select the most efficacious trigger regions across the length of any target sequence. The trigger polynucleotides are constructed as sense or anti-sense ssDNA or ssRNA, dsRNA, or dsDNA, or dsDNA/RNA hybrids and combined with an organosilicone based transfer agent to provide a polynucleotide preparation. The polynucleotides are combined into sets of two to three polynucleotides per set, using 4-8 nmol of each polynucleotide. Each polynucleotide set is prepared with the transfer agent and applied to a plant or a field of plants in combination with a glyphosate containing herbicide, or followed by a glyphosate treatment one to three days after the polynucleotide application, to determine the effect on the plant's susceptibility to glyphosate. The effect is measured as stunting the growth and/or killing of the plant and is measured 8-14 days after treatment with the polynucleotide set and glyphosate. The most efficacious sets are identified and the individual polynucleotides are tested in the same methods as the sets are and the most efficacious single 200-mer identified. The 200-mer sequence is divided into smaller sequences of 50-70-mer regions with 10-15 polynucleotide overlapping regions and the polynucleotides tested individually. The most efficacious 50-70-mer is further divided into smaller sequences of 25-mer regions with a 12 to 13 polynucleotide overlapping region and tested for efficacy in combination with HPPD inhibitor treatment. By this method it is possible to identify an oligonucleotide or several oligonucleotides that are the most efficacious trigger molecule to effect plant sensitivity to glyphosate or modulation of HPPD gene expression. The modulation of HPPD gene expression is determined by the detection of HPPD siRNA molecules specific to HPPD gene or by an observation of a reduction in the amount of HPPD RNA transcript produced relative to an untreated plant or by merely observing the anticipated phenotype of the application of the trigger with the glyphosate containing herbicide. Detection of siRNA can be accomplished, for example, using kits such as mirVana (Ambion, Austin Tex.) and mirPremier (Sigma-Aldrich, St Louis, Mo.).

The target DNA sequence isolated from genomic (gDNA) and coding DNA (cDNA) from the various weedy plant species for the HPPD gene and the assembled contigs as set forth in SEQ ID NOs 1-32 were divided into polynucleotide fragments as set forth in SEQ ID NO:33-596

The gene sequences and fragments of Table 1 are compared and 21-mers of contiguous polynucleotides are identified that have homology across the various HPPD gene sequences. The purpose is to identify trigger molecules that are useful as herbicidal molecules or in combination with an HPPD inhibitor herbicide across a broad range of weed species. The sequences (SEQ ID NO: 597-1082 represent the 21-mers that are present in the HPPD gene of at least six of the weed species of Table 1. It is contemplated that additional 21-mers can be selected from the sequences of Table 1 that are specific for a single weed species or a few weeds species within a genus or trigger molecules that are at least 18 contiguous nucleotides, at least 19 contiguous nucleotides, at least 20 contiguous nucleotides or at least 21 contiguous nucleotides in length and at least 85 percent identical to an HPPD gene sequence selected from the group consisting of SEQ ID NO:1-32.

By this method it is possible to identify an oligonucleotide or several oligonucleotides that are the most efficacious trigger molecule to effect plant sensitivity to glyphosate or modulation of HPPD gene expression. The modulation of HPPD gene expression is determined by the detection of HPPD siRNA molecules specific to HPPD gene or by an observation of a reduction in the amount of HPPD RNA transcript produced relative to an untreated plant. Detection of siRNA can be accomplished, for example, using kits such as mirVana (Ambion, Austin Tex.) and mirPremier (Sigma-Aldrich, St Louis, Mo.).

The target DNA sequence isolated from genomic (gDNA) and coding DNA (cDNA) from the various weedy plant species for the HPPD gene and the assembled contigs as set forth in SEQ ID NOs 1-32 were divided into fragments as set forth in SEQ ID NO: 597-1082.

### Example 3

### Methods Used in the Invention Related to Treating Plants or Plant Parts with a Topical Mixture of the Trigger Molecules

Glyphosate-sensitive Palmer amaranth (A. palmeri R-22) plants were grown in the greenhouse (30/20 C day/night T; 14 hour photoperiod) in 4 inch square pots containing Sun Gro® Redi-Earth and 3.5 kg/cubic meter Osmocote® 14-14-14 fertilizer. Palmer amaranth plants at 5 to 10 cm in height were pre-treated with a mixture of eight 8 short (21-22mer) single-strand antisense oligo DNA polynucleotides (ssDNAas) targeting HPPD shown in Table 2 as HPPD_OLIGO1-8 (SEQ ID NO: 1083-1090, respectively) at two concentrations, 16 nmol and 80 nmol, formulated in 10 millimolar sodium phosphate buffer (pH 6.8) containing 2% ammonium sulfate and 0.5% Silwet L-77. Plants were treated manually by pipetting 10 μL of polynucleotide solution on four fully expanded mature leaves, for a total of 40 microliters of solution per plant. Twenty-four and forty-eight hours later, the plants were treated with mesotrione (Callisto®, 4 lb ai per gallon; HPPD inhibitor) at 13 g ai/ha, or atrazine (Aatrex® Nine-0®, 90% w/w ai; Photosystem II inhibitor) at 170 g ai/ha using a track-sprayer equipped with a 9501E nozzle and calibrated to deliver 93 liters of solution per hectare. Crop oil concentrate (COC) at 1% was added to the herbicide treatments. Four replications of each treatment was conducted. Plant height was determined just before ssDNA treatment and at intervals upto twelve days after herbicide treatments to determine effect of the oligonucleotide and herbicide treatments.

TABLE 2
ssDNA HPPD oligonucleotides

| | | |
|---|---|---|
| HPPD_ | TCCGTAGCTTACATACCGAAG | CTTCGGTATGTAAGCTACGGA |
| OLIGO1 | | |
| HPPD_ | TCCAAGTGAATAGGAGAAACA | TGTTTCTCCTATTCACTTGGA |
| OLIGO2 | | |
| HPPD_ | AGCAGCTTCtgCGTCTTCTAC | GTAGAAGACGcaGAAGCTGCT |
| OLIGO3 | | |
| HPPD_ | ACAGCACGCACGCCAAGACCG | CGGTCTTGGCGTGCGTGCTGT |
| OLIGO4 | | |
| HPPD_ | CGaTGTAAGGAATTTGGtAAA | TTTaCCAAATTCCTTACAtCG |
| OLIGO5 | | |
| HPPD_ | CGAGGGGATTGCAGCAGAAGA | TCTTCTGCTGCAATCCCCTCG |
| OLIGO6 | | |
| HPPD_ | GTAGGAGaATacGGTGAAGTA | TACTTCACCgtATtCTCCTAC |
| OLIGO7 | | |
| HPPD_ | GACCCCAAGaAAATCGTCTGC | GCAGACGATTTtCTTGGGGTC |
| OLIGO8 | | |
| HPPD-T67 | ATTGAGGAGTACGAGAAGACT | AGTCTTCTCGTACTCCTCAAT |
| HPPD-T68 | CTTGAACGTAAACAGGTTCCA | TGGAACCTGTTTACGTTCAAG |

The results of the treatments demonstrated that plants treated only with 16 nmol and 80 nmol of the ssDNA oligonucleotides that targets HPPD showed growth stunting relative to the buffer control of 35 percent and 46 percent, respectively. Four days after treatment the plants treated with ssDNA followed by mesotrione or atrazine at 24 hours showed greater growth stunting than plants treated with the herbicide only. Thus, plants treated with ssDNA at 16 nmol and 80 nmol followed by mesotrione resulted in 77 and 75 percent growth reduction, respectively, relative to the buffer control. Plants treated with ssDNA at 16 nmol and 80 nmol followed by atrazine, resulted in 85 and 83 percent growth reduction, respectively, relative to the buffer control.

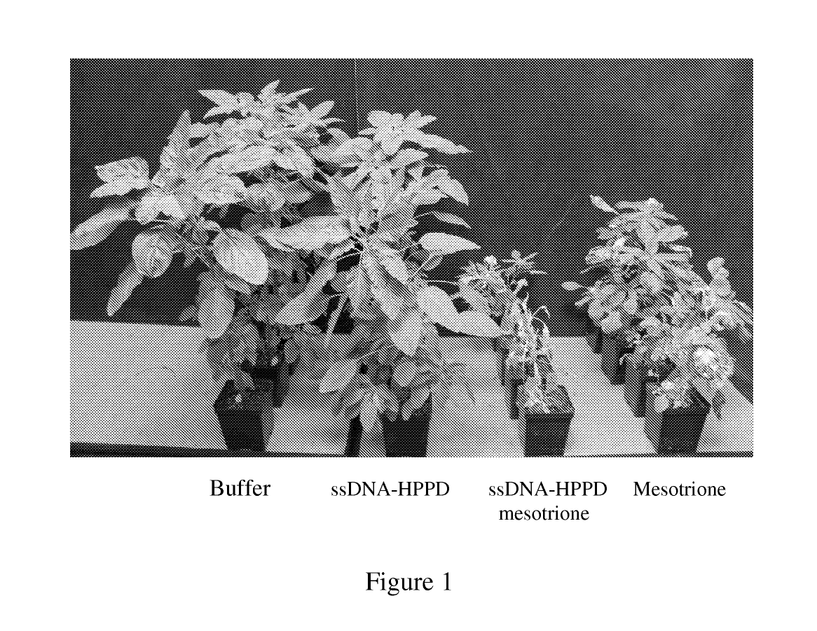

Twelve days after treatment the ssDNA at 16 nmol and 80 nmol provided 6 percent and 20 percent reduction in plant growth, the treatments that included mesotrione showed 91 and 89 percent growth reduction, compared to 48 percent control by mesotrione alone (FIG. 1). Plants treated with ssDNA at 16 nmol and 80 nmol followed by atrazine at 24 hours showed 50 and 74 percent growth reduction, compared to 29 percent control by atrazine alone. Thus, mesotrione and atrazine efficacy in Palmer amaranth can increase significantly by treating the plants with ssDNA that targets HPPD.

In another similar test, two pools of 5 double stand DNA oligonucleotides were tested, pool 1 contained HPPD-T67 (SEQ ID NO: 1091), HPPD-T68 (SEQ ID NO: 1092) and OLIGO1-3 of Table 2. Pool 2 contained OLIGO 4-8 of Table 2. Plants were treated with 10 nmoles of each oligonucleotide and sprayed with Diruon (DCMU (3-(3,4-dichlorophenyl)-1,1-dimethylurea, Bayer) and scored 14 days after treatment for effect on plant growth and development. The results indicate that the oligonucleotides increased the diuron sensitivity of the treated plants upto 22 percent.

### Example 4

### A Method to Control Weeds in a Field

A method to control weeds in a field comprises the use of trigger polynucleotides that can modulate the expression of an HPPD gene in one or more target weed plant species. An analysis of HPPD gene sequences from thirteen plant species provided a collection of 21-mer polynucleotides (SEQ ID NO:597-1082) that can be used in compositions to affect the growth or develop or sensitivity to glyphosate herbicide to control multiple weed species in a field. A composition containing 1 or 2 or 3 or 4 or more of the polynucleotides (SEQ ID NO:597-1082) would enable broad activity of the composition against the multiple weed species that occur in a field environment.

The method includes creating a composition that comprises components that include at least one polynucleotide of (SEQ ID NO:597-1082) or any other effective gene expression modulating polynucleotide essentially identical or essentially complementary to SEQ ID NO:1-32 or fragment thereof, a transfer agent that mobilizes the polynucleotide into a plant cell and a HPPD inhibiting herbicide and optionally a polynucleotide that modulates the expression of an essential gene and optionally a herbicide that has a different mode of action relative to an HPPD inhibitor. The polynucleotide of the composition includes a dsRNA, ssDNA or dsDNA or a combination thereof. A composition containing a polynucleotide can have a use rate of about 1 to 30 grams or more per acre depending on the size of the polynucleotide and the number of polynucleotides in the composition. The composition may include one or more additional herbicides as needed to provide effective multi-species weed control. A field of crop plants in need of weed plant control is treated by spray application of the composition. The composition can be provided as a tank mix, a sequential treatment of components (generally the polynucleotide followed by the herbicide), a simultaneous treatment or mixing of one or more of the components of the composition from separate containers. Treatment of the field can occur as often as needed to provide weed control and the components of the composition can be adjusted to target specific weed species or weed families.

TABLE 1
HPPD gene polynucleotide sequences

| SEQ ID NO | SPECIES | TYPE | LENGTH | Polynucleotide Sequence |
|---|---|---|---|---|
| 1 | Abutilon | cDNA | 713 | TCCATAATCCAACGCAGACATTTCAGAGATTCATCCCCGA |
| | theophrasti | Contig | | AGAAATCAAAAATCCCTCAACAAAATGATTCAGATATGC |
| | | | | TTAAACAAAATATGAATTGAGCACAATATCATATTTGAC |
| | | | | ATTTATGTTGAAGTAGCAAAGGTTCGTATAGTTTATGTAT |
| | | | | GTGCAATTAAAACTAACAGTAACGGAATTCATTCTGACA |
| | | | | CTCCCTATATGCTGCTAGCAACTACCTCGATCTCTCAAGC |
| | | | | CTCGACAACGGTGGTGGATTCTGTAGATTGTTTGGCTTC |
| | | | | AAGAGACTTCTCGTATTCTTCAATGGATTTGAAGAGCTC |
| | | | | GGAAAAGTTGCCTTTCCCGAAACCTCCGCATCCTCCTTTC |
| | | | | TGGTATTGCTTTCCTTCTTCATCCTTCACCATGCACCCTAA |
| | | | | TCTCTGTATTATTTCTATGAATATCGTAGGCCTATCTCCA |
| | | | | ATGGGCTTAGTGAAAATCTGAAGCAGAGTGCCTTGATCA |
| | | | | TCTCTGTCAACCAAAATCCCCAACTCTTCACACTCCTTAAT |
| | | | | CTGCTCATCGCTCAAAATGTCCCCTGCCCTTTGCTTCAAT |
| | | | | TTCTTATAGTAAGTGGGCGGCGGCGATGGCATGAACTC |
| | | | | GAAACCACCCACCAAACTTCTCTTCCTCATTTCTCTCAGC |
| | | | | GTTCTGAATATATCTTCACTCACCAGAGCCAAATGTTGAA |
| | | | | CCCCGGCACCTTCGTTGTGTTCTAAATACGTTTGGATTT |
| 2 | Amaranthus | cDNA | 1234 | CTTCTTCCCCGTCTGAGTTTTTATTACTTCACTTTCTCTCTC |
| | graecizans | Contig | | ATCATCTAACATGGGAACTTTGAAACCCGAAACTCAACC |
| | | | | CGACTCCGAATTCAAACTCGTGGGTTACTCCAACTTCATT |
| | | | | CGGGTTAACCCCAAATCTGACCGTTTTACTGTTAAGCGTT |
| | | | | TCCATCATATAGAGTTCTGGTGTGGCGATGCAACCAATG |
| | | | | TTAGCAGACGCTTTTCTTGGGGTCTTGGAATGCCTACCG |
| | | | | TTGCTAAATCTGACCTTTCTACTGGAAACTCTGTTCACGC |
| | | | | TTCTTTTCTTCTTCGTTCCGGTGACCTTTCTTTCCTCTTTAC |
| | | | | TTCACCTTACTCTCCTACCATGTCCATCCCTTCTTCTGCTG |
| | | | | CAATCCCCTCGTTTGATTTCAATCATTTTACCAAATTTGTT |
| | | | | ACATCGCACGGTCTTGGCGTGCGTGCTGTTGCCGTCGAA |
| | | | | GTAGAAGACGCAGAAGCTGCTTTTAATATCAGCGTTTCG |
| | | | | CACGGGGCTATCCCCTGTGTTTCTCCTATTCACTTGGAAA |
| | | | | ACGGTGTCGTTTTATCTGAGGTTCATTTATATGGGGATG |
| | | | | TTGTGCTTCGTTATGTAAGCTACGGAAATGAATGTGGGG |
| | | | | ATGTGTTTTTTCTTCCTGGGTTTGAGCAAATGCCCGAGG |
| | | | | AATCCTCGTTTCGAGGACTTGATTTCGGCCTTCGAAGGTT |
| | | | | GGATCATGCTGTAGGGAATGTCCCTGAGTTGGCTCCTGC |
| | | | | AATTGCTTATTTGAAGAAGTTTACTGGGTTTCATGAGTTT |
| | | | | GCTGAGTTTACAGCTGAAGATGTTGGAACGAGTGAAAG |
| | | | | TGGGTTGAATTCAGCCGTATTGGCAAATAATGATGAAAT |
| | | | | GGTGTTGTTTCCAATGAATGAACCTGTGTATGGGACAAA |
| | | | | AAGGAAGAGTCAAATTCAAACTTATTTGGAGCATAATGA |
| | | | | AGGAGCTGGTGTACAACATTTGGCTTTGATGAGTGAAG |
| | | | | ATATATTTTGGACTTTAAGGGAGATGAGGAAGAGAAGT |
| | | | | GGTCTTGGTGGGTTTGAGTTTATGCCGTCGCCGCCTCCG |
| | | | | ACTTATTACCGGAATTTGAGGAACAGAGCTGCTGATGTA |
| | | | | TTGAGTGAGGAGCAGATGAAGGAGTGTGAAGAGTTGG |
| | | | | GGATTTTGGTGGATAAAGATGATCAGGGTACTTTGCTTC |
| | | | | AAATCTTCACTAAGCCTATTGGTGACAGGCCAACCATATT |
| | | | | TATCGAGATTATACAAAGAATCGGTTGCATGATGAAAGA |
| | | | | TGAAGACGGCAAG |
| 3 | Amaranthus | cDNA | 547 | TCTTCCTGGGTTTGAGGAAATGCCGGAGGAATCGTCGTT |
| | hybridus | Contig | | TCGAGGACTTGATTTCGGCCTTCGAAGGTTGGATCATGC |
| | | | | TGTAGGGAATGTTCCCGAGTTGGCTCCTGCAATTGCTTA |
| | | | | TTTGAAGAAGTTTACTGGGTTTCATGAGTTTGCTGAGTTT |
| | | | | ACAGCTGAAGATGTTGGGACGAGTGAAAGTGGGTTGAA |
| | | | | TTCAGCCGTATTGGCAAATAATGATGAAATGGTGTTGTT |
| | | | | TCCAATGAATGAACCTGTGTATGGGACAAAAAGGAAGA |
| | | | | GCCAAATTCAAACTTATTTGGAGCATAATGAAGGAGCTG |
| | | | | GTGTACAACATTTGGCTTTGATGAGTGAAGATATATTTT |
| | | | | GGACTTTAAGGGAGATGAGGAAGAGAAGTGGTCTTGGT |
| | | | | GGGTTTGAGTTTATGCCGTCGCCGCCTCCGACTTATTACC |
| | | | | GGAATTTGAGGAATAGAGCTGCTGATGTATTGAGTGAG |
| | | | | GAGCAGATGAAGGAGTGTGAAGAGTTGGGGATTTTGGT |
| | | | | GGATAAAGATGATCAGGGTACTTTGCTTCAAATCTTCAC |
| | | | | TAAG |
| 4 | Amaranthus | cDNA | 1265 | AGATGTGTATAAGAGACAGGTCTTGGCGTGCGTGCTGTT |
| | palmeri | Contig | | GCCGTCGAAGTAGAAGACGCCGAAGCTGCTTTTAATATC |
| | | | | AGCGTTTCGCACGGGGCTATCCCCTGTGTTTCTCCTATTC |
| | | | | ACTTGGAAAACGGTGTCGTTTTATCTGAGGTTCATTTATA |
| | | | | TGGGGATGTTGTGCTTCGGTATGTAAGCTACGGAAATG |
| | | | | AATGTGGGGATGTGTTTTTCTTCCTGGGTTTGAGGAAAT |
| | | | | GCCGGAGGAATCATCGTTTCGAGGACTTGATTTTGGCAT |
| | | | | TCGAAGGTTGGATCATGCTGTAGGGAATGTCCCTGAGTT |
| | | | | GGCTCCTGCAATTGCTTATTTGAAGAAGTTTACTGGGTTT |
| | | | | CATGAGTTTGCTGAGTTTACAGCTGAAGATGTTGGGACG |
| | | | | AGTGAAAGTGGATTGAATTCAGCCGTATTGGCAAACAAT |
| | | | | GATGAAATGGTGTTGTTTCCAATGAATGAACCTGTGTAT |
| | | | | GGGACAAAAAGGAAGAGCCAAATTCAAACTTATTTGGA |
| | | | | GCATAATGAAGGAGCTGGTGTACAGCATTTGGCTTTGAT |
| | | | | GAGTGAAGACATATTTTGGACTTTAAGGGAGATGAGGA |
| | | | | AGAGAAGTGGTCTTGGTGGGTTTGAGTTTATGCCGTCGC |
| | | | | CGCCTCCGACTTATTACCGGAATTTGAGGAACAGAGCTG |
| | | | | CTGATGTATTGAGTGAGGAGCAGATGAAGGAGTGTGAA |
| | | | | GAGTTGGGGATTTTGGTGGATAAAGATGATCAGGGTAC |
| | | | | TTTGCTTCAAATCTTCACCAAACCTATTGGAGACAGGCCA |
| | | | | ACCATATTCATCGAGATTATCCAAAGAATTGGTTGCATG |
| | | | | ATGAAAGATGAAGACGGCAAGATGTACCAAAAGGGTG |
| | | | | GTTGCGGAGGATTCGGAAAGGGAAACTTTTCAGAGCTG |
| | | | | TTCAAATCAATAGAGGAGTACGAGAAGACTCTTGAACGT |
| | | | | AAACAGGTTCCAGATACAGCTGCTGCATGATGAGCAGA |
| | | | | CTAAAATATTGTTGTCTTGCTGATGAAATGATAGAAAAG |
| | | | | GTTTGTTTCTTGGTACAATGCTCAACTTCAAAATTTTCTTT |
| | | | | ATTAAATAATGAAGTGTAAACTTATACAAACTGTGTCAT |
| | | | | ATATGGTGATTGGTGATCATGGTTTATGTAGAATGTATA |
| | | | | ACATAATTGATAATCTGTGTATATGCTGAATACTTACATA |
| | | | | CTGTGAAATCATTCTGATGGAAACATACAATTGGTCAGT |
| | | | | AGCTGAGGCTGGTAGCTCCTCAGATTAGTTTTTTTCAGTC |
| | | | | AAAATCGTAGATGTATAT |
| 5 | Amaranthus | gDNA | 3245 | TTTTCTCTTTTATTTTTATCCAAAAATATCATGGGATGTCT |
| | palmeri | Contig | | AGATTGGAATAATTAGGGAGTAAAAAGTACCCCTTGATT |
| | | | | ATGCAGAGCAAAAATAAATTGTCTGAATTTGAAATAAAT |
| | | | | ATCTACAAGAGTAAATTTTTCCATCTTATTCAAAGGTAAA |
| | | | | TGTTTGATCCACCTACCTCTATAGATATATTTCAGGGAAC |
| | | | | TAAATTGTCTTCACCATTAAATTTGGTTACTTGGTCTTTAA |
| | | | | AACCCAAATCATAGAAATTAATGATAAAATCAAAATAAA |
| | | | | AAAGATATTTAAATTCAAATTCAAACTAACTAATTTTAAA |
| | | | | TTACAAAATGAATATCTGTAATTTACAAAAGAAAGTATC |
| | | | | AAAAACATATGAAAATCTCAACATCTGAAAATTACAAAC |
| | | | | AAGTATTCTGTTTTTTCATTTTTTTTTTCTTTTTCGCTATTT |
| | | | | CCTTTCAAAAATAAAAGTAAATAAAAATATTCAAAAGCA |
| | | | | ATTCCATAAACAAAATCTTAGATATGTAAATCACAAAAA |
| | | | | CATTAGATCTAGAAAAAAAAAATTTCTTCCATTGCAAACC |
| | | | | CTTTTTCAACCTTCATAACTTCCACTACCATAATGAGGCC |
| | | | | AGTAAAGAGACAAAAGTCATTGAGTTGTTGTTGTGCAGT |
| | | | | TGATGATAAATGATGATAGAAGGGTTTATTTTTTTTTTGA |
| | | | | AATGAATGGTTAGATTTTCTGACTTTTTATTTACCCTATA |
| | | | | ATGAATATCAAACAATTAACTCTATAAATTATTTAATACA |
| | | | | TTAAAATGTTTCATGTAATATGTCTCCTATATTATTTACCC |
| | | | | TTTAATTTTTAAGTGGGAACCAAGTATGTCTTAATTATCT |
| | | | | TTATTTTAATCAAATACGCGGTATACATGAAATAATCAAC |
| | | | | AAATGCAATTACTATGCTCGGACGAGAGTAAATATAATG |
| | | | | GGAGGAAGTTGTACATACAATTACGAAATAGTCTAAATA |
| | | | | AATAACGATAATTTGTAATATAAACAAACAAATCACACTT |
| | | | | ATATAAACAGATTTTATAGGGTGGAATCATTAGGATTCT |
| | | | | AATTTATCTTTTTTCTTCTTTTGTTTACTTTGCTGATATTTA |
| | | | | TTTTGTATTTTTCCTATTTCTCAAAAGGAAGACTAACACT |
| | | | | CAAATAAAATGATATTGAAATACAAAGCATCACCGGCCA |
| | | | | AGCCGAGATGACGAAACTATTTTGAATAATTATGATGAT |
| | | | | TTACAACTCCAAATAGAAGTAATTGATCAAGACTTTAGG |
| | | | | ACTTGGAAGTGTTGGGCAAAATCTTCCAGAGTCCAGGAT |
| | | | | AAGTGATAAGTGACGTATTTCCGTTACTCTTAAGTGTTAA |
| | | | | CAGCTTTTTGTCACGCAAGGAAAAGAAGACCGTGGACG |
| | | | | TCAACGATGACGTTGAATGTTCATCTTTACAGTCGCAGTC |
| | | | | AATCAATCTCTTTTTAGATCGATCTTCCACCTCAATTCTCC |
| | | | | GTTACAAATCAAATTCCATCTAGAACTTCTTTTTTATTATT |
| | | | | TTGACTCATAAATTCCCCCAAAAATACTTCTATTTTATTAT |
| | | | | AAATAAATTCCAATTTCTATGTTCTCCATTCATTACCACCC |
| | | | | ATTACTCCGTTTTCCAAACCACCATTTTCTCTCTCCTCCTTT |
| | | | | ACCGCTAACGCTACCACCATTTTCGCTTCTTCCCCGTCTG |
| | | | | AATTTTATTACTTCGCTTTCTCTATCATCATCTGACATGGG |
| | | | | TACTTTGAAACCCGAAACTCAACCCGATTCCGAATTCAAA |
| | | | | CTCGTGGGTTACTCCAACTTCGTTCGGGTTAACCCCAAAT |
| | | | | CTGACCGTTTTGCTGTTAAGCGTTTCCACCATATAGAGTT |
| | | | | TTGGTGTGGCGATGCAACCAATGTTAGCAGACGATTTTC |
| | | | | TTGGGGTCTTGGAATGCCTACCGTTGCTAAATCTGACCTT |
| | | | | TCTACAGGAAACTCTGTTCACGCTTCTTTTCTTCTTCGTTC |
| | | | | CGGTGACCTTTCTTTTCTCTTTACTTCACCTTACTCTCCTA |
| | | | | CCATGTCCATCCCTTCTTCTGCTGCAATCCCCTCGTTTGAT |
| | | | | TTCAATCATTTTACCAAATTCCTTACATCGCACGGTCTTG |
| | | | | GCGTGCGTGCTGTTGCCGTCGAAGTAGAAGACGCGGAA |
| | | | | GCTGCTTTTAACATCAGCGTTTCGCATGGGGCTATTCCCT |
| | | | | GTGTTTCTCCTATTCAATTGGAAAACGGTGTCGTTTTATC |
| | | | | TGAGGTTCATTTATATGGGGATGTTGTGCTTCGGTATGT |
| | | | | AAGCTACGGAAATGAATGTGGGGATGTGTTTTTTCTTCC |
| | | | | TGGGTTTGAGGAAATGCCGGAGGAATCATCGTTTCGAG |
| | | | | GACTTGATTTTGGCATTCGAAGGTTGGATCATGCTGTAG |
| | | | | GGAATGTCCCTGAGTTGGCTCCTGCAATTGCTTATTTGA |
| | | | | AGAAGTTTACTGGGTTTCATGAGTTTGCTGAGTTTACAG |
| | | | | CTGAAGATGTTGGGACGAGTGAAAGTGGATTGAATTCA |
| | | | | GCCGTATTGGCAAACAATGATGAAATGGTGTTGTTTCCA |
| | | | | ATGAATGAACCTGTGTATGGGACAAAAAGGAAGAGCCA |
| | | | | AATTCAAACTTATTTGGAGCATAATGAAGGAGCTGGTGT |
| | | | | ACAGCATTTGGCTTTGATGAGTGAAGACATATTTTGGAC |
| | | | | TTTAAGGGAGATGAGGAAGAGAAGTGGTCTTGGTGGGT |
| | | | | TTGAATTTATGCCGTCGCCGCCTCCGACTTATTACCGGAA |
| | | | | TTTGAGGAGCAGAGCTGCTGATGTATTGAGTGAGGAGC |
| | | | | AGATGAAGGAGTGTGAAGAGTTGGGGATTTTGGTGGAT |
| | | | | AAAGATGATCAGGGCACTTTGCTTCAAATCTTCACCAAA |
| | | | | CCTATTGGAGACAGGCCAACCATATTCATCGAGATTATC |
| | | | | CAAAGAATTGGTTGCATGATGAAAGATGAAGACGGCAA |
| | | | | GATGTACCAAAAGGGTGGTTGCGGAGGATTTGGAAAGG |
| | | | | GAAACTTTTCAGAGTTGTTCAAATCAATTGAGGAGTACG |
| | | | | AGAAGACTCTTGAACGTAAACAGGTTCCAGATACAGCTG |
| | | | | CTGCATGATGAGCAGACTGAAATATTGCTGTCTTGCTGG |
| | | | | TGGAAGCCATATAATGGTAATATGATAGAAAAGGTTTGT |
| | | | | TGCTCAAAATTTTCTTTATTAAATAATGAAGTGTAAACTT |
| | | | | ATACAAACTGTGTCATATATGGTGATTGATGATCATGCA |
| | | | | TGGTTATGTAGAATGTATAACATAATTGATAATCTGTGT |
| | | | | ATATGCTGAAAACTTACATACTGTGAAATCATTCTGATAG |
| | | | | AAACATACAATTGGTGAGTAGCTGTCTCTTATACACATCT |
| 6 | Amaranthus | gDNA | 3416 | GGAGGAATTTTTTTGTGCATGTAAAATGTTTTCTCTCTAT |
| | palmeri | Contig | | TTTTTGATTTATGCTATTTTTTCTCTTTTATTTTTATCCAAA |
| | | | | AATATCATGGGATGTCTAGATTGGAATAATTAGGGAGTA |
| | | | | AAAAGTACCCCTTGATTATGCAGAGCAAAAATAAATTGT |
| | | | | CTGAATTTGAAATAAATATCTACAAGAGTAAATTTTTCCA |
| | | | | TCTTATTCAAAGGTAAATGTTTGATCCACCTACCTCTATA |
| | | | | GATATATTTCAGGGAACTAAAATTGTCTTCACCATTATAT |
| | | | | TTAGTTACTTGGTCTTTAAAACCCAAATCATAGAAATTAA |
| | | | | CGATAAAATCAAAATATAAAGATATTTAAATTCCAATTCA |
| | | | | AACTAACTAATTTTAAATTACAAAATGAATATCTGTAATT |
| | | | | TACAAAAGAAAGTATCAAAAACATATGAAAATCTCAACA |
| | | | | TCTGAAAATTAAAAAACAAGTATTTTGTTTCTTCATTTTTT |
| | | | | TCTTTTTCGCTATTTCCTTTCAAAAATAAAAGTAAATAAA |
| | | | | AATATTCAAAAGCAATTCCATAAACAAAATCTTAGATAT |
| | | | | GTAAATCACAAAAACATTAGATCTAGAAAAAAAAAATTT |
| | | | | CTTCCATTGCAAACCCTTTTTCAACCTTCATAACTTCCACT |
| | | | | ACCATAATGAGGCCAGTAAAGAGACAAAAGTCATTGAG |
| | | | | TTGTTGTTGTGCAGTTGATGATAAATGATGATAGAAGGG |
| | | | | TTTATTTTTTTTTTGAAATGAATGGTTAGATTTTCTGACTT |
| | | | | TTTATTTACCCTATATAGAATATCAAACAATTAACTCTATA |
| | | | | AATTATTTAATACATTAAAATGTTTCATGTAATATGTCTC |
| | | | | CTATATTATTTACCCTTTAATTTTTAAGTGGGAACCAAGT |
| | | | | ATGTCTTAATTATCTTTATTTTAATCAAATACGCGGTATA |
| | | | | CATGAAATAATCAACAAATGCAATTACTATGCTCGGACG |
| | | | | AGAGTAAATATAATGGGAGGAAGTTGTACATACAATTAC |
| | | | | GAAATAGTCTAAATAAATAACGATAATTTGTAATATAAA |
| | | | | CAAACAAATCACACTTATATAAACAGATTTTATAGGGTG |
| | | | | GAATCATTAGGATTCTAATTTATCTTTTTTCTTCTTTTGTTT |
| | | | | ACTTTGCTGATATTTATTTTGTATTTTTCCTATTTCTCAAA |
| | | | | AGGAAGACTAACACTCAAATAAAATGATATTGAAATACA |
| | | | | AAGCATCACCAGCCAAGCCGAGATGACAAAACTATTGG |
| | | | | CTAAGTGATAACTGATAAGTGACGTATTTCCGTTACTCTC |
| | | | | AAGTCTTAACAGCTTTTTGTCACGCAAGGAAAAGAAGAC |
| | | | | CGTGGACGTCAACGGTGACGTTGAATGTTCATCTTTACA |
| | | | | GTCGCAGTCAATCAATCTCTTTTTAGATCGATCTTCCACC |
| | | | | TCAATTCTCCGTTACAAATCAAATTCCATCTAGAACTTCTT |
| | | | | TTTAATTATTTTGACTCATAAATTCCCCCAAAAATACTTCT |
| | | | | ATTTTATTATAAATAAATTCCAATTTCTATGTTCTCCATTC |
| | | | | ATCACCACCCATTACTCCGTTTTCCAAACCACCATTTTCTC |
| | | | | TCTCCTCCATTACCCCTAACACAACTACCATTTTCGCTTCT |
| | | | | TCCCCGTCTGAGTCTTATTACATCGCTTTCTCTCTCATCAT |
| | | | | CTGACATGGGAACTTTGAAACCCGAAACTCAACCCGATT |
| | | | | CCGAATTCAAACTCGTGGGTTACTCCAACTTCGTTCGGG |
| | | | | TTAACCCCAAATCTGACCGTTTTGCTGTTAAGCGTTTCCA |
| | | | | CCATATAGAGTTTTGGTGTGGCGATGCAACCAATGTTAG |
| | | | | CAGACGATTTTCTTGGGGTCTTGGAATGCCTATCGTCGC |
| | | | | TAAATCTGACCTGTCTACAGGAAACTCTGTTCACGCTTCT |
| | | | | TTTCTTCTTCGTTCCGGTGACCTTTCTTTTCTCTTTACTTCA |
| | | | | CCGTATTCTCCTACCATGTCCATCCCTTCTTCTGCTGCAAT |
| | | | | CCCCTCGTTTGATTTCAATCATTTTACCAAATTCCTTACAT |
| | | | | CGCACGGTCTTGGCGTGCGTGCTGTTGCCGTCGAAGTAG |
| | | | | AAGACGCCGAAGCTGCTTTTAATATCAGCGTTTCGCACG |
| | | | | GGGCTATCCCCTGTGTTTCTCCTATTCACTTGGAAAACGG |
| | | | | TGTCGTTTTATCTGAGGTTCATTTATATGGGGATGTTGTG |
| | | | | CTTCGGTATGTAAGCTACGGAAATGAATGTGGGGATGT |
| | | | | GTTTTTTCTTCCTGGGTTTGAGGAAATGCCGGAGGAATC |
| | | | | ATCGTTTCGAGGACTTGATTTTGGCATTCGAAGGTTGGA |
| | | | | TCATGCTGTAGGGAATGTCCCTGAGTTGGCTCCTGCAAT |
| | | | | TGCTTATTTGAAGAAGTTTACTGGGTTTCATGAGTTTGCT |
| | | | | GAGTTTACAGCTGAAGATGTTGGGACGAGTGAAAGTGG |
| | | | | ATTGAATTCAGCCGTATTGGCAAACAATGATGAAATGGT |
| | | | | GTTGTTTCCAATGAATGAACCTGTGTATGGGACAAAAAG |
| | | | | GAAGAGCCAAATTCAAACTTATTTGGAGCATAATGAAGG |
| | | | | AGCTGGTGTACAGCATTTGGCTTTGATGAGTGAAGACAT |
| | | | | ATTTTGGACTTTAAGGGAGATGAGGAAGAGAAGTGGTC |
| | | | | TTGGTGGGTTTGAATTTATGCCGTCGCCGCCTCCGACTTA |
| | | | | TTACCGGAATTTGAGGAGCAGAGCTGCTGATGTATTGA |
| | | | | GTGAGGAGCAGATGAAGGAGTGTGAAGAGTTGGGGAT |
| | | | | TTTGGTGGATAAAGATGATCAGGGCACTTTGCTTCAAAT |
| | | | | CTTCACCAAACCTATTGGAGACAGGTAGATTTTAATCTTG |
| | | | | CTTTCAATTGCTTTTGCTTGATTGATTGACTAGCCAATTTT |
| | | | | GATTGCATTTTGTTGCTTATATGACTTGATGATAATAGAT |
| | | | | GGTTTACCTTTCTCAGCTGTTCATTTGTAGCCAGTATAGA |
| | | | | TTCGTTCTAAAATATTTGCAACTGATTATGACATGTAGTA |
| | | | | GCAGAAAATGTCCCTATATTGGATGTTTGGCATAAATTA |
| | | | | AGCTTGGTTTTGCACTTATCCTCATTTATTTATAAATTCTA |
| | | | | AAACTTGTTAGTTGTAATTAAGTTAATGAGAACAAAGCC |
| | | | | TTAATATTCCTTCAAGGTGATTGTAGTTGGGGCACTAGTT |
| | | | | CTAACAATGGAAATTTGGAAATCTATTCCAACTGGTCCC |
| | | | | AAGTTAATCTTTGTTTGCAAGCCTGATTGGTTCAAATTAA |
| | | | | GGTTATTGTATTCTTGTATGAATTCGACTCAATGTAAATT |
| | | | | TGTTTAATGGAGCATCAATTTTTAATAGTTTTCGACCAAG |
| | | | | CAGTATTAGATATATTCGGGATTGAAATAATGCATCTAT |
| | | | | GAGTGTATAAAACCAAATGGCACATTTGATTAGAATAAA |
| | | | | AGAGAGTATAAGGCTAATTTCGTTTACCTAATATTTAAA |
| | | | | GCGACCCCTAAAATTCAATTGGCCTAAACCCATAAAGTT |
| | | | | CAAA |
| 7 | Amaranthus | gDNA | 3818 | GAGTTCAAGATTAAAAGTTAAATTACATTTATGTGGGTT |
| | palmeri | Contig | | TTATATAGGCAATGATTGCATTATATTGTTTTTCTTTTGGT |
| | | | | GGGAAGATTTTCCTTTTTAAAAAATTTTAATTTCCCTACA |
| | | | | TTTTCAAATGATGGAGGAATTTTTTTGTGCATGTAAAATG |
| | | | | TTTTCTCTCTATTTTTTGATTTATGCTATTTTTTCTCTTTTAT |
| | | | | TTTTATCCAAAAATATCATGGGATGTCTAGATTGGAATA |
| | | | | ATTAGGGAGTAAAAAGTACCCCTTGATTATGCAGAGCAA |
| | | | | AAATAAATTGTCTGAATTTGAAATAAATATCTACAAGAG |
| | | | | TAAATTTTTCCATCTTATTCAAAGGTAAATGTTTGATCCA |
| | | | | CCTACCTCTATAGATATATTTCAGGGAACTAAATTGTCTT |
| | | | | CACCATTAAATTTGGTTACTTGGTCTTTAAAACCCAAATC |
| | | | | ATAGAAATTAATGATAAAATCAAAATAAAAAAGATATTT |
| | | | | AAATTCAAATTCAAACTAACTAATTTTAAATTACAAAATG |
| | | | | AATATCTGTAATTTACAAAAGAAAGTATCAAAAACATAT |
| | | | | GAAAATCTCAACATCTGAAAATTAAAAAACAAGTATTTT |
| | | | | GTTTCTTCATTTTTTTCTTTTTCGCTATTTCCTTTCAAAAAT |
| | | | | AAAAGTAAATAAAAATATTCAAAAGCAATTCCATAAACA |
| | | | | AAATCTTAGATATGTAAATCACAAAAACATTAGATCTAG |
| | | | | AAAAAAAAAATTTCTTCCATTGCAAACCCTTTTTCAACCT |
| | | | | TCATAACTTCCACTACCATAATGAGGCCAGTAAAGAGAC |
| | | | | AAAAGTCATTGAGTTGTTGTTGTGCAGTTGATGATAAAT |
| | | | | GATGATAGAAGGGTTTATTTTTTTTTTGAAATGAATGGTT |
| | | | | AGATTTTCTGACTTTTTATTTACCCTATATAGAATATCAAA |
| | | | | CAATTAACTCTATAAATTATTTAATACATTAAAATGTTTCA |
| | | | | TGTAATATGTCTCCTATATTATTTACCCTTTAATTTTTAAG |
| | | | | TGGGAACCAAGTATGTCTTAATTATCTTTATTTTAATCAA |
| | | | | ATACGCGGTATACATGAAATAATCAACAAATGCAATTAC |
| | | | | TATGCTCGGACGAGAGTAAATATAATGGGAGGAAGTTG |
| | | | | TACATACAATTACGAAATAGTCTAAATAAATAACGATAA |
| | | | | TTTGTAATATAAACAAACAAATCACACTTATATAAACAGA |
| | | | | TTTTATAGGGTGGAATCATTAGGATTCTAATTTATCTTTT |
| | | | | TTCTTCTTTTGTTTACTTTGCTGATATTTATTTTGTATTTTT |
| | | | | CCTATTTCTCAAAAGGAAGACTAACACTCAAATAAAATG |
| | | | | ATATTGAAATACAAAGCATCACCAGCCAAGCCGAGATGA |
| | | | | CAAAACTATTGGCTAAGTGATAACTGATAAGTGACGTAT |
| | | | | TTCCGTTACTCTCAAGTCTTAACAGCTTTTTGTCACGCAA |
| | | | | GGAAAAGAAGACCGTGGACGTCAACGGTGACGTTGAAT |
| | | | | GTTCATCTTTACAGTCGCAGTCAATCAATCTCTTTTTAGA |
| | | | | TCGATCTTCCACCTCAATTCTCCGTTACAAATCAAATTCCA |
| | | | | TCTAGAACTTCTTTTTTATTATTTTGACTCATAAATTCCCC |
| | | | | CAAAAATACTTCTATTTTATTATAAATAAATTCCAATTTCT |
| | | | | ATGTTCTCCATTCATTACCACCCATTACTCCGTTTTCCAAA |
| | | | | CCACCATTTTCTCTCTCCTCCTTTACCGCTAACGCTACCAC |
| | | | | CATTTTCGCTTCTTCCCCGTCTGAATTTTATTACTTCGCTT |
| | | | | TCTCTATCATCATCTGACATGGGAACTTTAAAACCCGAAA |
| | | | | CTCAACCCGATTCCGAATTCAAACTCGTGGGTTACTCCAA |
| | | | | CTTCGTCCGGGTTAACCCCAAATCTGACCGTTTTGCTGTT |
| | | | | AAGCGTTTCCACCATATAGAGTTTTGGTGTGGCGATGCA |
| | | | | ACCAATGTTAGCAGACGATTTTCTTGGGGTCTTGGAATG |
| | | | | CCTACCGTCGCTAAATCTGACCTGTCTACAGGAAACTCTG |
| | | | | TTCACGCTTCTTTTCTTCTTAGTTCCGGTGACCTTTCTTTT |
| | | | | CTCTTTACTTCACCTTACTCTCCTACCATGTCCATCCCTTCT |
| | | | | TCTGCTGCAATCCCCTCGTTTGATTTCAATCATTTTACCAA |
| | | | | ATTCCTTACATCGCACGGTCTTGGCGTGCGTGCTGTTGCC |
| | | | | GTCGAAGTAGAAGACGCAGAAGCTGCTTTTAATATCAGC |
| | | | | GTTTCGCACGGGGCTATCCCCTGTGTTTCTCCTATTCACT |
| | | | | TGGAAAACGGTGTCGTTTTATCTGAGGTTCATTTATATG |
| | | | | GGGATGTTGTGCTTCGGTATGTAAGCTACGGAAATGAAT |
| | | | | GTGGGGATGTGTTTTTTCTTCCTGGGTTTGAGGAAATGC |
| | | | | CGGAGGAATCATCGTTTCGAGGACTTGATTTTGGCATTC |
| | | | | GAAGGTTGGATCATGCTGTAGGGAATGTCCCTGAGTTG |
| | | | | GCTCCTGCAATTGCTTATTTGAAGAAGTTTACTGGGTTTC |
| | | | | ATGAGTTTGCTGAGTTTACAGCTGAAGATGTTGGGACGA |
| | | | | GTGAAAGTGGGTTGAATTCAGCCGTATTGGCAAACAAT |
| | | | | GATGAAATGGTGTTGTTTCCGATGAATGAACCTGTGTAT |
| | | | | GGGACAAAAAGGAAGAGCCAAATTCAAACTTATTTGGA |
| | | | | GCATAATGAAGGAGCTGGTGTACAGCATTTGGCTTTGAT |
| | | | | GAGTGAAGACATATTTTGGACTTTAAGGGAGATGAGGA |
| | | | | AGAGAAGTGGTCTTGGTGGGTTTGAGTTTATGCCGTCGC |
| | | | | CGCCTCCGACTTATTACCGGAATTTGAGGAACAGAGCTG |
| | | | | CTGATGTATTGAGTGAGGAGCAGATGAAGGAGTGTGAA |
| | | | | GAGTTGGGGATTTTGGTGGATAAAGATGATCAGGGCAC |
| | | | | TTTGCTTCAAATCTTCACCAAACCTATTGGAGACAGGTAG |
| | | | | ATTTTAATCTTGCTTTCAATTGCTTTTGCTTGATGGATTGA |
| | | | | CTAGCCAATTTGATTGCATTTTGTTGCTTATATGACTTGA |
| | | | | TGCTAGATAGTTTACCTTTCTCAGCTGTTAAGTTGTAGCA |
| | | | | AGTATCTAATACATTCGTTCTGAAATATCTGAAATATTTG |
| | | | | CAACTGATTATGACATGTAGCGGGAGAAAATGTCCGTTT |
| | | | | GGCATAAATTAAGCTTGGTTTTGCACTTATCCTCATTTAT |
| | | | | TTATAAATTCTAAAACTTGTTAGTTGTAAGCTCCTTTCAG |
| | | | | TTGTCCTGAATTTAATTAAGTTAATGAGAACAAAGCCTTA |
| | | | | ATATTCCTTCAAGGTGATTGTAGTTGGGGCACTAGTTCT |
| | | | | AACAATGGAAATTTGGAAATCTATTCCAACTGGTCCCAA |
| | | | | GTTAATCTTTGTTTGCAAGCCTGATTGGTTCAAATTAAGG |
| | | | | TTATTGTATTCTTGTATGAATTCGACTCAATGTAAATTTG |
| | | | | TTTAATGGAGTATCAATTTTTAATAGTTTTCAACCAAGTA |
| | | | | GTATTAGATATATTCGGGATTGAAATAATGCATCTATGA |
| | | | | GTGTACAAAACCAAATGGCACATTTGATTAGAATAAAAG |
| | | | | AGAGTATAAGGCTAATTTCGTTTACCTAATATTTAAAGCG |
| | | | | ACCCCTAAAATTCAATTGGCCTAAACCCATAAAGTTCAAA |
| | | | | AGCAGAGAAGAACATAGAATAGTGCAGGTCCATTGGTA |
| | | | | ATGCACTAGGAGTTGGAGCTTTTATGGGTACAAGTGTGT |
| | | | | GGCTAGTTGGGGATGACTGTCTAGCATTGTCTAGGTGAA |
| | | | | AAGCTGAAGCCTTAAGCCATGAAGGTTTTGAGTAGAGG |
| | | | | TGTTCATTTGGGTCATCGGGTTGATTTCGGGTCAGATGT |
| | | | | TTCGGGTCGGTTTAAAATCGGGTTTTGTGTTCACATTGGT |
| | | | | TTTTACGTAATT |
| 8 | Amaranthus | cDNA | 775 | AAATGAATGTGGGGATGTGTTTTTTCTTCCTGGGTTTGA |
| | rudis | Contig | | GGAAATGCCGGAGGAATCATCGTTTCGAGGACTTGATTT |
| | | | | CGGCCTTCGAAGGTTGGATCATGCTGTAGGGAATGTCCC |
| | | | | TGAGTTGGCTCCTGCAATTGCTTATTTGAAGAAGTTTACT |
| | | | | GGGTTTCATGAGTTTGCTGAGTTTACAGCTGAAGATGTT |
| | | | | GGGACGAGTGAAAGTGGGTTGAATTCAGCCGTATTGGC |
| | | | | AAATAATGATGAAATGGTGTTGTTTCCAATGAATGAACC |
| | | | | AGTGTATGGGACAAAAAGGAAGAGTCAAATTCAAACTT |
| | | | | ATTTGGAGCATAATGAAGGAGCTGGTGTACAACATTTGG |
| | | | | CTTTGATGAGTGAAGATATATTCTGGACTTTAAGGGAGA |
| | | | | TGAGGAAGAGAAGTGGTCTTGGTGGGTTTGAGTTTATG |
| | | | | CCGTCGCCGCCTCCGACTTATTACCGGAATTTGAGGAAC |
| | | | | AGAGCTGCTGATGTATTGAGTGAGGAGCAGATGAAGGA |
| | | | | GTGTGAAGAGTTGGGGATTTTGGTGGATAAAGATGATC |
| | | | | AGGGTACTTTGCTTCAAATCTTCACCAAACCTATTGGAGA |
| | | | | CAGGCCAACTATATTTATCGAGATCATCCAAAGAATTGG |
| | | | | TTGCATGATGAAAGATGAAGACGGCAAGATGTACCAAA |
| | | | | AGGGTGGTTGCGGAGGATTTGGAAAGGGAAACTTTTCG |
| | | | | GAGCTTTTCAAATCAATTGAGGAGTACGAGAAGACTCTT |
| | | | | GAACGTAAACAGGTTCCAGATACAGCTGCTGCATGAGTT |
| 9 | Amaranthus | cDNA | 1204 | CTTACATCGCACGGTCTTGGTGTGCGTGCTGTTGCTGTC |
| | rudis | Contig | | GAAGTAGAGGACGCAGAAGCTGCTTTTAATATCAGCGTT |
| | | | | TCCAACGGGGCTATTCCCTGTGTTTCTCCTATTCAATTGG |
| | | | | AAAACGGTGTCGTTTTATCTGAGGTTCATTTATATGGGG |
| | | | | ATGTTGTGCTTCGCTATGTAAGCTACGGAAATGAACGTG |
| | | | | GGGATGTGTTTTTTCTTCCTGGGTTTGAGGAAATGCCGG |
| | | | | AGGAATCGTCGTTTCGAGGACTTGATTTCGGCCTTCGAA |
| | | | | GGTTGGATCATGCTGTAGGGAATGTTCCCGAGTTGGCTC |
| | | | | CTGCAATTGCTTATTTGAAGAAGTTTACTGGGTTTCATGA |
| | | | | GTTTGCTGAGTTTACAGCTGAAGATGTTGGGACGAGTG |
| | | | | AAAGTGGGTTGAATTCAGCCGTATTGGCAAATAATGATG |
| | | | | AAATGGTGTTGTTTCCAATGAATGAACCTGTGTATGGGA |
| | | | | CAAAAAGGAAGAGTCAAATTCAAACTTATTTGGAGCATA |
| | | | | ATGAAGGAGCTGGTGTACAACATTTGGCTTTGATGAGTG |
| | | | | AAGATATATTTTGGACTTTAAGGGAGATGAGGAAGAGA |
| | | | | AGTGGTCTTGGTGGGTTTGAGTTTATGCCGTCGCCGCCT |
| | | | | CCGACTTATTACCGGAATTTGAGGAATAGAGCTGCTGAT |
| | | | | GTATTGAGTGAGGAGCAGATGAAGGAGTGTGAAGAGTT |
| | | | | GGGGATTTTGGTGGATAAAGATGATCAGGGTACTTTGCT |
| | | | | TCAAATCTTCACTAAGCCTATTGGTGACAGGCCAACTATA |
| | | | | TTTATCGAGATCATCCAAAGAATTGGTTGCATGATGAAA |
| | | | | GATGAAGACGGCAAGATGTACCAAAAGGGTGGTTGCGG |
| | | | | AGGATTTGGAAAGGGAAACTTTTCGGAGCTTTTCAAATC |
| | | | | AATTGAGGAGTATGAGAAGACTCTTGAACGTAAACAGG |
| | | | | TTCCAGATACAGCTGCTGCATGAGCAGACTAAAATATTG |
| | | | | CTGAAACGCAGGCTGCAGCCATATGTTAGAACAGTCATT |
| | | | | CTGATGGAAACACTCAAGCGGTGAGTAGCTGAGGTTGG |
| | | | | TGATGCTGAAGTCGAGTCGGTATTTGGATCATCTTACAA |
| | | | | TTACAGTGCAAGGATAGTAATGAAGCATGTAAACAGCTC |
| | | | | CTCAGATTAGTTTTTTCCAGTCATAATCGTAGATGTATAT |
| | | | | GAGAAAATTTAAATTGCTCTTTTAAGTTAATGAAAA |
| 10 | Amaranthus | gDNA | 511 | TCATCAACAACAAAAGAGTTGAAATTCTAATAATAATCC |
| | rudis | Contig | | CAGTAAGCAGTGAGTAAGATCAAATGGAGAGTCAGTTA |
| | | | | GTAGCTAATCATACAAAACCATTAAAGCTACAAAGTTAC |
| | | | | TCTAATTTCTTAAGATCAAACCCAAAATCTGATCGTTTCA |
| | | | | AAGTGAAAAGGTTCCACCACATTGAGTTCTGGTGTGGTG |
| | | | | ATGCAACCAACACTAGCCTTTTATTTTCGTTAGGGCTTGG |
| | | | | CATGCCTATGGTTGCCAAATCCGATCTCTCCACAGGCAA |
| | | | | CCTTATACATGCCTCCTACGTCTTACGTAGTGGCGAACTT |
| | | | | TGTTTCGTATTCACGGCTCCTTACTCTCCGTCATCCATGCT |
| | | | | CACAACTGCATCAATTCCTTCGTTTGACTACAGTGCGCAC |
| | | | | GCCTCTTTTGTGTCCTGTCATGGCCTTGGTGTTCGCGCTG |
| | | | | TGGCCCTTGAGGTGGAGAACGCTGAATCCGCCTTTAGG |
| | | | | ATTAGTGTTGCAGCAGGAGCTCATCCGTCAGCCCCACC |
| 11 | Amaranthus | gDNA | 770 | AATCATTTTACCAAATTCCTTACATCGCACGGTCTTGGCG |
| | rudis | Contig | | TGCGTGCTGTTGCTGTCGAAGTAGAAGACGCAGAAGCT |
| | | | | GCTTTTAATATCAGCGTTTCCAACGGGGCTATTCCCTGTG |
| | | | | TTTCTCCTATTCACTTGGAAAACGGTGTCGTTTTATCTGA |
| | | | | GGTTCATTTATATGGGGATGTTGTGCTTCGGTATGTAAG |
| | | | | CTACGGAAATGAATGTGGGGATGTGTTTTTTCTTCCTGG |
| | | | | GTTTGAGGAAATGCCGGAGGAATCATCGTTTCGAGGAC |
| | | | | TAGATTTCGGCCTTCGAAGGTTGGATCATGCTGTAGGGA |
| | | | | ATGTCCCTGAGTTGGCTCCTGCAATTGCTTATTTGAAGAA |
| | | | | GTTTACTGGGTTTCATGAGTTTGCTGAGTTTACAGCTGA |
| | | | | AGATGTTGGGACGAGTGAAAGTGGGTTGAATTCAGCCG |
| | | | | TATTGGCAAATAATGATGAAATGGTGTTGTTTCCAATGA |
| | | | | ATGAACCAGTGTATGGGACAAAAAGGAAGAGTCAAATT |
| | | | | CAAACTTATTTGGAGCATAATGAAGGAGCTGGTGTACAA |
| | | | | CATTTGGCTTTGATGAGTGAAGATATATTCTGGACTTTAA |
| | | | | GGGAGATGAGGAAGAGAAGTGGTCTTGGTGGGTTTGA |
| | | | | GTTTATGCCGTCGCCGCCTCCGACTTATTACCGGAATTTG |
| | | | | AGGAACAGAGCTGCTGATGTATTGAGTGAGGAGCAGAT |
| | | | | GAAGGAGTGTGAAGAGTTGGGGATTTTGGTGGATAAAG |
| | | | | ATGATCAGGGTACTTTGCTTCAAATCTTCAC |
| 12 | Amaranthus | gDNA | 1412 | ACCACCATTTTCGTTTCTTCCCCGTCTGAGTTTTATTACTT |
| | rudis | Contig | | CACTTTCTCTCTCATCATCTGACATGGGAACTTTGAAACC |
| | | | | CGAAAATCAACCCGATTCCGAATTCAAACTCGTGGGTTA |
| | | | | CTCCAACTTTGTTCGGGTTAACCCCAAATCTGACCGTTTT |
| | | | | ACTGTTAAGCGTTTCCATCATATAGAGTTTTGGTGTGGC |
| | | | | GACGCAACCAATGTTAGCAGACGATTTTCTTGGGGTCTT |
| | | | | GGAATGCCTACCGTCGCTAAATCTGACCTTTCTACGGGA |
| | | | | AACTCTGTTCACGCTTCTTTTCTTCTTCGTTCCGGTGACCT |
| | | | | TTCTTTCCTTACTTCACCTTACTCCCCTACCATGTCCATCC |
| | | | | CTTCTTCTGCTGCAATCCCCTCGTTTGATTTCAATCATTTT |
| | | | | ACCAAATTCCTTACATCGCACGGTCTTGGCGTGCGTGCT |
| | | | | GTTGCTGTCGAAGTAGAAGACGCAGAAGCTGCTTTTAAT |
| | | | | ATCAGCGTTTCCAACGGGGCTATTCCCTGTGTTTCTCCTA |
| | | | | TTCACTTGGAAAACGGTGTCGTTTTATCTGAGGTTCATTT |
| | | | | ATATGGGGATGTTGTGCTTCGGTATGTAAGCTACGGAAA |
| | | | | TGAATGTGGGGATGTGTTTTTTCTTCCTGGGTTTGAGGA |
| | | | | AATGCCGGAGGAATCTTCGTTTCGAGGACTTGATTTCGG |
| | | | | CCTTCGAAGGTTGGATCATGCTGTAGGGAATGTCCCTGA |
| | | | | GTTGGCTCCTGCAATTGCTTATTTGAAGAAGTTTACTGG |
| | | | | GTTTCATGAGTTTGCTGAGTTTACAGCTGAAGATGTTGG |
| | | | | GACGAGTGAAAGTGGGTTGAATTCAGCCGTATTGGCAA |
| | | | | ATAATGATGAAATGGTGTTGTTTCCAATGAATGAACCAG |
| | | | | TGTATGGGACAAAAAGGAAGAGTCAAATTCAAACTTATT |
| | | | | TGGAGCATAATGAAGGAGCTGGTGTACAACATTTGGCTT |
| | | | | TGATGAGTGAAGATATATTCTGGACTTTAAGGGAGATGA |
| | | | | GGAAGAGAAGTGGTCTTGGTGGGTTTGAGTTTATGCCG |
| | | | | TCGCCGCCTCCGACTTATTACCGGAATTTGAGGAACAGA |
| | | | | GCTGCTGATGTATTGAGTGAGGAGCAGATGAAGGAGTG |
| | | | | TGAAGAGTTGGGGATTTTGGTGGATAAAGATGATCAGG |
| | | | | GTACTTTGCTTCAAATCTTCACCAAACCTATTGGAGACAG |
| | | | | GTAGTTTTTAATCTTGCTTTCAATTGCTTTTGATTAATTGA |
| | | | | TTGATTAGCCAATTTGATGATTGCATTTTGTTGCTTGTAT |
| | | | | GACTTGATGATATATGGTTTACCTTTTCTCAGCTGTTCAG |
| | | | | TTGTAGCAAGTATTTCTAATCCGTTCTGAAATACTCCATT |
| | | | | CGCAACTGATTGTGACATGTTGTGCAGAAAATTATGGAA |
| | | | | AATGAGAAAATGTCCCTATATTGGAAGATTGGT |
| 13 | Amaranthus | cDNA | 707 | CCCGAGTTGGCTCCTGCAATTGCTTATTTGAAGAAGTTTA |
| | thunbergii | Contig | | CTGGGTTTCATGAGTTTGCTGAGTTTACAGCTGAAGATG |
| | | | | TTGGGACGAGTGAAAGTGGGTTGAATTCAGCCGTATTG |
| | | | | GCAAATAATGATGAAATGGTGTTGTTTCCAATGAATGAA |
| | | | | CCTGTGTATGGGACAAAAAGGAAGAGCCAAATTCAAAC |
| | | | | TTATTTGGAGCATAATGAAGGAGCTGGTGTACAACATTT |
| | | | | GGCTTTGATGAGTGAAGATATATTTTGGACTTTAAGGGA |
| | | | | GATGAGGAAGAGAAGTGGTCTTGGTGGGTTTGAGTTTA |
| | | | | TGCCGTCGCCGCCTCCGACTTATTACCGGAATTTGAGGA |
| | | | | ATAGAGCTGCTGATGTATTGAGTGAGGAGCAGATGAAG |
| | | | | GAGTGTGAAGAGTTGGGGATTTTGGTGGATAAAGATGA |
| | | | | TCAGGGTACTTTGCTTCAAATCTTCACTAAGCCTATTGGT |
| | | | | GACAGGCCAACCATATTTATCGAGATTATCCAAAGAATT |
| | | | | GGATGCATGATGAAAGATGAGGACGGCAAGATGTACCA |
| | | | | AAAGGGTGGTTGTGGAGGATTTGGAAAGGGAAACTTTT |
| | | | | CGGAGCTGTTCAAATCAATTGAGGAGTATGAGAAGACT |
| | | | | CTTGAACGTAAACAGGTTCCAGATACAGCTGCTGCATGA |
| | | | | GCAGACTAAAATATTGCTGAAACGCAGGCTGCAGCCATA |
| | | | | TGTTAGAACAG |
| 14 | Amaranthus | cDNA | 1267 | AACCACCATTTTCTCTCTCCTCCATTACCACTAACACTCCC |
| | viridis | Contig | | ACCATTTTCGTTTCTTCCCCGTCTGAGTCTTATTACTTCGC |
| | | | | TTTCTCTCACATCATCTGACATGGGAACTTTGAAACCCGA |
| | | | | AACTCAGCCCGATTCCGAATTCAAACTCGTGGGATACTC |
| | | | | CAACTTCGTTCGGGTTAACCCCAAATCTGACCGTTTTACT |
| | | | | GTTAAGCGTTTTCATCATATAGAGTTTTGGTGTGGCGAT |
| | | | | GCAACCAATGTTAGCAGACGATTTTCTTGGGGTCTTGGA |
| | | | | ATGCCTACCGTTGCTAAATCTGACCTTTCTACTGGAAACT |
| | | | | CTGTTCACGCTTCTTTTCTTCTTCGTTCCGGTGACCTTTCT |
| | | | | TTTCTCTTTACTTCACCTTACTCTCCTACCATGTCCGTCCCT |
| | | | | TCTTCTGCTGCAATCCCCTCGTTTGATTTCAATCATTTTAC |
| | | | | CAAATTCCTTACATCGCACGGTCTTGGTGTGCGTGCTGTT |
| | | | | GCTGTCGAAGTAGAAGACGCAGAAGCTGCTTTTAATATC |
| | | | | AGCGTTTCCAACGGGGCTATTCCCTGTGTTTCTCCTATTC |
| | | | | AATTGGAAAACGGTGTCGTTTTATCTGAGGTTCATTTATA |
| | | | | TGGGGATGTTGTGCTTCGGTATGTAAGCTACGGAAATG |
| | | | | AATGTGGGGATGTGTTTTTCTTCCTGGGTTTGAGGAAAT |
| | | | | GCCGGAGGAATCGTCGTTTCGAGGACTTGATTTCGGCCT |
| | | | | TCGAAGGTTGGATCATGCTGTAGGGAATGTTCCCGAGTT |
| | | | | GGCTCCTGCAATTGCTTATTTGAAAAAGTTTACTGGGTTT |
| | | | | CATGAGTTTGCTGAGTTTACAGCTGAAGATGTTGGGACG |
| | | | | AGTGAAAGTGGGTTGAATTCAGCCGTATTGGCAAATAAT |
| | | | | GATGAAATGGTGTTGTTTCCAATGAATGAACCTGTGTAT |
| | | | | GGGACAAAAAGGAAGAGCCAAATTCAAACTTATTTGGA |
| | | | | GCATAATGAAGGAGCTGGTGTACAACATTTGGCTTTGAT |
| | | | | GAGTGAAGATATATTTTGGACTTTAAGGGAGATGAGGA |
| | | | | AGAGAAGTGGTCTTGGTGGGTTTGAGTTTATGCCGTCGC |
| | | | | CGCCTCCGACTTATTACCGGAATTTGAGGAATAGAGCTG |
| | | | | CTGATGTATTGAGTGAGGAGCAGATGAAGGAGTGTGAA |
| | | | | GAGTTGGGGATTTTGGTTGACAGAGATGATCAAGGCAC |
| | | | | TCTGCTTCAAATTTTCACTAAGCCCATTGGAGATAGGCCT |
| | | | | ACGATATTCATAGAAATAATACAGAGATTAGGGTGCATG |
| | | | | GTGAA |
| 15 | Ambrosia | cDNA | 637 | CCGGTTTACGGGACGAAGAGGAAGAGTCAGATACAGAC |
| | trifida | Contig | | ATATTTGGAACATAATGAAGGGGCAGGGGTGCAGCATT |
| | | | | TGGCGTTGGCGAGTGAGGATATATTTAGGACATTGAGG |
| | | | | GAGATGAGGAAAAGGAGTGGGGTGGGTGGGTTTGAGT |
| | | | | TTATGCCATCTCCGCCGCCTACTTATTATCGGAATTTGAA |
| | | | | GAACAGGGCGGGCGATGTGTTGTCGGATGAGCAGATTA |
| | | | | AGGAGTGTGAGGAGTTGGGGATATTGGTGGATAAGGA |
| | | | | TGATCAGGGGACTTTGCTTCAGATTTTTACCAAGCCTGTT |
| | | | | GGTGATAGGCCGACGATATTCATAGAGATAATTCAGAG |
| | | | | AGTAGGATGTATGATGAAGGATGAAGAAGGAAAGGTG |
| | | | | CAGCAGAAGGCGGGCTGTGGAGGATTTGGTAAAGGGA |
| | | | | ACTTTTCGGAGCTTTTTAAATCGATTGAGGAATATGAAA |
| | | | | AGACACTGGAAGCAAGAACCTGAAGCCACATGAAAACC |
| | | | | ACACACAAATAATCTTTCATGAGATTTTAAAATCTAATGA |
| | | | | TTATGCATCTGTGGATTCTATACAACAAACAGATCCTGAA |
| | | | | AATATAGGATATCAACTTTACAATAAGTTATGTAATATGC |
| | | | | ATCTCATGATTATCAATGTATT |
| 16 | Ambrosia | cDNA | 718 | TACCTCACAAACCCAATCCTCCATACTTCCGTGTTCTCTAC |
| | trifida | Contig | | TACTGTTCCACTTTATTAATCTTACCCAACCCCTTCTCATC |
| | | | | TATTTCCCCCCTACCTCACAAACATAACCCAAATAAAAAA |
| | | | | TAAAATTAAAATAAAAAATAAAATGGAACTGAACCCACC |
| | | | | CTCGTTGACCCCGTCGTCGCCGGAGACAACTCCGACCAC |
| | | | | TCCAACCACCCCATCTCCGCCTTCAAGCTAGTCGGCTTCA |
| | | | | AAAACTTCATCCGTAACAACCCTAAGTCCGACAAATTCTC |
| | | | | CGTCAAATCCTTCCACCACATCGAGTTCTGGTGCTCCGAC |
| | | | | GCCACCAACGCCGCCCGTCGCTTCTCATGGGGTCTCGGC |
| | | | | ATGCCTATTACTTACAAATCCGACCTTTCTACCGGTAACC |
| | | | | ATACCCACGCCTCTTATCTCCTTAACTCCGGCCACCTTAA |
| | | | | CTTCCTCTTCACCGCCCCTTATTCCCCCACCATCTCCACCA |
| | | | | CCACCACCACCGCCTCCATCCCGTCGTTCTCCCACTCCGC |
| | | | | CTGCCGTCACTTCACCGCCTCCCACGGCCTTGCTGTCCGT |
| | | | | GCCATCGCCGTTGAAGTTCAAGACGCTGAAATCGCTTTC |
| | | | | TCCGTTAGTGTCGCTAACGGCGCTAAACCCTCATCTCCAC |
| | | | | CTATCACCCTTGGCCACGACGACGTCGTTTTGTCAGAAG |
| | | | | TTCAACTCTACGGTGACGTCGTTTTACGTTATATTAGTTA |
| 17 | Ambrosia | gDNA | 54 | CTCCGAGGCGTTTTGGGTCCAAAATCCGAAGCGCGGGG |
| | trifida | Contig | | CTTAAAGCGCGAGGCG |
| 18 | Ambrosia | gDNA | 719 | CTTCATGTAGTTCAGCGCTTTTTGGCAAAAAAAGCGTTTT |
| | trifida | Contig | | TCTTGGAGCCTTGTGCCTCAAACGCTTAAAGTGCGCCCC |
| | | | | AGGCGAGCTTTTTTAAACCAAGGAACACGCACAAGCACT |
| | | | | CCACCAATAGTTCAATGCCGTGCTAACCAGTGGGATTAA |
| | | | | CTAGTGGAAGCTGGATTCAAACATGCTAGAGTGGAAAT |
| | | | | ATATTTTTTTTAATGTTTGGATATGCATATTTAACCATTAT |
| | | | | AGCATAATATTAATGACAAGGGTTGTAAATTGGTATGCA |
| | | | | AATTGTTTGAGCAGGCCGACGATATTCATAGAGATAATT |
| | | | | CAGAGAGTAGGATGTATGATGAAGGATGATGAAGGAA |
| | | | | AGGTGCAGCAGAAGGCGGGTTGTGGAGGATTTGGTAAA |
| | | | | GGGAACTTTTCGGAGCTTTTTAAATCGATTGAGGAATAT |
| | | | | GAAAAGACACTGGAAGCAAGAACCTGAAGCCACATGAA |
| | | | | AACCACACAAATAATCTTTAATGAGATCTTAAAATCTAAT |
| | | | | GATTATGCATCTGTGGATTCTATACAACAAACAGATCCT |
| | | | | GAAAATATAGGATATCAACTTTACAATAAGTTATGTAAT |
| | | | | ATGCATCTCATGATTATCAATGTATTAATTTATTTTTATGT |
| | | | | TGCTGTTTTCGGTTTAGTTTTTGTTCGTTGGTGTTGGGCC |
| | | | | GGCTCTTCTTCTATTTCGTTCTTGATCCATTTGAAGTTGAA |
| | | | | GGCGAAATTTAGG |
| 19 | Ambrosia | gDNA | 845 | GCAGTTGAGCTCTACGGCTCACTGAAACAGACCTTTAAA |
| | trifida | Contig | | CCATGAACAAGGTTTATGTGTAGGTAAACTAACTTTGGA |
| | | | | CCAAAAAGCTGTTTTTTTAACCTTCACAATTTGTTTCATG |
| | | | | GAAGGTTTTCCTTTTTGTTCAGTTGACATGTTTCTAAACG |
| | | | | GGACAAATAAACATACATCTATGCCTATTGCACATATAG |
| | | | | CATCAACCACCTCACTCCCATGTGGCCCTCACAACAAGGT |
| | | | | TGTAAAAATCGGGACTAGTCGCCGACTAGTCCCCGATAA |
| | | | | ATCGGTGATCGGAGGTCTACTGATTAATTTTTCATTAATC |
| | | | | ACTCAATTAGTCGGAATCGGCCCAAGCCGGTCCAAGCCG |
| | | | | GCCAAAGTCGGCCGAAGTCGGAGCTAGTCGCCAAAGAA |
| | | | | TTCCGTTTGAAGTTGGCTTAAAAACATGCTTTCATCCCAC |
| | | | | AAACTGATCCATTACCCCAAAAAACCAAAGTTAAAAACC |
| | | | | TGACCTGGATCCCTAAATGCTTCATAATCCGGCAAACCTT |
| | | | | TCTGTTCCGGCGAACCTTTCTGTTTCCGGCGACCCATTTG |
| | | | | GAGCAGGTGGAGTTGGTTTTGTTCAGTCTTTTGCGGCGT |
| | | | | ATAATGAAAGTGGTAGTGAGTAGGGTTTTTAAGTTTGGA |
| | | | | TCTTTTTATATTTAACCCCTTATAGTTTTACTAGTTTATATT |
| | | | | TTGTCCCTTAACTTATATATTTATACATAATAAGCATAAA |
| | | | | GTTAATGTTATATATATACAAATTAACCCTCTACATCTTTA |
| | | | | CTAGTCTATATTTGGTCCTCTAACTTATACATCTATACAA |
| | | | | AAAAAGCATAAAATTAATGTTATATATATATAAATTTATA |
| | | | | AAATTATACATA |
| 20 | Ambrosia | gDNA | 1024 | TCGTCGTTTCAGGAGCTGGATTACGGTATCCGGCGGCTA |
| | trifida | Contig | | GATCACGCTGTGGGGAACGTACCGGAGTTAGCACCAGC |
| | | | | AGTGGAATATATAAAATCGTTTACCGGGTTTCACGAGTT |
| | | | | TGCTGAGTTTACAGCAGAGGATGTGGGAACGAGTGAGA |
| | | | | GTGGACTCAACTCGGTGGTGTTGGCTTGCAATAGTGAG |
| | | | | ATGGTATTAATACCTCTGAATGAGCCGGTTTACGGGACG |
| | | | | AAGAGGAAGAGTCAGATACAGACATATTTGGAACATAA |
| | | | | TGAAGGGGCAGGGGTGCAGCATTTGGCGTTGGCGAGT |
| | | | | GAGGATATATTTAGGACATTGAGGGAGATGAGGAAAAG |
| | | | | GAGTGGGGTGGGTGGGTTTGAGTTTATGCCGTCTCCGC |
| | | | | CGCCTACTTATTATCGGAATTTGAAGAATAGGGCGGGCG |
| | | | | ATGTGTTGTCGGATGAGCAGATTAAGGAGTGTGAGGAG |
| | | | | TTGGGGATATTGGTGGATAAGGATGATCAGGGGACTTT |
| | | | | GCTTCAGATTTTTACCAAGCCTGTTGGTGATAGGTATTGT |
| | | | | TTTTCGTTAATGTAGCGTTACTTGAATTCGTAATTATGTG |
| | | | | TTTAAACTTTAAACTAGATAGTATTAAGGCTTATGCAATA |
| | | | | TGTATTGTTCCCCTAGAGGACGAGGGTTCTAGACTCTAG |
| | | | | TTCCGTAATGTAGTGATAAAGGTGTAGATTAAGAGTAGG |
| | | | | ATTAGCGGGTGTGTTAGTTAGCGGTTTTAACAAGAAAAA |
| | | | | CACATAATATGTTGAAAGAACAATAACAAGAATGGCAA |
| | | | | ACATAAAAAAGGAAAAGCCATAAAGGTTGTCATTCTATA |
| | | | | ATTTGCTTGCATTCAAGATTCTTACTAGGATTGTATTATT |
| | | | | ATGTGGTTACTAAACTCTATTCAAGTATAAAAACATGAA |
| | | | | ACCCCTTCACATAAGGAAAAAGAAATATGTATGTTTTAA |
| | | | | AATGGATCATCTTTTCCCTTGTGTCACGATTGCCAATGTT |
| | | | | TTTTTTGTTGACCATGAGAACCGTTGGTGAACACGCCTT |
| | | | | GGTTTAAAAAAGCGCG |
| 21 | Conyza | cDNA | 1610 | TATGGTATTGCATTGTTTCATTCAATTGTACATAATCCAC |
| | canadensis | Contig | | AAATACATATGGTGATAATCATGTTCTTTGAGTCAAGAA |
| | | | | GATATAAACATTATACATAGGCAGTTTTCATGCAGTGGC |
| | | | | AGTTGGTTCAGTGGTGTTTCGTGCTTCCAGCGTCTTCTCG |
| | | | | TATTCTTCAATAGATTTGAAGAGCTCTGAGAAATTGCCCT |
| | | | | TGCCAAATCCTCCACAGCCTGGCTTCTGTTGCACCTTGCC |
| | | | | TTCATCATCTTTCAGCATACACCCTACTCTCTGAATAATCT |
| | | | | CTATGAATATAGTTGGCCTATCACCAACAGGCTTGGTGA |
| | | | | AAATCTGAAGCAAAGTTCCCTGATCATCTCTATCAACCAA |
| | | | | AATCCCCAATTCCTCACATTCCTTAATCTGTTCGTCACTCA |
| | | | | ACACGTCCCCGACCCTATTCTTCAAATTCTTATAATAAGT |
| | | | | AGGCGGTGGAGACGGCATAAACTCAAAACCACCGACAC |
| | | | | TACTCCGTTTTCTCATCTCTCTCAAAGTCCTAAAAATATCC |
| | | | | TCACTCGCCAACGCCAAATGCTGCACACCAGCTCCCTCAT |
| | | | | TATGTTCCAAATACGTCTGTATCTGGCTCTTCCTTTTCGTT |
| | | | | CCATAAACTGGCTCGTTCATCGGTATCAAAACCGTTTCAC |
| | | | | TATTACACGCTAATACAACCGAATTAAGCCCGCTCTCACT |
| | | | | CGTTCCTACATCCTCTGCCGTAAATTCAGCAAATTCGTGA |
| | | | | AATCCAGTGAAGGACTTCACGTAGTCTACTGCCGGAGCT |
| | | | | AGCTCCGGCACGTTTCCTACGGCGTGATCTAACTTATGA |
| | | | | ATTCCGTAATTAAGTTCATGAAACGACGACGTTGCGTCC |
| | | | | ACAGGCTCAAAACCCGGTAGGAAATTAGTAGTAACATCA |
| | | | | TAATTAGTGTTTTTAAAACTAACATATCTCAAAACGACAT |
| | | | | CGCCGTACAGTTTAACTTCAGCCAAAACGACGTCGTTATT |
| | | | | TCCTAGAGTGACCGGAGCTGACGACGGTTTAGCACCGT |
| | | | | GAGCGATACTAACAGAATAAGCGATTTCAGCGTCTTCAA |
| | | | | CTTCAACGGCGATAGACCGTACAGCGAGGCCGTGAGTG |
| | | | | GCGGTGAAGTTCCGGCAAGCAGTGTGAGAGAAAGTAG |
| | | | | GAATAGACGATGTGGAACCGGTGGTGGAGATGGTGGT |
| | | | | GGAATAAGGTGCGGTGAAGAGGAAGTTGAGCTGGCCG |
| | | | | GAGTTAATAAGGTAAGAAGCGTGAGTGTTGTTACCGGT |
| | | | | GGAGAGATCGGATTTGTAAAGAAGCGGCATGCCGAGAC |
| | | | | CCCAAGAGAAGCGGCGGGCGGTGTTGGTGGCGTCGGA |
| | | | | ACACCAGAATTCGACGTGGTGGAATTTTTTGACGGTGAA |
| | | | | TTTGTCGGATTTAGGGTTTTGACGGATGAAGTTTTTGAA |
| | | | | GCCGACGAGTTTGTAAGTGGCGGAGGTGGTGGTAGTGG |
| | | | | TTTGTTGTTGTCCGGTGAGTGTGTTTCCGTTTGCTGCTTC |
| | | | | AGTTACCATTTTGTTTTTATTAATTTGAATGTATAGATAG |
| | | | | AAATATGAAGTAGATATATAGAATGATAGATATAGATAC |
| | | | | AGTACAAGTAGTTGAGGGGTTGGATGATGGAGATGAAG |
| | | | | AAGAACACGGAAGTGGTTTATTTTCTTTGGGTGCTATTA |
| | | | | ATTG |
| 22 | Conyza | gDNA | 4963 | AACACGAATGGGCTAGCTAGCTGATGAATTTAATGTATA |
| | canadensis | Contig | | TATGTCTTTAAAAACCACTTTCATGGGCTACTTGTGTTTTT |
| | | | | GATGAGTTTTTCAACATCATTCTCATCTTACCTTCTTACAA |
| | | | | TGGTTCACCATATTGCTTCGTTTGTCGTCTCCTGCATGGT |
| | | | | TATCGAATTTATTTTTTCAAATTATATTACTATTATCTTTTT |
| | | | | GTTTTCTGATTTCTCACATGTAATTTACATTATGAGTTTCA |
| | | | | CCATCCGTATGTAAATTGTAAAATAAATCACAAATTTGGT |
| | | | | TTTTAACCATCAACTAAATTCATTGAAATCCTTAAGTTAT |
| | | | | AACCCATTTGAGGTTATAGATTATGAGAGTAGATTCCTA |
| | | | | TTTTCTCTTTGGCTTATGTATCCTTAATTATGTTTGAATTG |
| | | | | ATTCTAACTTTATTATATTTTAGGAATTACTTAACTTCAAA |
| | | | | AATCTCAACACGTCATATAATGCTTTTTGTGGATGGATGC |
| | | | | TTTTGACCCATTTCGTTGAAAACTTATGTCGACACACATA |
| | | | | TCTGACAAACTTGTACTCGTATATCCTTTATCGCATATGC |
| | | | | TATAGTATAATATTCGAAATTTAATCTCAAGATAATTTTC |
| | | | | CCAAAAGGTCATATTAATTGAATTGAGATTAATTCCTACC |
| | | | | TAACATATATAGATATATTGTGTTGGTGTTATCCTCTTTG |
| | | | | GAGTTTTAGTCGCGCATGTATTCTAAGAAAATGGTAATT |
| | | | | TTGAACTTATATTGATGGTCTAAATTTTATCCTATCACTA |
| | | | | AAATTTCAGTCATGTTTCCTAGCTAATTAATTTGGGTTCA |
| | | | | ATCCAGGTATTCTAAATAAGTTTTGTAAATACATGTCTAT |
| | | | | TATCATAATGAAACATAGAAAAACCCGTAAAGTAAAAAT |
| | | | | GTCTATATTTTTAGTGGCAAATTATTAATGTTGCTATAAT |
| | | | | GAAAAGAGAAGATTCAACCTTTTGTACATCATAAAAAAT |
| | | | | GTTATTCTACATTATGCTAATTTTACATTAGACTTCATAGT |
| | | | | TTTGAAAACTTTCAAATGAAGGGACTTAAATTTTAAATTT |
| | | | | AAAGATGATGTCATAAATTAGTTAGATAAAAGTTAAATA |
| | | | | ATTAATAAGAAAAGGTCTAATGATACCAAATAAAATTTT |
| | | | | CTTAAAAAAGTATTAATGTCATTACAATTTTATTTTATTTA |
| | | | | TATAAGAGAAGATAATAGATTTTCTTGTATAAAAAACTT |
| | | | | ACCTGTAAACTTCTATATAAACTTTTTAGAAAAATAATTT |
| | | | | AAAACGGTCTCAAATATCGTTATTTCCGAACATACAAAA |
| | | | | ACCCAAAACCTAATCCATTTAATTATATTGTAAGACCTCG |
| | | | | ATTAGCTTAATTTATTTTGAAACCTATTTACAAATGAGTT |
| | | | | TGGCATGATATGAACTTTTTTGCATTTACATATGGCTGTA |
| | | | | AACGAACTGAACAGTTCACGAACAATTCATGAACCGTTC |
| | | | | GGCGGGAGGTTCGTTCGTGTTAGTTCGTTTAGTTAAATG |
| | | | | AATGAACATGAACAAAGCTCTCGTTCGTTCATTTACGTTC |
| | | | | ATGAACGTTCGATAGTCTGTTCGTGAACTTTAGTTCGTCT |
| | | | | ATGTTCATGAACTGTCGTTCGTGAACAATAATTTGATTAT |
| | | | | GTTCATTTACACACACATACATAATCAACTGTACTATAAA |
| | | | | AATAGTTTAAATATAAATATTTTTTTATATAATATATATTA |
| | | | | TTAATATCTAATTATTTAAGAAAAAGTTTAAATAAAATAC |
| | | | | TTAATTTATAAATATTTCGTTCGTTTGTGTTCGTGTACATT |
| | | | | TGTTCATGAACACAAATGAACGAACATGAACAAGTCATT |
| | | | | ATGTTCGTTTATCTGTTCGTTAAGTTAAATGAACGAACGA |
| | | | | ACACGAACAAGGTCTCGTTTGTGTTCGTTCGGTTCGTTTA |
| | | | | CGGCCCTTAATTACATGTGAAGTTAATAAATAAAAATAA |
| | | | | AGTTCATCAAAATTAAACCTATGGTTGGAGATAAAACCA |
| | | | | GTCAGCCTTACGTGTGGAAACTATCCTACTCTACAATTAA |
| | | | | TAGCACCCAAAGAAAATAAACCACTTCCGTGTTCTTCTTC |
| | | | | ATCTCCATCATCCAACCCCTCAACTACTTGTACTGTATCTA |
| | | | | TATCTATCATTCTATATATCTACTTCATATTTCTATCTATAC |
| | | | | ATTCAAATTAATAAAAACAAAATGGTAACTGAAGCAGCA |
| | | | | AACGGAAACACACTCACCGGACAACAACAAACCACTACC |
| | | | | ACCACCTCCGCCACTTACAAACTCGTCGGCTTCAAAAACT |
| | | | | TCATCCGTCAAAACCCTAAATCCGACAAATTCACCGTCAA |
| | | | | AAAATTCCACCACGTCGAATTCTGGTGTTCCGACGCCAC |
| | | | | CAACACCGCCCGCCGCTTCTCTTGGGGTCTCGGCATGCC |
| | | | | GCTTCTTTACAAATCCGATCTCTCCACCGGTAACAACACT |
| | | | | CACGCTTCTTACCTTATTAACTCCGGCCAGCTCAACTTCC |
| | | | | TCTTCACCGCACCTTATTCCACCACCATCTCCACCACCGG |
| | | | | TTCCACATCGTCTATTCCTACTTTCTCTCACACTGCTTGCC |
| | | | | GGAACTTCACCGCCACTCACGGCCTCGCTGTACGGTCTA |
| | | | | TCGCCGTTGAAGTTGAAGACGCTGAAATCGCTTATTCTG |
| | | | | TTAGTATCGCTCACGGTGCTAAACCGTCGTCAGCTCCGG |
| | | | | TCACTCTAGGAAATAACGACGTCGTTTTGGCTGAAGTTA |
| | | | | AACTGTACGGCGATGTCGTTTTGAGATATGTTAGTTTTA |
| | | | | AAAACACTAATTATGATGTTACTACTAATTTCCTACCGGG |
| | | | | TTTTGAGCCTGTGGACGCAACGTCGTCGTTTCATGAACTT |
| | | | | AATTACGGAATTCATAAGTTAGATCACGCCGTAGGAAAC |
| | | | | GTGCCGGAGCTAGCTCCGGCAGTAGACTACGTGAAGTC |
| | | | | CTTCACTGGATTTCACGAATTTGCTGAATTTACGGCAGA |
| | | | | GGATGTAGGAACGAGTGAGAGCGGGCTTAATTCGGTTG |
| | | | | TATTAGCGTGTAATAGTGAAACGGTTTTGATACCGATGA |
| | | | | ACGAGCCAGTTTATGGAACGAAAAGGAAGAGCCAGATA |
| | | | | CAGACGTATTTGGAACATAATGAGGGAGCTGGTGTGCA |
| | | | | GCATTTGGCGTTGGCGAGTGAGGATATTTTTAGGACTTT |
| | | | | GAGAGAGATGAGAAAACGGAGTAGTATCGGTGGTTTTG |
| | | | | AGTTTATGCCGTCTCCACCGCCTACTTATTATAAGAATTT |
| | | | | GAAGAATAGGGTCGGGGACGTGTTGAGTGACGAACAG |
| | | | | ATTAAGGAATGTGAGGAATTGGGGATTTTGGTTGATAG |
| | | | | AGATGATCAGGGAACTTTGCTTCAGATTTTCACCAAGCC |
| | | | | TGTTGGTGATAGGTATGTCTATGTTAACTTTATCAGTGAT |
| | | | | TGTGCATTTGTCCATTTTGTTGAATTCGTAAAACATGAGA |
| | | | | TTAAATACCTGCAATATGTTTTGCTTACATTGAATCTAGG |
| | | | | CCAGGTTTTCAGTGAACAAGTACCATAAATGTATAATGT |
| | | | | AGGTCTAGTCTTGTTAATGATCAGTGATCAATAGTGCAT |
| | | | | ACATTGAATGACGAATTGACAACTAGTTTCTAATAGAAT |
| | | | | GTGAAATATAAAGTGAATGTCACAAAAGCTGTTATCTTA |
| | | | | TCTATTTTAGTTGTAATGGACCAATGGTTGGACTGTTTGA |
| | | | | AAGTGTGCTTACAATCAAGATTCGAGATTGTTACTAGGA |
| | | | | TGCTAGCGCTAGGTATATATCATATGGTGTCTGAGCTCT |
| | | | | ATGGTAGTATAAATTGTGAATACACTAAACATCTCCCAA |
| | | | | AAATATTTGAGTAACCGATCAATCTACTAGTTATTGCAAT |
| | | | | GCCAAACATCTCTTTAAACAACTACTTATTGTAAAGCCAT |
| | | | | ACTACATCAACACACCAAAAATCTATCTCCCACAACCCCC |
| | | | | GTTTTACTTGGGTTATGAACTTAGTATAGTTATTCAAAAT |
| | | | | AATCACTTTCAAAATGGATCCAACACATCTGAAACTGGT |
| | | | | CACATTTGAGTTCTTATCTTTCAAATAATTTGTAATTCATA |
| | | | | TCAGGCTCAGCTTCATGGGGTCTGGCCTTTGTCTGTGTG |
| | | | | TTTGTGTTGTGTCTTTTCTGGTGTGATCTAATTCGGTATG |
| | | | | CATATTGTTTGAGCAGGCCAACTATATTCATAGAGATTAT |
| | | | | TCAGAGAGTAGGGTGTATGCTGAAAGATGATGAAGGCA |
| | | | | AGGTGCAACAGAAGCCAGGCTGTGGAGGATTTGGCAAG |
| | | | | GGCAATTTCTCAGAGCTCTTCAAATCTATTGAAGAATAC |
| | | | | GAGAAGACGCTGGAAGCACGAAACACCACTGAACCAAC |
| | | | | TGCCACTGCATGAAAACTGCCTATGTATAATGTTTATATC |
| | | | | TTCTTGACTCAAAGAACATGATTATCACCATATGTATTTG |
| | | | | TGGATTATGTACAATTGAATGAAACAATGCAATACCATA |
| | | | | TGTGATGTGATTTATATAGAATAACAATAGATGTCATTCA |
| | | | | TATATGCCGTTCCTGTTTTAGTAATTGTGTGTTGGTGGTG |
| | | | | TGTTGCAAGTTCCAATCAGCTGTATATAATGCCTAAATAT |
| | | | | TCAATTTGACCTCCATTAAGGATCCATTGTGCAGATTCTT |
| | | | | TGTTTCTGTCTTAAAAGTGTGGGAGACTTGAAACACATC |
| | | | | TGTAAAACTGAACCAGATCTGAATCGAGCTGAATGGAA |
| | | | | GCGGCGCAGGTCTGCTCTGTGGTTCCACTTACAACAAAC |
| | | | | AATTGTTCATGGTACGTCGCCTCTTCATAGCCAGTGTTTA |
| | | | | TTGTAAGAAAGCATTGTTTAGGCGAAAAAGGAAAAGGT |
| | | | | GATTTATGTAAGAAAAGGTAGTGCTATTAAGGTTATGTT |
| | | | | CCCCATGAACAAACAGGGTAAATTCATACAGGATCAACT |
| | | | | TTTAAATGATACTATTAAAGGTCATCTACTTCTGTTTCCC |
| | | | | ATGCTTGTGCCAAGCTCAAATTTAGACGAAACCAAAACA |
| | | | | GGATGAGAAAACTAATTAACACATATTTTAGTAAGCAAT |
| | | | | TACAGCTATATATCATACTATCATTAACAATTACATCAAC |
| | | | | AATCTGAAAAGTTGCAT |
| 23 | Conyzag | DNA | 5610 | ATTAAAAACACGAATGGGCTAGCTAGCTGATGAATTTAA |
| | canadensis | Contig | | TGTATATATGTCTTTAAAAACCACTTTCATGGGCTACTTG |
| | | | | TGTTTTTGATGAGTTTTTCAACATCATTCTCATCTTACCTT |
| | | | | CTTACAATGGTTCACCATATTGCTTCGTTTGTCGTCTCCT |
| | | | | GCATGGTTATCGAATTTATTTTTTCAAATTATATTACTATT |
| | | | | ATCTTTTTGTTTTCTGATTTCTCACATGTAATTTACATTAT |
| | | | | GAGTTTCACCATCCGTATGTAAATTGTAAAATAAATCACA |
| | | | | AATTTGGTTTTTAACCATCAACTAAATTCATTGAAATCCT |
| | | | | TAAGTTATAACCCATTTGAGGTTATAGATTATGAGAGTA |
| | | | | GATTCCTATTTTCTCTTTGGCTTATGTATCCTTAATTATGT |
| | | | | TTGAATTGATTCTAACTTTATTATATTTTAGGAATTACTTA |
| | | | | ACTTCAAAAATCTCAACACGTCATATAATGCTTTTTGTGG |
| | | | | ATGGATGCTTTTGACCCATTTCGTTGAAAACTTATGTCGA |
| | | | | CACACATATCTGACAAACTTGTACTCGTATATCCTTTATC |
| | | | | GCATATGCTATAGTATAATATTCGAAATTTAATCTCAAGA |
| | | | | TAATTTTCCCAAAAGGTCATATTAATTGAATTGAGATTAA |
| | | | | TTCCTACCTAACATATATAGATATATTGTGTTGGTGTTAT |
| | | | | CCTCTTTGGAGTTTTAGTCGCGCATGTATTCTAAGAAAAT |
| | | | | GGTAATTTTGAACTTATATTGATGGTCTAAATTTTATCCT |
| | | | | ATCACTAAAATTTCAGTCATGTTTCCTAGCTAATTAATTT |
| | | | | GGGTTCAATCCAGGTATTCTAAATAAGTTTTGTAAATACA |
| | | | | TGTCTATTATCATAATGAAACATAGAAAAACCCGTAAAG |
| | | | | TAAAAATGTCTATATTTTTAGTGGCAAATTATTAATGTTG |
| | | | | CTATAATGAAAAGAGAAGATTCAACCTTTTGTACATCAT |
| | | | | AAAAAATGTTATTCTACATTATGCTAATTTTACATTAGAC |
| | | | | TTCATAGTTTTGAAAACTTTCAAATGAAGGGACTTAAATT |
| | | | | TTAAATTTAAAGATGATGTCATAAATTAGTTAGATAAAA |
| | | | | GTTAAATAATTAATAAGAAAAGGTCTAATGATACCAAAT |
| | | | | AAAATTTTCTTAAAAAAGTATTAATGTCATTACAATTTTA |
| | | | | TTTTATTTATATAAGAGAAGATAATAGATTTTCTTGTATA |
| | | | | AAAAACTTACCTGTAAACTTCTATATAAACTTTTTAGAAA |
| | | | | AATAATTTAAAACGGTCTCAAATATCGTTATTTCCGAACA |
| | | | | TACAAAAACCCAAAACCTAATCCATTTAATTATATTGTAA |
| | | | | GACCTCGATTAGCTTAATTTATTTTGAAACCTATTTACAA |
| | | | | ATGAGTTTGGCATGATATGAACTTTTTTGCATTTACATAT |
| | | | | GGCTGTAAACGAACTGAACAGTTCACGAACAATTCATGA |
| | | | | ACCGTTCGGCGGGAGGTTCGTTCGTGTTAGTTCGTTTAG |
| | | | | TTAAATGAATGAACATGAACAAAGCTCTCGTTCGTTCATT |
| | | | | TACGTTCATGAACGTTCGATAGTCTGTTCGTGAACTTTAG |
| | | | | TTCGTCTATGTTCATGAACTGTCGTTCGTGAACAATAATT |
| | | | | TGATTATGTTCATTTACACACACATACATAATCAACTGTA |
| | | | | CTATAAAAATAGTTTAAATATAAATATTTTTTTATATAATA |
| | | | | TATATTATTAATATCTAATTATTTAAGAAAAAGTTTAAAT |
| | | | | AAAATACTTAACTTATAAATATTTCGTTCGTTTGTGTTCG |
| | | | | TGTACATTTGTTCATGAACACAAATGAACGAACATGAAC |
| | | | | AAGTCATTATGTTCGTTTATCTGTTTGTTAAGTTAAATGA |
| | | | | ACGAACGAACACGAACAAGGTCTCGTTTGTGTTCGTTCG |
| | | | | GTTCGTTTACGGCCCTTAATTACATGTGAAGTTAATAAAT |
| | | | | AAAAATAAAGTTCATCAAAATTAAACCTATGGTTGGAGA |
| | | | | TAAAACCAGTCAGCCTTACGTGTGGAAACTATCCTACTCT |
| | | | | ACAATTAATAGCACCCAAAGAAAATAAACCACTTCCGTG |
| | | | | TTCTTCTTCATCTCCATCATCCAACCCCTCAACTACTTGTA |
| | | | | CTGTATCTATATCTATCATTCTATATATCTACTTCATATTT |
| | | | | CTATCTATACATTCAAATTAATAAAAACAAAATGGTAACT |
| | | | | GAAGCAGCAAACGGAAACACACTCACCGGACAACAACA |
| | | | | AACCACTACCACCACCTCCGCCACTTACAAACTCGTCGGC |
| | | | | TTCAAAAACTTCATCCGTCAAAACCCTAAATCCGACAAAT |
| | | | | TCACCGTCAAAAAATTCCACCACGTCGAATTCTGGTGTTC |
| | | | | CGACGCCACCAACACCGCCCGCCGCTTCTCTTGGGGTCT |
| | | | | CGGCATGCCGCTTCTTTACAAATCCGATCTCTCCACCGGT |
| | | | | AACAACACTCACGCTTCTTACCTTATTAACTCCGGCCAGC |
| | | | | TCAACTTCCTCTTCACCGCACCTTATTCCACCACCATCTCC |
| | | | | ACCACCGGTTCCACATCGTCTATTCCTACTTTCTCTCACAC |
| | | | | TGCTTGCCGGAACTTCACCGCCACTCACGGCCTCGCTGT |
| | | | | ACGGTCTATCGCCGTTGAAGTTGAAGACGCTGAAATCGC |
| | | | | TTATTCTGTTAGTATCGCTCACGGTGCTAAACCGTCGTCA |
| | | | | GCTCCGGTCACTCTAGGAAATAACGACGTCGTTTTGGCT |
| | | | | GAAGTTAAACTGTACGGCGATGTCGTTTTGAGATATGTT |
| | | | | AGTTTTAAAAACACTAATTATGATGTTACTACTAATTTCC |
| | | | | TACCGGGTTTTGAGCCTGTGGACGCAACGTCGTCGTTTC |
| | | | | ATGAACTTAATTACGGAATTCATAAGTTAGATCACGCCG |
| | | | | TAGGAAACGTGCCGGAGCTAGCTCCGGCAGTAGACTAC |
| | | | | GTGAAGTCCTTCACTGGATTTCACGAATTTGCTGAATTTA |
| | | | | CGGCAGAGGATGTAGGAACGAGTGAGAGCGGGCTTAA |
| | | | | TTCGGTTGTATTAGCGTGTAATAGTGAAACGGTTTTGAT |
| | | | | ACCGATGAACGAGCCAGTTTATGGAACGAAAAGGAAGA |
| | | | | GCCAGATACAGACGTATTTGGAACATAATGAGGGAGCT |
| | | | | GGTGTGCAGCATTTGGCGTTGGCGAGTGAGGATATTTTT |
| | | | | AGGACTTTGAGAGAGATGAGAAAACGGAGTAGTATCGG |
| | | | | TGGTTTTGAGTTTATGCCGTCTCCACCGCCTACTTATTAT |
| | | | | AAGAATTTGAAGAATAGGGTCGGGGACGTGTTGAGTGA |
| | | | | CGAACAGATTAAGGAATGTGAGGAATTGGGGATTTTGG |
| | | | | TTGATAGAGATGATCAGGGAACTTTGCTTCAGATTTTCA |
| | | | | CCAAGCCTGTTGGTGATAGGAGGCCAACTATATTCATAG |
| | | | | AGATTATTCAGAGAGTAGGGTGTATGCTGAAAGATGAT |
| | | | | GAAGGCAAGGTGCAACAGAAGCCAGGCTGTGGAGGAT |
| | | | | TTGGCAAGGGCAATTTCTCAGAGCTCTTCAAATCTATTG |
| | | | | AAGAATACGAGAAGACGCTGGAAGCACGAAACACCACT |
| | | | | GAACCAACTGCCACTGCATGAAAACTGCCTATGTATAAT |
| | | | | GTTTATATCTTCTTGACTCAAAGAACATGATTATCACCAT |
| | | | | ATGTATTTGTGGATTATGTACAATTGAATGAAACAATGC |
| | | | | AATACCATATGTGATGTGATTTATATAGAATAACAATAG |
| | | | | ATGTCATTCATATATGCCGTTCCTGTTTTAGTAATTGTGT |
| | | | | GTTGGTGGTGTGTTGCAAGTTCCAATCAGCTGTATATAA |
| | | | | TGCCTAAATATTCAATTTGACCTCCATTAAGGATCCATTG |
| | | | | TGCAGATTCTTTGTTTCTGTCTTAAAAGTGTGGGAGACTT |
| | | | | GAAACACATCTGTAAAACTGAACCAGATCTGAATCGAGC |
| | | | | TGAATGGAAGCGGCGCAGGTCTGCTCTTTGGTTCCACTT |
| | | | | ACAACAAACAATTGTTCATGGTACGTCGCCTCTTCATGGC |
| | | | | CAGTGTTTATTGTAAGAAAGCATTGTTTAGGCGAAAAAG |
| | | | | GAAAAGGTGATTTATGTAAGAAAAGGTAGTGCTATTAA |
| | | | | GGTTATGTTCCCCATGAACAAACAGGGTAAATTCATACA |
| | | | | GGATCAACTTTTAAATGATACTATTAAAGGTCATCTACTT |
| | | | | CTGTTTCCCATGCTTGTGCCAAGCTCAAATTTAGACGAAA |
| | | | | CCAAAACAGGATGAAAAAACTAATTAACACATATTTTAG |
| | | | | TAAGCAATTACAGCTATATATCATACTATCATCAACAATC |
| | | | | TGAAAAGTTGCATACAATTTATCGTTTATTCCTGTCAAGT |
| | | | | GATGACATGAAAAATACCATTTAGTTTACACGTACTCAG |
| | | | | ATATGCCAATAATAGAGGGACTTTTTCTATATGAGAACA |
| | | | | TACATGATGTTCATAATTCATAATTTACTTGATACGGCAA |
| | | | | GGTACTTGGTATGGCGTCCTTGTGCAATTGTTTTTCCGGT |
| | | | | CTTTTTGTTCCTTAATTCAACGGTCACAACAGCAATAGCC |
| | | | | TTCCCAACACGCAATGTTTTTGCCTCTATCTCAATCTCGTC |
| | | | | CTGTAAGAAAATTGCATCCTTAACAAAATTTTACTCAAAG |
| | | | | ACGCATCTATGGTACGAAGTACAAACCAAAGATTTTCAG |
| | | | | TAAGTCGTGCAAAACCTTTATGCATACTGCAACTACTCAA |
| | | | | ACTTCACATCTTATGCCTAAAATCAAAGACCCCAAAGAA |
| | | | | GCCTTGAGTTCAAAATCCAAGAATTTCTGTCAGCTACAG |
| | | | | GGGACAGGTTCAACCCATTTACATACAAATGGGTCAAAA |
| | | | | TGGGTTATGTTATTCTGTGTTTCTGCCAAACCAGACCTGG |
| | | | | CACGTACTAAAACTCACCCATTACCCAACCCACCTACCCA |
| | | | | GCCCCCCTTTTTACCAACATGCATTTTGCTTTTGCAACCCT |
| | | | | GTTGCTTCAAGCATTCAACTATTCAAGTAGCAAGATCGA |
| | | | | AAGAACTTATACAAAAAGTTGCCATTTATGATTAAGTGA |
| | | | | AGCCTTAATGACACCAAATCAGTTATTATAGCATTACAAT |
| | | | | GGGAGAGCTTCAACAGAAAAGTTATCAAGTCTTCTCCAA |
| | | | | CAAGATCATGAGCTCATTTTCAATTTTATATCAAACGGTA |
| | | | | GCAATAACGAAAGTAAATCTGGTGGCTCAAGGTAAAAC |
| | | | | CTCATAACCTTTGATGGCATAATAACTTCCAGACCAACTA |
| | | | | AACACCAAGGTGGAAGTTATTACTTTGTGCTAATGATAG |
| | | | | CAGTTACAAACATAAAAAATCTGGTCGGCTTCGTTCAGT |
| | | | | TTTCTGAAATACCAATATCGTCTGACAAGGTGCCATCTTC |
| | | | | ATATTCAGAGTCACTTCATGATCAACTCTTGTGGGGTTTC |
| | | | | AAACTCAAGAACGTTTTAAATACATTCATTTAATATTTTT |
| | | | | ATCTCCAGAATACTACTCAACACTTTCCTGTAAGGTCTTC |
| | | | | AATTTCAGGGGGAAAAAAACCCAAAATTCAAGGAATCCT |
| | | | | GCTATTAGTGTATCGTTTCTTGATAGCAAAGCTTTCATTT |
| | | | | ACAATCTCTAACAACATTTTTACTTCAAAATTTTATCATAA |
| | | | | TGCTCTTTAGGAAAATGTTGACGGTATGAATATGAGATC |
| | | | | AGGATATCAAAAGAACAAAATATTTATTTTGTTCTTAAGT |
| | | | | TGCAAACCGCACAAGAGTTGATCATGCAGTGTTCGCGAA |
| | | | | TTTGAAGATGGCACCTTGTTAGGCAACATTGG |
| 24 | Euphorbia | cDNA | 386 | TTGTTTCTGCCGAAATTCGAGCCGGTAGATGAGGCGTCG |
| | heterophylla | Contig | | TCGTTTCCGTTGGATTACGGGATTCGACGGCTAGATCAC |
| | | | | GCGGTTGGAAATGTACCGGATCTTGCTCCGGCGGTTTCG |
| | | | | TATGTGAAGAAGTTCACCGGATTCCACGAGTTCGCTGAG |
| | | | | TTCACGGCGGAGGATGTAGGGACGAGTGAGAGCGGAT |
| | | | | TGAACTCGGTGGTGTTGGCTAACAACGAAGAAACGGTA |
| | | | | CTGCTGCCGATGAATGAACCGGTGTTTGGGACGAAGAG |
| | | | | GAAAAGTCAGATACAGACGTATTTGGAGCACAATGAAG |
| | | | | GAGCTGGAGTACAGCATTTGGCGCTTGTGAGTGCTGAT |
| | | | | ATTTTCAATACTTTGAGAGAGATGAGGAGGAGGAGTGC |
| | | | | GAT |
| 25 | Euphorbia | gDNA | 2639 | GACAATTAATAAAAAAAACGTAAAGACTACCTTTAATTG |
| | heterophylla | Contig | | GAGGAGAGGAAAACACCACGTTTAAAAATCCCGTTGTTA |
| | | | | TCCGATTGATGAAAAAAAAGATTAACACGTTACGACTTC |
| | | | | TCCATTCAATAATCCATTTTCTTTATCTTATAAATAATTTG |
| | | | | AAATCCCATCCTCCTCGTTCTCCGTTCACCAGAAAAAACA |
| | | | | GAAATGGGAAAAGACACATCAGCTGCCGCCGAAGCATT |
| | | | | CAAGCTCGTCGGATTTTCCAATTTCGTCAGAATCAATCCC |
| | | | | CGGTCCGACCGTTTCCCGGTCAAGCGCTTCCACCACGTC |
| | | | | GAGTTCTGGTGCTCCGACGCAACCAACACCGCTCGCCGC |
| | | | | TTCTCATGGGGCCTCGGGATGCCGTTCGTCGCCAAATCG |
| | | | | GATCTCTCCACCGGCAACGTCACCCACGCCTCCTACCTCC |
| | | | | TCCGCTCCGGCGACCTCAATTTCCTCTTCACAGCTCCCTA |
| | | | | CTCTCCCTCCATAGCCGCCATGGAGAATCTCTCCGATACT |
| | | | | GCTACCGCATCAATCCCTACTTTCTCCCGCGACGTTTTCC |
| | | | | TCGATTTCTCCGCCAAACACGGCCTCGCCGTCCGAGCTA |
| | | | | TAGCAATCGAAGTGGAGGACGCTGCAGTTGCCTTCAATA |
| | | | | CTAGTGTTGCTCAAGGCGCGGTTCCGGTGGCCGGACCT |
| | | | | GTAGTGCTCGATAATCGCGCTTCGGTAGCGGAGGTTCAC |
| | | | | TTGTACGGCGACGTCGTTTTGCGGTACGTCAGTTACCTA |
| | | | | AACTCTGATGACTGTTTGTTTCTGCCGAAATTTGAGGCG |
| | | | | GTAGATGAGGAGGCGTCGTTTCCGTTGGATTACGGGAT |
| | | | | CCGGCGGCTAGATCACGCGGTTGGAAATGTACCGGATC |
| | | | | TTGCTCCGGCGGTTTCGTATGTGAAGAAGTTCACCGGAT |
| | | | | TCCACGAGTTCGCTGAGTTCACGGCGGAGGATGTAGGG |
| | | | | ACGAGTGAGAGCGGATTGAACTCGGTGGTGTTGGCTAA |
| | | | | CAACGAAGAAACGGTACTGCTGCCGATGAATGAACCGG |
| | | | | TGTTTGGGACGAAGAGGAAAAGTCAGATACAGACGTAT |
| | | | | TTGGAGCACAATGAAGGAGCTGGAGTACAGCATTTGGC |
| | | | | GCTTGTGAGTGCTGATATTTTCAATACTTTGAGAGAGAT |
| | | | | GAGGAGGAGGAGTGCGATTGGGGGATTTGAGTTTATGC |
| | | | | CGTCTCCTCCGCCGACATATTACCGGAATTTGAAGAAGA |
| | | | | GAGTTGGAGATATTTTGAGTGATGAACAGATTAAGGAG |
| | | | | TGTGAAGAATTAGGGATTCTGGTGGACAGGGATGATCA |
| | | | | AGGGACCTTGCTTCAGATTTTCACTAAACCTGTGGGAGA |
| | | | | TAGGTATTTCTATCTTCTTCTTCATTGTTTTCACTTACAAT |
| | | | | ACCTTTTTCCATTGAAAATTCTTTGTTTCTTGTGTTTCTCTT |
| | | | | ATATGTGTTGGCTGCTATACTATGGTTGATAGAGAATTA |
| | | | | GAATTTAGCGTGTTCAAAACTCAATTCTTTATCTTTATCTT |
| | | | | TAGCCTTAGGTCATTTTGGGATTGCTTTCAAACTGATTTG |
| | | | | ATTATGTTACTAATTAATTAGTACGAAGGACTAACATGT |
| | | | | GTTGTTCTAAGCTAAAATATATTCTATAAAGCTAAAATAT |
| | | | | CTTAATAAGCAATACCAAACTAGCCCTTAAATGCATAGTT |
| | | | | AGACCCTAAATTTGACAACTTTTATAACTTGGCGCCTCTA |
| | | | | ATTTACAAATGTCTTCGCCAACCTCTTGAACTGGCAAAAG |
| | | | | TTTTCACTGCAACCCCTAGGTGCCACGTGTCACCTTGTGA |
| | | | | TTGATTTGACTTTTCAAAGATGTCAAAATTTTTTATTTTCC |
| | | | | TCCCTCTAATCAAAAGGTGACAAGTGATATTGAGTAGGG |
| | | | | GTCTGTAGTGAATACTTTCCCCAAGTTGAGACATCCAGA |
| | | | | AATCGGAGGCGTCATGTGATAAAAGTTGCTTAGTTAAGG |
| | | | | GAGCTTAATATGTATTAAGCCTTTTCGCTACCTTGCCATT |
| | | | | TAAATCCTTAGTCCAAGCAAAATATACAAAAATTAGTTA |
| | | | | GAACTTTATGATTCAGAACCTACGTAACTTTAATTTGACC |
| | | | | AGTAGTTCTATCTTATGCATGCCAGCCATGTTGACTGGT |
| | | | | GAACTTATATTCAATTTTGCTATTCAAGTAAATCCTTATCC |
| | | | | ATCAATCCGAATCAAAAGATTTTAATGTTAGGTTAAACTT |
| | | | | GTACGATTCAAAACTTAGGTTGTACTTTTTGACTAGTAGT |
| | | | | TCTATTATGTATGTGTATATATAATGGGGTTTTTTTTCTGT |
| | | | | AGGCCAACGATTTTCGTAGAGATAATACAGAGAGTAGG |
| | | | | GTGTATGCTAAAGGACGAAGCAGGGAGAGAATACCAGA |
| | | | | AGGGTGGATGTGGCGGTTTTGGTAAGGGAAACTTTTCG |
| | | | | GAGTTATTCAAATCCATTGAAGAATACGAGAAAACACTG |
| | | | | GAAGCCAAACGAACTGCACAAGCATAGAGGTTAGTAGG |
| | | | | GAATTTGATGATACCCATAAATAATGGTTGTTACTTGTTG |
| | | | | CCTTGTAATAAGTTTAGTTAAATGATGACTGTGCCTGCA |
| | | | | AATTGGAATGTTCTCTAATTGTTGGTTGATATTGTTGTAA |
| | | | | AATGTAAACTATGATGTTACTATAAAGTATAGTTGTGGT |
| | | | | TATCGTGTCTCTTCTTTTGGAATCTGAAGTTTCGATATTTT |
| | | | | TTTGGGG |
| 26 | Euphorbia | gDNA | 6569 | TGACTGAAATTAAATTTTAATTATTTGCAAAGCTTACGGT |
| | heterophylla | Contig | | GAAATTAAACTCTAACCATTAAAATTTTAATGAGTCAAAA |
| | | | | TTGACCGAAATGTTCATGTCATAAATTTAGATAATCCTTT |
| | | | | CAGATATGTACGTGAAACCATGTACCTAAATTGTAGTCA |
| | | | | GATTGCTAAACCCCCTTCGAAAATGAATGAAATGATATG |
| | | | | ATATGCTATGCTATGTATTTTAAGGATTATTTTATAGTAA |
| | | | | CTTTGTTTATAATTTACTTTATATGATCATCATCCAATTAA |
| | | | | CTTTCACCTCACTAATTCAATGATTGAAATGGACTAAGTA |
| | | | | ATTTTACTTAATAAAAAAATAAAATCACTATAACCTACAC |
| | | | | ATATTTTTAAAAATAACACCATAATTTATTAAAGGACATT |
| | | | | TAATTGAAGTAGAAAATTATATAATTACTCGCTAATAAAT |
| | | | | TTTCAATTGGAGGAGAGGAAAACACCACGTTTAAAAATC |
| | | | | CCGTTGTTATCCGATTGACGAAAAAAAAGATTAACACGT |
| | | | | CACGACTTCTCCATTCAATAATCCATTTTCTTTATCTTATA |
| | | | | AATAATTTGAAATCCCATCCTCCTCGTTCTCCGTTCACCA |
| | | | | GAAAAAACAGAAATGGGAAAAGACACGTCAGCCGCTGC |
| | | | | CGAAGCATTCAAGCTCGTCGGATTTTCCAATTTCGTCAG |
| | | | | AATCAATCCCCGGTCCGACCGTTTCCCGGTCAAGCGCTT |
| | | | | CCACCACGTCGAGTTCAGGTGCTCCGACGCAACCAACAC |
| | | | | CGCTCGCCGCTTCTCATGGGGCCTCGGGATGCCGTTCGT |
| | | | | CGCCAAATCGGATCTCTCCACCGGCAACGTCACCCACGC |
| | | | | CTCCTACCTCCTCCGCTCCGGCGACCTCAATTTCCTCTTCA |
| | | | | CAGCTCCCTACTCTCCCTCCATAGCCGCCATGGAGAATCT |
| | | | | CTCCGATACTGCCACCGCATCAATCCCTACTTTCTCCCGC |
| | | | | GACGTTTTCCTCGATTTCTCCGCCAAACACGGCCTCGCCG |
| | | | | TCCGAGCTATAGCCATTGAAGTGGAAGATGCTGCGATTG |
| | | | | CTTTCACTACCAGCGTTGCTCAAGGCGCGATTCCGGTGG |
| | | | | CCGGACCTATTGTGCTCGATAATCGTGCTTCAGTTGCGG |
| | | | | AGGTCCACTTGTACGGCGACGTCGTTTTGCGGTACGTCA |
| | | | | GTTATCTAAACTCCGATAGTTGCTTGTTTCTGCCGAAATT |
| | | | | CGAGCCGGTAGATGAGGCGTCGTCTTTCCCGTTGGATTA |
| | | | | CGGGATTCGACGGCTAGATCACGCGGTTGGAAATGTGC |
| | | | | CGGAATTATCTCCGGCGGTTTCGTATGTGAAACAGTTCA |
| | | | | CCGGATTCCATGAGTTCGCCGAGTTCACGGCGGAGGAT |
| | | | | GTGGGGACGAGTGAGAGCGGATTGAACTCGGTGGTGTT |
| | | | | GGCTAATAACGAAGAAACGGTTTTACTACCGTTGAATGA |
| | | | | ACCGGTGTATGGCACAAAGAGGAAAAGTCAGATACAAA |
| | | | | CGTATTTGGAGCACAACGAAGGGGCTGGAGTACAGCAT |
| | | | | TTAGCACTTGTGAGTGCGGATATATTTAACACTTTGAGA |
| | | | | GAGATGAGGAAGAGGAGTGGCGTTGGGGGATTTGAGT |
| | | | | TTATGCCGTCTCCTCCGCCCACATATTACCGGAATTTGAA |
| | | | | GAAGAGAGTCGGGGATATTTTGAGCGATGAACAGATTA |
| | | | | AGGAATGTGAAGAATTAGGGATTTTGGTGGATAGGGAT |
| | | | | GATCAAGGGACCTTGCTTCAGATTTTCACTAAACCTGTG |
| | | | | GGAGATAGGTATGTCAATCAATCTTGTGAAATGATTAGT |
| | | | | TGTTCTATAATTACTTATGTGATTTACATTCTTAGAGTCTG |
| | | | | TAAATTCTTGTGATCATAAATTTGTTGGCTTGGATACCAT |
| | | | | ATTAATATTATCCTTAATTTAGAATGTCAAAGTTATGTTC |
| | | | | TGTCTTTACAATGCCGTTGAAATCGTTATTTATCAAGTCC |
| | | | | AAGCAAACGGTCCTAAAATGTCCTTTAACTTACTGGTTCG |
| | | | | GAATCTCATCTGTTCAATTTGATTAGTAGTTCCATCTTTTA |
| | | | | GTATATACGCCAGGCGCCAGACATGTTGATTTGTGAGTT |
| | | | | TTTGTTATTTGGAATCTCAAAGTTGAGTTTTTTCTATCTAT |
| | | | | AAAAAGTAGCTCAAATCCTTAGTTATCAAGTCAAAACCC |
| | | | | AAAGATCCTATTATTAACTTGGATCTGAAATTATATCGCT |
| | | | | TTCTGGTTTATTTCTAAATTTGGTGGTCACTTGATTTCTCA |
| | | | | TCTTGACAAGTGAATTCATCTGGACATAGTTGGTCTTTTA |
| | | | | TGGGATGAATGGATGTGCTGTTTTTGCAGGCCAACCATT |
| | | | | TTCGTTGAAATAATTCAGCGAGTCGGGTGTATGGTCAAG |
| | | | | GATGAAGCAGGGAGAGAATACCAGAAGGGTGGGTGTG |
| | | | | GCGGTTTCGGTAAGGGAAACTTCTCGGAGTTATTCAAAT |
| | | | | CAATCGAGGAATATGAGAAAACATTGGAGGCCAAACGA |
| | | | | ACTGCAGAAGCAGCTCGAGCATAAAATAAAGGTTAGTA |
| | | | | TGGAAGATGATATGCATACATAAATAAAGGTTGGCTGTT |
| | | | | GTTTATTACGTTGTAATATTCTACTCCAATGATGCTGTTA |
| | | | | TTGTCTGAAAATACCAGTGCCTGCGAATTGGAATGTTCT |
| | | | | CTAATTGTTAGGAACAACTGGAATCATTGTATATCGTAA |
| | | | | ACTATGGTTCATTAAAAGTTAAAAACATATATATGATATG |
| | | | | ATTAATATAGAATTACTCCCTTTATTTGGAATCTAAAGAT |
| | | | | TCATTTCTCAGTTTCTTTCTGGGCGATTTTGAGCATTTCTG |
| | | | | TACTTAAATTCTGCGCGAATACACCCACCAAGCCCTAGCT |
| | | | | AGCTTGGTGGTTACCTTTTAAGGGTGTTGGAAACCTAAA |
| | | | | TTAGAAAAGATTTCAGTTTAATTTATGTCATTTTATATAT |
| | | | | AAAAGTTTGAAACTTTTTTATTCAAAATAAGTTGATTGAT |
| | | | | AAATTGAAATCAATTAGCTTTGTAGCAGATGTTCACCCA |
| | | | | AATCTAATATAAATATATTATAAATCTAATATATTTCTGTT |
| | | | | AACAAACTTATAATATATTAATAAATACAATTGCAAAGA |
| | | | | CAAAGAGACTTATATTAGAATTATAACATAATTTTTTATA |
| | | | | AATATATTAGATACGTATATCTTATAATTGTAACATATAA |
| | | | | AAGATTTTATAGTAAATATGTGTTATAAGTCTAAAATAA |
| | | | | GCCTTTTTAAGTTTAATACAAGTCTCTTTGTGTAACACCC |
| | | | | CGGGTTCGACCGATTGACCCCCACAAACCAACACAAGTC |
| | | | | TTTCTAGTAAGCTTTGCCTTGAGATTGTTCCAAGTCAAGG |
| | | | | ACGCTCAACTTTGGAGTTCTCCCATATAAGCTCCTGAAAA |
| | | | | GAAGGTGTAACTTGTTGGTATAGGCAGCAGTACCAATCA |
| | | | | AACCTTTTTAAGTCCCTTTCCACTTTAATGTCCCTTTCTAT |
| | | | | TGATATAATTTTATTGACTTTATGTAATCAAATGTTTATTC |
| | | | | ATCCCGTGTTTTTTTATTCATTATGTTTTAATTTTTTAATTA |
| | | | | GTCAAAATATAATATCTAAAAAATCTATCAAATTTGGGCT |
| | | | | CGGCTCGGCTTGACTCATTAACATCCGACTCTCTACCTCT |
| | | | | TACTTTTATTAAATACATTTGTGTTTTTCAGTTGTACGTTC |
| | | | | CTTCTCATATTTACATTTCCCTTTTTTCTTTTCTTTTTAATTT |
| | | | | ATTTTTTATTTATTTCGCAAATCGTTGATTTTCTCGGTATG |
| | | | | CTGCTTTATAATTTTTTGTTCGTCTCGTTTTTATCTTCGAA |
| | | | | AGTATATTCATGTTGATATACTTTTTCAAGTTCTTATTTTC |
| | | | | AAAGATATGTAACAAATGACATAAACATTATCCAGCAAA |
| | | | | AATCAGTAAATTACTAGCATTTCACCAACAAAATACTAGC |
| | | | | ATTTTACCCGTGACGAAAAGTTTGAAAAAAATATATAAA |
| | | | | AAAAATCAAACAAAAATCATTTTTATACCAACATTATACC |
| | | | | ATGTATGAAAAAATAAAACTGATCATAAACATAAAGAAA |
| | | | | TCAAACAAAATTCACCTTATCTTAATATTTTACCAAGAGT |
| | | | | GAAGGACCAAACATAAACATGTTTTACGAAAAAAATCAA |
| | | | | ACAAAAATCATTTCACACTAGCATTTTATTAACAAAATCT |
| | | | | CGATATATTATCAGTAAAATACCAACATTTTATCAACAAA |
| | | | | ATACCATCATTTGAGAGGAGGTTCGAGTGCGCCTTCGCC |
| | | | | GTTCTGCGGCGGCTTCTTTGACGGTGGAGAAAGCATAG |
| | | | | CTGCGTGATGGAGGCAGAAGAGGGGTTGATGAAATTGG |
| | | | | GTTGGATTGAAGTGGAATTGAGTCAATGAGGCTAGGTG |
| | | | | ATAGGGTGAGATGATGCATGACTCGAATTGGTGGTTGA |
| | | | | TCGCAGAGGTGGAGGTGGCGATTGGCCGGCGGAGAAG |
| | | | | ACGGCAAATAGATGTTTTTGGGCGGAGTACAAAGCGCC |
| | | | | TTAAATCCTAGCAAAGGCTGATGAAAATAAAAAAGGGT |
| | | | | AATGCTGCACTTTTATCTACTTTGTGCAATATCTATATAC |
| | | | | AGTATTTATTATCATCTTATTCCTTTTATTTATTTCTTTCAT |
| | | | | CTTTTATGGGAGTTGAAATTCTTTCTAGATATTCTTTTCTT |
| | | | | TTTGCTAAATTTTTCTGCTCATTGTCATACACTTGCTTGGT |
| | | | | AAACTGAATCTCAAACCAAATAATGAAATCTGAAGTATG |
| | | | | TATCCACAGAATCGCAATGCTCATCGACGGAAGTAGAAG |
| | | | | GGGAAAACTGCTGTTGGAGTTGAGGTATATAATCTGCA |
| | | | | GTTGAAATCGACGTTGTGTATGTAATTTTGAATTGAACA |
| | | | | AGAGCGAAACCTAGTTTTCTATGATCTGTACCTGGCGTA |
| | | | | TAAGAATTCCATCAATATCGGCAGGGCGATGCGTTAATA |
| | | | | TTGCATTAACACCGGTGGTGAGAAGGGAGCTAATTGCTT |
| | | | | GAAATCCGCTGGCATGCCGCAATGTTGATATGTCACATA |
| | | | | AATGCGACTAAGTGTGACTTATCTTGAAGAGGAGTGAA |
| | | | | ATTGGGGAAACGACTAAGAGTGACTTATCAAGAAGTGT |
| | | | | GATTACCATGCAACTAAGTGCGACACGGTGAAACGACTC |
| | | | | GACTCCTGCAGCCACCACCAAATTTCACAATTTCATATAG |
| | | | | GTAAAAGGGAGACGATCAGAAAGAGGGGAAACAAAAC |
| | | | | ACAAAGAGAGGGAAAAGAAATGTTGGAGGGACTGAAA |
| | | | | TTATAGAAGGGGTAAAATTAGAAACAAAAAAAATTATA |
| | | | | AGGTGTTGTAAGTAGAAATTAAATTACGAGCAAAAGTTG |
| | | | | GTTGAAAATAAAAATGAAGATATTGTGATAAATTATTTC |
| | | | | TTTTTTTTTATATTTGACCAACTTACATTGTAAAGCCTGTA |
| | | | | ATTTTTATTAATTTCGCATTATAGCCATAAAAAGTTATTCC |
| | | | | AATCTATGAAGTTGTGACTTTGCGGAACATATTTTCCGA |
| | | | | AAAACTTATTTTTCAAGTTTTTTAAATAGTAAGAAAAAGG |
| | | | | AAATTTTAAATTATAATAACATAATAGGTAGTAAATATAA |
| | | | | ATAGTATATATTTATAATTCAAATAATTATCAGGAAATCT |
| | | | | TATTTATTTTATGAAATTTCTAGGTAAAATTAATTTAGTC |
| | | | | ATGACTAAGTGTTGTTTCTCACAAAATTGTAATCATAATC |
| | | | | AAACTAAATTTTGATATCGAGAAACACAAAATGAGCATT |
| | | | | TGTTGAAAGAGACTTATAGGTCGAGTCTTAACAAACTTG |
| | | | | ATAAGATTATAAAATTGTCTAGCATAAACTCCTCATGCAG |
| | | | | CGAAACAATAATGTATGATTACAATCATAAGCCAATGTT |
| | | | | CATGTTGGATAAACCACACGATAACTAATTCTAGAATCTC |
| | | | | TCAAGAGTCTAAATAATTTCTATGCACACTTTTAAATTTA |
| | | | | TTTAAGCTTCATAGTGAAATATTAGCTTGTGCTACACTAA |
| | | | | TTTACATTGTACGTTATTTTCAATGGAATGACAACTTTAT |
| | | | | TTTGTGCCCATAGTGCATAACAGTAAAATTTCAGTTCAAT |
| | | | | GATTTCATATATTATTTTTTATATTACGAAGTTTTAGGTTT |
| | | | | TTGGATTTTAGATTACAATCGAGACCATAGCAAGAGTTG |
| | | | | AAACATGCAAATGAGATTTAAGCTTAAAAAAATGTTATA |
| | | | | AAATTATTAACATTTAGCACTTATAGTAGCTACAAAAATT |
| | | | | TGAAGGATTGAATATTTCTAAATATTACAAAGTATAAACT |
| | | | | TAAATATCAACAAATATGATTCATGGAGTTTGAGTTAAG |
| | | | | TATAATTTAACAATATATCAATGATCGAGTTTTTTGCAAA |
| | | | | AATCATAAATATTAGCTCAATTTGAAGAACTTAACAAAG |
| | | | | CACTCAAAACTTTAATTGAAAACAAAAATTTAAGGTTAA |
| | | | | GGGCTTATATATATAAAAAAAAATTCAAAGTATGAAAGC |
| | | | | AATTTCGGACTTCAATTTTTATAATTTTTGATTCATAACGA |
| | | | | ACTATTAAATTATTGATAAATATACTTCTGCATAGAGTCC |
| | | | | AAAAAATTATAAGGCCGGCCTAATAAAGCCTATGATAGA |
| | | | | ATCCTATAGAAACATTGTTGACCCAAGCACAGGCCGAGT |
| | | | | GAACTCGTGGCTGAGTTTCTATGCTAAGAGTTTTGATCA |
| | | | | TGTGTAAGAATAAACTAAGTAAGAATAACATTAAATAAA |
| | | | | TAATTGAATCTTTACATA |
| 27 | Xanthium | cDNA | 1567 | AGAAATAAAAGACACATATGTTCGTGAGGTTATGACACC |
| | strumarium | Contig | | TTTGGTTGATGTTGTAGCCAGTGATTCTAGTGCAACACT |
| | | | | AATCGATTTCCACACCTTGTGGGTAAGTCATCAATATTCA |
| | | | | AGGGTACCTGTTTTTGAACAACGTGTTGATAATATAGTC |
| | | | | GGTATTGCATATGCAATGGATCTATTAGACTATGTTCAA |
| | | | | AAGGGAGACCTTTTAGAAAGTACCACGGTGGGGGATAT |
| | | | | GGCTCATAAACCTGCTTATTTTGTGCCTGATTCAATGTCG |
| | | | | GTATGGAATCTTCTTAGAGAATTTCGCATTAGGAAGGTA |
| | | | | CACATGGCTGTTGTCCTGAACGAATACGGTGGGACAGTT |
| | | | | GGAATTGTAACACTAGAAGACGTGGTTGAAGAAATTGTT |
| | | | | GGTGAAATTTCTTCAACCCACGCCTCTTATCTCCTTAAAT |
| | | | | CCGGCCAACTTAACTTCCTCTTCACCGCCCCTTATTCCCCT |
| | | | | TCCATCTCATCCACCACAACCACCGCCTCCATCCCTTCCTT |
| | | | | TTCTCACTCCACCTGTCGCCACTTCACCTCCTCCCACGGC |
| | | | | CTCGCTGTTCGTGCCATCGCCGTTGAAGTCCATGACGCC |
| | | | | GAACTCGCTTTCTCCGTTAGCGTCGCTCACGGCGCTAAA |
| | | | | GCCTCCGGATACCATAATCCAGCTCTTCAAACGACGACG |
| | | | | TTTCTCCACAGCTTCAAACCCAGGCAAAACATATTGTTAT |
| | | | | TATTATTATTGTTTGTAACTAATATAACGCAAAACGACGT |
| | | | | CGCCGTAGAGTTGAACTTCTGATAAGACGACGTCGTTAT |
| | | | | GACCGAGGGTGATAATCCGGAGGGTTGACCACGCAGTG |
| | | | | GGGAACGTGCCGGAGTTAGCACCGGCAGTGGAATATAT |
| | | | | AAAATCATTTACTGGATTTCACGAGTTTGCTGAGTTTACG |
| | | | | GCGGAGGATGTGGGAACGAGTGAGAGTGGACTAAACT |
| | | | | CGGTGGTTTTGGCTTGCAATAGTGAGATGGTATTGATAC |
| | | | | CGATGAATGAACCGGTTTACGGGACGAAGAGGAAGAGT |
| | | | | CAGATACAGACGTATTTGGAACATAATGAAGGGGCGGG |
| | | | | GGTTCAGCATTTGGCGTTGGCTAGTGAGGATATATTTAG |
| | | | | GACGTTAAGGGAGATGAGGAAAAGGAGTGGGGTTGGT |
| | | | | GGCTTTGAGTTTATGCCGTCTCCCCCTCCTACTTATTATA |
| | | | | GGAATTTGAAGAATAGGGTGGGCGATGTGTTGTCTGAT |
| | | | | GAACAGATTAAGGAGTGTGAAGAATTGGGGATATTGGT |
| | | | | TGATAGAGATGATCAGGGGACTTTGCTTCAGATTTTTAC |
| | | | | CAAGCCTGTTGGTGACAGGCCGACGATATTCATAGAGAT |
| | | | | AATTCAGAGAGTAGGGTGTATGGTGAAGGATAATGAAG |
| | | | | GAAAGGAGCAGCAGAAGGCAGGGTGTGGAGGGTTTGG |
| | | | | CAAAGGGAACTTCTCAGAGCTTTTTAAATCCATTGAGGA |
| | | | | ATATGAGAAGACATTGGAAGCAAGAGCCACCACTGAAG |
| | | | | CCACTGCTGCTGCATGAAAACCACCCATGAATAATCTTCA |
| | | | | TGAGATTTTATAATCTAATGATTATGTATCTGTGGATTCT |
| | | | | ATACGAAC |
| 28 | Digitaria | cDNA | 892 | TTGTGAAGTTGGTGTCATATATTTCAAGATCTGTTCGTAT |
| | sanguinalis | Contig | | AGAATCCACAGATACATAATCATTAGATTATAAAATCTCA |
| | | | | TGAAGATTATTCATGGGTGGTTTTCATGCAGCAGCAGTG |
| | | | | GCTTCAGTGGTGGCTCTTGCTTCCAATGTCTTCTCATATT |
| | | | | CCTCAATGGATTTAAAAAGCTCTGAGAAGTTCCCTTTGCC |
| | | | | AAACCCTCCACACCCTGCCTTCTGCTGCTCCTTTCCTTCAT |
| | | | | TATCCTTCACCATACACCCTACTCTCTGAATTATCTCTATG |
| | | | | AATATCGTCGGCCTGTCACCAACAGGCTTGGTAAAAATC |
| | | | | TGAAGCAAAGTCCCCTGATCATCTCTATCAACCAATATCC |
| | | | | CCAATTCTTCACACTCCTTAATCTGTTCATCAGACAACAC |
| | | | | ATCGCCCACCCTATTCTTCAAATTCCTATAATAAGTAGGA |
| | | | | GGGGGAGACGGCATAAACTCAAAGCCACCAACCCCACT |
| | | | | CCTTTTCCTCATCTCCCTTAACGTCCTAAATATATCCTCAC |
| | | | | TAGCCAACGCCAAATGCTGAACCCCCGCCCCTTCATTATG |
| | | | | TTCCAAATACGTCTGTATCTGACTCTTCCTCTTCGTCCCGT |
| | | | | AAACCGGTTCATTCATCGGTATCAATACCATCTCACTATT |
| | | | | GCAAGCCAAAACCACCGAGTTTAGTCCACTCTCACTCGTT |
| | | | | CCCACATCCTCCGCCGTAAACTCAGCAAACTCGTGAAAT |
| | | | | CCAGTAAATGATTTTATATATTCCACTGCCGGTGCTAACT |
| | | | | CCGGCACGTTCCCCACTGCGTGGTCAAGCCTCCGGATAC |
| | | | | CATAATCCAGCTCTTCAAACGACGACGTTTTCTCCACAGC |
| | | | | TTCAAACCCAGGCAAAAACATATTGTTATTATTATTATTG |
| | | | | TTTTTGTAACTAAT |
| 29 | Digitaria | cDNA | 975 | GCCGCCACCGCCTCCCTCCCCTCCTTCTCCGCCCCCGCCG |
| | sanguinalis | Contig | | CGCGCCGCTTCGCCTCCGACCACGGCCTCGCCGTGCGCG |
| | | | | CCGTAGCGCTCCGCGTCGCCGACGCCGAGGACGCCTTCC |
| | | | | GCGCCAGCGTCGCCAACGGGGCGCGCCCGGCGTTCGAG |
| | | | | CCCGTCGAGCTCGGCCTCGGCTTCCGCCTCGCCGAGGTC |
| | | | | GAGCTCTACGGCGACGTCGTGCTCCGCTACGTCAGCTAC |
| | | | | CCGGACGCCGCGGATTTGCCCTTCCTGCCGGGCTTCGAG |
| | | | | GACGTCGTCATCAGCAACCCAGGGGCGGTGGACTACGG |
| | | | | GCTGAGGCGCTTCGACCACATCGTCGGCAACGTCTCGGA |
| | | | | GCTGGCGCCGGTGGCCGCGTACGTCGCCGGATTCACGG |
| | | | | GGTTCCACGAGTTCGCCGAGTTCACGGCGGAGGACGTG |
| | | | | GGCACGGCGGAGAGCGGGCTCAATTCCGTGGTGCTCGC |
| | | | | CAACAACTCCGAGAACGTGCTGCTCCCGCTCAACGAGCC |
| | | | | GGTGCACGGCACCAAGCGCCGCAGCCAGATACAGACAT |
| | | | | ACCTGGACCACCACGGCGGCCCTGGAGTGCAGCACATC |
| | | | | GCGCTGGCTAGCGACGACGTGCTCAGGACACTGCGGGA |
| | | | | GATGCAGGCGCGCTCCGCCATGGGTGGGTTTGAGTTCA |
| | | | | TGGAGGCTCCGCCACCCACTTACTATGAGGGTGTGAGG |
| | | | | CGGCGCGCCGGGGACGTGCTCTCGGAGGAGCAGATAAA |
| | | | | GGAGTGCCAGAAACTGGGGGTGCTGGTGGACAGGGAT |
| | | | | GACCAGGGAGTGTTGCTCCAAATCTTCACCAAGCCAGTG |
| | | | | GGGGACCCAACGTTTTTCTTGGAGATAATCCAAAGGATT |
| | | | | GGGTGCATGGAGAAGGATGAGCAGGGAAAGGACTACC |
| | | | | AGAAGGGCGGCTGTGGCGGGTTTGGCAAGGGAAACTTC |
| | | | | TCACAGCTGTTCAAGTCTATTGAGGAGTATGAGAAGTCC |
| | | | | CTTGAAGCGAAGCAA |
| 30 | Kochia | cDNA | 1266 | ACCACCGCCTCAACCGAGTTCAAGCTGGTGGGTTACTCC |
| | scoparia | Contig | | AACTTCGTCCGAGTTAACCCCATGTCCGACCTCTTCTCCG |
| | | | | TCAAAAAATTCCACCACATCGAGTTCTGGTGCGGCGACG |
| | | | | CGACCAACGTCAGCCGTCGCTTCTCATGGGGTCTAGGCA |
| | | | | TGCCGGCGGTCGCTAAATCCGACCTCTCCACCGGAAACT |
| | | | | CCGTACACGCTTCGTACCTCCTTCGCTCAGGCGACCTCTC |
| | | | | CTTCCTCTTCACCTCCCCGTACTCCCCTTCTCTCTCCTCCCC |
| | | | | CTCTTCCGCTGCAATACCCACGTTTGATTTCTCTCTCTTCT |
| | | | | CCTCTTTTATCACCTCCCACGGCATCGGGGTTCGCGCCGT |
| | | | | TGCACTCGAGGTCGACGATGCCGAGGTTGCTTTCAATAC |
| | | | | GAGCGTCTCCCACGGCGCAATCCCTGGTTCTCCCCCGAT |
| | | | | TAAGCTCGGAAACGGCGTCGTTTTGTCCGAGGTCAGCCT |
| | | | | CTACGGCGACGTCGTTCTTCGCTACGTGAGCTACGGAGG |
| | | | | TGAGACAGAGAAAGCGGATACGAATTCGAATTCATGGT |
| | | | | TTCTTCCTGGGTTCGAGGAAATGCCGGAGGAATCGTCGT |
| | | | | TTCGAGGGCTCGATTTCGGGCTCCGGAGACTAGACCATG |
| | | | | CGGTGGGGAATGTCCCGGAGCTAGGGAAAGCAATCGA |
| | | | | GTATGTGAAGAAGTTCACTGGGTTTCACGAATTTGCTGA |
| | | | | GTTTACAGCTGACGATGTTGGGACGAGTGAAAGTGGGC |
| | | | | TGAATTCGGCTGTGCTGGCGAACAACTCGGAGACGGTG |
| | | | | TTGATTCCGATGAACGAGCCGGTTTACGGGACGAAGAG |
| | | | | GAAGAGTCAAATTCAGACATACTTGGAGCACAATGAAG |
| | | | | GAGCTGGGGTTCAGCATTTGGCATTGATGAGTGAGGAT |
| | | | | ATATTCTGGACTCTTAGGGAGATGAGGAAGCGGAGCGG |
| | | | | GCTCGGTGGGTTCGAGTTTATGCCAGCACCGCCGCCTAC |
| | | | | GTATTATCGGAACCTGAGGAATCGTGTCGGGGATGTGTT |
| | | | | GAGTGAAGAACAGATGAAGGAGTGTGAAGAGTTGGGG |
| | | | | ATATTGGTTGATAAGGATGATCAGGGCACTCTGCTCCAG |
| | | | | ATTTTTACTAAGCATATTGGTGACCCAACTATGTTCATTG |
| | | | | AGATTATCCAAAGAATTGGCTGCATGATGAAAGATGAA |
| | | | | GAAGGCAAGGTGTACCAAAAGGGAGGCTGTGGAGGAT |
| | | | | TTGGGAAGGGAAACTTTTCAGAGCTTTTCAAATCTATCG |
| | | | | AAGAGTATGAGAAGACACTTGAA |
| 31 | Lolium | cDNA | 783 | CTCGGCCACGGGTTTGGCTTCGCGGAGGTGGAGCTAGC |
| | multiflorum | Contig | | CGGGGACAGCGTTCTCCGCTTCGTGAGCTACCCGGACG |
| | | | | GCACCGACGTGTCCTTCCTGCCGGGGTTCCAGGACGTGG |
| | | | | CGAGCGCCGGCGGGGCGCCGGACTTCGGGCTCACCCGG |
| | | | | TTTGACCACGTCGACGTTAACATCCCGGAGCTGGCACCC |
| | | | | GTCGCCGCCAATGTTGCCGGCTTCACCGGGTTCCACAAA |
| | | | | TTCTGGGAGTTCACCGCGGACGACGTGTGCCCGGAAGA |
| | | | | GAGCGGGGTGAACGGCGTGGTGATCGCCAACAACTCAG |
| | | | | AGAACGTGCTGCTCAGTATCTTGGAGCCGGTGTTCGGCA |
| | | | | CCAAGCTGCGGAGCCATGTCGAGACGTTCCTGGACCACC |
| | | | | ACGGTGGCCCGGGCATACAGCACCTGGCAATGACCAGC |
| | | | | CACGACATCCTTGGCGCGCTCAGGAAAATCCGAGCTCGG |
| | | | | TCCTCCATGGGCGGGTTTGAGCTCCTGCCGCCGCCGCCG |
| | | | | GCCAGCTACTATGACGGTGTAAGGCAGCGCGCCGGGGA |
| | | | | CGTGCTGTCGGAAGAACAGATCAAGGAGTGCCAAGAGC |
| | | | | TGGGCGTGCGGGTGGACAGAGGGTATGAGGACGGAGT |
| | | | | TGTGCTCCAAGTCTTCACCAAACCGGCGGGAGACCCAAC |
| | | | | CTTACTGTTAGAGTTTATCCAAAGAATCGGGTGCATGGT |
| | | | | CAAGGACGAGAACCAGCAGGAATACCAGAGAGGTGGA |
| | | | | TGTGGCGGGTTTGCCAAAGGGAACGTTTCTGAACTCATC |
| | | | | AAGGACATTGAGGAC |
| 32 | Lolium | cDNA | 1041 | ACCGATCGCTTCCACGTGATGGATTTCCACCACGTCGAG |
| | multiflorum | Contig | | TTTTGGTGCGCCGATGCCGCCTCGGCCGCCGCACGGTTC |
| | | | | TCCTTCGGGCTCGGCGTGCCACTCGCCGCGCAGTCCGAC |
| | | | | CTCTCCACGGGGAACACTGCGCACGCCTCACGCCTACTA |
| | | | | CGCGCACGCTCGGGCTCTCTCTCGTTCCTCTTCACCGCGC |
| | | | | CGTACGCGCCGCACGTCGCCGACTCGGCGACCACCGCG |
| | | | | TCCCTGCCCTCCTTCTCGGCGGACGCCGCGCGGCGCTTC |
| | | | | ACGGGCACCCACGGCGGCCTGGCCGTGCGTGCCGTGGC |
| | | | | CGTCCGCGTCGCTGACGCGGCCGAGGCCTTCGTCGCGA |
| | | | | GCGTGGACGCCGGAGCGCGGCCAGCCTGCGCCCCGACT |
| | | | | GATCTCGGCCACGGGTTTGGCTTCGCGGAGGTGGAGCT |
| | | | | AGCCGGGGACAGCGTTCTCCGCTTCGTGAGCTACCCGG |
| | | | | ACGGCACCGACGTGTCCTTCCTGCCGGGGTTCCAGGACG |
| | | | | TGGCGAGCGCCGGCGGGGCGCCGGACTTCGGGCTCACC |
| | | | | CGGTTTGACCACGTCGACGTTAACATCCCGGAGCTGGCA |
| | | | | CCCGTCGCCGCCAATGTTGCCGGCTTCACCGGGTTCCAC |
| | | | | AAATTCTGGGAGTTCACCGCGGACGACGTGTGCCCGGA |
| | | | | AGAGAGCGGGGTGAACGGCGTGGTGATCGCCAACAACT |
| | | | | CAGAGAACGTGCTGCTCAGTATCTTGGAGCCGGTGTTCG |
| | | | | GCACCAAGCTGCGGAGCCATGTCGAGACGTTCCTGGAC |
| | | | | CACCACGGTGGCCCGGGCATACAGCACCTGGCAATGAC |
| | | | | CAGCCACGACATCCTTGGCGCGCTCAGGAAAATCCGAGC |
| | | | | TCGGTCCTCCATGGGCGGGTTTGAGCTCCTGCCGCCGCC |
| | | | | GCCGGCCAGCTACTATGACGGTGTAAGGCAGCGCGCCG |
| | | | | GGGACGTGCTGTCGGAAGAACAGATCAAGGAGTGCCAA |
| | | | | GAGCTGGGCGTGCGGGTGGACAGAGGGTATGAGGACG |
| | | | | GAGTTGTGCTCCAAGTCTTCACCAAACCGGCGGGAGACA |
| | | | | GG |

We claim:

1. A method of plant control comprising: treating a plant with a composition comprising a polynucleotide and a transfer agent, wherein said polynucleotide is essentially identical or essentially complementary to a HPPD gene sequence or fragment thereof, or to an RNA transcript of said HPPD gene sequence or fragment thereof, wherein said HPPD gene sequence is selected from the group consisting of SEQ ID NO:1-32 or a polynucleotide fragment thereof, whereby said plant growth or development or reproductive ability is reduced or said plant is more sensitive to a HPPD inhibitor herbicide relative to a plant not treated with said composition.

2. The method as claimed in claim 1, wherein said transfer agent is an organosilicone surfactant composition or compound contained therein.

3. The method as claimed in claim 1, wherein said polynucleotide fragment is 18 contiguous, 19 contiguous nucleotides, 20 contiguous nucleotides or at least 21 contiguous nucleotides in length and at least 85 percent identical to a HPPD gene sequence selected from the group consisting of SEQ ID NO:1-32.

4. The method as claimed in claim 3, wherein said polynucleotide fragment is selected from the group consisting of sense or anti-sense ssDNA or ssRNA, dsRNA, or dsDNA, or dsDNA/RNA hybrids.

5. The method as claimed in claim 1, wherein said plant is selected from the group consisting of Amaranthus palmeri, Amaranthus rudis, Amaranthus thunbergii, Amaranthus graecizans, Amaranthus hybridus, Amaranthus viridis, Ambrosia trifida, Kochia scoparia, Abutilon theophrasti, Conyza candensis, Digitaria sanguinalis, Euphorbia heterophylla, Lolium multiflorum and Xanthium strumarium.

6. The method as claimed in claim 1, wherein said composition further comprises said HPPD inhibitor herbicide and external application to a plant with said composition.

7. The method as claimed in claim 6, wherein said composition further comprises one or more herbicides different from said HPPD inhibitor herbicide.

8. The method as claimed in claim 3, wherein said composition comprises any combination of two or more of said polynucleotide fragments and external application to a plant with said composition.

9. A composition comprising a polynucleotide and a transfer agent, wherein said polynucleotide is essentially identical or essentially complementary to a HPPD gene sequence or fragment thereof, or to an RNA transcript of said HPPD gene sequence or fragment thereof, wherein said HPPD gene sequence is selected from the group consisting of SEQ ID NO:1-32 or a polynucleotide fragment thereof, and whereby a plant treated with said composition has its growth or development or reproductive ability regulated, suppressed or delayed or said plant is more sensitive to a HPPD inhibitor herbicide as a result of said polynucleotide containing composition relative to a plant not treated with said composition.

10. The composition of claim 9, wherein said transfer agent is an organosilicone composition.

11. The composition of claim 9, wherein said polynucleotide fragment is 18 contiguous, 19 contiguous nucleotides, 20 contiguous nucleotides or at least 21 contiguous nucleotides in length and at least 85 percent identical to a HPPD gene sequence selected from the group consisting of SEQ ID NO:1-32.

12. The composition of claim 9, wherein said polynucleotide is selected from the group consisting of SEQ ID NO:33-596.

13. The composition of claim 9, wherein said polynucleotide is selected from the group consisting of SEQ ID NO: 597-1082.

14. The composition of claim 9, further comprising a HPPD inhibitor herbicide.

15. The composition of claim 14, wherein said HPPD inhibitor molecule is selected from the group consisting of mesotrione, tefuryltrione, tembotrione, sulcotrione; isoxachlortole, pyrasulfotole, isoxaflutole, benzofenap, pyrazolynate, topramezone and pyrazoxyfen.

16. The composition of claim 14, further comprising a co-herbicide.

17. A method of reducing expression of a HPPD gene in a plant comprising: external application to a plant of a composition comprising a polynucleotide and a transfer agent, wherein said polynucleotide is essentially identical or essentially complementary to an HPPD gene sequence or fragment thereof, or to the RNA transcript of said HPPD gene sequence or fragment thereof, wherein said HPPD gene sequence is selected from the group consisting of SEQ ID NO:1-32 or a polynucleotide fragment thereof, whereby said expression of said HPPD gene is reduced relative to a plant in which the composition was not applied.

18. The method as claimed in claim 17, wherein said transfer agent is an organosilicone compound.

19. The method as claimed in claim 17, wherein said polynucleotide fragment is 19 contiguous nucleotides, 20 contiguous nucleotides or at least 21 contiguous nucleotides in length and at least 85 percent identical to an HPPD gene sequence selected from the group consisting of SEQ ID NO:1-32.

20. The method as claimed in 17, wherein said polynucleotide molecule is selected from the group consisting of sense or anti-sense ssDNA or ssRNA, dsRNA, or dsDNA, or dsDNA/RNA hybrids.

21. A microbial expression cassette comprising a polynucleotide fragment of 18 contiguous, 19 contiguous nucleotides, 20 contiguous nucleotides or at least 21 contiguous nucleotides in length and at least 85 percent identical to a HPPD gene sequence selected from the group consisting of SEQ ID NO:1-32.

22. A method of making a polynucleotide comprising a) transforming the microbial expression cassette of claim 21 into a microbe; b) growing said microbe; c) harvesting a polynucleotide from said microbe, wherein said polynucleotide is 18 contiguous, 19 contiguous nucleotides, 20 contiguous nucleotides or at least 21 contiguous nucleotides in length and at least 85 percent identical to a HPPD gene sequence selected from the group consisting of SEQ ID NO:1-32.

23. A method of identifying polynucleotides useful in modulating HPPD gene expression when externally treating a plant comprising: a) providing a plurality of polynucleotides that comprise a region essentially identical or essentially complementary to a polynucleotide fragment of 18 contiguous, 19 contiguous nucleotides, 20 contiguous nucleotides or at least 21 contiguous nucleotides in length and at least 85 percent identical to a HPPD gene sequence selected from the group consisting of SEQ ID NO:1-32; b) externally treating said plant with one or more of said polynucleotides and a transfer agent; c) analyzing said plant or extract for modulation of HPPD gene expression, and whereby a plant treated with said composition has its growth or development or reproductive ability regulated, suppressed or delayed or said plant is more sensitive to a HPPD inhibitor herbicide as a result of said polynucleotide containing composition relative to a plant not treated with said composition.

24. The method as claimed in 23, wherein said plant is selected from the group consisting of Amaranthus palmeri, Amaranthus rudis, Amaranthusthunbergii, Amaranthus graecizans, Amaranthus hybridus, Amaranthus viridis, Ambrosia trifida, Kochia scoparia, Abutilon theophrasti, Conyza candensis, Digitaria sanguinalis, Euphorbia heterophylla, Lolium multiflorum and Xanthium strumarium.

25. The method as claimed in 23, wherein said HPPD gene expression is reduced relative to a plant not treated with said polynucleotide fragment and a transfer agent.

26. The method as claimed in 23, wherein said transfer agent is an organosilicone compound.

27. An agricultural chemical composition comprising an admixture of a polynucleotide and a HPPD inhibitor and a co-herbicide, wherein said polynucleotide is essentially identical or essentially complementary to a portion of an HPPD gene sequence, or to a portion of an RNA transcript of said HPPD gene sequence, wherein said HPPD gene sequence is selected from the group consisting of SEQ ID NO:1-32 or a polynucleotide fragment thereof, and whereby a plant treated with said composition has its growth or development or reproductive ability regulated, suppressed or delayed or said plant is more sensitive to a HPPD inhibitor herbicide as a result of said polynucleotide containing composition relative to a plant not treated with said composition.

28. The agricultural chemical composition of claim 27, wherein said co-herbicide is selected from the group consisting of amide herbicides, arsenical herbicides, benzothiazole herbicides, benzoylcyclohexanedione herbicides, benzofuranyl alkylsulfonate herbicides, cyclohexene oxime herbicides, cyclopropylisoxazole herbicides, dicarboximide herbicides, dinitroaniline herbicides, dinitrophenol herbicides, diphenyl ether herbicides, dithiocarbamate herbicides, glycine herbicides, halogenated aliphatic herbicides, imidazolinone herbicides, inorganic herbicides, nitrile herbicides, organophosphorus herbicides, oxadiazolone herbicides, oxazole herbicides, phenoxy herbicides, phenylenediamine herbicides, pyrazole herbicides, pyridazine herbicides, pyridazinone herbicides, pyridine herbicides, pyrimidinediamine herbicides, pyrimidinyloxybenzylamine herbicides, quaternary ammonium herbicides, thiocarbamate herbicides, thiocarbonate herbicides, thiourea herbicides, triazine herbicides, triazinone herbicides, triazole herbicides, triazolone herbicides, triazolopyrimidine herbicides, uracil herbicides, and urea herbicides.

29. An agricultural chemical composition comprising an admixture of a polynucleotide and a HPPD inhibitor herbicide and a pesticide, wherein said polynucleotide is essentially identical or essentially complementary to a portion of a HPPD gene sequence, or to a portion of an RNA transcript of said HPPD gene sequence, wherein said HPPD gene sequence is selected from the group consisting of SEQ ID NO:1-32 or a polynucleotide fragment thereof, whereby a field of crop plants in need of weed and pest control are treated with said composition, and whereby a plant treated with said composition has its growth or development or reproductive ability regulated, suppressed or delayed or said plant is more sensitive to a HPPD inhibitor herbicide as a result of said polynucleotide containing composition relative to a plant not treated with said composition.

30. The agricultural chemical composition of claim 29, wherein said pesticide is selected from the group consisting of insecticides, fungicides, nematocides, bactericides, acaricides, growth regulators, chemosterilants, semiochemicals, repellents, attractants, pheromones, feeding stimulants, and biopesticides.

31. A herbicide composition comprising a HPPD inhibitor herbicide and a polynucleotide and a transfer agent, wherein said polynucleotide is selected from the group consisting of SEQ ID NO: 1083-1092 or a complement or polynucleotide fragment thereof, and whereby a plant treated with said composition has its growth or development or reproductive ability regulated, suppressed or delayed or said plant is more sensitive to an HPPD inhibitor herbicide as a result of said polynucleotide containing composition relative to a plant not treated with said composition.
